(19) United States
(12) Patent Application Publication
(10) Pub. No.: US 2023/0391748 A1
(43) Pub. Date: Dec. 7, 2023

(54) BICYCLIC INHIBITORS OF IRAK

(71) Applicant: Rigel Pharmaceuticals, Inc., South San Francisco, CA (US)

(72) Inventors: Yan Chen, South San Francisco, CA (US); Esteban Masuda, South San Francisco, CA (US); Chrystelle Lamagna, South San Francisco, CA (US)

(21) Appl. No.: 18/204,304

(22) Filed: May 31, 2023

Related U.S. Application Data
(60) Provisional application No. 63/347,920, filed on Jun. 1, 2022.

(51) Int. Cl. C07D 401/14; C07D 401/12; C07D 405/14; G01N 33/574

(52) U.S. Cl. CPC C07D 401/14; C07D 401/12; C07D 405/14; G01N 33/57484; G01N 2333/91205

(57) ABSTRACT

Disclosed embodiments concern novel interleukin receptor associated kinases (IRAK) inhibitor compounds and compositions comprising such compounds. The compounds may have a structure according to Formula I

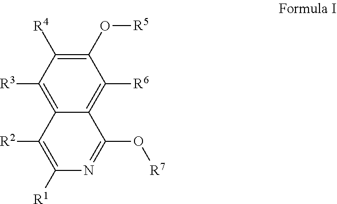

Also disclosed are methods of making and using the compounds and compositions. The disclosed compounds and/or compositions may be used to treat or prevent an IRAK-associated disease or condition.

### CROSS REFERENCE TO RELATED APPLICATION

This application claims the benefit of the earlier filing date of U.S. provisional patent application No. 63/347,920, filed Jun. 1, 2022, which is incorporated herein by reference in its entirety.

### FIELD

This disclosure concerns bicyclic compounds, and embodiments of a method for making and using the compounds, such as for inhibiting interleukin receptor-associated kinase (IRAK), and for treating diseases and conditions related to IRAK.

### BACKGROUND

Interleukin-1 receptor-associated kinases (IRAKs) are important mediators of signaling processes, such as toll-like receptors (TLR) and interleukin-1 receptor (IL-1R) signaling processes. IRAKs have been implicated in modulating signaling networks that control inflammation, apoptosis, and cellular differentiation. Four IRAK genes have been identified in the human genome (IRAK1, IRAK2, IRAK3 and IRAK4), and studies have revealed distinct, non-redundant biological roles. IRAK1 and IRAK4 have been shown to exhibit kinase activity.

### SUMMARY

Disclosed herein are embodiments of a compound having a structure according to Formula I or a pharmaceutically acceptable salt or solvate thereof:

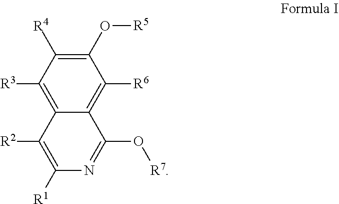

With respect to Formula I, each of R¹, R³ and R⁶ independently are H or C₁₋₆alkyl, R² is alkynyl, heteroaryl, or halogen, R⁴ is —C(O)NH₂, R⁵ is C₁₋₆alkyl, R⁷ is —(CH₂)ₙ-heterocycloaliphatic, and n is 0, 1 or 2. In some embodiments, each of R¹, R³ and R⁶ is H.

In some embodiments, R⁵ is isopropyl, but in other embodiments, R⁵ is methyl.

In some embodiments, R² is halogen, for example, Br. But in other embodiments, R² is

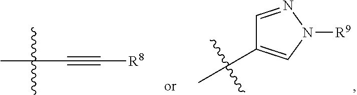

where R⁸ is C₁₋₆alkyl optionally substituted with 1, 2 or 3 substituents selected from OH or 4- to 6-membered heterocycloaliphatic, 4- to 6-membered heterocycloaliphatic optionally substituted with 1, 2, or 3 substituents selected from R^{a}, or C₃₋₆ cycloalkyl optionally substituted with 1, 2 or 3 R^{b}. And R⁹ is C₃₋₆cycloalkyl optionally substituted with 1, 2 or 3 R^{b}, C₁₋₆alkyl optionally substituted with 4- to 6-membered heterocycloaliphatic, or 4- to 6-membered heterocycloaliphatic optionally substituted with 1, 2, or 3 R^{a}. R^{a} is OH, C(O)OR^{c}, C₁₋₆alkyl, or C(O)NH(R^{c}). R^{b} is OR^{c}, C₁₋₆alkyl, or N(R^{c})₂. And R^{c} is H or C₁₋₆alkyl.

R⁷ may be —(CH₂)ₙ-(5-membered or 6-membered heterocycloaliphatic) optionally substituted with oxo (═O), —C(O)CH₂CN, halogen (such as F), C₁₋₆alkyl, —C(O)O—C₁₋₆alkyl), or a combination thereof. The 5-membered or 6-membered heterocycloaliphatic may be a 5-membered or 6-membered nitrogen-containing heterocycloaliphatic. In certain embodiments, R⁷ is

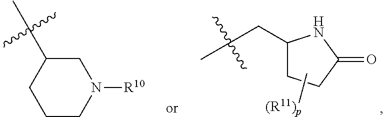

where R¹⁰ is H, C(O)OC₁₋₆alkyl, or C(O)CH₂CN, each R¹¹ independently is halogen or C₁₋₆alkyl, and p is from 0 to 4. In some embodiments, p is 0, 1 or 2. And in certain embodiments, each R¹¹ independently is F or ethyl.

In some embodiments, the compound has a structure according to Formula III or Formula IV, or a pharmaceutically acceptable salt or solvate thereof.

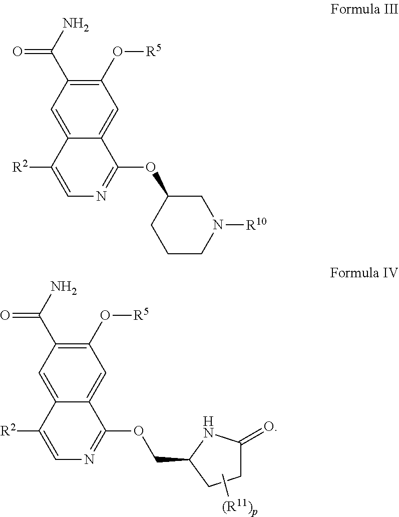

Also disclosed herein is a pharmaceutical composition comprising a compound disclosed herein, and a pharmaceutically acceptable excipient.

A method for inhibiting an IRAK enzyme is disclosed herein. The method may comprise contacting the enzyme with an effective amount of a compound disclosed herein. In some embodiments, contacting the enzyme comprises administering the compound to a subject.

Also disclosed herein is a method for treating a subject for a disease or condition wherein an IRAK inhibitor is indicated. The method may comprise administering to the subject an effective amount of a compound disclosed herein, or a pharmaceutical composition thereof. The disease or condition may comprise an auto-immune disease, inflammatory disorder, cardiovascular disease, neurodegenerative disorder, allergic disorder, multi-organ failure, kidney disease, platelet aggregation, a hyperproliferative disorder, transplantation, sperm motility, erythrocyte deficiency, graft rejection, lung injury, respiratory disease, ischemic condition, bacterial infection, viral infection, immune regulatory disorder, sickle cell disease, a chemical- or radiation-induced lung injury, hemorrhagic fever, or a combination thereof.

In any embodiments, the method may further comprise identifying the subject having from the lymphoid neoplasm. In certain embodiments, the lymphoid neoplasm is chronic myelomonocytic leukemia and identifying the subject comprises identifying a subject having a persistent peripheral blood monocytosis of ≥1×10⁹/L and monocytes accounting for ≥10% of the white blood cell (WBC) differential count, and rearrangements in the PDGFRA, PDGFRB or FGFR1 genes and the PCM1-JAK2 fusion gene are not observed.

The foregoing and other objects, features, and advantages of the disclosure will become more apparent from the following detailed description.

### DETAILED DESCRIPTION

### I. Definitions

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

When chemical structures are depicted or described, unless explicitly stated otherwise, all carbons are assumed to include hydrogen so that each carbon conforms to a valence of four. For example, in the structure on the left-hand side of the schematic below there are nine hydrogen atoms implied. The nine hydrogen atoms are depicted in the right-hand structure.

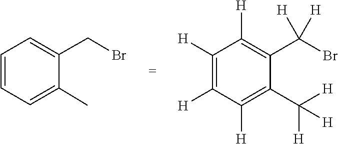

Sometimes a particular atom in a structure is described in textual formula as having a hydrogen or hydrogen atoms, for example —CH₂CH₂—. It will be understood by a person of ordinary skill in the art that the aforementioned descriptive techniques are common in the chemical arts to provide brevity and simplicity to description of organic structures.

A person of ordinary skill in the art will appreciate that the definitions may be combined to further describe a particular compound. For example, hydroxyaliphatic refers to an aliphatic group substituted with an hydroxy (—OH) group, and haloalkylheteroaryl refers to a heteroaryl group substituted with an alkyl group, where the alkyl group too is substituted with a halogen, and where the point of attachment to the parent structure is via the heteroaryl moiety since aryl is the base name of the substituent.

As used herein, the term “substituted” refers to all subsequent modifiers in a term, for example in the term “substituted heteroarylC₁₋₈alkyl,” substitution may occur on the “C₁₋₈alkyl” portion, the “heteroaryl” portion or both portions of the heteroarylC₁₋₈alkyl group. Also by way of example, alkyl includes substituted alkyl groups.

“Substituted,” when used to modify a specified group or moiety, means that at least one, and perhaps two or more, hydrogen atoms of the specified group or moiety is independently replaced with the same or different substituent groups as defined below. In a particular embodiment, a group, moiety or substituent may be substituted or unsubstituted, unless expressly defined as either “unsubstituted” or “substituted.” Accordingly, any of the groups specified herein may be unsubstituted or substituted. In particular embodiments, the substituent may or may not be expressly defined as substituted, but is still contemplated to be optionally substituted. For example, an “alkyl” substituent may be unsubstituted or substituted, but an “unsubstituted alkyl” may not be substituted.

In one embodiment, a group that is substituted has 1 substituent, 2 substituents, substituents, or 4 substituents.

Additionally, in embodiments where a group or moiety is substituted with a substituted substituent, the nesting of such substituted substituents is limited to three, thereby preventing the formation of polymers. Thus, in a group or moiety comprising a first group that is a substituent on a second group that is itself a substituent on a third group, which is attached to the parent structure, the first (outermost) group can only be substituted with unsubstituted substituents. For example, in a group comprising -(heteroaryl-1)-(heteroaryl-2)-(heteroaryl-3), heteroaryl-3 can only be substituted with substituents that are not themselves substituted.

Example substituents include, but are not limited to, alkyl, halogen, cycloalkyl, heteroaryl, heterocycloaliphatic, —C(O)NH₂, —C(O)Oalkyl, OH, oxo (═O), alkynyl, —C(O)CH₂CN), —Oalkyl, or —N(alkyl)₂, unless otherwise specified herein.

“Alkyl” refers to a saturated aliphatic hydrocarbyl group having from 1 to 25 carbon atoms, typically 1 to 10 carbon atoms such as 1 to 6 carbon atoms (C₁-C₆alkyl). An alkyl moiety may be substituted or unsubstituted. This term includes, by way of example, linear and branched hydrocarbyl groups, unless otherwise specified. Example alkyl groups include, but are not limited to methyl (CH₃), ethyl (—CH₂CH₃), n-propyl (—CH₂CH₂CH₃), isopropyl (—CH(CH₃)₂), n-butyl (—CH₂CH₂CH₂CH₃), isobutyl (—CH₂CH₂(CH₃)₂), sec-butyl (—CH(CH₃)(CH₂CH₃), t-butyl (—C(CH₃)₃), n-pentyl (—CH₂CH₂CH₂CH₂CH₃), or neopentyl (—CH₂C(CH₃)₃).

“Cycloalkyl” refers to a cyclic aliphatic group having from 3 to 15 carbon atoms, typically, from 3 to 8 carbon atoms, from 3 to 6 carbon atoms or from 3 to 4 carbon atoms. A cycloalkyl group may be a single ring (e.g., cyclohexyl), or may comprise multiple rings, such as in a fused, bridged or spirocyclic system, at least one of which is aliphatic, provided that the point of attachment is through an atom of an aliphatic region of the cycloalkyl group. Example cycloalkyl groups include, but are not limited to cyclopropyl, cyclopropyl, cyclobutyl, cyclopentyl, cyclohexyl, or cycloheptyl.

“Alkynyl” refers to an aliphatic hydrocarbyl group having from 2 to 25 carbon atoms, typically 2 to 10 carbon atoms such as 2 to 6 carbon atoms (C₂-C₆alkynyl) and at least one triple bond. An alkynyl moiety may be substituted or unsubstituted. This term includes, by way of example, linear and branched alkynyl groups, unless otherwise specified. Example alkynyl groups include, but are not limited to ethynyl, propynyl, 1-butynyl, or 1-pentynyl.

“Halo,” “halide” or “halogen” refers to fluoro, chloro, bromo or iodo.

“Haloalkyl” refers to an alkyl moiety substituted with one or more halogens. An exemplary haloalkyl moiety is CF₃.

“Heteroaryl” refers to an aromatic group or moiety of, unless specified otherwise, from 5 to 15 ring atoms comprising at least one carbon atom and at least one heteroatom, such as N, S, O, P, or Si. A heteroaryl group or moiety may comprise a single ring (e.g., pyridinyl, pyrimidinyl or pyrazolyl) or multiple condensed rings (e.g., indolyl, benzopyrazolyl, or pyrazolopyridinyl). Heteroaryl groups or moiety may be, for example, monocyclic, bicyclic, tricyclic or tetracyclic. Unless otherwise stated, a heteroaryl group or moiety may be substituted or unsubstituted.

“Heterocycloaliphatic,” refer to a stable three- to fifteen-membered non-aromatic ring moiety comprising at least one carbon atom, and typically plural carbon atoms, and at least one, such as from one to five, heteroatoms. The heteroatom(s) may be nitrogen, phosphorus, oxygen, silicon or sulfur atom(s). The heterocycloaliphatic moiety may be a monocyclic moiety, or may comprise multiple rings, such as in a bicyclic or tricyclic ring system, provided that at least one of the rings contains a heteroatom. Such a multiple ring moiety can include fused or bridged ring systems as well as spirocyclic systems; and any nitrogen, phosphorus, carbon, silicon or sulfur atoms in the heterocycloaliphatic moiety can be optionally oxidized to various oxidation states, unless expressly excluded or excluded by context. For convenience, nitrogens, particularly, but not exclusively, those defined as annular aromatic nitrogens, are meant to include their corresponding N-oxide form, although not explicitly defined as such in a particular example. In addition, annular nitrogen atoms can be optionally quaternized.

Examples of heterocycloaliphatic groups include, but are not limited to, tetrahydroisoquinolyl, piperidinyl, piperazinyl, 2-oxopiperazinyl, 2-oxopiperidinyl, pyrrolidinyl, 4-piperidonyl, dihydropyridinyl, tetrahydropyridinyl, morpholinyl, diazabicycloheptane, diazapane, diazepine, tetrahydrofuryl, tetrahydropyranyl, oxetanyl, or azetidinyl.

“Patient” or “Subject” refers to mammals and other animals, particularly humans. Thus disclosed methods are applicable to both human therapy and veterinary applications.

“Pharmaceutically acceptable excipient” refers to a substance, other than the active ingredient, that is included in a formulation of the active ingredient. As used herein, an excipient may be incorporated within particles of a pharmaceutical composition, or it may be physically mixed with particles of a pharmaceutical composition. An excipient can be used, for example, to dilute an active agent and/or to modify properties of a pharmaceutical composition. Excipients can include, but are not limited to, antiadherents, binders, coatings, enteric coatings, disintegrants, flavorings, sweeteners, colorants, lubricants, glidants, sorbents, preservatives, adjuvants, carriers or vehicles. Excipients may be starches and modified starches, cellulose and cellulose derivatives, saccharides and their derivatives such as disaccharides, polysaccharides and sugar alcohols, protein, synthetic polymers, crosslinked polymers, antioxidants, amino acids or preservatives. Exemplary excipients include, but are not limited to, magnesium stearate, stearic acid, vegetable stearin, sucrose, lactose, starches, hydroxypropyl cellulose, hydroxypropyl methylcellulose, xylitol, sorbitol, maltitol, gelatin, polyvinylpyrrolidone (PVP), polyethyleneglycol (PEG), tocopheryl polyethylene glycol 1000 succinate (also known as vitamin E TPGS, or TPGS), carboxy methyl cellulose, dipalmitoyl phosphatidyl choline (DPPC), vitamin A, vitamin E, vitamin C, retinyl palmitate, selenium, cysteine, methionine, citric acid, sodium citrate, methyl paraben, propyl paraben, sugar, silica, talc, magnesium carbonate, sodium starch glycolate, tartrazine, aspartame, benzalkonium chloride, sesame oil, propyl gallate, sodium metabisulphite or lanolin.

An “adjuvant” is an excipient that modifies the effect of other agents, typically the active ingredient. Adjuvants are often pharmacological and/or immunological agents. An adjuvant may modify the effect of an active ingredient by increasing an immune response. An adjuvant may also act as a stabilizing agent for a formulation. Exemplary adjuvants include, but are not limited to, aluminum hydroxide, alum, aluminum phosphate, killed bacteria, squalene, detergents, cytokines, paraffin oil, and combination adjuvants, such as Freund's complete adjuvant or Freund's incomplete adjuvant.

“Pharmaceutically acceptable carrier” refers to an excipient that is a carrier or vehicle, such as a suspension aid, solubilizing aid, or aerosolization aid. Remington: The Science and Practice of Pharmacy, The University of the Sciences in Philadelphia, Editor, Lippincott, Williams, & Wilkins, Philadelphia, PA, 21^{st} Edition (2005), incorporated herein by reference, describes exemplary compositions and formulations suitable for pharmaceutical delivery of one or more therapeutic compositions and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In some examples, the pharmaceutically acceptable carrier may be sterile to be suitable for administration to a subject (for example, by parenteral, intramuscular, or subcutaneous injection). In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

“Pharmaceutically acceptable salt” refers to pharmaceutically acceptable salts of a compound that are derived from a variety of organic and inorganic counter ions as will be known to a person of ordinary skill in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like. “Pharmaceutically acceptable acid addition salts” are a subset of “pharmaceutically acceptable salts” that retain the biological effectiveness of the free bases while formed by acid partners. In particular, the disclosed compounds form salts with a variety of pharmaceutically acceptable acids, including, without limitation, inorganic acids such as hydrochloric acid, hydrobromic acid, sulfuric acid, nitric acid, phosphoric acid, and the like, as well as organic acids such as formic acid, acetic acid, adipic acid, aspartic acid, trifluoroacetic acid, propionic acid, gentisic acid, glycolic acid, pyruvic acid, oxalic acid, maleic acid, malic acid, malonic acid, succinic acid, fumaric acid, tartaric acid, citric acid, benzoic acid, cinnamic acid, mandelic acid, benzene sulfonic acid, isethionic acid, lactic acid, methanesulfonic acid, ethanesulfonic acid, naphthalene-1,5-disulfonic acid, naphthalene-2-sulfonic acid, p-toluenesulfonic acid, salicylic acid, xinafoic acid and the like. “Pharmaceutically acceptable base addition salts” are a subset of “pharmaceutically acceptable salts” that are derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, tris(hydroxymethyl)aminomethane (Tris), ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, tris(hydroxymethyl)aminomethane (Tris), ethanolamine, trimethylamine, dicyclohexylamine, choline, and caffeine. (See, for example, S. M. Berge, et al., “Pharmaceutical Salts,” J. Pharm. Sci., 1977; 66:1-19 which is incorporated herein by reference.) In particular disclosed embodiments, the pyrazole compound may be a formate or sodium salt.

“Effective amount” with respect to a compound or composition refer to an amount of the compound or composition sufficient to achieve a particular desired result, such as to inhibit a protein or enzyme, particularly an interleukin-1 receptor-associated kinase; to elicit a desired biological or medical response in a tissue, system, subject or patient; to treat or prevent a specified disorder or disease; to ameliorate or eradicate one or more of its symptoms; and/or to prevent the occurrence of the disease or disorder. The amount of a compound which constitutes an “effective amount” may vary depending on the compound, the desired result, the disease state and its severity, the age of the patient to be treated, and the like.

“Solvate” refers to a complex formed by combination of solvent molecules with molecules or ions of a solute. The solvent can be an organic solvent, an inorganic solvent, or a mixture of both. Exemplary solvents include, but are not limited to, alcohols, such as methanol, ethanol, propanol; amides such as N,N-dialiphatic amides, such as N,N-dimethylformamide; tetrahydrofuran; alkylsulfoxides, such as dimethylsulfoxide; water; and combinations thereof. The compounds described herein can exist in un-solvated as well as solvated forms when combined with solvents, pharmaceutically acceptable or not, such as water, ethanol, and the like. Solvated forms of the presently disclosed compounds are within the scope of the embodiments disclosed herein.

“Treating” or “treatment” as used herein concerns treatment of a disease or condition of interest in a patient or subject, particularly a human having the disease or condition of interest, and includes by way of example, and without limitation:
(i) inhibiting the disease or condition, for example, arresting or slowing its development;
(ii) relieving the disease or condition, for example, causing regression of the disease or condition or a symptom thereof; or
(iii) stabilizing the disease or condition.

“Preventing” as used herein concerns preventing the disease or condition from occurring in a patient or subject, in particular, when such patient or subject is predisposed to the condition but has not yet been diagnosed as having it.

As used herein, the terms “disease,” “disorder” and “condition” can be used interchangeably or can be different in that the particular malady or condition may not have a known causative agent (so that etiology has not yet been determined) and it is therefore not yet recognized as a disease but only as an undesirable condition or syndrome, where a more or less specific set of symptoms have been identified by clinicians.

### II. Compounds

Disclosed herein are bicyclic compounds, methods of making the compounds, and methods of using the compounds. In one embodiment, the disclosed compounds are kinase inhibitors, particularly tyrosine kinase inhibitors. In a particular embodiment the compounds are useful in blocking one or more cytokine signaling pathways, such as the IL-17 signaling pathway. For certain embodiments, the disclosed compounds are useful for treating conditions in which inhibition of an interleukin-1 receptor-associated kinase (IRAK) pathway is therapeutically useful. In some embodiments, the compounds directly inhibit an IRAK protein, such as IRAK1, IRAK2, IRAK3 and/or IRAK4.

Exemplary compounds within the scope of the present disclosure have a general Formula I

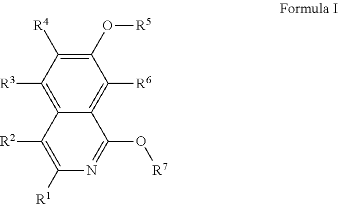

or a pharmaceutically acceptable salt or solvate thereof. With respect to Formula I, each of R¹, R³ and R⁶ independently are H or C₁₋₆alkyl. In some embodiments, R¹, R³ and R⁶ are each H.
R² is alkynyl, heteroaryl, or halogen.
R⁴ is —C(O)NH₂.
R⁵ is C₁₋₆alkyl, and may be methyl, ethyl, n-propyl, isopropyl, n-butyl, sec-butyl, isobutyl, or tert-butyl. In some embodiments, R⁵ is isopropyl. In other embodiments, R⁵ is methyl.
R⁷ is —(CH₂)ₙ-heterocycloaliphatic, such as 5-membered or 6-membered heterocycloaliphatic, such as 5-membered or 6-membered nitrogen-containing heterocycloaliphatic, where n is from 0 to 2, such as 0 or 1 or 2. R⁷ may be optionally substituted with oxo (═O), —C(O)CH₂CN, halogen (such as F), C₁₋₆alkyl, —C(O)O—C₁₋₆alkyl), or a combination thereof.

In some embodiments, R² is halogen, such as Br,

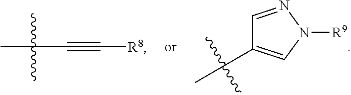

R⁸ is C₁₋₆alkyl optionally substituted with 1, 2 or 3 substituents selected from OH or 4- to 6-membered heterocycloaliphatic; 4- to 6-membered heterocycloaliphatic optionally substituted with 1, 2, or 3 substituents selected from R^{a}; or C₃₋₆cycloalkyl optionally substituted with 1, 2 or 3 R^{b}.
R⁹ is C₃₋₆cycloalkyl optionally substituted with 1, 2 or 3 R^{b}; C₁₋₆alkyl optionally substituted with 4- to 6-membered heterocycloaliphatic; or 4- to 6-membered heterocycloaliphatic optionally substituted with 1, 2, or 3 R^{a}.
R^{a} is OH, —C(O)OR^{c}, C₁₋₆alkyl, or —C(O)NH(R^{c}).
R^{b} is OR^{c}, C₁₋₆alkyl, or —N(R^{c})₂.
R^{c} is H or C₁₋₆alkyl.

In some embodiments, R⁷ is

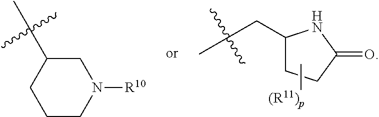

R¹⁰ is H, —C(O)OC₁₋₆alkyl, or —C(O)CH₂CN.
Each R¹¹ independently is halogen or C₁₋₆alkyl. In some embodiments, each R¹¹ independently is F or C₁₋₄alkyl, such as F or ethyl.
p is from 0 to 4, and may be 0, 1 or 2.

In certain embodiments, R⁷ is

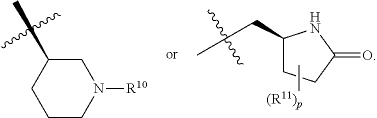

In some embodiments, R⁷ is selected from

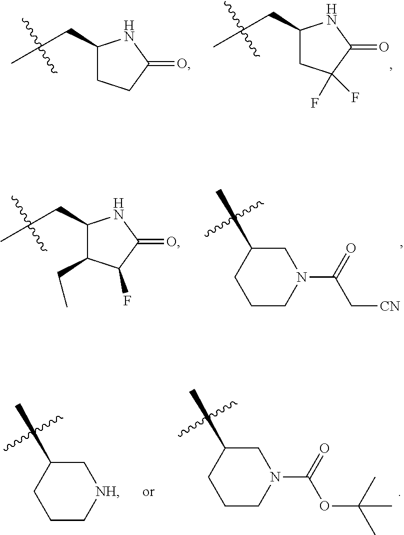

In some embodiments, R¹, R³ and R⁶ are each H, R⁴ is —C(O)NH₂ and the compound has a structure according to Formula II

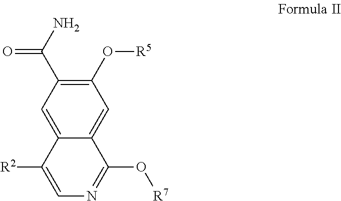

or a pharmaceutically acceptable salt or solvate thereof. With respect to Formula II, R², R⁵ and R⁷ are as defined for Formula I.

In some embodiments, the compound has a structure according to Formula III or Formula IV, or a pharmaceutically acceptable salt or solvate thereof:

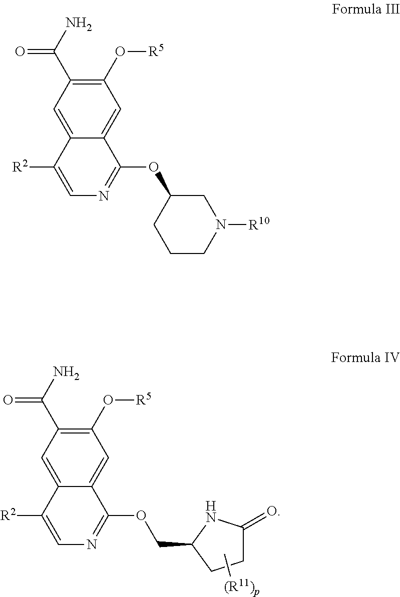

With respect to Formulas III and IV, R², R⁵, R¹⁰ and R¹¹ are as defined for Formulas I and II. In certain embodiments, R⁵ is isopropyl.

In some embodiments of Formulas I-IV, R² is halogen, such as Br. In other embodiments of Formulas I-IV, R² is

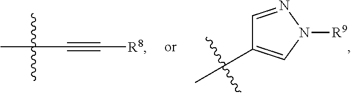

and may be selected from

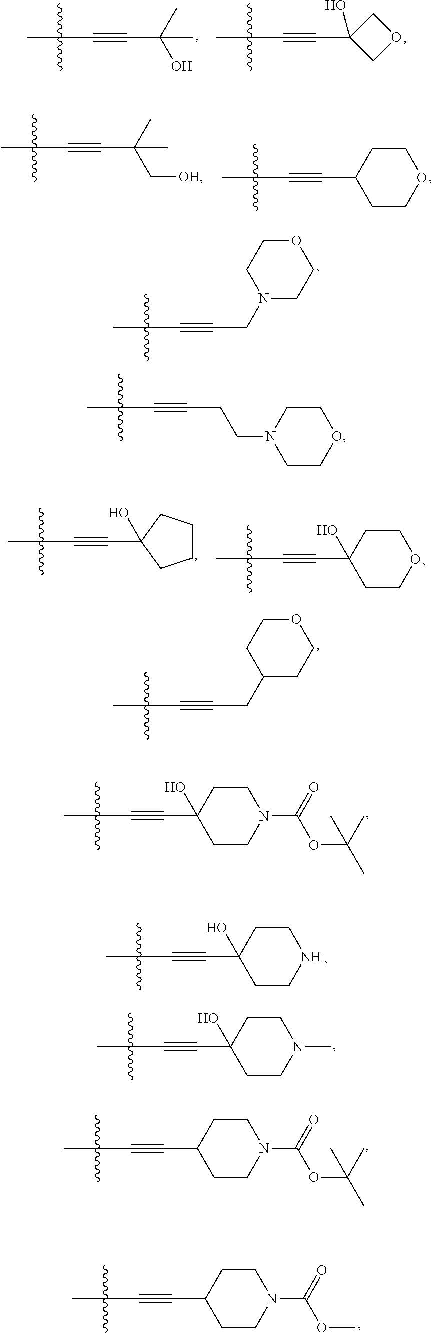

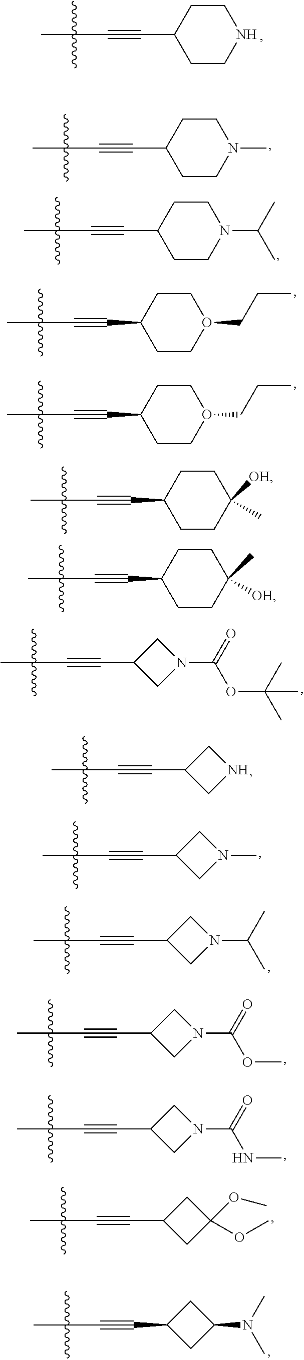

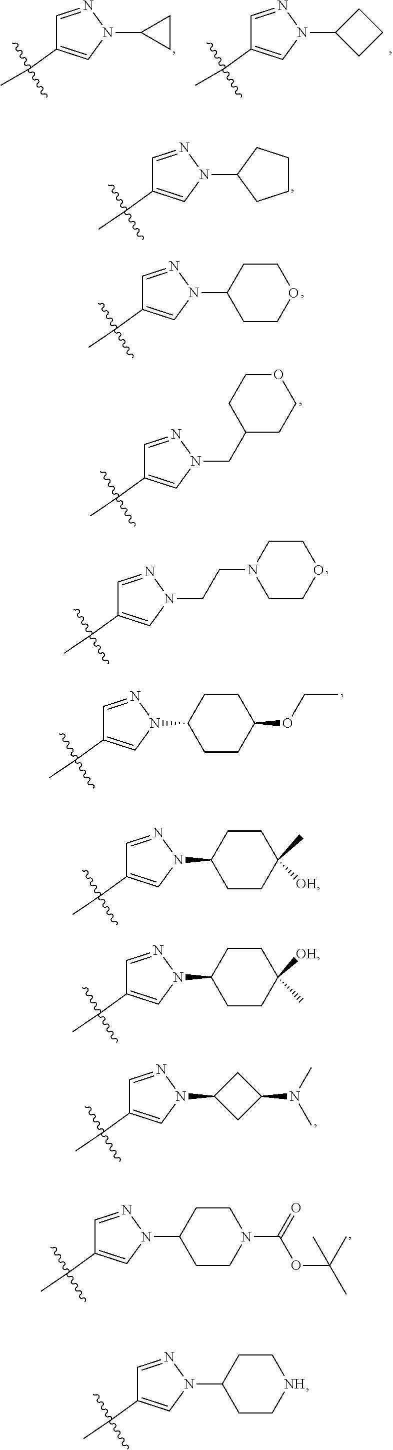

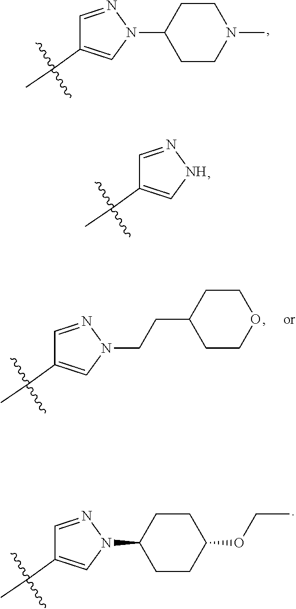 [SMILES: *c1cnn(C2CCN(C)CC2)c1 ; *c1cn[nH]c1 ; *c1cnn(CCC2CCOCC2)c1 ; *c1cnn([C@H]2CC[C@H](OCC)CC2)c1]

Exemplary compounds according to Formulas I-IV include, but are not limited to:

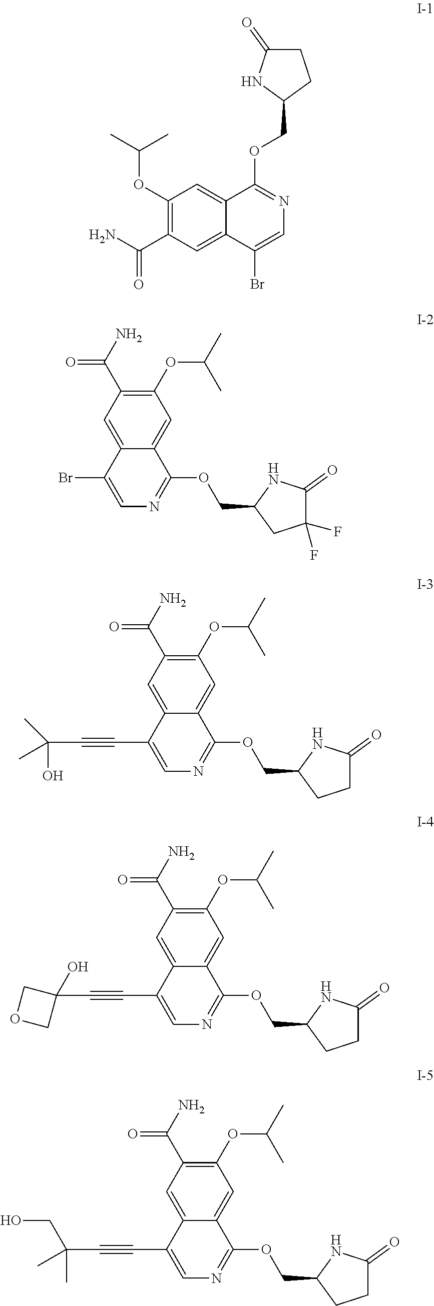 [SMILES: CC(C)Oc1cc2c(OC[C@@H]3CCC(=O)N3)ncc(Br)c2cc1C(N)=O ; CC(C)Oc1cc2c(OC[C@@H]3CC(F)(F)C(=O)N3)ncc(Br)c2cc1C(N)=O ; CC(C)Oc1cc2c(OC[C@@H]3CCC(=O)N3)ncc(C#CC(C)(C)O)c2cc1C(N)=O ; CC(C)Oc1cc2c(OC[C@@H]3CCC(=O)N3)ncc(C#CC3(O)COC3)c2cc1C(N)=O ; CC(C)Oc1cc2c(OC[C@@H]3CCC(=O)N3)ncc(C#CC(C)(C)CO)c2cc1C(N)=O]

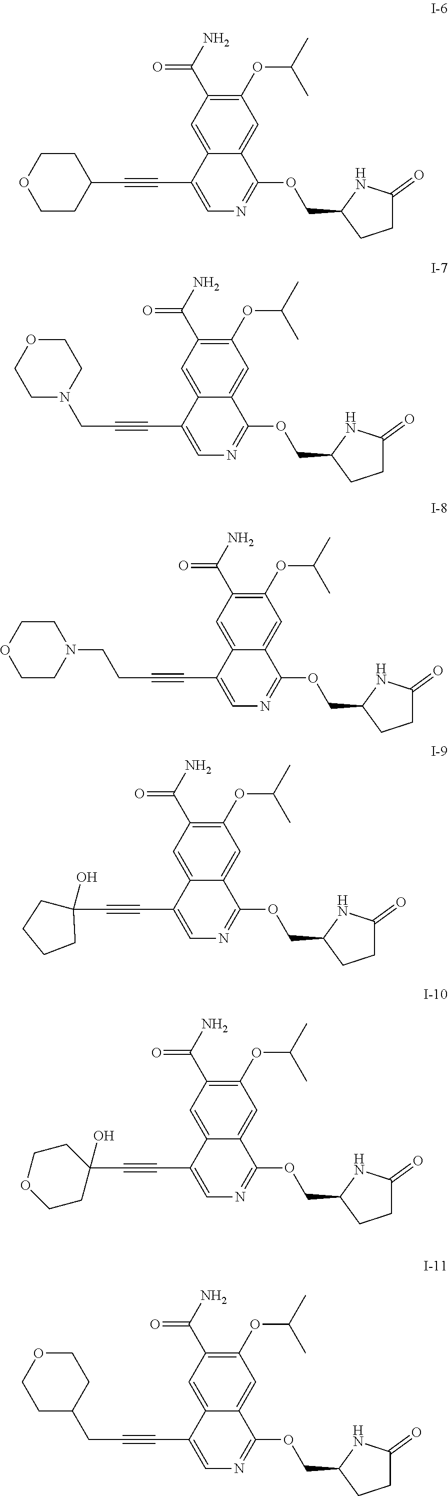 [SMILES: CC(C)Oc1cc2c(OC[C@@H]3CCC(=O)N3)ncc(C#CC3CCOCC3)c2cc1C(N)=O ; CC(C)Oc1cc2c(OC[C@@H]3CCC(=O)N3)ncc(C#CCN3CCOCC3)c2cc1C(N)=O ; CC(C)Oc1cc2c(OC[C@@H]3CCC(=O)N3)ncc(C#CCCN3CCOCC3)c2cc1C(N)=O ; CC(C)Oc1cc2c(OC[C@@H]3CCC(=O)N3)ncc(C#CC3(O)CCCC3)c2cc1C(N)=O ; CC(C)Oc1cc2c(OC[C@@H]3CCC(=O)N3)ncc(C#CC3(O)CCOCC3)c2cc1C(N)=O ; CC(C)Oc1cc2c(OC[C@@H]3CCC(=O)N3)ncc(C#CCC3CCOCC3)c2cc1C(N)=O]

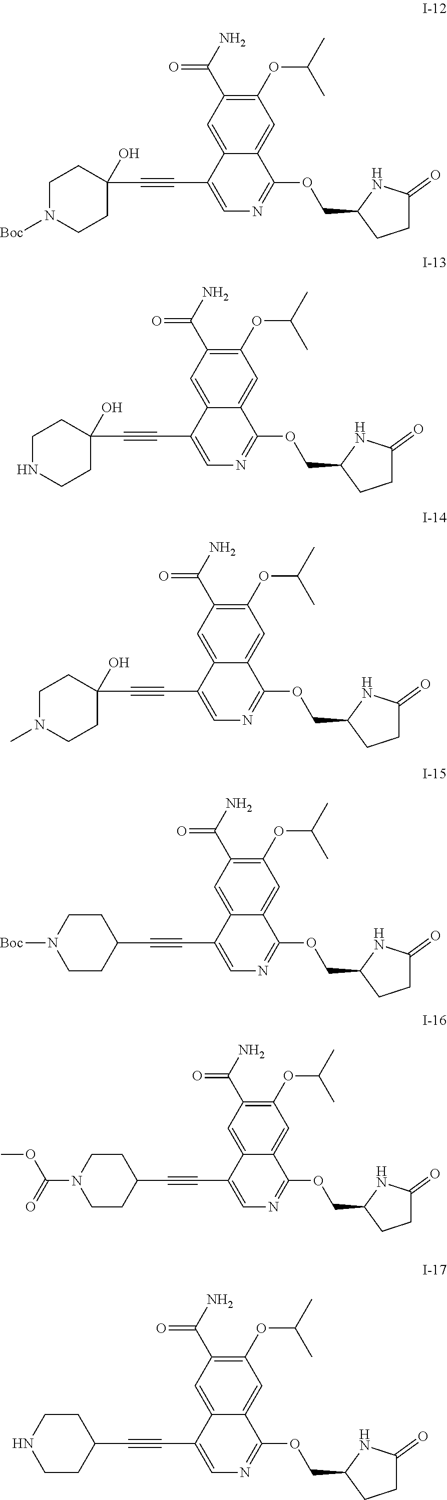 [SMILES: CC(C)Oc1cc2c(OC[C@@H]3CCC(=O)N3)ncc(C#CC3(O)CCN(C(=O)OC(C)(C)C)CC3)c2cc1C(N)=O ; CC(C)Oc1cc2c(OC[C@@H]3CCC(=O)N3)ncc(C#CC3(O)CCNCC3)c2cc1C(N)=O ; CC(C)Oc1cc2c(OC[C@@H]3CCC(=O)N3)ncc(C#CC3(O)CCN(C)CC3)c2cc1C(N)=O ; CC(C)Oc1cc2c(OC[C@@H]3CCC(=O)N3)ncc(C#CC3CCN(C(=O)OC(C)(C)C)CC3)c2cc1C(N)=O ; COC(=O)N1CCC(C#Cc2cnc(OC[C@@H]3CCC(=O)N3)c3cc(OC(C)C)c(C(N)=O)cc23)CC1 ; CC(C)Oc1cc2c(OC[C@@H]3CCC(=O)N3)ncc(C#CC3CCNCC3)c2cc1C(N)=O]

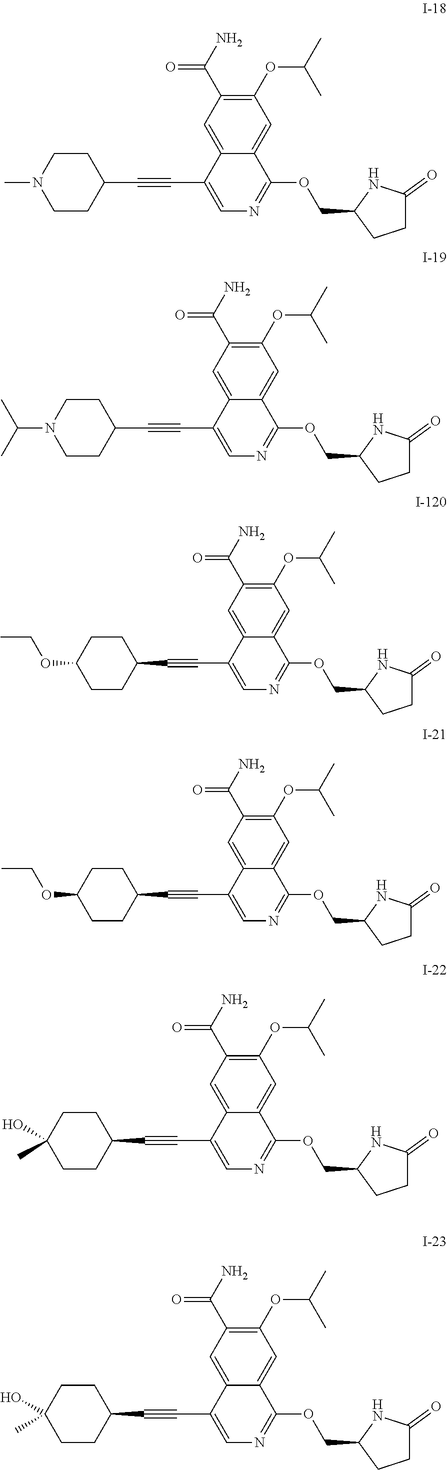

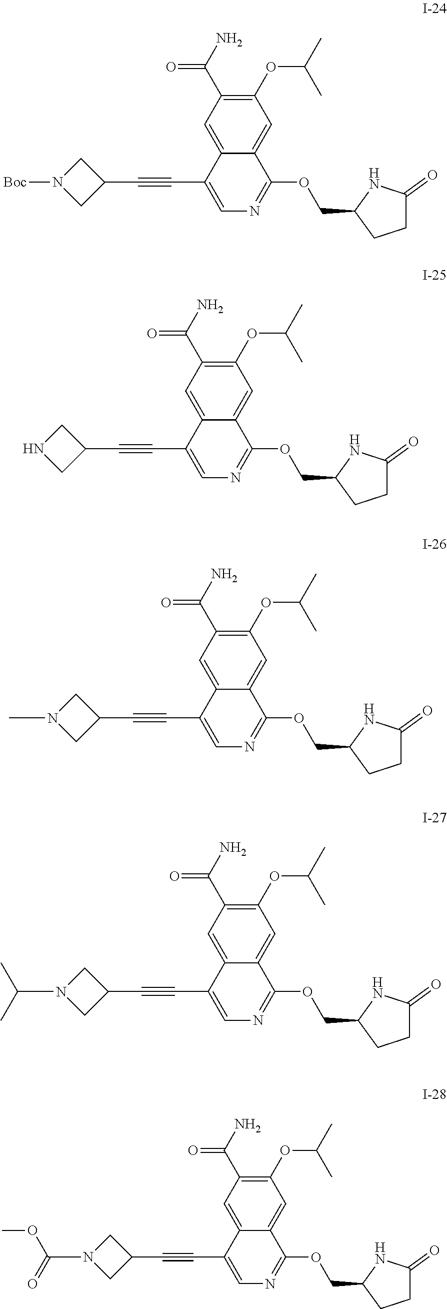 [SMILES: CC(C)Oc1cc2c(OC[C@@H]3CCC(=O)N3)ncc(C#CC3CN(C(=O)OC(C)(C)C)C3)c2cc1C(N)=O ; CC(C)Oc1cc2c(OC[C@@H]3CCC(=O)N3)ncc(C#CC3CNC3)c2cc1C(N)=O ; CC(C)Oc1cc2c(OC[C@@H]3CCC(=O)N3)ncc(C#CC3CN(C)C3)c2cc1C(N)=O ; CC(C)Oc1cc2c(OC[C@@H]3CCC(=O)N3)ncc(C#CC3CN(C(C)C)C3)c2cc1C(N)=O ; COC(=O)N1CC(C#Cc2cnc(OC[C@@H]3CCC(=O)N3)c3cc(OC(C)C)c(C(N)=O)cc23)C1]

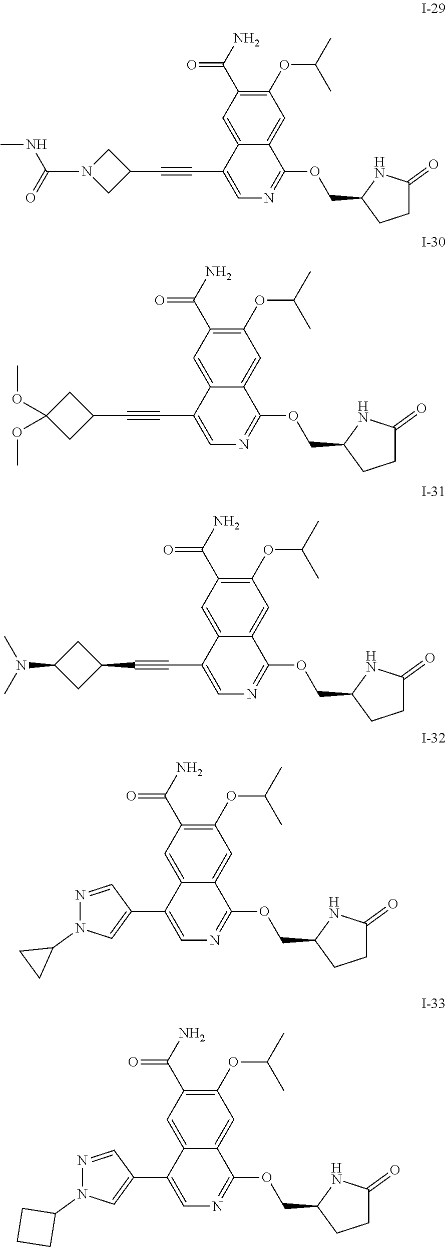 [SMILES: CNC(=O)N1CC(C#Cc2cnc(OC[C@@H]3CCC(=O)N3)c3cc(OC(C)C)c(C(N)=O)cc23)C1 ; COC1(OC)CC(C#Cc2cnc(OC[C@@H]3CCC(=O)N3)c3cc(OC(C)C)c(C(N)=O)cc23)C1 ; CC(C)Oc1cc2c(OC[C@@H]3CCC(=O)N3)ncc(C#C[C@H]3C[C@@H](N(C)C)C3)c2cc1C(N)=O ; CC(C)Oc1cc2c(OC[C@@H]3CCC(=O)N3)ncc(-c3cnn(C4CC4)c3)c2cc1C(N)=O ; CC(C)Oc1cc2c(OC[C@@H]3CCC(=O)N3)ncc(-c3cnn(C4CCC4)c3)c2cc1C(N)=O]

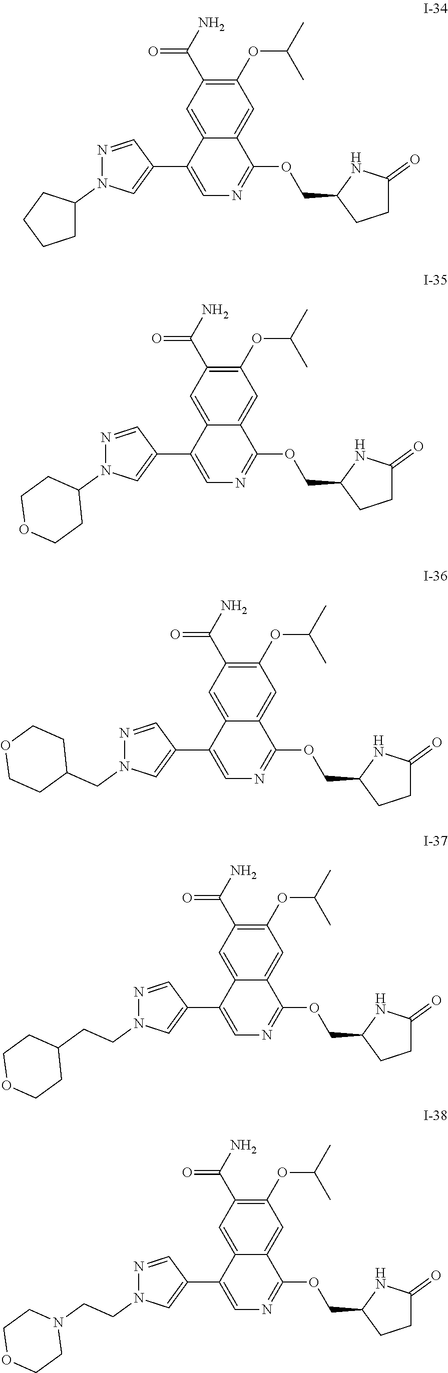 [SMILES: CC(C)Oc1cc2c(OC[C@@H]3CCC(=O)N3)ncc(-c3cnn(C4CCCC4)c3)c2cc1C(N)=O ; CC(C)Oc1cc2c(OC[C@@H]3CCC(=O)N3)ncc(-c3cnn(C4CCOCC4)c3)c2cc1C(N)=O ; CC(C)Oc1cc2c(OC[C@@H]3CCC(=O)N3)ncc(-c3cnn(CC4CCOCC4)c3)c2cc1C(N)=O ; CC(C)Oc1cc2c(OC[C@@H]3CCC(=O)N3)ncc(-c3cnn(CCC4CCOCC4)c3)c2cc1C(N)=O ; CC(C)Oc1cc2c(OC[C@@H]3CCC(=O)N3)ncc(-c3cnn(CCN4CCOCC4)c3)c2cc1C(N)=O]

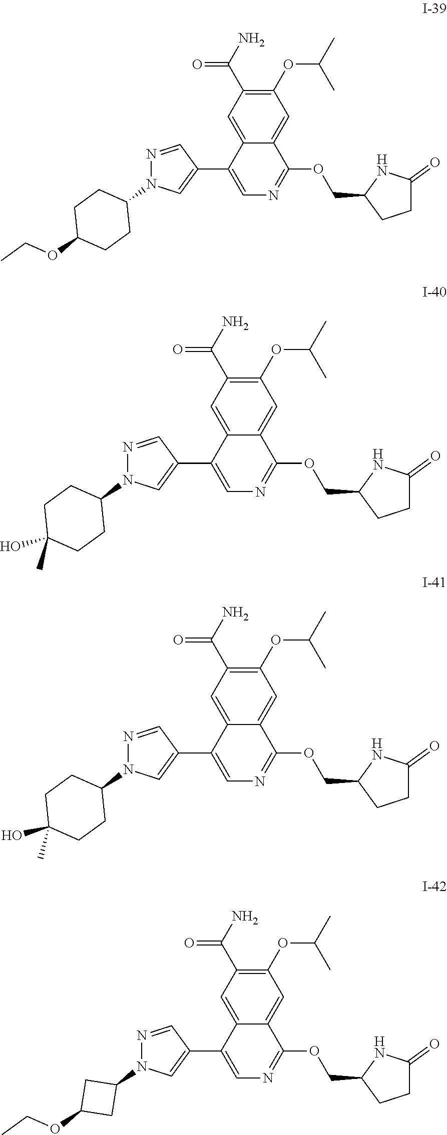 [SMILES: CCO[C@H]1CC[C@H](n2cc(-c3cnc(OC[C@@H]4CCC(=O)N4)c4cc(OC(C)C)c(C(N)=O)cc34)cn2)CC1 ; CC(C)Oc1cc2c(OC[C@@H]3CCC(=O)N3)ncc(-c3cnn([C@H]4CC[C@](C)(O)CC4)c3)c2cc1C(N)=O ; CC(C)Oc1cc2c(OC[C@@H]3CCC(=O)N3)ncc(-c3cnn([C@H]4CC[C@@](C)(O)CC4)c3)c2cc1C(N)=O ; CCO[C@H]1C[C@@H](n2cc(-c3cnc(OC[C@@H]4CCC(=O)N4)c4cc(OC(C)C)c(C(N)=O)cc34)cn2)C1]

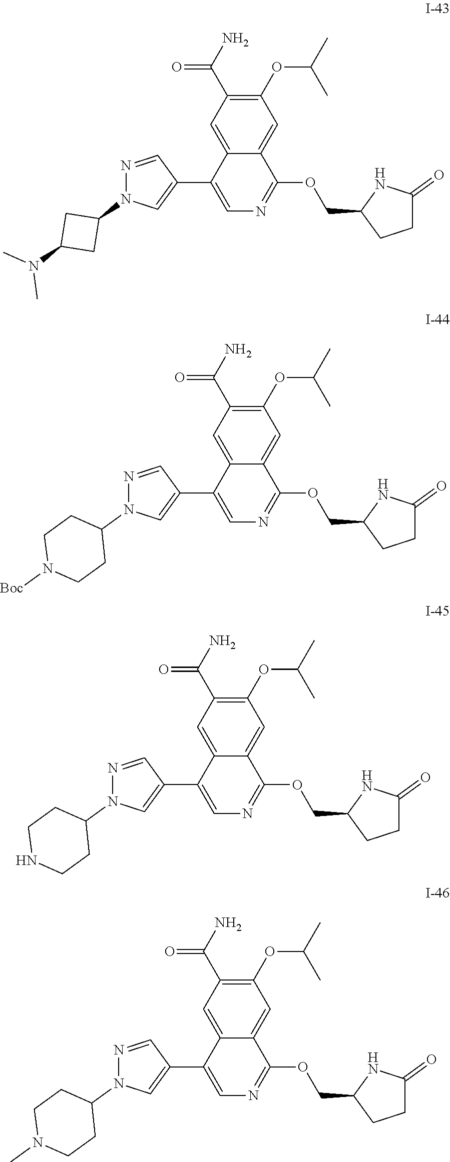 [SMILES: CC(C)Oc1cc2c(OC[C@@H]3CCC(=O)N3)ncc(-c3cnn([C@H]4C[C@@H](N(C)C)C4)c3)c2cc1C(N)=O ; CC(C)Oc1cc2c(OC[C@@H]3CCC(=O)N3)ncc(-c3cnn(C4CCN(C(=O)OC(C)(C)C)CC4)c3)c2cc1C(N)=O ; CC(C)Oc1cc2c(OC[C@@H]3CCC(=O)N3)ncc(-c3cnn(C4CCNCC4)c3)c2cc1C(N)=O ; CC(C)Oc1cc2c(OC[C@@H]3CCC(=O)N3)ncc(-c3cnn(C4CCN(C)CC4)c3)c2cc1C(N)=O]

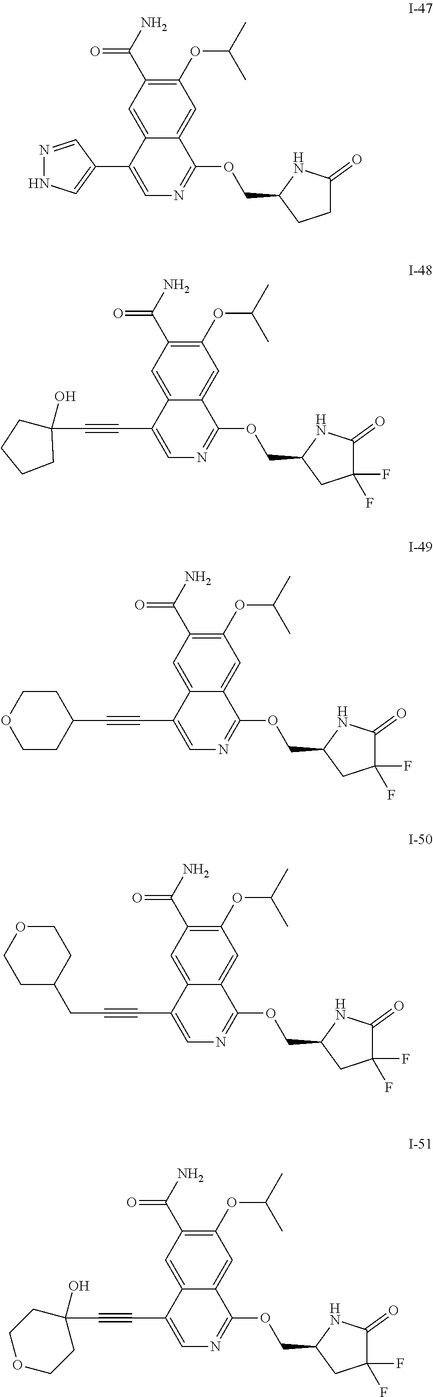 [SMILES: CC(C)Oc1cc2c(OC[C@@H]3CCC(=O)N3)ncc(-c3cn[nH]c3)c2cc1C(N)=O ; CC(C)Oc1cc2c(OC[C@@H]3CC(F)(F)C(=O)N3)ncc(C#CC3(O)CCCC3)c2cc1C(N)=O ; CC(C)Oc1cc2c(OC[C@@H]3CC(F)(F)C(=O)N3)ncc(C#CC3CCOCC3)c2cc1C(N)=O ; CC(C)Oc1cc2c(OC[C@@H]3CC(F)(F)C(=O)N3)ncc(C#CCC3CCOCC3)c2cc1C(N)=O ; CC(C)Oc1cc2c(OC[C@@H]3CC(F)(F)C(=O)N3)ncc(C#CC3(O)CCOCC3)c2cc1C(N)=O]

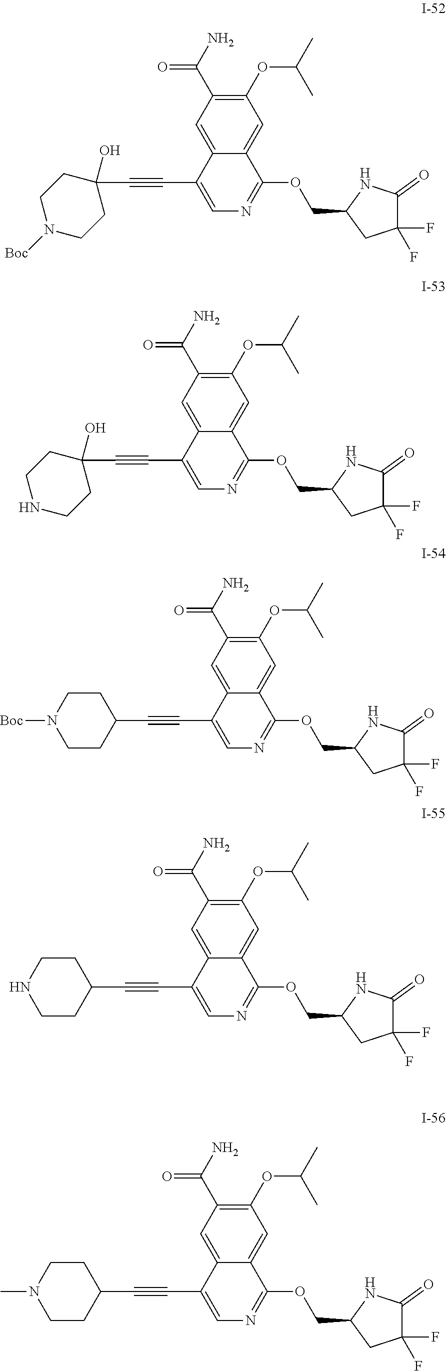 [SMILES: CC(C)Oc1cc2c(OC[C@@H]3CC(F)(F)C(=O)N3)ncc(C#CC3(O)CCN(C(=O)OC(C)(C)C)CC3)c2cc1C(N)=O ; CC(C)Oc1cc2c(OC[C@@H]3CC(F)(F)C(=O)N3)ncc(C#CC3(O)CCNCC3)c2cc1C(N)=O ; CC(C)Oc1cc2c(OC[C@@H]3CC(F)(F)C(=O)N3)ncc(C#CC3CCN(C(=O)OC(C)(C)C)CC3)c2cc1C(N)=O ; CC(C)Oc1cc2c(OC[C@@H]3CC(F)(F)C(=O)N3)ncc(C#CC3CCNCC3)c2cc1C(N)=O ; CC(C)Oc1cc2c(OC[C@@H]3CC(F)(F)C(=O)N3)ncc(C#CC3CCN(C)CC3)c2cc1C(N)=O]

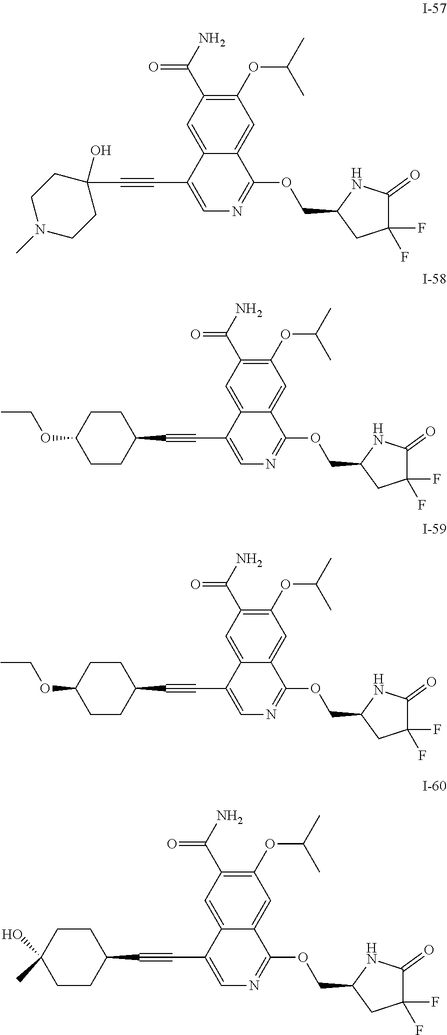 [SMILES: CC(C)Oc1cc2c(OC[C@@H]3CC(F)(F)C(=O)N3)ncc(C#CC3(O)CCN(C)CC3)c2cc1C(N)=O ; CCO[C@H]1CC[C@H](C#Cc2cnc(OC[C@@H]3CC(F)(F)C(=O)N3)c3cc(OC(C)C)c(C(N)=O)cc23)CC1 ; CCO[C@H]1CC[C@@H](C#Cc2cnc(OC[C@@H]3CC(F)(F)C(=O)N3)c3cc(OC(C)C)c(C(N)=O)cc23)CC1 ; CC(C)Oc1cc2c(OC[C@@H]3CC(F)(F)C(=O)N3)ncc(C#C[C@H]3CC[C@](C)(O)CC3)c2cc1C(N)=O]

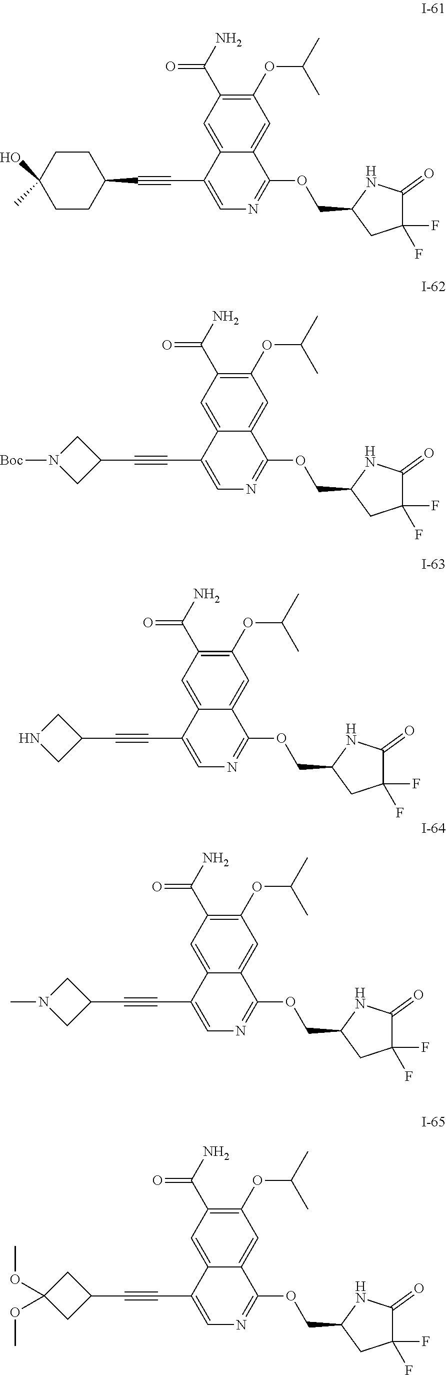 [SMILES: CC(C)Oc1cc2c(OC[C@@H]3CC(F)(F)C(=O)N3)ncc(C#C[C@H]3CC[C@@](C)(O)CC3)c2cc1C(N)=O ; CC(C)Oc1cc2c(OC[C@@H]3CC(F)(F)C(=O)N3)ncc(C#CC3CN(C(=O)OC(C)(C)C)C3)c2cc1C(N)=O ; CC(C)Oc1cc2c(OC[C@@H]3CC(F)(F)C(=O)N3)ncc(C#CC3CNC3)c2cc1C(N)=O ; CC(C)Oc1cc2c(OC[C@@H]3CC(F)(F)C(=O)N3)ncc(C#CC3CN(C)C3)c2cc1C(N)=O ; COC1(OC)CC(C#Cc2cnc(OC[C@@H]3CC(F)(F)C(=O)N3)c3cc(OC(C)C)c(C(N)=O)cc23)C1]

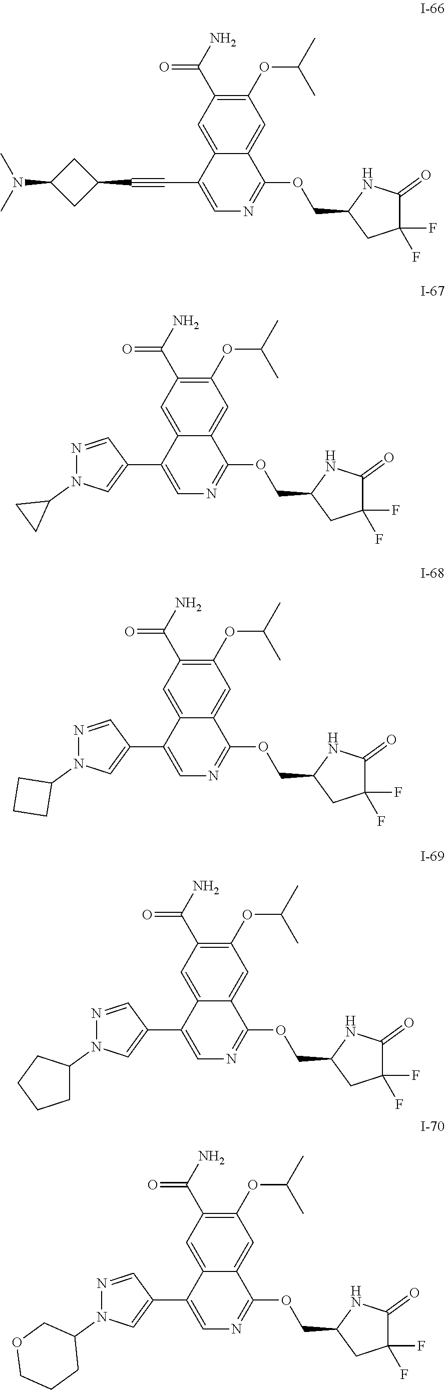 [SMILES: CC(C)Oc1cc2c(OC[C@@H]3CC(F)(F)C(=O)N3)ncc(C#C[C@H]3C[C@@H](N(C)C)C3)c2cc1C(N)=O ; CC(C)Oc1cc2c(OC[C@@H]3CC(F)(F)C(=O)N3)ncc(-c3cnn(C4CC4)c3)c2cc1C(N)=O ; CC(C)Oc1cc2c(OC[C@@H]3CC(F)(F)C(=O)N3)ncc(-c3cnn(C4CCC4)c3)c2cc1C(N)=O ; CC(C)Oc1cc2c(OC[C@@H]3CC(F)(F)C(=O)N3)ncc(-c3cnn(C4CCCC4)c3)c2cc1C(N)=O ; CC(C)Oc1cc2c(OC[C@@H]3CC(F)(F)C(=O)N3)ncc(-c3cnn(C4CCCOC4)c3)c2cc1C(N)=O]

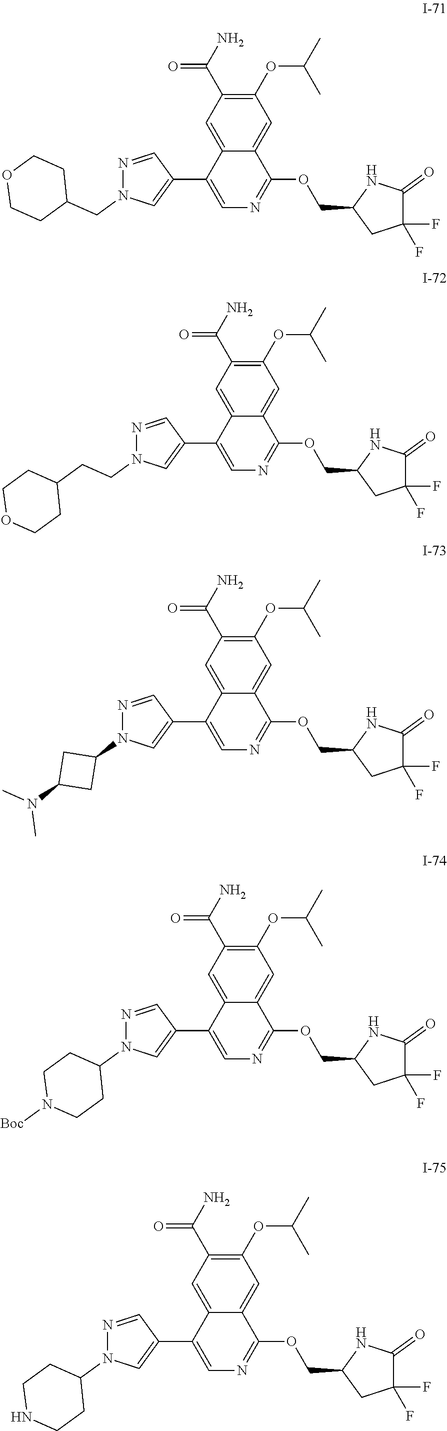 [SMILES: CC(C)Oc1cc2c(OC[C@@H]3CC(F)(F)C(=O)N3)ncc(-c3cnn(CC4CCOCC4)c3)c2cc1C(N)=O ; CC(C)Oc1cc2c(OC[C@@H]3CC(F)(F)C(=O)N3)ncc(-c3cnn(CCC4CCOCC4)c3)c2cc1C(N)=O ; CC(C)Oc1cc2c(OC[C@@H]3CC(F)(F)C(=O)N3)ncc(-c3cnn([C@H]4C[C@@H](N(C)C)C4)c3)c2cc1C(N)=O ; CC(C)Oc1cc2c(OC[C@@H]3CC(F)(F)C(=O)N3)ncc(-c3cnn(C4CCN(C(=O)OC(C)(C)C)CC4)c3)c2cc1C(N)=O ; CC(C)Oc1cc2c(OC[C@@H]3CC(F)(F)C(=O)N3)ncc(-c3cnn(C4CCNCC4)c3)c2cc1C(N)=O]

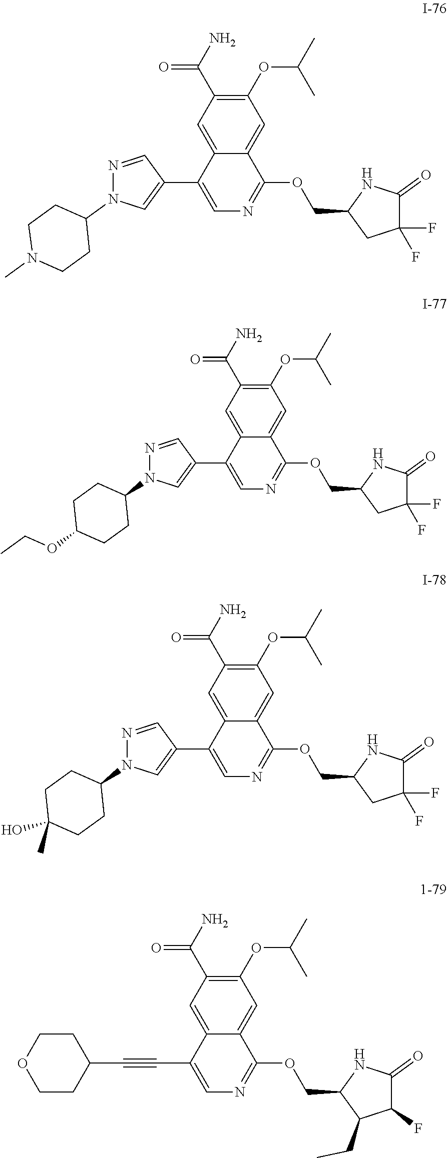 [SMILES: CC(C)Oc1cc2c(OC[C@@H]3CC(F)(F)C(=O)N3)ncc(-c3cnn(C4CCN(C)CC4)c3)c2cc1C(N)=O ; CCO[C@H]1CC[C@H](n2cc(-c3cnc(OC[C@@H]4CC(F)(F)C(=O)N4)c4cc(OC(C)C)c(C(N)=O)cc34)cn2)CC1 ; CC(C)Oc1cc2c(OC[C@@H]3CC(F)(F)C(=O)N3)ncc(-c3cnn([C@H]4CC[C@](C)(O)CC4)c3)c2cc1C(N)=O ; CC[C@@H]1[C@H](F)C(=O)N[C@@H]1COc1ncc(C#CC2CCOCC2)c2cc(C(N)=O)c(OC(C)C)cc12]

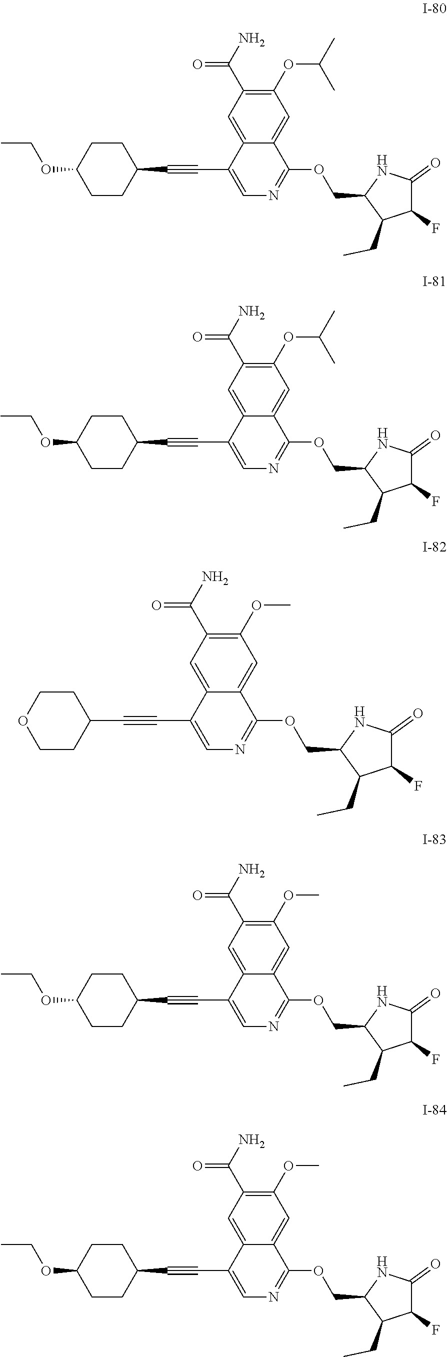 [SMILES: CCO[C@H]1CC[C@H](C#Cc2cnc(OC[C@H]3NC(=O)[C@@H](F)[C@H]3CC)c3cc(OC(C)C)c(C(N)=O)cc23)CC1 ; CCO[C@H]1CC[C@@H](C#Cc2cnc(OC[C@H]3NC(=O)[C@@H](F)[C@H]3CC)c3cc(OC(C)C)c(C(N)=O)cc23)CC1 ; CC[C@@H]1[C@H](F)C(=O)N[C@@H]1COc1ncc(C#CC2CCOCC2)c2cc(C(N)=O)c(OC)cc12 ; CCO[C@H]1CC[C@H](C#Cc2cnc(OC[C@H]3NC(=O)[C@@H](F)[C@H]3CC)c3cc(OC)c(C(N)=O)cc23)CC1 ; CCO[C@H]1CC[C@@H](C#Cc2cnc(OC[C@H]3NC(=O)[C@@H](F)[C@H]3CC)c3cc(OC)c(C(N)=O)cc23)CC1]

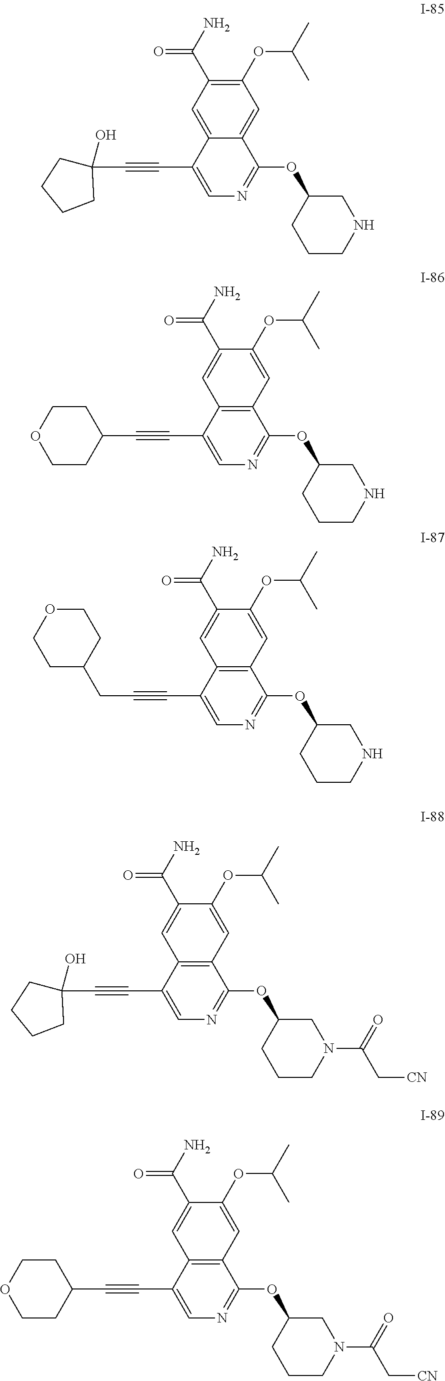

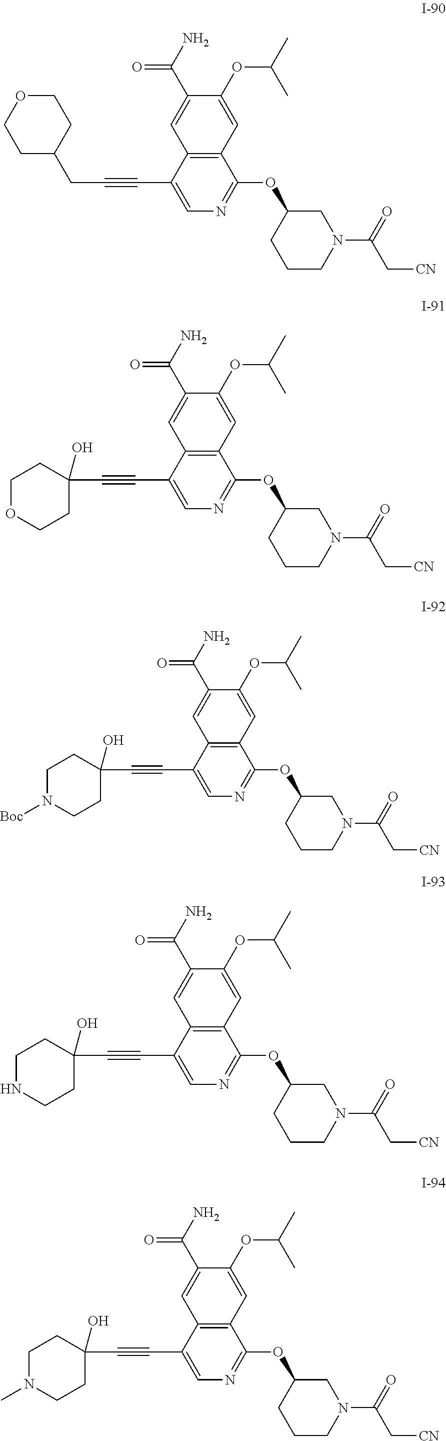 [SMILES: CC(C)Oc1cc2c(O[C@@H]3CCCN(C(=O)CC#N)C3)ncc(C#CCC3CCOCC3)c2cc1C(N)=O ; CC(C)Oc1cc2c(O[C@@H]3CCCN(C(=O)CC#N)C3)ncc(C#CC3(O)CCOCC3)c2cc1C(N)=O ; CC(C)Oc1cc2c(O[C@@H]3CCCN(C(=O)CC#N)C3)ncc(C#CC3(O)CCN(C(=O)OC(C)(C)C)CC3)c2cc1C(N)=O ; CC(C)Oc1cc2c(O[C@@H]3CCCN(C(=O)CC#N)C3)ncc(C#CC3(O)CCNCC3)c2cc1C(N)=O ; CC(C)Oc1cc2c(O[C@@H]3CCCN(C(=O)CC#N)C3)ncc(C#CC3(O)CCN(C)CC3)c2cc1C(N)=O]

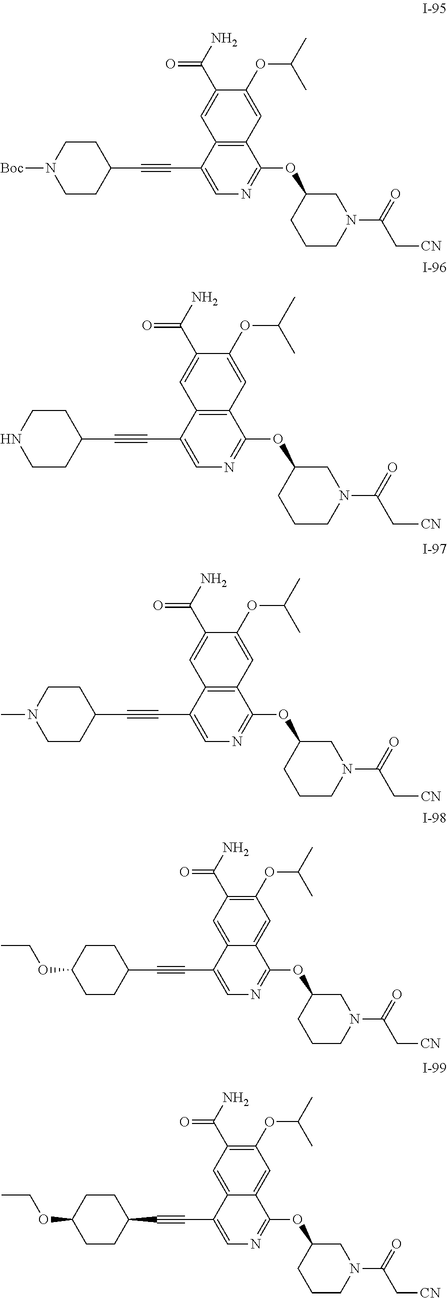

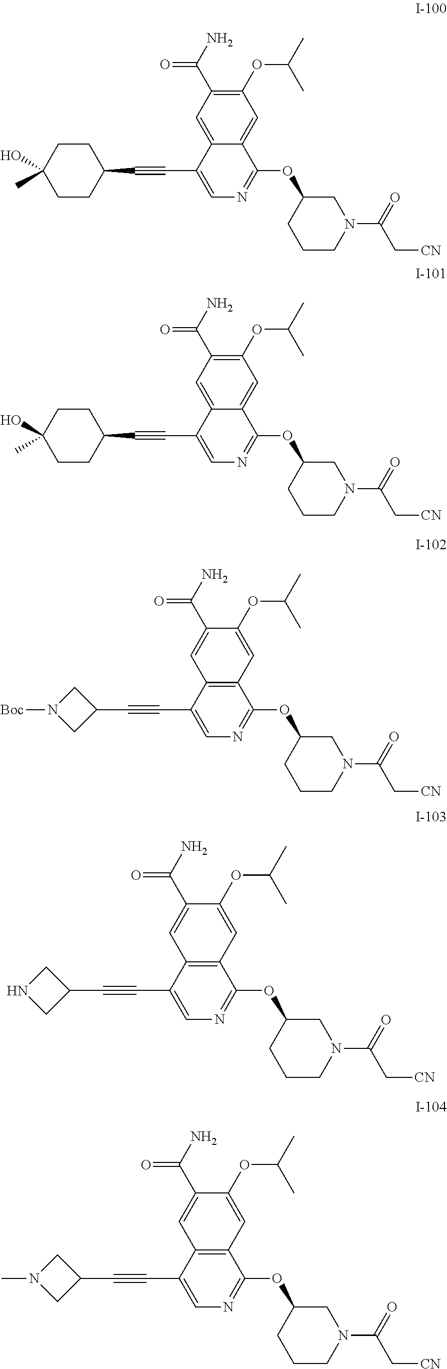 [SMILES: CC(C)Oc1cc2c(O[C@@H]3CCCN(C(=O)CC#N)C3)ncc(C#C[C@H]3CC[C@](C)(O)CC3)c2cc1C(N)=O ; CC(C)Oc1cc2c(O[C@@H]3CCCN(C(=O)CC#N)C3)ncc(C#C[C@H]3CC[C@@](C)(O)CC3)c2cc1C(N)=O ; CC(C)Oc1cc2c(O[C@@H]3CCCN(C(=O)CC#N)C3)ncc(C#CC3CN(C(=O)OC(C)(C)C)C3)c2cc1C(N)=O ; CC(C)Oc1cc2c(O[C@@H]3CCCN(C(=O)CC#N)C3)ncc(C#CC3CNC3)c2cc1C(N)=O ; CC(C)Oc1cc2c(O[C@@H]3CCCN(C(=O)CC#N)C3)ncc(C#CC3CN(C)C3)c2cc1C(N)=O]

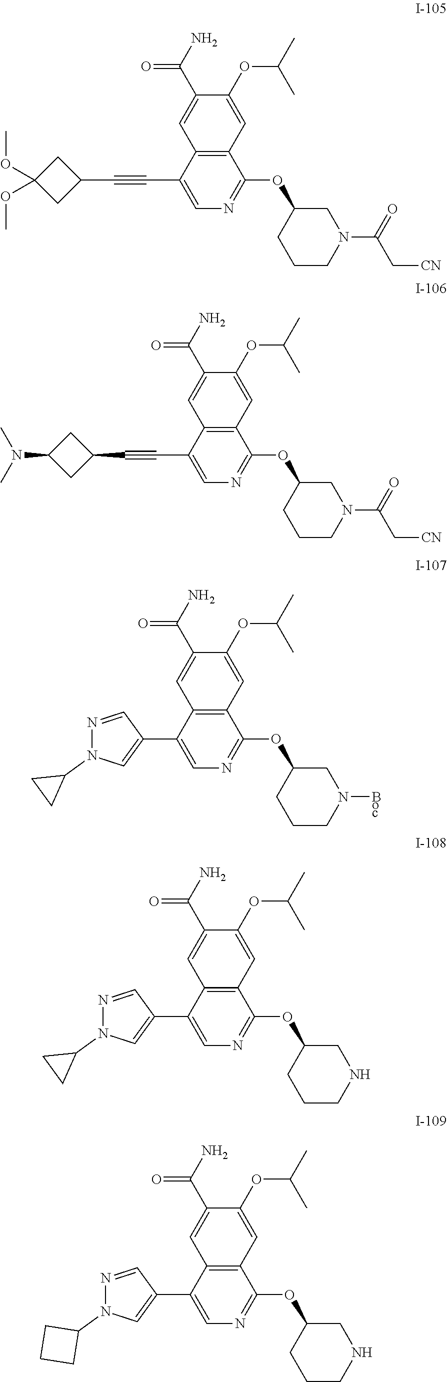 [SMILES: COC1(OC)CC(C#Cc2cnc(O[C@@H]3CCCN(C(=O)CC#N)C3)c3cc(OC(C)C)c(C(N)=O)cc23)C1 ; CC(C)Oc1cc2c(O[C@@H]3CCCN(C(=O)CC#N)C3)ncc(C#C[C@H]3C[C@@H](N(C)C)C3)c2cc1C(N)=O ; CC(C)Oc1cc2c(O[C@@H]3CCCN(C(=O)OC(C)(C)C)C3)ncc(-c3cnn(C4CC4)c3)c2cc1C(N)=O ; CC(C)Oc1cc2c(O[C@@H]3CCCNC3)ncc(-c3cnn(C4CC4)c3)c2cc1C(N)=O ; CC(C)Oc1cc2c(O[C@@H]3CCCNC3)ncc(-c3cnn(C4CCC4)c3)c2cc1C(N)=O]

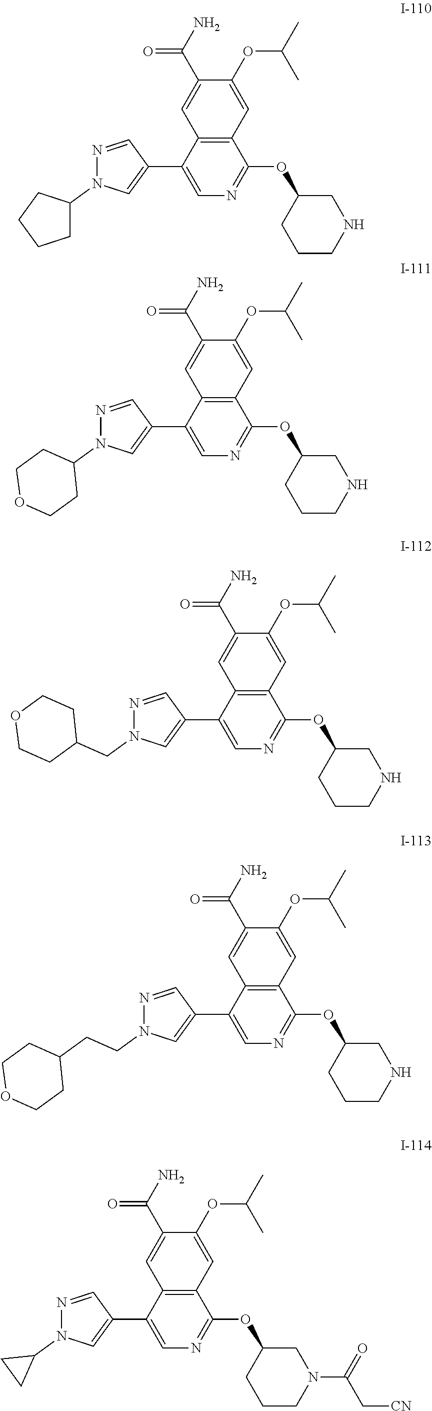

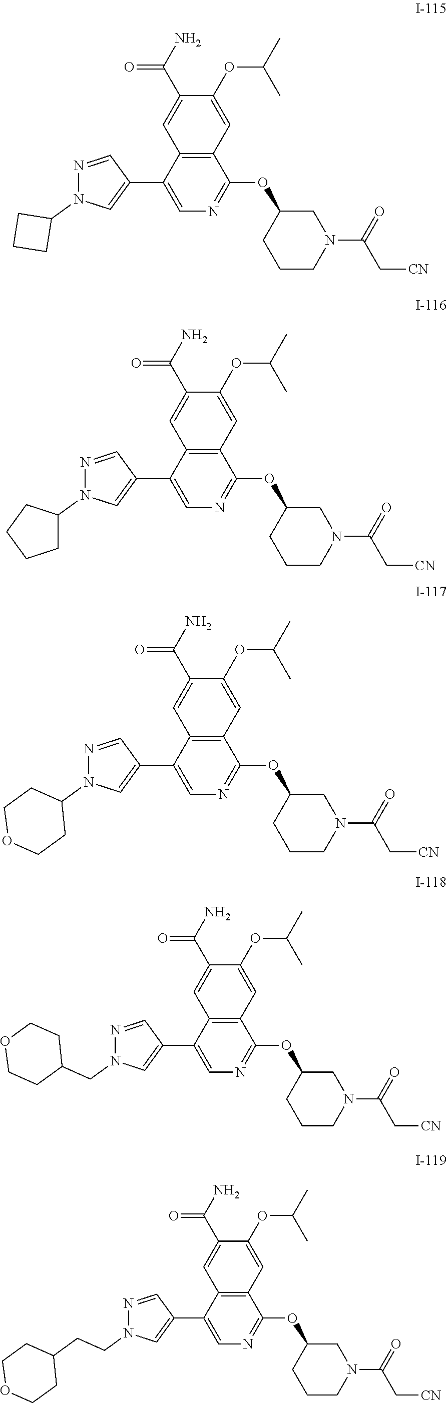 [SMILES: CC(C)Oc1cc2c(O[C@@H]3CCCN(C(=O)CC#N)C3)ncc(-c3cnn(C4CCC4)c3)c2cc1C(N)=O ; CC(C)Oc1cc2c(O[C@@H]3CCCN(C(=O)CC#N)C3)ncc(-c3cnn(C4CCCC4)c3)c2cc1C(N)=O ; CC(C)Oc1cc2c(O[C@@H]3CCCN(C(=O)CC#N)C3)ncc(-c3cnn(C4CCOCC4)c3)c2cc1C(N)=O ; CC(C)Oc1cc2c(O[C@@H]3CCCN(C(=O)CC#N)C3)ncc(-c3cnn(CC4CCOCC4)c3)c2cc1C(N)=O ; CC(C)Oc1cc2c(O[C@@H]3CCCN(C(=O)CC#N)C3)ncc(-c3cnn(CCC4CCOCC4)c3)c2cc1C(N)=O]

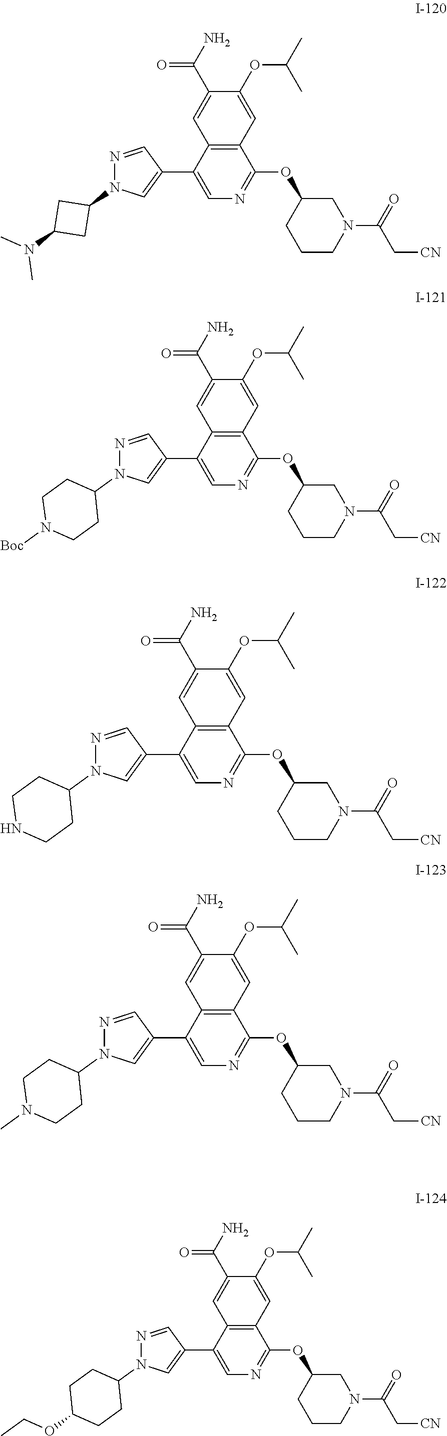

Some exemplary compounds according to Formulas I-IV include:
I:1 (S)-4-bromo-7-isopropoxy-1-((5-oxopyrrolidin-2-yl)methoxy)isoquinoline-6-carboxamide;
I-2: (S)-4-bromo-1-((4,4-difluoro-5-oxopyrrolidin-2-yl)methoxy)-7-isopropoxyisoquinoline-6-carboxamide;
I-3: (S)-4-(3-hydroxy-3-methylbut-1-yn-1-yl)-7-isopropoxy-1-((5-oxopyrrolidin-2-yl)methoxy)isoquinoline-6-carboxamide;
I-4: (S)-4-((3-hydroxyoxetan-3-yl)ethynyl)-7-isopropoxy-1-((5-oxopyrrolidin-2-yl)methoxy)isoquinoline-6-carboxamide;
I-5: (S)-4-(4-hydroxy-3,3-dimethylbut-1-yn-1-yl)-7-isopropoxy-1-((5-oxopyrrolidin-2-yl)methoxy)isoquinoline-6-carboxamide;
I-6: (S)-7-isopropoxy-1-((5-oxopyrrolidin-2-yl)methoxy)-4-((tetrahydro-2H-pyran-4-yl)ethynyl)isoquinoline-6-carboxamide;
I-7: (S)-7-isopropoxy-4-(3-morpholinoprop-1-yn-1-yl)-1-((5-oxopyrrolidin-2-yl)methoxy)isoquinoline-6-carboxamide;
I-8: (S)-7-isopropoxy-4-(4-morpholinobut-1-yn-1-yl)-1-((5-oxopyrrolidin-2-yl)methoxy)isoquinoline-6-carboxamide;
I-9: (S)-4-((1-hydroxycyclopentyl)ethynyl)-7-isopropoxy-1-((5-oxopyrrolidin-2-yl)methoxy)isoquinoline-6-carboxamide;
I-10: (S)-4-((4-hydroxytetrahydro-2H-pyran-4-yl)ethynyl)-7-isopropoxy-1-((5-oxopyrrolidin-2-yl)methoxy)isoquinoline-6-carboxamide;
I-11: (S)-7-isopropoxy-1-((5-oxopyrrolidin-2-yl)methoxy)-4-(3-(tetrahydro-2H-pyran-4-yl)prop-1-yn-1-yl)isoquinoline-6-carboxamide;
I-12: tert-butyl (S)-4-((6-carbamoyl-7-isopropoxy-1-((5-oxopyrrolidin-2-yl)methoxy)isoquinolin-4-yl)ethynyl)-4-hydroxypiperidine-1-carboxylate;
I-13: (S)-4-((4-hydroxypiperidin-4-yl)ethynyl)-7-isopropoxy-1-((5-oxopyrrolidin-2-yl)methoxy)isoquinoline-6-carboxamide;
I-14: (S)-4-((4-hydroxy-1-methylpiperidin-4-yl)ethynyl)-7-isopropoxy-1-((5-oxopyrrolidin-2-yl)methoxy)isoquinoline-6-carboxamide;
I-15: tert-butyl (S)-4-((6-carbamoyl-7-isopropoxy-1-((5-oxopyrrolidin-2-yl)methoxy)isoquinolin-4-yl)ethynyl)piperidine-1-carboxylate;
I-16: methyl (S)-4-((6-carbamoyl-7-isopropoxy-1-((5-oxopyrrolidin-2-yl)methoxy)isoquinolin-4-yl)ethynyl)piperidine-1-carboxylate;
I-17: (S)-7-isopropoxy-1-((5-oxopyrrolidin-2-yl)methoxy)-4-(piperidin-4-ylethynyl)isoquinoline-6-carboxamide;
I-18: (S)-7-isopropoxy-4-((1-methylpiperidin-4-yl)ethynyl)-1-((5-oxopyrrolidin-2-yl)methoxy)isoquinoline-6-carboxamide;
I-19: (S)-7-isopropoxy-4-((1-isopropylpiperidin-4-yl)ethynyl)-1-((5-oxopyrrolidin-2-yl)methoxy)isoquinoline-6-carboxamide;
I-20: 4-(((1r,4S)-4-ethoxycyclohexyl)ethynyl)-7-isopropoxy-1-(((S)-5-oxopyrrolidin-2-yl)methoxy)isoquinoline-6-carboxamide;
I-21: 4-(((1s,4R)-4-ethoxycyclohexyl)ethynyl)-7-isopropoxy-1-(((S)-5-oxopyrrolidin-2-yl)methoxy)isoquinoline-6-carboxamide;
I-22: 4-(((1r,4S)-4-hydroxy-4-methylcyclohexyl)ethynyl)-7-isopropoxy-1-(((S)-5-oxopyrrolidin-2-yl)methoxy)isoquinoline-6-carboxamide;
I-23: 4-(((1s,4R)-4-hydroxy-4-methylcyclohexyl)ethynyl)-7-isopropoxy-1-(((S)-5-oxopyrrolidin-2-yl)methoxy)isoquinoline-6-carboxamide;
I-24: tert-butyl (S)-3-((6-carbamoyl-7-isopropoxy-1-((5-oxopyrrolidin-2-yl)methoxy)isoquinolin-4-yl)ethynyl)azetidine-1-carboxylate;
I-25: (S)-4-(azetidin-3-ylethynyl)-7-isopropoxy-1-((5-oxopyrrolidin-2-yl)methoxy)isoquinoline-6-carboxamide;
I-26: (S)-7-isopropoxy-4-((1-methylazetidin-3-yl)ethynyl)-1-((5-oxopyrrolidin-2-yl)methoxy)isoquinoline-6-carboxamide;
I-27: (S)-7-isopropoxy-4-((1-isopropylazetidin-3-yl)ethynyl)-1-((5-oxopyrrolidin-2-yl)methoxy)isoquinoline-6-carboxamide;
I-28: methyl (S)-3-((6-carbamoyl-7-isopropoxy-1-((5-oxopyrrolidin-2-yl)methoxy)isoquinolin-4-yl)ethynyl)azetidine-1-carboxylate;
I-29: (S)-7-isopropoxy-4-((1-(methylcarbamoyl)azetidin-3-yl)ethynyl)-1-((5-oxopyrrolidin-2-yl)methoxy)isoquinoline-6-carboxamide;
I-30: (S)-4-((3,3-dimethoxycyclobutyl)ethynyl)-7-isopropoxy-1-((5-oxopyrrolidin-2-yl)methoxy)isoquinoline-6-carboxamide;
I-31: 4-(((1s,3R)-3-(dimethylamino)cyclobutyl)ethynyl)-7-isopropoxy-1-(((S)-5-oxopyrrolidin-2-yl)methoxy)isoquinoline-6-carboxamide;
I-32: (S)-4-(1-cyclopropyl-1H-pyrazol-4-yl)-7-isopropoxy-1-((5-oxopyrrolidin-2-yl)methoxy)isoquinoline-6-carboxamide;
I-33: (S)-4-(1-cyclobutyl-1H-pyrazol-4-yl)-7-isopropoxy-1-((5-oxopyrrolidin-2-yl)methoxy)isoquinoline-6-carboxamide;
I-34: (S)-4-(1-cyclopentyl-1H-pyrazol-4-yl)-7-isopropoxy-1-((5-oxopyrrolidin-2-yl)methoxy)isoquinoline-6-carboxamide;
I-35: (S)-7-isopropoxy-1-((5-oxopyrrolidin-2-yl)methoxy)-4-(1-(tetrahydro-2H-pyran-4-yl)-1H-pyrazol-4-yl)isoquinoline-6-carboxamide;
I-36: (S)-7-isopropoxy-1-((5-oxopyrrolidin-2-yl)methoxy)-4-(1-((tetrahydro-2H-pyran-4-yl)methyl)-1H-pyrazol-4-yl)isoquinoline-6-carboxamide;
I-37: (S)-7-isopropoxy-1-((5-oxopyrrolidin-2-yl)methoxy)-4-(1-(2-(tetrahydro-2H-pyran-4-yl)ethyl)-1H-pyrazol-4-yl)isoquinoline-6-carboxamide;
I-38: (S)-7-isopropoxy-4-(1-(2-morpholinoethyl)-1H-pyrazol-4-yl)-1-((5-oxopyrrolidin-2-yl)methoxy)isoquinoline-6-carboxamide;
I-39: 4-(1-((1r,4S)-4-ethoxycyclohexyl)-1H-pyrazol-4-yl)-7-isopropoxy-1-(((S)-5-oxopyrrolidin-2-yl)methoxy)isoquinoline-6-carboxamide;
I-40: 4-(1-((1r,4S)-4-hydroxy-4-methylcyclohexyl)-1H-pyrazol-4-yl)-7-isopropoxy-1-(((S)-5-oxopyrrolidin-2-yl)methoxy)isoquinoline-6-carboxamide;
I-41: 4-(1-((1s,4R)-4-hydroxy-4-methylcyclohexyl)-1H-pyrazol-4-yl)-7-isopropoxy-1-(((S)-5-oxopyrrolidin-2-yl)methoxy)isoquinoline-6-carboxamide;
I-42: 4-(1-((1s,3R)-3-ethoxycyclobutyl)-1H-pyrazol-4-yl)-7-isopropoxy-1-(((S)-5-oxopyrrolidin-2-yl)methoxy)isoquinoline-6-carboxamide;
I-43: 4-(1-((1s,3R)-3-(dimethylamino)cyclobutyl)-1H-pyrazol-4-yl)-7-isopropoxy-1-(((S)-5-oxopyrrolidin-2-yl)methoxy)isoquinoline-6-carboxamide;
I-44: tert-butyl (S)-4-(4-(6-carbamoyl-7-isopropoxy-1-((5-oxopyrrolidin-2-yl)methoxy)isoquinolin-4-yl)-1H-pyrazol-1-yl)piperidine-1-carboxylate;
I-45: (S)-7-isopropoxy-1-((5-oxopyrrolidin-2-yl)methoxy)-4-(1-(piperidin-4-yl)-1H-pyrazol-4-yl)isoquinoline-6-carboxamide;
I-46: (S)-7-isopropoxy-4-(1-(1-methylpiperidin-4-yl)-1H-pyrazol-4-yl)-1-((5-oxopyrrolidin-2-yl)methoxy)isoquinoline-6-carboxamide;
I-47: (S)-7-isopropoxy-1-((5-oxopyrrolidin-2-yl)methoxy)-4-(1H-pyrazol-4-yl)isoquinoline-6-carboxamide;
I-48: (S)-1-((4,4-difluoro-5-oxopyrrolidin-2-yl)methoxy)-4-((1-hydroxycyclopentyl)ethynyl)-7-isopropoxyisoquinoline-6-carboxamide;
I-49: (S)-1-((4,4-difluoro-5-oxopyrrolidin-2-yl)methoxy)-7-isopropoxy-4-((tetrahydro-2H-pyran-4-yl)ethynyl)isoquinoline-6-carboxamide;
I-50: (S)-1-((4,4-difluoro-5-oxopyrrolidin-2-yl)methoxy)-7-isopropoxy-4-(3-(tetrahydro-2H-pyran-4-yl)prop-1-yn-1-yl)isoquinoline-6-carboxamide;
I-51: (S)-1-((4,4-difluoro-5-oxopyrrolidin-2-yl)methoxy)-4-((4-hydroxytetrahydro-2H-pyran-4-yl)ethynyl)-7-isopropoxyisoquinoline-6-carboxamide;
I-52: tert-butyl (S)-4-((6-carbamoyl-1-((4,4-difluoro-5-oxopyrrolidin-2-yl)methoxy)-7-isopropoxyisoquinolin-4-yl)ethynyl)-4-hydroxypiperidine-1-carboxylate;
I-53: (S)-1-((4,4-difluoro-5-oxopyrrolidin-2-yl)methoxy)-4-((4-hydroxypiperidin-4-yl)ethynyl)-7-isopropoxyisoquinoline-6-carboxamide;
I-54: tert-butyl (S)-4-((6-carbamoyl-1-((4,4-difluoro-5-oxopyrrolidin-2-yl)methoxy)-7-isopropoxyisoquinolin-4-yl)ethynyl)piperidine-1-carboxylate;
I-55: (S)-1-((4,4-difluoro-5-oxopyrrolidin-2-yl)methoxy)-7-isopropoxy-4-(piperidin-4-ylethynyl)isoquinoline-6-carboxamide;
I-56: (S)-1-((4,4-difluoro-5-oxopyrrolidin-2-yl)methoxy)-7-isopropoxy-4-((1-methylpiperidin-4-yl)ethynyl)isoquinoline-6-carboxamide;
I-57: (S)-1-((4,4-difluoro-5-oxopyrrolidin-2-yl)methoxy)-4-((4-hydroxy-1-methylpiperidin-4-yl)ethynyl)-7-isopropoxyisoquinoline-6-carboxamide;
I-58: 1-(((S)-4,4-difluoro-5-oxopyrrolidin-2-yl)methoxy)-4-(((1r,4S)-4-ethoxycyclohexyl)ethynyl)-7-isopropoxyisoquinoline-6-carboxamide;
I-59: 1-(((S)-4,4-difluoro-5-oxopyrrolidin-2-yl)methoxy)-4-(((1s,4R)-4-ethoxycyclohexyl)ethynyl)-7-isopropoxyisoquinoline-6-carboxamide;
I-60: 1-(((S)-4,4-difluoro-5-oxopyrrolidin-2-yl)methoxy)-4-(((1r,4S)-4-hydroxy-4-methylcyclohexyl)ethynyl)-7-isopropoxyisoquinoline-6-carboxamide;
I-61: 1-(((S)-4,4-difluoro-5-oxopyrrolidin-2-yl)methoxy)-4-(((1s,4R)-4-hydroxy-4-methylcyclohexyl)ethynyl)-7-isopropoxyisoquinoline-6-carboxamide;
I-62: tert-butyl (S)-3-((6-carbamoyl-1-((4,4-difluoro-5-oxopyrrolidin-2-yl)methoxy)-7-isopropoxyisoquinolin-4-yl)ethynyl)azetidine-1-carboxylate;
I-63: (S)-4-(azetidin-3-ylethynyl)-1-((4,4-difluoro-5-oxopyrrolidin-2-yl)methoxy)-7-isopropoxyisoquinoline-6-carboxamide;
I-64: (S)-1-((4,4-difluoro-5-oxopyrrolidin-2-yl)methoxy)-7-isopropoxy-4-((1-methylazetidin-3-yl)ethynyl)isoquinoline-6-carboxamide;
I-65: (S)-1-((4,4-difluoro-5-oxopyrrolidin-2-yl)methoxy)-4-((3,3-dimethoxycyclobutyl)ethynyl)-7-isopropoxyisoquinoline-6-carboxamide;
I-66: 1-(((S)-4,4-difluoro-5-oxopyrrolidin-2-yl)methoxy)-4-(((1s,3R)-3-(dimethylamino)cyclobutyl)ethynyl)-7-isopropoxyisoquinoline-6-carboxamide;
I-67: (S)-4-(1-cyclopropyl-1H-pyrazol-4-yl)-1-((4,4-difluoro-5-oxopyrrolidin-2-yl)methoxy)-7-isopropoxyisoquinoline-6-carboxamide;
I-68: (S)-4-(1-cyclobutyl-1H-pyrazol-4-yl)-1-((4,4-difluoro-5-oxopyrrolidin-2-yl)methoxy)-7-isopropoxyisoquinoline-6-carboxamide;
I-69: (S)-4-(1-cyclopentyl-1H-pyrazol-4-yl)-1-((4,4-difluoro-5-oxopyrrolidin-2-yl)methoxy)-7-isopropoxyisoquinoline-6-carboxamide;
I-70: (S)-1-((4,4-difluoro-5-oxopyrrolidin-2-yl)methoxy)-7-isopropoxy-4-(1-(tetrahydro-2H-pyran-4-yl)-1H-pyrazol-4-yl)isoquinoline-6-carboxamide;
I-71: (S)-1-((4,4-difluoro-5-oxopyrrolidin-2-yl)methoxy)-7-isopropoxy-4-(1-((tetrahydro-2H-pyran-4-yl)methyl)-1H-pyrazol-4-yl)isoquinoline-6-carboxamide;
I-72: (S)-1-((4,4-difluoro-5-oxopyrrolidin-2-yl)methoxy)-7-isopropoxy-4-(1-(2-(tetrahydro-2H-pyran-4-yl)ethyl)-1H-pyrazol-4-yl)isoquinoline-6-carboxamide;
I-73: 1-(((S)-4,4-difluoro-5-oxopyrrolidin-2-yl)methoxy)-4-(1-((1s,3R)-3-(dimethylamino)cyclobutyl)-1H-pyrazol-4-yl)-7-isopropoxyisoquinoline-6-carboxamide;
I-74: tert-butyl (S)-4-(4-(6-carbamoyl-1-((4,4-difluoro-5-oxopyrrolidin-2-yl)methoxy)-7-isopropoxyisoquinolin-4-yl)-1H-pyrazol-1-yl)piperidine-1-carboxylate;
I-75: (S)-1-((4,4-difluoro-5-oxopyrrolidin-2-yl)methoxy)-7-isopropoxy-4-(1-(piperidin-4-yl)-1H-pyrazol-4-yl)isoquinoline-6-carboxamide;
I-76: (S)-1-((4,4-difluoro-5-oxopyrrolidin-2-yl)methoxy)-7-isopropoxy-4-(1-(1-methylpiperidin-4-yl)-1H-pyrazol-4-yl)isoquinoline-6-carboxamide;
I-77: 1-(((S)-4,4-difluoro-5-oxopyrrolidin-2-yl)methoxy)-4-(1-((1r,4S)-4-ethoxycyclohexyl)-1H-pyrazol-4-yl)-7-isopropoxyisoquinoline-6-carboxamide;
I-78: 1-(((S)-4,4-difluoro-5-oxopyrrolidin-2-yl)methoxy)-4-(1-((1r,4S)-4-hydroxy-4-methylcyclohexyl)-1H-pyrazol-4-yl)-7-isopropoxyisoquinoline-6-carboxamide;
I-79: 1-(((2S,3S,4S)-3-ethyl-4-fluoro-5-oxopyrrolidin-2-yl)methoxy)-7-isopropoxy-4-((tetrahydro-2H-pyran-4-yl)ethynyl)isoquinoline-6-carboxamide;
I-80: 4-(((1r,4S)-4-ethoxycyclohexyl)ethynyl)-1-(((2S,3S,4S)-3-ethyl-4-fluoro-5-oxopyrrolidin-2-yl)methoxy)-7-isopropoxyisoquinoline-6-carboxamide;
I-81: 4-(((1s,4R)-4-ethoxycyclohexyl)ethynyl)-1-(((2S,3S,4S)-3-ethyl-4-fluoro-5-oxopyrrolidin-2-yl)methoxy)-7-isopropoxyisoquinoline-6-carboxamide;
I-82: 1-(((2S,3S,4S)-3-ethyl-4-fluoro-5-oxopyrrolidin-2-yl)methoxy)-7-methoxy-4-((tetrahydro-2H-pyran-4-yl)ethynyl)isoquinoline-6-carboxamide;
I-83: 4-(((1r,4S)-4-ethoxycyclohexyl)ethynyl)-1-(((2S,3S,4S)-3-ethyl-4-fluoro-5-oxopyrrolidin-2-yl)methoxy)-7-methoxyisoquinoline-6-carboxamide;
I-84: 4-(((1s,4R)-4-ethoxycyclohexyl)ethynyl)-1-(((2S,3S,4S)-3-ethyl-4-fluoro-5-oxopyrrolidin-2-yl)methoxy)-7-methoxyisoquinoline-6-carboxamide;
I-85: (R)-4-((1-hydroxycyclopentyl)ethynyl)-7-isopropoxy-1-(piperidin-3-yloxy)isoquinoline-6-carboxamide;
I-86: (R)-7-isopropoxy-1-(piperidin-3-yloxy)-4-((tetrahydro-2H-pyran-4-yl)ethynyl)isoquinoline-6-carboxamide;
I-87: (R)-7-isopropoxy-1-(piperidin-3-yloxy)-4-(3-(tetrahydro-2H-pyran-4-yl)prop-1-yn-1-yl)isoquinoline-6-carboxamide;
I-88: (R)-1-((1-(2-cyanoacetyl)piperidin-3-yl)oxy)-4-((1-hydroxycyclopentyl)ethynyl)-7-isopropoxyisoquinoline-6-carboxamide;
I-89: (R)-1-((1-(2-cyanoacetyl)piperidin-3-yl)oxy)-7-isopropoxy-4-((tetrahydro-2H-pyran-4-yl)ethynyl)isoquinoline-6-carboxamide;
I-90: (R)-1-((1-(2-cyanoacetyl)piperidin-3-yl)oxy)-7-isopropoxy-4-(3-(tetrahydro-2H-pyran-4-yl)prop-1-yn-1-yl)isoquinoline-6-carboxamide;
I-91: (R)-1-((1-(2-cyanoacetyl)piperidin-3-yl)oxy)-4-((4-hydroxytetrahydro-2H-pyran-4-yl)ethynyl)-7-isopropoxyisoquinoline-6-carboxamide;
I-92: tert-butyl (R)-4-((6-carbamoyl-1-((1-(2-cyanoacetyl)piperidin-3-yl)oxy)-7-isopropoxyisoquinolin-4-yl)ethynyl)-4-hydroxypiperidine-1-carboxylate;
I-93: (R)-1-((1-(2-cyanoacetyl)piperidin-3-yl)oxy)-4-((4-hydroxypiperidin-4-yl)ethynyl)-7-isopropoxyisoquinoline-6-carboxamide;
I-94: (R)-1-((1-(2-cyanoacetyl)piperidin-3-yl)oxy)-4-((4-hydroxy-1-methylpiperidin-4-yl)ethynyl)-7-isopropoxyisoquinoline-6-carboxamide;
I-95: tert-butyl (R)-4-((6-carbamoyl-1-((1-(2-cyanoacetyl)piperidin-3-yl)oxy)-7-isopropoxyisoquinolin-4-yl)ethynyl)piperidine-1-carboxylate;
I-96: (R)-1-((1-(2-cyanoacetyl)piperidin-3-yl)oxy)-7-isopropoxy-4-(piperidin-4-ylethynyl)isoquinoline-6-carboxamide;
I-97: (R)-1-((1-(2-cyanoacetyl)piperidin-3-yl)oxy)-7-isopropoxy-4-((1-methylpiperidin-4-yl)ethynyl)isoquinoline-6-carboxamide;
I-98: 1-(((R)-1-(2-cyanoacetyl)piperidin-3-yl)oxy)-4-(((1r,4R)-4-ethoxycyclohexyl)ethynyl)-7-isopropoxyisoquinoline-6-carboxamide;
I-99: 1-(((R)-1-(2-cyanoacetyl)piperidin-3-yl)oxy)-4-(((1s,4S)-4-ethoxycyclohexyl)ethynyl)-7-isopropoxyisoquinoline-6-carboxamide;
I-100: 1-(((R)-1-(2-cyanoacetyl)piperidin-3-yl)oxy)-4-(((1r,4R)-4-hydroxy-4-methylcyclohexyl)ethynyl)-7-isopropoxyisoquinoline-6-carboxamide;
I-101: 1-(((R)-1-(2-cyanoacetyl)piperidin-3-yl)oxy)-4-(((1s,4S)-4-hydroxy-4-methylcyclohexyl)ethynyl)-7-isopropoxyisoquinoline-6-carboxamide;
I-102: tert-butyl (R)-3-((6-carbamoyl-1-((1-(2-cyanoacetyl)piperidin-3-yl)oxy)-7-isopropoxyisoquinolin-4-yl)ethynyl)azetidine-1-carboxylate;
I-103: (R)-4-(azetidin-3-ylethynyl)-1-((1-(2-cyanoacetyl)piperidin-3-yl)oxy)-7-isopropoxyisoquinoline-6-carboxamide;
I-104: (R)-1-((1-(2-cyanoacetyl)piperidin-3-yl)oxy)-7-isopropoxy-4-((1-methylazetidin-3-yl)ethynyl)isoquinoline-6-carboxamide;
I-105: (R)-1-((1-(2-cyanoacetyl)piperidin-3-yl)oxy)-4-((3,3-dimethoxycyclobutyl)ethynyl)-7-isopropoxyisoquinoline-6-carboxamide;
I-106: 1-(((R)-1-(2-cyanoacetyl)piperidin-3-yl)oxy)-4-(((1s,3S)-3-(dimethylamino)cyclobutyl)ethynyl)-7-isopropoxyisoquinoline-6-carboxamide;
I-107: tert-butyl (R)-3-((6-carbamoyl-4-(1-cyclopropyl-1H-pyrazol-4-yl)-7-isopropoxyisoquinolin-1-yl)oxy)piperidine-1-carboxylate;
I-108: (R)-4-(1-cyclopropyl-1H-pyrazol-4-yl)-7-isopropoxy-1-(piperidin-3-yloxy)isoquinoline-6-carboxamide;
I-109: (R)-4-(1-cyclobutyl-1H-pyrazol-4-yl)-7-isopropoxy-1-(piperidin-3-yloxy)isoquinoline-6-carboxamide;
I-110: (R)-4-(1-cyclopentyl-1H-pyrazol-4-yl)-7-isopropoxy-1-(piperidin-3-yloxy)isoquinoline-6-carboxamide;
I-111: (R)-7-isopropoxy-1-(piperidin-3-yloxy)-4-(1-(tetrahydro-2H-pyran-4-yl)-1H-pyrazol-4-yl)isoquinoline-6-carboxamide;
I-112: (R)-7-isopropoxy-1-(piperidin-3-yloxy)-4-(1-((tetrahydro-2H-pyran-4-yl)methyl)-1H-pyrazol-4-yl)isoquinoline-6-carboxamide;
I-113: (R)-7-isopropoxy-1-(piperidin-3-yloxy)-4-(1-(2-(tetrahydro-2H-pyran-4-yl)ethyl)-1H-pyrazol-4-yl)isoquinoline-6-carboxamide;
I-114: (R)-1-((1-(2-cyanoacetyl)piperidin-3-yl)oxy)-4-(1-cyclopropyl-1H-pyrazol-4-yl)-7-isopropoxyisoquinoline-6-carboxamide;
I-115: (R)-1-((1-(2-cyanoacetyl)piperidin-3-yl)oxy)-4-(1-cyclobutyl-1H-pyrazol-4-yl)-7-isopropoxyisoquinoline-6-carboxamide;
I-116: (R)-1-((1-(2-cyanoacetyl)piperidin-3-yl)oxy)-4-(1-cyclopentyl-1H-pyrazol-4-yl)-7-isopropoxyisoquinoline-6-carboxamide;
I-117: (R)-1-((1-(2-cyanoacetyl)piperidin-3-yl)oxy)-7-isopropoxy-4-(1-(tetrahydro-2H-pyran-4-yl)-1H-pyrazol-4-yl)isoquinoline-6-carboxamide;
I-118: (R)-1-((1-(2-cyanoacetyl)piperidin-3-yl)oxy)-7-isopropoxy-4-(1-((tetrahydro-2H-pyran-4-yl)methyl)-1H-pyrazol-4-yl)isoquinoline-6-carboxamide;
I-119: (R)-1-((1-(2-cyanoacetyl)piperidin-3-yl)oxy)-7-isopropoxy-4-(1-(2-(tetrahydro-2H-pyran-4-yl)ethyl)-1H-pyrazol-4-yl)isoquinoline-6-carboxamide;
I-120: 1-(((R)-1-(2-cyanoacetyl)piperidin-3-yl)oxy)-4-(1-((1s,3S)-3-(dimethylamino)cyclobutyl)-1H-pyrazol-4-yl)-7-isopropoxyisoquinoline-6-carboxamide;
I-121: tert-butyl (R)-4-(4-(6-carbamoyl-1-((1-(2-cyanoacetyl)piperidin-3-yl)oxy)-7-isopropoxyisoquinolin-4-yl)-1H-pyrazol-1-yl)piperidine-1-carboxylate;
I-122: (R)-1-((1-(2-cyanoacetyl)piperidin-3-yl)oxy)-7-isopropoxy-4-(1-(piperidin-4-yl)-1H-pyrazol-4-yl)isoquinoline-6-carboxamide;
I-123: (R)-1-((1-(2-cyanoacetyl)piperidin-3-yl)oxy)-7-isopropoxy-4-(1-(1-methylpiperidin-4-yl)-1H-pyrazol-4-yl)isoquinoline-6-carboxamide; or
I-124: 1-(((R)-1-(2-cyanoacetyl)piperidin-3-yl)oxy)-4-(1-((1r,4R)-4-ethoxycyclohexyl)-1H-pyrazol-4-yl)-7-isopropoxyisoquinoline-6-carboxamide.

### III. Synthesis

Disclosed compounds can be prepared as exemplified below, and as will be understood by a person of ordinary skill in the art in organic synthesis. An exemplary synthesis may include the following 1^{st} reaction step according to Scheme 1:

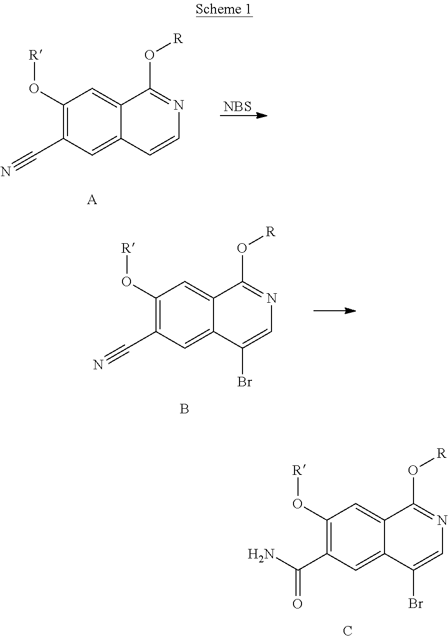

Compound A is treated with N-bromosuccinimide (NBS) to form compound B at a temperature suitable to facilitate a reaction, such as a temperature from zero to 25° C. The reaction is performed in a suitable solvent, such as an aprotic solvent, for example, DMF.

Compound B then is treated with an oxidizing agent, such as hydrogen peroxide, in the presence of a suitable base, to form compound C. Suitable bases include, but are not limited to, carbonate bases, such as potassium carbonate, cesium carbonate, or sodium carbonate. The reaction typically proceeds at room temperature, and is performed in a suitable solvent, such as an aprotic solvent that is stable in the presence of the oxidizing agent, such as DMSO.

A 2^{nd} reaction step in the exemplary synthesis is provided below according to Scheme 2:

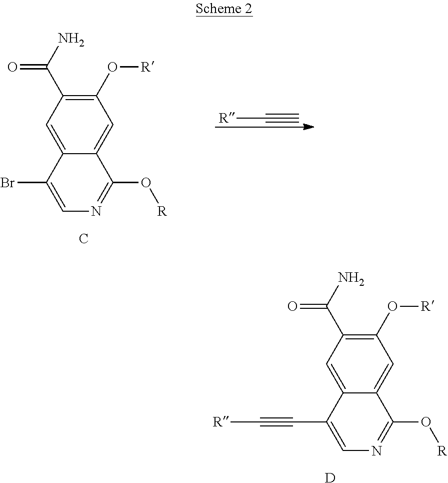

Compound C is treated with an alkyne compound in the presence of a suitable catalyst to form compound D. The catalyst may be any catalyst suitable to facilitate the reaction, such as a palladium catalyst, for example Pd(PPh₃)₄. The reaction may be performed in the presence of a suitable base, such as an organic base, for example, triethylamine, trimethylamine, or diisopropylethylamine. Additionally, the reaction may proceed in the presence of a halide compound, such as an iodide compound, for example CuI. The reaction is performed in a suitable solvent, such as an aprotic solvent, for example DMF, THF, Acetonitrile, or a combination thereof, and may be performed at a temperature suitable to facilitate the reaction, such as from 50° C. to 120° C. or more.

Alternatively, a 2^{nd} step in the exemplary synthesis may proceed according to Scheme 3.

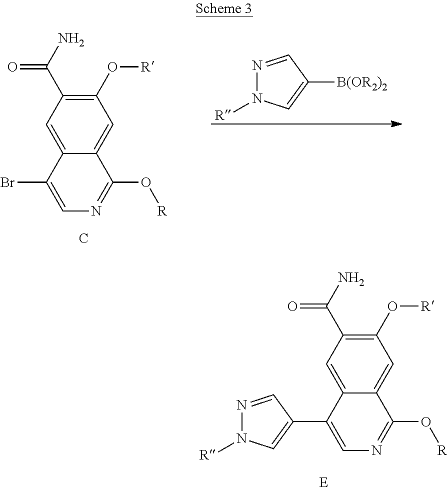

Compound C is treated with a boronic ester or acid to form compound E. The reaction proceeds in the presence of a catalyst, typically a palladium catalyst such as Pd(PPh₃)₄, and in the presence of a base, such as an organic base, for example, triethylamine, or an inorganic base, such as a carbonate base, for example potassium carbonate, cesium carbonate, or sodium carbonate. The reaction is performed at a temperature suitable to facilitate the reaction, such as from 50° C. to 120° C. or more.

A 3^{rd} step in the exemplary synthesis may proceed according to Scheme 4.

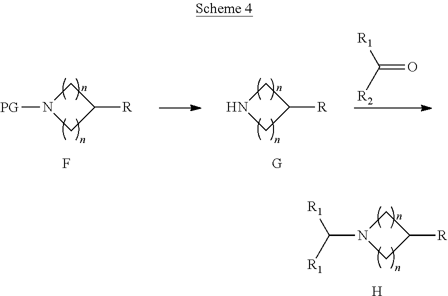

Protected amine compound F is treated with a suitable reagent to remove the protecting group PG and form amine compound G. Suitable protecting groups and the reagents used to remove them are known to persons of ordinary skill in the art, and additional information can be found in Peter G. M. Wuts and Theodora W. Greene, Greene's Protective Groups in Organic Synthesis, 4th Edition, (2006). In a disclosed example, the protecting group is BOC and trifluoroacetic acid is used to remove the BOC group. The reaction is performed in a suitable solvent, such as a chlorinated solvent, for example, chloroform or dichloromethane.

Amine compound G then is treated with a carbonyl compound to form compound H. The reaction proceeds in the presence of a suitable reducing agent, such as a borohydride reagent, for example, sodium triacetoxyborohydride, or sodium borohydride. The reaction is performed in a suitable solvent, such as an alcohol, for example, methanol, ethanol or propanol. And the reaction is typically performed at a temperature of from 10° C. to 30° C. or more, such as room temperature.

An additional reaction step in the exemplary synthesis may proceed according to Scheme 5.

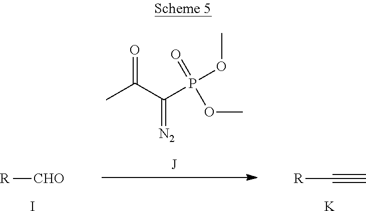

Aldehyde compound I is treated with compound J to form alkyne compound K. The reaction is performed in a suitable solvent, such as an alcohol, for example, methanol, ethanol or propanol, and typically proceeds in the presence of a suitable base. Bases suitable to facilitate the reaction include, but are not limited to, inorganic bases such as carbonate bases, for example, potassium carbonate, cesium carbonate, or sodium carbonate.

An additional or alternative step in the exemplary synthesis may proceed according to Scheme 6.

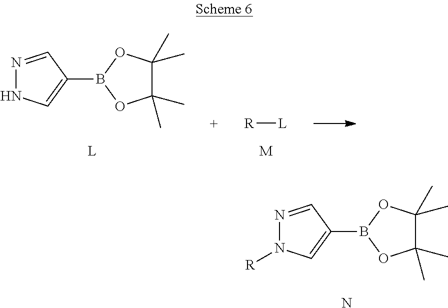

Boronic ester compound L is treated with compound M to form boronic ester compound N. The reaction proceeds in the presence of a suitable base and in a suitable solvent. Bases suitable to facilitate the reaction include, but are not limited to, inorganic bases such as carbonate bases, for example, potassium carbonate, cesium carbonate, or sodium carbonate. Suitable solvents include, but are not limited to, aprotic solvents, such as DMF, THF, acetonitrile, or combinations thereof. The reaction proceeds at a temperature suitable to facilitate the reaction, such as a temperature of from 25° C. to 100° C.

Another additional or alternative step in the exemplary synthesis may proceed according to Scheme 7.

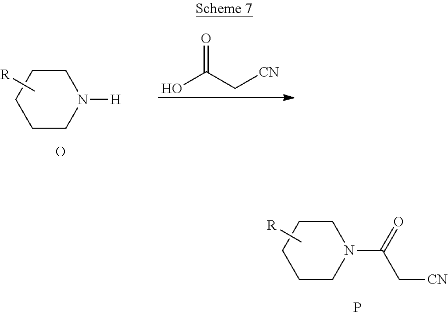

Amine compound O is treated with cyanoacetic acid to form compound P. The reaction proceeds in the presence of a suitable coupling agent, such as HATU, DCC, or EDCI, and a suitable base. Suitable bases include, but are not limited to, organic bases, such as N-ethyl-N-isopropylpropan-2-amine, diisopropylethylamine, or triethylamine. The reaction proceeds at a temperature suitable to facilitate the reaction, such as room temperature. A person of ordinary skill in the art understands that compound P could alternatively be prepared from compound O using any activated analog of cyanoacetic acid, for example, cyanoacetyl chloride.

### IV. Combinations of Therapeutic Agents

The compounds of the present disclosure may be used alone, in combination with one another, in separate pharmaceutical compositions, together in a single composition, or as an adjunct to, or in combination with, other established therapies. The compound or compounds may be administered once, or more likely plural administrations. In another aspect, the compounds of the present disclosure may be used in combination with other therapeutic agents useful for the disorder or condition being treated. These compounds and/or agents may be administered simultaneously, sequentially in any order, by the same route of administration, or by a different route. For sequential administration, the compound(s) and/or agent(s) may be administered such that an effective time period of at least one compound and/or agent overlaps with an effective time period of at least one other compound and/or agent. In an exemplary embodiment of a combination comprising four components, the effective time period of the first component administered may overlap with the effective time periods of the second, third and fourth components, but the effective time periods of the second, third and fourth components independently may or may not overlap with one another. In another exemplary embodiment of a combination comprising four components, the effective time period of the first component administered overlaps with the effective time period of the second component, but not that of the third or fourth; the effective time period of the second component overlaps with those of the first and third components; and the effective time period of the fourth component overlaps with that of the third component only. In some embodiments, the effective time periods of all compounds and/or agents overlap with each other.

In some embodiments, disclosed compounds are administered with another therapeutic agent, such as an analgesic, an antibiotic, an anticoagulant, an antibody, an anti-inflammatory agent, an immunosuppressant, a guanylate cyclase-C agonist, an intestinal secretagogue, an antiviral, anticancer, antifungal, or a combination thereof. In certain embodiments, the second therapeutic is an anti-inflammatory agent, an immunosuppressant and/or may be a steroid.

The anti-inflammatory agent may be a steroid or a nonsteroidal anti-inflammatory agent. In certain embodiments, the nonsteroidal anti-inflammatory agent is selected from aminosalicylates, cyclooxygenase inhibitors, diclofenac, etodolac, famotidine, fenoprofen, flurbiprofen, ketoprofen, ketorolac, ibuprofen, indomethacin, meclofenamate, mefenamic acid, meloxicam, nambumetone, naproxen, oxaprozin, piroxicam, salsalate, sulindac, tolmetin, or a combination thereof. In some embodiments, the immunosuppressant is mercaptopurine, a corticosteroid, an alkylating agent, a calcineurin inhibitor, an inosine monophosphate dehydrogenase inhibitor, antilymphocyte globulin, antithymocyte globulin, an anti-T-cell antibody, or a combination thereof. In one embodiment, the antibody is infliximab.

In some embodiments, the present compounds may be used with anti-cancer or cytotoxic agents. Various classes of anti-cancer and anti-neoplastic compounds include, but are not limited to, alkylating agents, antimetabolites, BCL-2 inhibitors, vinca alkyloids, taxanes, antibiotics, enzymes, cytokines, platinum coordination complexes, proteasome inhibitors, substituted ureas, kinase inhibitors, hormones and hormone antagonists, and hypomethylating agents, for example DNMT inhibitors, such as azacitidine and decitabine. Exemplary alkylating agents include, without limitation, mechlorothamine, cyclophosphamide, ifosfamide, melphalan, chlorambucil, ethyleneimines, methylmelamines, alkyl sulfonates (e.g., busulfan), and carmustine. Exemplary antimetabolites include, by way of example and not limitation, folic acid analog methotrexate; pyrmidine analog fluorouracil, cytosine arbinoside; purine analogs mercaptopurine, thioguanine, and azathioprine. Exemplary vinca alkyloids include, by way of example and not limitation, vinblastine, vincristine, paclitaxel, and colchicine. Exemplary antibiotics include, by way of example and not limitation, actinomycin D, daunorubicin, and bleomycin. An exemplary enzyme effective as an anti-neoplastic agent includes L-asparaginase. Exemplary coordination compounds include, by way of example and not limitation, cisplatin and carboplatin. Exemplary hormones and hormone related compounds include, by way of example and not limitation, adrenocorticosteroids prednisone and dexamethasone; aromatase inhibitors amino glutethimide, formestane, and anastrozole; progestin compounds hydroxyprogesterone caproate, medroxyprogesterone; and anti-estrogen compound tamoxifen.

These and other useful anti-cancer compounds are described in Merck Index, 13th Ed. (O'Neil M. J. et al., ed.) Merck Publishing Group (2001) and Goodman and Gilman's The Pharmacological Basis of Therapeutics, 12th Edition, Brunton L. L. ed., Chapters 60-63, McGraw Hill, (2011), both of which are incorporated by reference herein.

Among the CTLA 4 antibodies that can be used in combination with the presently disclosed inhibitors is ipilimumab, marketed as YERVOY® by Bristol-Myers Squibb.

Other chemotherapeutic agents for combination include immunooncology agents, such as checkpoint pathway inhibitors, for example, PD-1 inhibitors, such as nivolumab and lambrolizumab, and PD-L1 inhibitors, such as pembrolizumab, MEDI-4736 and MPDL3280A/RG7446. Additional checkpoint inhibitors for combination with the compounds disclosed herein include, Anti-LAG-3 agents, such as BMS-986016 (MDX-1408).

Further chemotherapeutic agents for combination with the presently disclosed inhibitors include Anti-SLAMF7 agents, such as the humanized monoclonal antibody elotuzumab (BMS-901608), anti-KIR agents, such as the anti-KIR monoclonal antibody lirilumab (BMS-986015), and anti-CD137 agents, such as the fully human monoclonal antibody urelumab (BMS-663513).

Additional anti-proliferative compounds useful in combination with the compounds of the present disclosure include, by way of example and not limitation, antibodies directed against growth factor receptors (e.g., anti-Her2); and cytokines such as interferon-α and interferon-γ, interleukin-2, and GM-CSF.

Additional chemotherapeutic agents useful in combination with the present compounds include proteasome inhibitors, such as bortezomib, carfilzomib, marizomib and the like.

Examples of kinase inhibitors that are useful in combination with the presently disclosed compounds, particularly in treating malignancies include: Btk inhibitors, such as ibrutinib; CDK inhibitors, such as palbociclib; EGFR inhibitors, such as afatinib, erlotinib, gefitinib, lapatinib, osimertinib and vandetinib; Mek inhibitors, such as trametinib; Raf inhibitors, such as dabrafenib, sorafenib and vemurafenib; VEGFR inhibitors, such as axitinib, lenvatinib, nintedanib, pazopanib; BCR-Abl inhibitors, such as bosutinib, dasatinib, imatinib and nilotinib; Syk inhibitors, such as fostamatinib; and JAK inhibitors, such as ruxolitinib.

In other embodiments, the second therapeutic agent may be selected from any of the following:
analgesics-morphine, fentanyl, hydromorphone, oxycodone, codeine, acetaminophen, hydrocodone, buprenorphine, tramadol, venlafaxine, flupirtine, meperidine, pentazocine, dextromoramide, dipipanone;
antibiotics-aminoglycosides (e.g., amikacin, gentamicin, kanamycin, neomycin, netilmicin, tobramycin, and paromycin), carbapenems (e.g., ertapenem, doripenem, imipenem, cilastatin, and meropenem), cephalosporins (e.g., cefadroxil, cefazolin, cefalotin, cephalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, and cefobiprole), glycopeptides (e.g., teicoplanin, vancomycin, and telavancin), lincosamides (e.g., clindamycin and incomysin), lipopeptides (e.g., daptomycin), macrolides (azithromycin, clarithromycin, dirithromycin, erythromycin, roxithromycin, troleandomycin, telithromycin, and spectinomycin), monobactams (e.g., aztreonam), nitrofurans (e.g., furazolidone and nitrofurantoin), penicillins (e.g., amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, and ticarcillin), penicillin combinations (e.g., amoxicillin/clavulanate, ampicillin/sulbactam, piperacillin/tazobactam, and ticarcillin/clavulanate), polypeptides (e.g., bacitracin, colistin, and polymyxin B), quinolones (e.g., ciprofloxacin, enoxacin, gatifloxacin, levofloxacin, lomefloxacin, moxifloxacin, nalidixic acid, norfloxacin, ofloxacin, trovafloxacin, grepafloxacin, sparfloxacin, and temafloxacin), sulfonamides (e.g., mafenide, sulfonamidochrysoidine, sulfacetamide, sulfadiazine, silver sulfadiazine, sulfamethizole, sulfamethoxazole, sulfanilimide, sulfasalazine, sulfisoxazole, trimethoprim, and trimethoprim-sulfamethoxaxzole), tetracyclines (e.g., demeclocycline, doxycycline, minocycline, oxytetracycline, and tetracycline), antimycobacterial compounds (e.g., clofazimine, dapsone, capreomycin, cycloserine, ethambutol, ethionamide, isoniazid, pyrazinamide, rifampicin (rifampin), rifabutin, rifapentine, and streptomycin), and others, such as arsphenamine, chloramphenicol, fosfomycin, fusidic acid, linezolid, metronidazole, mupirocin, platensimycin, quinuprisin/dalfopristin, rifaximin, thiamphenicol, tigecycline, and timidazole;
antibodies-anti-TNF-α antibodies, e.g., infliximab (Remicade™), adalimumab, golimumab, certolizumab; anti-B cell antibodies, e.g., rituximab; anti-IL-6 antibodies, e.g., tocilizumab; anti-IL-1 antibodies, e.g., anakinra; anti PD-1 and/or anti-PD-L1 antibodies, e.g. nivolumab, pembrolizumab, pidilizumab, BMS-936559, MPDL3280A, AMP-224, MEDI4736; ixekizumab, brodalumab, ofatumumab, sirukumab, clenoliximab, clazakiumab, fezakinumab, fletikumab, mavrilimumab, ocrelizumab, sarilumab, secukinumab, toralizumab, zanolimumab;
anticoagulants-warfarin (Coumadin™), acenocoumarol, phenprocoumon, atromentin, phenindione, heparin, fondaparinux, idraparinux, rivaroxaban, apixaban, hirudin, lepirudin, bivalirudin, argatrobam, dabigatran, ximelagatran, batroxobin, hementin;
anti-inflammatory agents-steroids, e.g., budesonide, nonsteroidal anti-inflammatory agents, e.g., aminosalicylates (e.g., sulfasalazine, mesalamine, olsalazine, and balsalazide), cyclooxygenase inhibitors (COX-2 inhibitors, such as rofecoxib, celecoxib), diclofenac, etodolac, famotidine, fenoprofen, flurbiprofen, ketoprofen, ketorolac, ibuprofen, indomethacin, meclofenamate, mefenamic acid, meloxicam, nambumetone, naproxen, oxaprozin, piroxicam, salsalate, sulindac, tolmetin;
immunosuppressants-mercaptopurine, corticosteroids such as dexamethasone, hydrocortisone, prednisone, methylprednisolone and prednisolone, alkylating agents such as cyclophosphamide, calcineurin inhibitors such as cyclosporine, sirolimus and tacrolimus, inhibitors of inosine monophosphate dehydrogenase (IMPDH) such as mycophenolate, mycophenolate mofetil and azathioprine, and agents designed to suppress cellular immunity while leaving the recipient's humoral immunologic response intact, including various antibodies (for example, antilymphocyte globulin (ALG), antithymocyte globulin (ATG), monoclonal anti-T-cell antibodies (OKT3)) and irradiation. Azathioprine is currently available from Salix Pharmaceuticals, Inc. under the brand name Azasan; mercaptopurine is currently available from Gate Pharmaceuticals, Inc. under the brand name Purinethol; prednisone and prednisolone are currently available from Roxane Laboratories, Inc.; Methyl prednisolone is currently available from Pfizer; sirolimus (rapamycin) is currently available from Wyeth-Ayerst under the brand name Rapamune; tacrolimus is currently available from Fujisawa under the brand name Prograf; cyclosporine is current available from Novartis under the brand name Sandimmune and Abbott under the brand name Gengraf; IMPDH inhibitors such as mycophenolate mofetil and mycophenolic acid are currently available from Roche under the brand name Cellcept and Novartis under the brand name Myfortic; azathioprine is currently available from Glaxo Smith Kline under the brand name Imuran; and antibodies are currently available from Ortho Biotech under the brand name Orthoclone, Novartis under the brand name Simulect (basiliximab) and Roche under the brand name Zenapax (daclizumab); and
Guanylate cyclase-C receptor agonists or intestinal secretagogues, for example linaclotide, sold under the name Linzess.

In certain embodiments, the second therapeutic is, or comprises, a steroid, such as a corticosteroid, including, but not limited to, glucocorticoids and/or mineralocorticoids. Steroids suitable for use in combination with the disclosed compounds include synthetic and non-synthetic glucocorticoids. Exemplary steroids, such as glucocorticoids, suitable for use in the disclosed methods include, but are not limited to, alclomethasones, algestones, beclomethasones (e.g. beclomethasone dipropionate), betamethasones (e.g. betamethasone 17-valerate, betamethasone sodium acetate, betamethasone sodium phosphate, betamethasone valerate), budesonides, clobetasols (e.g. clobetasol propionate), clobetasones, clocortolones (e.g. clocortolone pivalate), cloprednols, corticosterones, cortisones, cortivazols, deflazacorts, desonides, desoximethasones, dexamethasones (e.g. dexamethasone 21-phosphate, dexamethasone acetate, dexamethasone sodium phosphate), diflorasones (e.g. diflorasone diacetate), diflucortolones, difluprednates, enoxolones, fluazacorts, flucloronides, fludrocortisones (e.g., fludrocortisone acetate), flumethasones (e.g. flumethasone pivalate), flunisolides, fluocinolones (e.g. fluocinolone acetonide), fluocinonides, fluocortins, fluocortolones, fluorometholones (e.g. fluorometholone acetate), fluperolones (e.g., fluperolone acetate), fluprednidenes, fluprednisolones, flurandrenolides, fluticasones (e.g. fluticasone propionate), formocortals, halcinonides, halobetasols, halometasones, halopredones, hydrocortamates, hydrocortisones (e.g. hydrocortisone 21-butyrate, hydrocortisone aceponate, hydrocortisone acetate, hydrocortisone buteprate, hydrocortisone butyrate, hydrocortisone cypionate, hydrocortisone hemisuccinate, hydrocortisone probutate, hydrocortisone sodium phosphate, hydrocortisone sodium succinate, hydrocortisone valerate), loteprednol etabonate, mazipredones, medrysones, meprednisones, methylprednisolones (methylprednisolone aceponate, methylprednisolone acetate, methylprednisolone hemi succinate, methylprednisolone sodium succinate), mometasones (e.g., mometasone furoate), paramethasones (e.g., paramethasone acetate), prednicarbates, prednisolones (e.g. prednisolone 25-diethylaminoacetate, prednisolone sodium phosphate, prednisolone 21-hemi succinate, prednisolone acetate; prednisolone farnesylate, prednisolone hemisuccinate, prednisolone-21 (beta-D-glucuronide), prednisolone metasulphobenzoate, prednisolone steaglate, prednisolone tebutate, prednisolone tetrahydrophthalate), prednisones, prednivals, prednylidenes, rimexolones, tixocortols, triamcinolones (e.g. triamcinolone acetonide, triamcinolone benetonide, triamcinolone hexacetonide, triamcinolone acetonide 21-palmitate, triamcinolone diacetate), or any combination thereof. Additional information concerning steroids, and the salts thereof, can be found, for example, in Remington's Pharmaceutical Sciences, A. Osol, ed., Mack Pub. Co., Easton, Pa. (16th ed. 1980).

In some examples, the steroid is a glucocorticoid, and may be selected from cortisone, dexamethasone, hydrocortisone, methylprednisolone, prednisolone, prednisone, or a combination thereof. In a particular example, the steroid is, or comprises, prednisone. In another particular example, the steroid is, or comprises, dexamethasone.

These various agents can be used in accordance with their standard or common dosages, as specified in the prescribing information accompanying commercially available forms of the drugs (see also, the prescribing information in the 2006 Edition of The Physician's Desk Reference), the disclosures of which are incorporated herein by reference.

### V. Compositions Comprising the Disclosed Compounds

The disclosed compounds may be used alone, in any combination, and in combination with, or adjunctive to, at least one second therapeutic agent. Furthermore, the disclosed compound or compounds, and/or the at least one second therapeutic, may be used in combination with any suitable excipient useful for forming compositions for administration to a subject. Excipients can be included in pharmaceutical compositions for a variety of purposes, such as to dilute a composition for delivery to a subject, to facilitate processing of the formulation, to provide advantageous material properties to the formulation, to facilitate dispersion from a delivery device, to stabilize the formulation (e.g., antioxidants or buffers), to provide a pleasant or palatable taste or consistency to the formulation, or the like. The pharmaceutically acceptable excipient(s) may include a pharmaceutically acceptable carrier(s) and/or a pharmaceutically acceptable adjuvant(s). Exemplary excipients include, but are not limited to: mono-, di-, and polysaccharides, sugar alcohols and other polyols, such as, lactose, glucose, raffinose, melezitose, lactitol, maltitol, trehalose, sucrose, mannitol, starch, or combinations thereof; surfactants, such as sorbitols, diphosphatidyl choline, and lecithin; bulking agents; buffers, such as phosphate and citrate buffers; anti-adherents, such as magnesium stearate; binders, such as saccharides (including disaccharides, such as sucrose and lactose,), polysaccharides (such as starches, cellulose, microcrystalline cellulose, cellulose ethers (such as hydroxypropyl cellulose)), gelatin, synthetic polymers (such as polyvinylpyrrolidone, polyalkylene glycols); coatings (such as cellulose ethers, including hydroxypropylmethyl cellulose, shellac, corn protein zein, and gelatin); release aids (such as enteric coatings); disintegrants (such as crospovidone, crosslinked sodium carboxymethyl cellulose, and sodium starch glycolate); fillers (such as dibasic calcium phosphate, vegetable fats and oils, lactose, sucrose, glucose, mannitol, sorbitol, calcium carbonate, and magnesium stearate); flavors and sweeteners (such as mint, cherry, anise, peach, apricot or licorice, raspberry, and vanilla); lubricants (such as minerals, exemplified by talc or silica, fats, exemplified by vegetable stearin, magnesium stearate or stearic acid); preservatives (such as antioxidants exemplified by vitamin A, vitamin E, vitamin C, retinyl palmitate, and selenium, amino acids, exemplified by cysteine and methionine, citric acid and sodium citrate, parabens, exemplified by methyl paraben and propyl paraben); colorants; compression aids; emulsifying agents; encapsulation agents; gums; granulation agents; and combinations thereof.

### VI. Methods of Use

A. Diseases/Disorders

The disclosed compounds, as well as combinations and/or compositions thereof, may be used to ameliorate, treat, and/or prevent a variety of diseases, conditions, and/or disorders. In particular embodiments, the disclosed compound, combinations of disclosed compounds, or compositions thereof, may be useful for treating conditions in which inhibition of an interleukin-1 receptor-associated kinase (IRAK) pathway is therapeutically useful. In some embodiments, the compounds directly inhibit an IRAK protein, such as IRAK1, IRAK2, IRAK3 and/or IRAK4. In certain embodiments, disclosed compounds are useful for treating, preventing, and/or ameliorating auto-immune diseases, inflammatory disorders, cardiovascular diseases, nerve disorders, neurodegenerative disorders, allergic disorders, asthma, pancreatitis, multi-organ failure, kidney diseases, platelet aggregation, cancer, transplantation, sperm motility, erythrocyte deficiency, graft rejection, lung injuries, respiratory diseases, ischemic conditions, and bacterial and viral infections.

In some embodiments, the disclosed compound, combinations of disclosed compounds, or compositions thereof, may be used to treat or prevent allergic diseases, amyotrophic lateral sclerosis (ALS), systemic lupus erythematosus, rheumatoid arthritis, type I diabetes mellitus, inflammatory bowel disease, biliary cirrhosis, uveitis, multiple sclerosis, Crohn's disease, ulcerative colitis, bullous pemphigoid, sarcoidosis, psoriasis, autoimmune myositis, Wegener's granulomatosis, ichthyosis, Graves ophthalmyopathy, or asthma.

The disclosed compound, combinations of disclosed compounds, or compositions thereof, may also be useful for ameliorating, treating, and/or preventing immune regulatory disorders related to bone marrow or organ transplant rejection or graft-versus-host disease. Examples of inflammatory and immune regulatory disorders that can be treated with the present compounds include, but are not limited to, transplantation of organs or tissue, graft-versus-host diseases brought about by transplantation, autoimmune syndromes including rheumatoid arthritis, lupus, including systemic lupus erythematosus, Hashimoto's thyroiditis, multiple sclerosis, systemic sclerosis, myasthenia gravis, type I diabetes, uveitis, posterior uveitis, allergic encephalomyelitis, glomerulonephritis, postinfectious autoimmune diseases including rheumatic fever and post-infectious glomerulonephritis, inflammatory and hyperproliferative skin diseases, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, alopecia areata, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, celiac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, chronic lymphocytic leukemia, arteriosclerosis, atherosclerosis, aortitis syndrome, polyarteritis nodosa, myocardosis, scleroderma, Wegener's granuloma, Sjdgren's syndrome, adiposis, eosinophilic fascitis, lesions of gingiva, periodontium, alveolar bone, substantia ossea dentis, glomerulonephritis, male pattern alopecia or alopecia senilis by preventing epilation or providing hair germination and/or promoting hair generation and hair growth, muscular dystrophy, pyoderma and Sezary's syndrome, Addison's disease, ischemia-reperfusion injury of organs which occurs upon preservation, transplantation or ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA ballous dermatitis and cement dermatitis, gingivitis, periodontitis, sepsis, pancreatitis, diseases caused by environmental pollution, aging, carcinogenesis, metastasis of carcinoma and hypobaropathy, disease caused by histamine or leukotriene-C4 release, Behcet's disease, autoimmune hepatitis, primary biliary cirrhosis, sclerosing cholangitis, partial liver resection, acute liver necrosis, necrosis caused by toxin, viral hepatitis, shock, or anoxia, B-virus hepatitis, non-A/non-B hepatitis, cirrhosis, alcoholic liver disease, including alcoholic cirrhosis, non-alcoholic steatohepatitis (NASH), hepatic failure, fulminant hepatic failure, late-onset hepatic failure, “acute-on-chronic” liver failure, augmentation of chemotherapeutic effect, cytomegalovirus infection, HCMV infection, AIDS, cancer, senile dementia, Parkinson's disease, trauma, chronic bacterial infection, palmoplantar pustulosis, hidradenitis suppurativa, cytokine release syndrome (CRS), acute respiratory distress syndrome (ARDS), acute kidney injury (AKI), kidney malfunction, or thrombosis.

In some embodiments, the disease or condition is hidradenitis suppurativa, or a lymphoid neoplasm selected from myeloproliferative neoplasms (MPN) excluding polycythemia vera, myeloid/lymphoid neoplasms with PDGFRA rearrangement, myeloid/lymphoid neoplasms with PDGFRB rearrangement, myeloid/lymphoid neoplasms with FGFR1 rearrangement, myeloid/lymphoid neoplasms with PCM1-JAK2, myelodysplastic/myeloproliferative neoplasms (MDS/MPN), myeloid sarcoma, myeloid proliferations related to Down syndrome, blastic plasmacytoid dendritic cell neoplasm, B-lymphoblastic leukemia/lymphoma; and/or T-lymphoblastic leukemia/lymphoma. In some embodiments, the lymphoid neoplasm is a myeloproliferative neoplasm selected from chronic myeloid leukemia (CML), chronic neutrophilic leukemia (CNL), primary myelofibrosis (PMF), essential thrombocythemia, chronic eosinophilic leukemia, or a combination thereof. In other embodiments, the lymphoid neoplasm is a myelodysplastic/myeloproliferative neoplasm selected from chronic myelomonocytic leukemia, atypical chronic myeloid leukemia (aCML), juvenile myelomonocytic leukemia (JMML), MDS/MPN with ring sideroblasts and thrombocytosis (MDS/MPN-RS-T), or a combination thereof.

In certain embodiments the present compounds are useful for treating nerve pain, including neuropathic pain and inflammation induced pain.

In certain embodiments, the disclosed compound, combinations of disclosed compounds, or compositions thereof, are useful for treating and/or preventing rheumatoid arthritis, psoriatic arthritis, osteoarthritis, systemic lupus erythematosus, lupus nephritis, ankylosing spondylitis, osteoporosis, systemic sclerosis, multiple sclerosis, psoriasis, in particular pustular psoriasis, type I diabetes, type II diabetes, inflammatory bowel disease (Crohn's disease and ulcerative colitis), hyperimmunoglobulinemia d and periodic fever syndrome, cryopyrin-associated periodic syndromes, Schnitzler's syndrome, systemic juvenile idiopathic arthritis, adult's onset Still's disease, gout, gout flares, pseudogout, sapho syndrome, Castleman's disease, sepsis, stroke, atherosclerosis, celiac disease, DIRA (deficiency of Il-1 receptor antagonist), Alzheimer's disease, or Parkinson's disease.

Proliferative diseases that may be treated by the disclosed compound, combinations of disclosed compounds, or compositions thereof, include benign or malignant tumors, solid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins, a mammary carcinoma, follicular carcinoma, undifferentiated carcinoma, papillary carcinoma, seminoma, melanoma, IL-1 driven disorders, a MyD88 driven disorder (such as ABC diffuse large B-cell lymphoma (DLBCL), Waldenström's macroglobulinemia, Hodgkin's lymphoma, primary cutaneous T-cell lymphoma or chronic lymphocytic leukemia), smoldering or indolent multiple myeloma, or hematological malignancies (including leukemia, acute myeloid leukemia (AML), DLBCL, ABC DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, myelodysplastic syndromes (MDS), myelofibrosis, polycythemia vera, Kaposi's sarcoma, Waldenström's macroglobulinemia (WM), splenic marginal zone lymphoma, multiple myeloma, plasmacytoma, intravascular large B-cell lymphoma). In particular, the presently disclosed compounds are useful in treating drug resistant malignancies, such as those resistant to JAK inhibitors ibrutinib resistant malignancies, including ibrutinib resistant hematological malignancies, such as ibrutinib resistant CLL and ibrutinib resistant Waldenström's macroglobulinemia.

Examples of allergic disorders that may be treated using the disclosed compound, combinations of disclosed compounds, or compositions thereof, include, but are not limited to, asthma (e.g. atopic asthma, allergic asthma, atopic bronchial IgE-mediated asthma, non-atopic asthma, bronchial asthma, non-allergic asthma, essential asthma, true asthma, intrinsic asthma caused by pathophysiologic disturbances, essential asthma of unknown or unapparent cause, emphysematous asthma, exercise-induced asthma, emotion-induced asthma, extrinsic asthma caused by environmental factors, cold air induced asthma, occupational asthma, infective asthma caused by or associated with bacterial, fungal, protozoal, or viral infection, incipient asthma, wheezy infant syndrome, bronchiolitis, cough variant asthma or drug-induced asthma), allergic bronchopulmonary aspergillosis (ABPA), allergic rhinitis, perennial allergic rhinitis, perennial rhinitis, vasomotor rhinitis, post-nasal drip, purulent or non-purulent sinusitis, acute or chronic sinusitis, and ethmoid, frontal, maxillary, or sphenoid sinusitis.

As another example, rheumatoid arthritis (RA) typically results in swelling, pain, loss of motion and tenderness of target joints throughout the body. RA is characterized by chronically inflamed synovium that is densely crowded with lymphocytes. The synovial membrane, which is typically one cell layer thick, becomes intensely cellular and assumes a form similar to lymphoid tissue, including dendritic cells, T-, B- and NK cells, macrophages and clusters of plasma cells. This process, as well as a plethora of immunopathological mechanisms including the formation of antigen-immunoglobulin complexes, eventually result in destruction of the integrity of the joint, resulting in deformity, permanent loss of function and/or bone erosion at or near the joint. The disclosed compound, combinations of disclosed compounds, or compositions thereof, may be used to treat, ameliorate, or prevent any single, several or all of these symptoms of RA. Thus, in the context of RA, the compounds are considered to provide therapeutic benefit when a reduction or amelioration of any of the symptoms commonly associated with RA is achieved, regardless of whether the treatment results in a concomitant treatment of the underlying RA and/or a reduction in the amount of circulating rheumatoid factor (“RF”).

The American College of Rheumatology (ACR) has developed criteria for defining improvement and clinical remission in RA. Once such parameter, the ACR20 (ACR criteria for 20% clinical improvement), requires a 20% improvement in the tender and swollen joint count, as well as a 20% improvement in 3 of the following 5 parameters: patient's global assessment, physician's global assessment, patient's assessment of pain, degree of disability, and level of acute phase reactant. These criteria have been expanded for 50% and 70% improvement in ACR50 and ACR70, respectively. Other criteria include Paulu's criteria and radiographic progression (e.g. Sharp score).

In some embodiments, therapeutic benefit in patients suffering from RA is achieved when the patient exhibits an ACR20. In specific embodiments, ACR improvements of ACRC50 or even ACR70 may be achieved.

Cytokine release syndrome (CRS) is a potentially life-threatening condition that may result from a variety of factors, including severe viral infections such as influenza, administration of antibodies that are used for immunotherapy, such as cancer immunotherapy, and non-protein-based cancer drugs such as oxaliplatin and lenalidomide. Immunotherapy can involve high levels of immune activation that exceed naturally occurring immune activation levels, and CRS is a non-antigen specific toxicity that can occur as a result. As immune-based therapies become more potent, CRS is becoming increasing diagnosed. CRS has also been observed in the setting of haploidentical donor stem cell transplantation, and graft-versus-host disease. Shimabukuro-Vornhagen et al., Journal for ImmunoTherapy of Cancer 6:56 (2018). CRS is associated with elevated circulating levels of several cytokines including interleukin (IL)-6 and interferon γ. Lee et al., Blood 124(2):188-195 (10 Jul. 2014; Epub 29 May 2014).

CRS typically is clinically observed when significant numbers of lymphocytes and/or myeloid cells are activated and release inflammatory cytokines. The cytokine release may be induced by chemo- or biotherapy, and/or may be associated with therapeutic antibody treatments, such as immunotherapy, for example, for cancer treatment. Exemplary immunotherapies that may result in CRS include, but are not limited to, therapies where the cells express recombinant receptors, such as chimeric antigen receptors (CARs) and/or other transgenic receptors such as T cell receptors (TCRs). CRS induced by CAR T therapy generally occurs within days of T cell infusion at the peak of CAR T cell expansion. Giavridis et al., Nat Med. 24(6):731-738 (June 2018; Epub 28 May 2018). Examples of CAR T therapy that can induce CRS include axicabtagene ciloleucel (marketed as YESCARTA®) and tisagenlecleucel (marketed as KYMRIAH®).

Highly elevated interleukin 6 (IL-6) levels have been observed in patients with CRS and also in murine models of the disease, indicating that IL-6 may have a role in CRS pathophysiology. Shimabukuro-Vornhagen, J Immunother Cancer 6(1), 56 (2018). IL-6 can signal via two different modes. Classical IL-6 signaling involves binding of IL-6 to a membrane-bound IL-6 receptor. However, the IL-6 receptor does not possess intracellular signaling domains. Instead, after soluble IL-6 binds to membrane-bound IL-6 receptors, the IL-6/IL-6 receptor complex binds to membrane-bound gp130, which initiates signaling through its intracellular domain. In trans-signaling, IL-6 binds to a soluble form of the IL-6 receptor, which is typically cleaved from the cell surface by metalloproteinases. The resulting soluble IL-6/IL-6 receptor complex binds to gp130 and therefore can also induce signaling in cell types that do not express membrane bound IL-6 receptors.

IL-6 contributes to many of the key symptoms of CRS. Via trans-signaling, IL-6 leads to characteristic symptoms of severe CRS, i.e. vascular leakage, and activation of the complement and coagulation cascade inducing disseminated intravascular coagulation (DIC). In addition, IL-6 likely contributes to cardiomyopathy that is often observed in patients with CRS by promoting myocardial dysfunction. In a murine model, CRS developed within 2-3 days of CAR T cell infusion and could be lethal. Giavridis et al., Nat Med. 24(6): 731-738 (2018). CRS symptoms may start within minutes or hours of the start of antibody treatment, and can include a fever, which may reach or exceed 40° C., nausea, fatigue, headache, tachycardia, hypotension, rash, shortness of breath, and/or myalgias. However, in certain cases, additional and potentially more serious complications may develop, including cardiac dysfunction, adult respiratory distress syndrome, neurological toxicity, renal and/or hepatic failure, and/or disseminated intravascular coagulation.

The National Cancer Institute Common Terminology Criteria for Adverse Events (CTCAE v. 5.0, pub. Nov. 27, 2017) includes a grading system for CRS.

Grade 1: Fever with or without constitutional symptoms.

Grade 2: Hypotension responding to fluids; hypoxia responding to <40% 02.

Grade 3: Hypotension managed with one pressor; hypoxia requiring ≥40% 02.

Grade 4: Life-threatening consequences; urgent intervention indicated.

Grade 5: Death.

The subject may not exhibit a sign or symptom of CRS and/or may be at risk of developing CRS. In such embodiments, administering the compound substantially prevents the onset of CRS, or prevents the onset of grade 2 or higher CRS.

Alternatively, the subject exhibits at least one sign or symptom of CRS and may exhibit at least one sign or symptom of grade 1 CRS. Or the subject may exhibit at least one sign or symptom of grade 2 or higher CRS, such as grade 3 or higher CRS. The disclosed compound(s) may be administered within 24 hours of the onset of the sign or symptom, and/or administering the compound(s) may ameliorate the sign or symptom of CRS, compared to the severity of the sign or symptom prior to administration of the compound(s), such as reducing the grade of CRS from 4 to 3, 2 or 1, or from 3, to 2 or 1, or from 2 to 1. Alternatively, CRS symptoms are substantially reduced to below grade 1 level, such that the subject no longer experiences symptoms associated with CRS. In some embodiments the sign or symptom is a fever and may be a fever of 40° C. or higher.

The disclosed compound(s)s may be administered to a subject that has previously be administered a first therapy for which CRS is a known, suspected, or potential side effect. Administration of the first therapy may be initiated from greater than zero to 10 days prior to administration of the compound(s). Alternatively, the compound(s) may be administered to a subject who will be, or is concurrently being, administered a first therapy for which CRS is a known, suspected, and/or potential side effect. The first therapy may comprise a cell therapy, including, but not limited to, chimeric antigen receptor (CAR)-expressing therapy and/or a transgenic receptor therapy. Cell-free antibodies are also known to elicit this syndrome, particularly those that activate T-cells, including, but not limited to, CAMPATH 1-H, blinatumomab, and/or rituximab.

A second therapeutic agent, for example, a steroid, an anti-inflammatory agent, an immunosuppressant, or a combination thereof, also may be administered to treat or prevent CRS. The steroid may be a corticosteroid, such as, for example, dexamethasone or prednisone, or a combination thereof. The disclosed compound(s) may be administered substantially simultaneously with the second therapeutic agent, or the compound(s) and second therapeutic agent may be administered sequentially in any order.

Acute respiratory distress syndrome (ARDS) is a syndrome characterized by a severe shortness of breath, labored and unusually rapid breathing, low blood pressure, confusion and extreme tiredness. This syndrome can be diagnosed based on a PaO2/FiO2 ratio of less than 300 mmHg despite a PEEP of more than 5 cm H2O (Fan et al JAMA. 319: 698-71).

ARDS occurs when fluid builds up in lung alveoli. The fluid prevents the lungs from filling with enough air, limiting the amount of oxygen that reaches the bloodstream which, in turn, deprives the organs of the oxygen they need to function. The symptoms of ARDS can vary in intensity, depending on its cause and severity. Severe shortness of breath—the hallmark of ARDS—usually develops within a few hours to a few days after the infection by some respiratory viruses, e.g., COVID-19 and influenza. Many people who develop ARDS do not survive, and the risk of death increases with age and severity of illness. Of the patients that survive ARDS, some completely recover while others have lasting damage to their lungs. ARDS may be referred to as Acute Lung Injury (ALI) in some publications.

Acute kidney injury (AKI), also known as acute renal injury (ARI) or acute renal failure (ARF), is a syndrome characterized by an abrupt reduction of renal function including, e.g., the ability to excrete waste from a patient's blood. AKI is characterized by a decline of glomerular filtration rate, urine output, or both. This loss of filtration capacity results in retention of nitrogenous (urea and creatinine) and non-nitrogenous waste products that are normally excreted by the kidney, a reduction in urine output, or both. AKI may be categorized as prerenal, intrinsic renal, or postrenal in causation. Intrinsic renal disease can be further divided into glomerular, tubular, interstitial, and vascular abnormalities. AKI is accompanied by an inflammatory response that if unchecked can lead to renal fibrosis and chronic renal failure. AKI usually occurs over a period of hours or days and is potentially reversible. AKI may be characterized as an abrupt (i.e., for example, within 14 days, within 7 days, within 72 hours, or within 48 hours) reduction in kidney function identified by an absolute increase in serum creatinine of greater than or equal to 0.3 mg/dl (≥26.4 μmol/l), a percentage increase in serum creatinine of greater than or equal to 50% (1.5-fold from baseline), or a reduction in urine output (documented oliguria of less than 0.5 ml/kg per hour for at least 6 hours). Risk factors include, for example, a subject undergoing or having undergone major vascular surgery, coronary artery bypass, or other cardiac surgery; a subject having pre-existing congestive heart failure, preeclampsia, eclampsia, diabetes mellitus, hypertension, coronary artery disease, proteinuria, renal insufficiency, glomerular filtration below the normal range, cirrhosis, serum creatinine above the normal range, or sepsis; or a subject exposed to NSAIDs, cyclosporines, tacrolimus, aminoglycosides, foscarnet, ethylene glycol, hemoglobin, myoglobin, ifosfamide, heavy metals, methotrexate, radiopaque contrast agents, or streptozotocin. This list is not meant to be limiting.

Kidney malfunction includes, but is not limited to, kidney disorders, kidney disease, kidney dysfunction, kidney cancer, absence of at least one kidney due to accidents, surgical removal or genetic disorders, or other conditions where one or both of the kidneys are not properly functioning. Kidney malfunction may include acute kidney injury.

Thrombosis is a clotting disorder to which an excess of platelets contributes. Thrombosis may refer to the formation of a thrombus (blood clot) inside a blood vessel. The term encompasses, without limitation, arterial and venous thrombosis, including deep vein thrombosis, portal vein thrombosis, jugular vein thrombosis, renal vein thrombosis, stroke, myocardial infarction, Budd-Chiari syndrome, Paget-Schroetter disease, and cerebral venous sinus thrombosis. In some embodiments, the patient is at heightened risk relative to the general population (e.g., as measured by recognized risk factors) of a thrombotic event. In some embodiments, a patient has one or more risk factors that make the patient have a high risk of developing thrombosis relative to the general population. Risk factors for thrombosis include, e.g., classical cardiovascular disease risk factors: hyperlipidemia, smoking, diabetes, hypertension, and abdominal obesity; strong classical venous thromboembolism risk factors: trauma or fractures, major orthopedic surgery, and oncological surgery; moderate classical venous thromboembolism risk factors: non-oncological surgery, oral contraceptives and hormone replacement therapy, pregnancy and puerperium, hypercoagulability, and previous venous thromboembolism; and weak classical venous thromboembolism risk factors: age, bed rest (>3 days), prolonged travel, and metabolic syndrome. Additional risk factors include inherited, acquired and mixed coagulation or metabolic risk factors for thrombosis such as, e.g., inherited: antithrombin deficiency, protein C deficiency, Protein S deficiency, Factor V Leiden, Prothrombin G20210A; acquired: antiphospholipid syndrome; mixed: hyperhomocysteinaemia, increased fibrinogen levels, increased factor VIII levels, increased factor IX levels. In some cases, the use of heparin may increase the risk of thrombosis including, e.g., heparin-induced thrombocytopenia (HIT). Diseases and conditions associated with thrombosis include, without limitation, acute venous thrombosis, pulmonary embolism, thrombosis during pregnancy, hemorrhagic skin necrosis, acute or chronic disseminated intravascular coagulation (DIC), sepsis induced coagulopathy (SIC), clot formation from surgery, long bed rest, long periods of immobilization, venous thrombosis, fulminant meningococcemia, acute thrombotic stroke, acute coronary occlusion, acute peripheral arterial occlusion, massive pulmonary embolism, axillary vein thrombosis, massive iliofemoral vein thrombosis, occluded arterial cannulae, occluded venous cannulae, cardiomyopathy, venoocclusive disease of the liver, hypotension, decreased cardiac output, decreased vascular resistance, pulmonary hypertension, diminished lung compliance, leukopenia, thrombocytopenia (e.g., immune thrombocytopenia), and immune thrombocytic purpura. in a subject at risk for thrombosis, the subject may be monitored using methods known to those of skill in the art of maintaining hemostasis in patients at risk for thrombosis. Examples of methods for monitoring patients at risk of thrombosis included, without limitation, digital subtraction angiography, in vitro assays or non-invasive methods. Examples of in vitro assays useful for identifying and monitoring subjects at risk for thrombosis and for treatment using the present methods include, without limitation, functional assays and antibody detection assays.

Thrombotic event refers to any disorder which involves a blockage or partial blockage of an artery or vein with a thrombosis. A thrombotic event includes, but is not limited to, thrombotic disorders such as myocardial infarction, unstable angina, stroke, pulmonary embolism, transient ischemic attack, deep vein thrombosis, thrombotic re-occlusion and peripheral vascular thrombosis. A thrombotic event also includes thrombotic re-occlusion which occurs subsequent to a coronary intervention procedure or thrombolytic therapy.

COVID-19 is a disease caused by infection by SARS-CoV-2 (previously known as 2019-nCoV) which first appeared in Wuhan, China.

COVID-19-associated ARDS refers to ARDS that is caused by infection by SARS-CoV-2. Patients having COVID-19-associated ARDS may have been diagnosed as having a COVID-19, may have been exposed to another person having a COVID19, or may be suspected of having a COVID-19 based on their symptoms.

COVID-19-associated AKI refers to AKI that is caused by infection by SARS-CoV-2. Patients having COVID-19-associated AKI may have been diagnosed as having a COVID-19, may have been exposed to another person having a COVID-19, or may be suspected of having a COVID-19 based on their symptoms. In some cases, COVID-19-associated AKI includes AKI with the symptoms described, e.g., in Batlle et al. J. AM. SOC. NEPHROL. 2020, 31(7): 1380-1383 and Gabarre et al. Intensive Care Med. 2020, 46(7): 1339-1348, the disclosures of which are incorporated herein by reference in their entireties.

COVID-19-associated thrombosis refers to thrombosis that is caused by infection by SARS-CoV-2. Patients having COVID-19-associated thrombosis may have been diagnosed as having a COVID-19, may have been exposed to another person having a COVID-19, or may be suspected of having a COVID-19 based on their symptoms. In some cases, COVID-19-associated thrombosis includes any of the symptoms described in, e.g., Connors et al. Blood 2020, 135(23): 2033-2040 and Bikdeli et al. J. Am. Coll. Cardiol. 2020, 75(23): 2950-73, the disclosures of which are incorporated herein by reference in their entireties.

The term “associated with COVID-19” refers to a symptom or indication that typically develops within 28 days of hospitalization due to/signs of COVID-19.

For COVID-19-associated ARDS, successful treatment may include a decrease in shortness of breath, less labored or less rapid breathing, higher blood pressure, decreased confusion and/or a decrease tiredness. A treatment may be administered prophylactically, i.e., before the onset of ARDS. A prophylactic treatment prevents ARDS and can be administered to patients that have or are suspected of having a COVID-19 infection, but without the severe symptoms of ARDS. For example, prophylactic treatment can be administered to patients that have a cough without the other symptoms of ARDS.

For COVID-19-associated AKI, successful treatment may include increased kidney function. Kidney function may be assessed by measuring serum creatinine levels, serum creatinine clearance, or blood urea nitrogen levels. In some cases, the successful treatment includes a reduction in metabolic acidosis, hyperkalaemia, oliguria or anuria, azotemia, restoration in body fluid balance, and improved effects on other organ systems. A treatment may be administered prophylactically, i.e., before the onset of AKI. A prophylactic treatment prevents AKI and can be administered to patients that have or are suspected of having a COVID-19 infection, but without the severe symptoms of AKI. For example, prophylactic treatment can be administered to patients that have one or more of increased serum or urine creatinine, hematuria, hypoproteinemia, decreased antithrombin III levels, hypalbuminaemia, leucozyturia, or proteinuria without the other symptoms of AKI.

For COVID-19-associated thrombosis, successful treatment may include improvement in the subject's coagulation profile, or preventing, slowing, delaying, or arresting, a worsening of the coagulation profile for which the subject is at risk. A coagulation profile may be assessed by measurement of one or more coagulation parameters including, e.g., a subject's serum level of one or more of D-dimer, Factor II, Factor V (e.g., Factor V Leiden), Factor VII, Factor VIII, Factor IX, Factor XI, Factor XII, Factor XIII, F/fibrin degradation products, thrombin-antithrombin 111 complex, fibrinogen, plasminogen, prothrombin, and von Willebrand factor. Additional coagulation parameters that may be measured for the coagulation profile include, e.g., prothrombin time, thromboplastin time, activated partial thromboplast time (aPTT), antithrombin activity, platelet count, protein C levels, and protein S levels. In addition, the levels of C reactive protein may also be assessed in the patient prior to treatment and if elevated this may be used as a further indicator as to an increased risk of thrombosis in the patient.

Sepsis is a clinical syndrome of life-threatening organ dysfunction caused by a dysregulated immune response to infection. The more severe form of sepsis “septic shock” is characterized by a critical reduction in tissue perfusion; acute failure of multiple organs, including the lungs, kidneys, and liver. Common causes in immunocompetent patients include many different species of gram-positive and gram-negative bacteria. Immunocompromised patients may have uncommon bacterial or fungal species as a cause. Signs include fever, hypotension, oliguria, and confusion. Diagnosis is primarily clinical combined with culture results showing infection; early recognition and treatment is critical. Treatment is aggressive fluid resuscitation, antibiotics, surgical excision of infected or necrotic tissue and drainage of pus, and supportive care.

Influenza is a disease generally known as the “flu.” Influenza is caused by a group of viruses that can be broken down into 4 separate groups: Influenza A, Influenza B, Influenza C and Influenza D which are separated based on their nuceloproteins and matrix proteins. Influenza causes viral respiratory infection resulting in fever, coryza, cough, headache, and malaise. Influenza A, B, and C all infect humans while there have been no documented cases of human Influenza D infection. Influenza C on the other hand does not cause typical influenza illness seen in individuals infected with Influenza A, B or C.

Influenza A strains are further classified based on two surface proteins, hemagglutinin (H) and neuraminidase (N). There are 18 different hemagglutinin subtypes and 11 different neuraminidase subtypes (H1 through H18 and N1 through N11, respectively). While there are potentially 198 different influenza A subtype combinations, only 131 subtypes have been detected in nature. Current subtypes of influenza A viruses that routinely circulate in people include: A(H1N1) and A(H3N2).

Cytokine release-related condition associated with influenza refers to any condition associated with influenza that leads to high levels of cytokine releases in the lungs and/or kidneys. Cytokine releases-related conditions, include without limitation, influenza-associated ARDS, influenza-associated AKI, influenza-associated thrombosis, influenza-associated sepsis, influenza-associated septic shock, etc.

Influenza-associated ARDS is ARDS that is caused by influenza infection. Patients having influenza-associated ARDS may have been diagnosed as having an influenza infection, may have been exposed to another person having an influenza infection, or may be suspected of having an influenza infection based on their symptoms.

Influenza-associated AKI is AKI that is caused by influenza infection. Patients having influenza-associated AKI may have been diagnosed as having an influenza infection, may have been exposed to another person having an influenza infection, or may be suspected of having an influenza infection based on their symptoms. In some cases, influenza-associated AKI includes AKI with the symptoms described, e.g., in Batlle et al. J. AM. SOC. NEPHROL. 2020, 31(7): 1380-1383 and Gabarre et al. Intensive Care Med. 2020, 46(7): 1339-1348, the disclosures of which are incorporated herein by reference in their entireties.

Influenza-associated thrombosis is thrombosis that is caused by influenza infection. Patients having influenza-associated thrombosis may have been diagnosed as having an influenza infection, may have been exposed to another person having an influenza infection, or may be suspected of having an influenza infection based on their symptoms. In some cases, influenza-associated thrombosis includes any of the symptoms described in, e.g., Connors et al. Blood 2020, 135(23): 2033-2040 and Bikdeli et al. J. Am. Coll. Cardiol. 2020, 75(23): 2950-73, the disclosures of which are incorporated herein by reference in their entireties.

Influenza-associated sepsis is sepsis that is caused by influenza infection. Patients having influenza-associated sepsis may have been diagnosed as having an influenza infection, may have been exposed to another person having an influenza infection, or may be suspected of having an influenza infection based on their symptoms. In some cases, influenza-associated thrombosis includes any of the symptoms described in, e.g., Florescu et al. Virulence. 2014 Jan. 1; 5(1): 137-142.and Gu et al. Eur Respir Rev. 2020 Jul. 21; 29(157):200038, the disclosures of which are incorporated herein by reference in their entireties.

The term “associated with influenza” refers to a symptom or indication that develops within 28 days of hospitalization/signs of influenza infection.

For influenza-associated ARDS, successful treatment may include a decrease in shortness of breath, less labored or less rapid breathing, higher blood pressure, decreased confusion and/or a decrease tiredness. A treatment may be administered prophylactically, i.e., before the onset of ARDS. A prophylactic treatment prevents ARDS and can be administered to patients that have or are suspected of having an influenza infection, but without the severe symptoms of ARDS. For example, prophylactic treatment can be administered to patients that have a cough without the other symptoms of ARDS.

For influenza-associated AKI, successful treatment may include increased kidney function. Kidney function may be assessed by measuring serum creatinine levels, serum creatinine clearance, or blood urea nitrogen levels. In some cases, the successful treatment includes a reduction in metabolic acidosis, hyperkalaemia, oliguria or anuria, azotemia, restoration in body fluid balance, and improved effects on other organ systems. A treatment may be administered prophylactically, i.e., before the onset of AKI. A prophylactic treatment prevents AKI and can be administered to patients that have or are suspected of having an influenza infection, but without the severe symptoms of AKI. For example, prophylactic treatment can be administered to patients that have one or more of increased serum or urine creatinine, hematuria, hypoproteinemia, decreased antithrombin III levels, hypalbuminaemia, leucozyturia, or proteinuria without the other symptoms of AKI.

For influenza-associated thrombosis, successful treatment may include improvement in the subject's coagulation profile, or preventing, slowing, delaying, or arresting, a worsening of the coagulation profile for which the subject is at risk. A coagulation profile may be assessed by measurement of one or more coagulation parameters including, e.g., a subject's serum level of one or more of D-dimer, Factor II, Factor V (e.g., Factor V Leiden), Factor VII, Factor VIII, Factor IX, Factor XI, Factor XII, Factor XIII, F/fibrin degradation products, thrombin-antithrombin 111 complex, fibrinogen, plasminogen, prothrombin, and von Willebrand factor. Additional coagulation parameters that may be measured for the coagulation profile include, e.g., prothrombin time, thromboplastin time, activated partial thromboplast time (aPTT), antithrombin activity, platelet count, protein C levels, and protein S levels. In addition, the levels of C reactive protein may also be assessed in the patient prior to treatment and if elevated this may be used as a further indicator as to an increased risk of thrombosis in the patient.

For influenza-associated sepsis or septic shock, successful treatment may include a reduction in fever, a reduction in high or moderately-high heartbeat (e.g. tachycardia), a reduction in sweating (i.e. diaphoresis), decreased confusion and/or a decrease tiredness, and/or a decrease in shortness of breath, less labored or less rapid breathing. A treatment may be administered prophylactically, i.e., before the onset of sepsis or septic shock. A prophylactic treatment prevents sepsis or septic shock and can be administered to patients that have or are suspected of having an influenza infection, but without the severe symptoms of sepsis or septic shock. For example, prophylactic treatment can be administered to patients that have a cough without the other symptoms of sepsis or septic shock.

Additionally, the disclosed compounds, combinations of disclosed compounds, or compositions thereof, may be used to treat sickle cell disease, particularly to reduce immunological responses that manifest in the disease. In some embodiments, the subject may exhibiting one or more of the following symptoms: anemia, sickle cell crisis, vaso-occlusive crisis, splenic sequestration crisis, splenic sequestration crises, acute chest syndrome, acute chest syndrome, aplastic crisis, hemolytic crisis, dactylitis, pneumonia, respiratory infection, bone-marrow embolization, or atelectasis.

Sickle cell disease (SCD) is a group of blood disorders typically inherited. The most common type is known as sickle cell anemia, which results in an abnormality in the oxygen carrying protein hemoglobin found in red blood cells. This leads to a rigid, sickle-like shape under certain circumstances. Problems in sickle cell disease typically begin around 5 to 6 months of age and a number of health problems may develop, such as attacks of pain (known as a sickle cell crisis), anemia, swelling in the hands and feet, bacterial infections and stroke. Long-term pain may develop as subjects age.

Sickle cell disease occurs when a person inherits two abnormal copies of the β-globin gene (HBB) that makes hemoglobin, one from each parent. That gene occurs in chromosome 11. Several subtypes exist, depending on the exact mutation in each hemoglobin gene. An attack can be set off by temperature changes, stress, dehydration, and high altitude.

The care of people with sickle cell disease may include infection prevention with vaccination and antibiotics, high fluid intake, folic acid supplementation, and pain medication. Other measures may include blood transfusion and the medication hydroxycarbamide (hydroxyurea). A small percentage of people can be cured by a transplant of bone marrow cells.

Patients with sickle cell disease may exhibit the following symptoms: Sickle cell crisis: The terms “sickle cell crisis” or “sickling crisis” may be used to describe several independent acute conditions occurring in subjects with SCD, which results in anemia and crises that could be of many types, including the vaso-occlusive crisis, aplastic crisis, splenic sequestration crisis, hemolytic crisis, and others. Most episodes of sickle cell crises last between five and seven days. Although infection, dehydration, and acidosis (all of which favor sickling) can act as triggers, in most instances, no predisposing cause is identified.

Vaso-occlusive crisis: The vaso-occlusive crisis is caused by sickle-shaped red blood cells that obstruct capillaries and restrict blood flow to an organ, resulting in ischemia, pain, necrosis, and often organ damage. The frequency, severity, and duration of these crises vary considerably. Painful crises are treated with hydration, analgesics, and blood transfusion; pain management requires opioid drug administration at regular intervals until the crisis has settled. For milder crises, a subgroup of subjects manages on nonsteroidal anti-inflammatory drugs such as diclofenac or naproxen. For more severe crises, most subjects require in-subject management for intravenous opioids; subject-controlled analgesia devices are commonly used in this setting. Vaso-occlusive crisis involving organs such as the penis or lungs are considered an emergency and treated with red blood cell transfusions. Incentive spirometry, a technique to encourage deep breathing to minimize the development of atelectasis, is recommended.

Splenic sequestration crisis: The spleen is frequently affected in sickle cell disease, as the sickle-shaped red blood cells cause narrowing of blood vessels and reduced function in clearing the defective cells. It is usually infarcted before the end of childhood in individuals with sickle cell anemia. This spleen damage increases the risk of infection from encapsulated organisms; preventive antibiotics and vaccinations are recommended for those lacking proper spleen function.

Splenic sequestration crises are acute, painful enlargements of the spleen, caused by intrasplenic trapping of red cells and resulting in a precipitous fall in hemoglobin levels with the potential for hypovolemic shock. Sequestration crises are considered an emergency. If not treated, subjects may die within 1-2 hours due to circulatory failure. Management is supportive, sometimes with blood transfusion. These crises are transient; they continue for 3-4 hours and may last for one day.

Acute chest syndrome: Acute chest syndrome is defined by at least two of these signs or symptoms: chest pain, fever, pulmonary infiltrate or focal abnormality, respiratory symptoms, or hypoxemia. It is the second-most common complication and it accounts for about 25% of deaths in subjects with SCD. Most cases present with vaso-occlusive crises, and then develop acute chest syndrome. Nevertheless, about 80% of people have vaso-occlusive crises during acute chest syndrome.

Aplastic crisis: Aplastic crises are instances of an acute worsening of the subject's baseline anemia, producing pale appearance, fast heart rate, and fatigue. This crisis is normally triggered by parvovirus B19, which directly affects production of red blood cells by invading the red cell precursors and multiplying in and destroying them. Parvovirus infection almost completely prevents red blood cell production for two to three days. In normal individuals, this is of little consequence, but the shortened red cell life of SCD subjects results in an abrupt, life-threatening situation. Reticulocyte counts drop dramatically during the disease (causing reticulocytopenia), and the rapid turnover of red cells leads to the drop in hemoglobin. This crisis takes 4 to 7 days to disappear. Most subjects can be managed supportively; some need a blood transfusion.

Hemolytic crisis: Hemolytic crises are acute accelerated drops in hemoglobin level. The red blood cells break down at a faster rate. This is particularly common in people with coexistent G6PD deficiency. Another influence of hemolytic crises in Sickle Cell Disease is oxidative stress on the erythrocytes, leukocytes, and platelets. When there is not enough red blood cell production in the bone marrow, the oxygen that the body receives, processes, and transports is unbalanced with the body's antioxidants. There is an imbalance in the oxygen reactive species in the cells, which leads to more production of red blood cells that are not properly oxygenated or formed. Oxidative stress may lead to anemia because of the imbalance of oxygen in the tissue. Management is supportive, sometimes with blood transfusions.

In addition, one of the earliest clinical manifestations is dactylitis, presenting as early as six months of age, and may occur in children with sickle cell trait. The crisis can last up to a month. Given that pneumonia and sickling in the lung can both produce symptoms of acute chest syndrome, the subject is treated for both conditions. It can be triggered by painful crisis, respiratory infection, bone-marrow embolization, or possibly by atelectasis, opiate administration, or surgery. Hematopoietic ulcers may also occur.

Additionally, the disclosed compounds, combinations of disclosed compounds, or compositions thereof, may be used to treat a lung injury. The lung injury may be a chemical- or radiation-induced lung injury.

In some embodiments, the subject may have inhaled or may be expected to be exposed to a pulmonary irritant. In some embodiments, the subject may have inhaled or may be expected to inhale a choking agent. A pulmonary agent, or choking agent, is a chemical agent designed to impede a subject's ability to breathe. These compounds generally operate by causing a build-up of fluids in the lungs, which then leads to suffocation. Inhalation of these agents cause burning of the throat, coughing, vomiting, headache, pain in chest, tightness in chest, and respiratory and circulatory failure. Examples of such agents include: chlorine gas, chloropicrin (PS), diphosgene (DP), phosgene (CG), disulfur decafluoride, perfluoroisobutene, acrolein, and piphenylcyanoarsine. Phosgene-induced acute lung injury (P-ALI) is commonly associated with short-term phosgene inhalation. Prolonged exposure can cause chronic hypoventilation, refractory pulmonary edema, and other associated lung injuries, ultimately resulting in ARDS. Chemical pneumonitis is inflammation of the lungs or breathing difficulty due to inhaling chemical fumes or breathing in and choking on certain chemicals.

Additionally, the disclosed compounds, combinations of disclosed compounds, or compositions thereof, may be used to treat or prevent acute inhalation injury (All) and e-cigarette, or vaping, product use-associated lung injury (EVALI).

In other embodiments, the subject has been exposed to or is expected to be exposed to ionizing radiation. In these embodiments, the subject may have or may be expected to develop radiation induced lung injury (RILI). In some embodiments, the subject may have radiation pneumonitis or radiation pulmonary fibrosis. In these embodiments, the subject may have received or is undergoing thoracic radiotherapy, may have inhaled a radioactive agent or may have had direct exposure to ionizing radiation. For example, the subject may have inhaled a radioactive agent or have had direct exposure to ionizing radiation as a result of a nuclear weapon or leak at a nuclear power plant, for example.

The disclosed compounds, combinations of disclosed compounds, or compositions thereof, also may be used to treat or prevent hemorrhagic fever, or symptoms thereof, including Ebola virus disease, Alkhurma hemorrhagic fever, Chapare hemorrhagic fever, Crimean-Congo hemorrhagic fever, Hantavirus Pulmonary Syndrome (HPS), Hemorrhagic fever with renal syndrome (HFRS), Kyasanur Forest Disease (KFD), Lassa fever, Lujo hemorrhagic fever, Marburg hemorrhagic fever, Omsk hemorrhagic fever, Rift Valley fever, Yellow Fever, or Dengue fever, such as severe dengue fever (dengue hemorrhagic fever).

B. Formulations and Administration

Pharmaceutical compositions comprising one or more active compounds of the disclosure may be manufactured by any suitable method, such as mixing, dissolving, granulating, dragee-making, levigating, emulsifying, encapsulating, entrapping or lyophilization processes. The compositions may be formulated using one or more physiologically acceptable excipients, diluents, carriers, adjuvants or auxiliaries to provide preparations which can be used pharmaceutically.

The active compound(s) may be formulated in the pharmaceutical compositions per se, or in the form of a hydrate, solvate, N-oxide or pharmaceutically acceptable salt. Typically, such salts are more soluble in aqueous solutions than the corresponding free acids and bases, but salts having lower solubility than the corresponding free acids and bases may also be formed.

Pharmaceutical compositions comprising the disclosed compound(s) may take a form suitable for virtually any mode of administration, including, for example, topical, ocular, oral, buccal, systemic, nasal, injection, such as i.v. or i.p., transdermal, rectal, vaginal, etc., or a form suitable for administration by inhalation or insufflation.

For topical administration, the active compound(s) (or a hydrate, solvate, N-oxide or pharmaceutically acceptable salt thereof) may be formulated as solutions, gels, ointments, creams, suspensions, etc. as are well-known in the art.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions, or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives.

Alternatively, the injectable formulation may be provided in powder form for reconstitution with a suitable vehicle, including but not limited to sterile, pyrogen-free water, buffer, dextrose solution, etc., before use. To this end, the active compound(s) maybe dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients, such as: binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); and/or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable excipients such as: suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the active compound, as is well known.

For buccal administration, the compositions may take the form of tablets or lozenges formulated in conventional manner.

For rectal and vaginal routes of administration, the active compound(s) may be formulated as solutions (for retention enemas) suppositories or ointments containing conventional suppository bases, such as cocoa butter or other glycerides.

For nasal administration or administration by inhalation or insufflation, the active compound(s), hydrate, solvate, N-oxide, or pharmaceutically acceptable salt can be conveniently delivered in the form of an aerosol spray from pressurized packs or a nebulizer with the use of a suitable propellant, e.g.,) dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

The pharmaceutical compositions can be in the form of a sterile injectable aqueous or oleagenous suspension. This suspension can be formulated according to the known art using those suitable dispersing or wetting agents and suspending agents which have been mentioned above. The sterile injectable preparation may also be a sterile injectable solution or suspension in a non-toxic parenterally-acceptable diluent or solvent. Among the acceptable vehicles and solvents that can be employed are water, Ringer's solution and isotonic sodium chloride solution.

According to the present disclosure, a form of the disclosed compound(s), solvates, N-oxides, pharmaceutically acceptable salts or prodrug(s) thereof, can also be delivered by any of a variety of inhalation devices and methods known in the art, including, for example: U.S. Pat. Nos. 6,241,969; 6,060,069; 6,238,647; 6,335,316; 5,364,838; 5,672,581; WO96/32149; WO95/24183; U.S. Pat. Nos. 5,654,007; 5,404,871; 5,672,581; 5,743,250; 5,419,315; 5,558,085; WO98/33480; U.S. Pat. Nos. 5,364,833; 5,320,094; 5,780,014; 5,658,878; 5,518,998; 5,506,203; 5,661,130; 5,655,523; 5,645,051; 5,622,166; 5,577,497; 5,492,112; 5,327,883; 5,277,195; U.S. Publication No. 20010041190; U.S. Publication No. 20020006901; and U.S. Publication No. 20020034477.

Included among the devices which can be used to administer a form of the active compound(s) are those well-known in the art, such as, metered dose inhalers, liquid nebulizers, dry powder inhalers, sprayers, thermal vaporizers, and the like. Other suitable technology for administration of particular 2,4-pyrimidinediamine compounds includes electrohydrodynamic aerosolizers.

In addition, the inhalation device is preferably practical, in the sense of being easy to use, small enough to carry conveniently, capable of providing multiple doses, and durable. Some specific examples of commercially available inhalation devices are Turbohaler (Astra, Wilmington, DE), Rotahaler (Glaxo, Research Triangle Park, NC), Diskus (Glaxo, Research Triangle Park, NC), the Ultravent nebulizer (Mallinckrodt), the Acorn II nebulizer (Marquest Medical Products, Totowa, NJ) the Ventolin metered dose inhaler (Glaxo, Research Triangle Park, NC), or the like. In one embodiment, the disclosed compound(s), solvates, N-oxides, pharmaceutically acceptable salts or prodrug(s) thereof can be delivered by a dry powder inhaler or a sprayer.

As those skilled in the art will recognize, the formulation of the form of the disclosed compound(s), solvates, N-oxides, pharmaceutically acceptable salts or prodrug(s) thereof, the quantity of the formulation delivered, and the duration of administration of a single dose depend on the type of inhalation device employed as well as other factors. For some aerosol delivery systems, such as nebulizers, the frequency of administration and length of time for which the system is activated will depend mainly on the concentration of the disclosed compound(s) in the aerosol. For example, shorter periods of administration can be used at higher concentrations the disclosed compound(s) in the nebulizer solution. Devices such as metered dose inhalers can produce higher aerosol concentrations, and can be operated for shorter periods to deliver the desired amount of active compound in some embodiments. Devices such as dry powder inhalers deliver active agent until a given charge of agent is expelled from the device. In this type of inhaler, the amount of the disclosed compound(s), solvates, N-oxides, pharmaceutically acceptable salts or prodrug(s) thereof in a given quantity of the powder determines the dose delivered in a single administration. The formulation of the disclosed compound(s) is selected to yield the desired particle size in the chosen inhalation device.

Formulations of a disclosed compound for administration from a dry powder inhaler may typically include a finely divided dry powder containing the disclosed compound(s), but the powder can also include a bulking agent, buffer, carrier, excipient, another additive, or the like. Additives can be included in a dry powder formulation, for example, to dilute the powder as required for delivery from the particular powder inhaler, to facilitate processing of the formulation, to provide advantageous powder properties to the formulation, to facilitate dispersion of the powder from the inhalation device, to stabilize to the formulation (e.g., antioxidants or buffers), to provide taste to the formulation, or the like. Typical additives include mono-, di-, and polysaccharides; sugar alcohols and other polyols, such as, for example, lactose, glucose, raffinose, melezitose, lactitol, maltitol, trehalose, sucrose, mannitol, starch, or combinations thereof; surfactants, such as sorbitols, diphosphatidyl choline, or lecithin; or the like.

Embodiments of the disclosed method can be conducted a pharmaceutical composition including the disclosed compound(s) suitable for administration by inhalation. For example, a dry powder formulation can be manufactured in several ways, using conventional techniques, such as described in any of the publications mentioned above and incorporated expressly herein by reference, and for example, Baker, et al., U.S. Pat. No. 5,700,904, the entire disclosure of which is incorporated expressly herein by reference. Particles in the size range appropriate for maximal deposition in the lower respiratory tract can be made by micronizing, milling, or the like. And a liquid formulation can be manufactured by dissolving the compound in a suitable solvent, such as water, at an appropriate pH, including buffers or other excipients.

A specific example of an aqueous suspension formulation suitable for nasal administration using commercially-available nasal spray devices includes the following ingredients: active compound (0.5 20 mg/ml); benzalkonium chloride (0.1 0.2 mg/mL); polysorbate 80 (TWEEN® 80; 0.5 5 mg/ml); carboxymethylcellulose sodium or microcrystalline cellulose (1 15 mg/ml); phenylethanol (1 4 mg/ml); and dextrose (20 50 mg/ml). The pH of the final suspension can be adjusted to range from about pH 5 to pH 7, with a pH of about pH 5.5 being typical.

Another specific example of an aqueous suspension suitable for administration of the compounds via inhalation contains 20 mg/mL of the disclosed compound(s), 1% (v/v) polysorbate 80 (TWEEN® 80), 50 mM citrate and/or 0.9% sodium chloride.

For ocular administration, the active compound(s) may be formulated as a solution, emulsion, suspension, etc. suitable for administration to the eye. A variety of vehicles suitable for administering compounds to the eye are known in the art. Specific non-limiting examples are described in U.S. Pat. Nos. 6,261,547; 6,197,934; 6,056,950; 5,800,807; 5,776,445; 5,698,219; 5,521,222; 5,403,841; 5,077,033; 4,882,150; and 4,738,851, which are incorporated herein by reference.

For prolonged delivery, the active compound(s) can be formulated as a depot preparation for administration by implantation or intramuscular injection. The active ingredient maybe formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the active compound(s) for percutaneous absorption may be used. To this end, permeation enhancers may be used to facilitate transdermal penetration of the active compound(s). Suitable transdermal patches are described in for example, U.S. Pat. Nos. 5,407,713; 5,352,456; 5,332,213; 5,336,168; 5,290,561; 5,254,346; 5,164,189; 5,163,899; 5,088,977; 5,087,240; 5,008,110; and 4,921,475, which are incorporated herein by reference.

Alternatively, other pharmaceutical delivery systems may be employed. Liposomes and emulsions are well-known examples of delivery vehicles that may be used to deliver active compound(s). Certain organic solvents, such as dimethylsulfoxide (DMSO), may also be employed, although usually at the cost of greater toxicity.

The pharmaceutical compositions may, if desired, be presented in a pack or dispenser device which may contain one or more unit dosage forms containing the active compound(s). The pack may, for example, comprise metal or plastic foil, such as a blister pack. The pack or dispenser device may be accompanied by instructions for administration.

C. Dosages

The disclosed compound or combinations of disclosed compounds will generally be used in an amount effective to achieve the intended result, for example, in an amount effective to treat, prevent or ameliorate a particular condition. The disclosed compound(s), or compositions thereof, can be administered therapeutically to achieve therapeutic benefit or prophylactically to achieve a prophylactic benefit. Therapeutic benefit means eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. For example, administration of a compound to a patient suffering from an allergy provides therapeutic benefit not only when the underlying allergic response is eradicated or ameliorated, but also when the patient reports a decrease in the severity or duration of the symptoms associated with the allergy following exposure to the allergen. As another example, therapeutic benefit in the context of asthma includes an improvement in respiration following the onset of an asthmatic attack or a reduction in the frequency or severity of asthmatic episodes. Therapeutic benefit also includes halting or slowing the progression of the disease, regardless of whether improvement is realized.

As known by those of ordinary skill in the art, the preferred dosage of disclosed compounds may depend on various factors, including the age, weight, general health, and severity of the condition of the patient or subject being treated. Dosage also may need to be tailored to the sex of the individual and/or the lung capacity of the individual, when administered by inhalation. Dosage may also be tailored to individuals suffering from more than one condition or those individuals who have additional conditions that affect lung capacity and the ability to breathe normally, for example, emphysema, bronchitis, pneumonia, and respiratory infections. Dosage, and frequency of administration of the disclosed compound(s) or compositions thereof, will also depend on whether the disclosed compound(s) are formulated for treatment of acute episodes of a condition or for the prophylactic treatment of a disorder. A person or ordinary skill in the art will be able to determine the optimal dose for a particular individual.

For prophylactic administration, the disclosed compound, combinations of disclosed compounds, or compositions thereof, can be administered to a patient or subject at risk of developing one of the previously described conditions. For example, if it is unknown whether a patient or subject is allergic to a particular drug, the disclosed compound, combinations of disclosed compounds, or compositions thereof, can be administered prior to administration of the drug to avoid or ameliorate an allergic response to the drug. Alternatively, prophylactic administration can be used to avoid or ameliorate the onset of symptoms in a patient diagnosed with the underlying disorder. For example, a disclosed compound(s), or composition thereof, can be administered to an allergy sufferer prior to expected exposure to the allergen. A disclosed compound, combinations of disclosed compounds, or compositions thereof, can also be administered prophylactically to healthy individuals who are repeatedly exposed to agents known to one of the above-described maladies to prevent the onset of the disorder. For example, a disclosed compound, combinations of disclosed compounds, or compositions thereof, can be administered to a healthy individual who is repeatedly exposed to an allergen known to induce allergies, such as latex, in an effort to prevent the individual from developing an allergy. Alternatively, a disclosed compound, combinations of disclosed compounds, or compositions thereof, can be administered to a patient suffering from asthma prior to partaking in activities which trigger asthma attacks to lessen the severity of, or avoid altogether, an asthmatic episode.

Effective dosages can be estimated initially from in vitro assays. For example, an initial dosage for use in subjects can be formulated to achieve a circulating blood or serum concentration of active compound that is at or above an IC₅₀ or EC₅₀ of the particular compound as measured in an in vitro assay. Dosages can be calculated to achieve such circulating blood or serum concentrations taking into account the bioavailability of the particular compound. Fingl & Woodbury, “General Principles,” In: Goodman and Gilman's The Pharmaceutical Basis of Therapeutics, Chapter 1, pages 1-46, Pergamon Press, and the references cited therein, provide additional guidance concerning effective dosages.

In some embodiments, the disclosed compounds have an EC₅₀ with respect to a kinase protein, such as an IRAK protein, of from greater than 0 to 20 μM, such as from greater than 0 to 10 μM, from greater than 0 to 5 μM, from greater than 0 to 1 μM, from greater than 0 to 0.5 μM, from greater than 0 to 0.1 μM, or from greater than 0 to 0.05 μM.

Initial dosages can also be estimated from in vivo data, such as animal models. Animal models useful for testing the efficacy of compounds to treat or prevent the various diseases described above are well-known in the art. Suitable animal models of hypersensitivity or allergic reactions are described in Foster, (1995) Allergy 50(21Suppl):6-9, discussion 34-38 and Tumas et al., (2001), J. Allergy Clin. Immunol. 107(6):1025-1033. Suitable animal models of allergic rhinitis are described in Szelenyi et al., (2000), Arzneimittelforschung 50(11):1037-42; Kawaguchi et al., (1994), Clin. Exp. Allergy 24(3):238-244 and Sugimoto et al., (2000), Immunopharmacology 48(1):1-7. Persons of ordinary skill in the art can adapt such information to determine dosages suitable for human administration.

Dosage amounts of disclosed compounds will typically be in the range of from about greater than 0 mg/kg/day, such as 0.0001 mg/kg/day or 0.001 mg/kg/day or 0.01 mg/kg/day, up to at least about 100 mg/kg/day. More typically, the dosage (or effective amount) may range from about 0.0025 mg/kg to about 1 mg/kg administered at least once per day, such as from 0.01 mg/kg to about 0.5 mg/kg or from about 0.05 mg/kg to about 0.15 mg/kg. The total daily dosage typically ranges from about 0.1 mg/kg to about 5 mg/kg or to about 20 mg/kg per day, such as from 0.5 mg/kg to about 10 mg/kg per day or from about 0.7 mg/kg per day to about 2.5 mg/kg/day. Dosage amounts can be higher or lower depending upon, among other factors, the activity of the disclosed compound, its bioavailability, the mode of administration, and various factors discussed above.

Dosage amount and dosage interval can be adjusted for individuals to provide plasma levels of the disclosed compound that are sufficient to maintain therapeutic or prophylactic effect. For example, the compounds can be administered once per day, multiple times per day, once per week, multiple times per week (e.g., every other day), one per month, multiple times per month, or once per year, depending upon, amongst other things, the mode of administration, the specific indication being treated, and the judgment of the prescribing physician. Persons of ordinary skill in the art will be able to optimize effective local dosages without undue experimentation.

Compositions comprising one or more of the disclosed compounds typically comprise from greater than 0 up to 99% of the disclosed compound, or compounds, and/or other therapeutic agent by total weight percent. More typically, compositions comprising one or more of the disclosed compounds comprise from about 1 to about 20 total weight percent of the disclosed compound and other therapeutic agent, and from about 80 to about 99 weight percent of a pharmaceutically acceptable excipient. Typical daily administrations may be in the range of 100-300 mg/day, e.g., 100, 150, 200, 250, or 300 mg/day. Administration may be once or twice daily or more, e.g., 100 or 150 mg BID. Accordingly, pharmaceutical dosage forms comprising a compound disclosed herein may contain from 50-300 mg of the disclosed compound, e.g., 50, 100, 150, 200, 250, 300 mg of the disclosed compound.

Preferably, the disclosed compound, combinations of disclosed compounds, or compositions thereof, will provide therapeutic or prophylactic benefit without causing substantial toxicity. Toxicity of the disclosed compound can be determined using standard pharmaceutical procedures. The dose ratio between toxic and therapeutic (or prophylactic) effect is the therapeutic index. Disclosed compounds that exhibit high therapeutic indices are preferred.

### VII. Examples

### Example 1

### General Procedure A

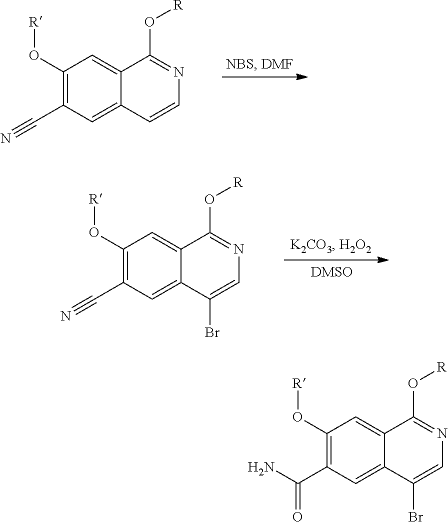

### Representative Example

### (S)-4-bromo-7-isopropoxy-1-((5-oxopyrrolidin-2-yl)methoxy)isoquinoline-6-carboxamide (I-1)

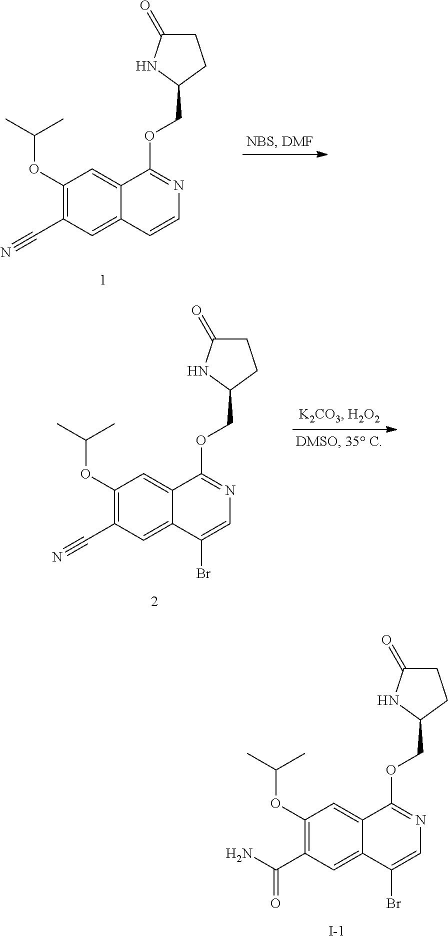 [SMILES: CC(C)Oc1cc2c(OC[C@@H]3CCC(=O)N3)nccc2cc1C#N ; CC(C)Oc1cc2c(OC[C@@H]3CCC(=O)N3)ncc(Br)c2cc1C#N ; CC(C)Oc1cc2c(OC[C@@H]3CCC(=O)N3)ncc(Br)c2cc1C(N)=O ; [I-]]

To a de-gassed DMF (15 mL) solution of (S)-7-isopropoxy-1-((5-oxopyrrolidin-2-yl)methoxy)isoquinoline-6-carbonitrile (1.2 g, 3.69 mmol) was added N-Bromo succinimide (NBS) (985 mg, 5.53 mmol) with cooling in an ice bath. After 5 minutes, the ice bath was removed and the reaction mixture was warmed to room temperature. After another 6 hours, the reaction went to completion as monitored by LCMS, and was quenched by addition of H₂O (80 mL). Precipitate was collected by filtration, washed with water, and was further dried in vacuo. Compound 2: (S)-4-bromo-7-isopropoxy-1-((5-oxopyrrolidin-2-yl)methoxy)isoquinoline-6-carbonitrile was obtained as a light yellow solid: 1.5 g, which was used in next step without further purification.

¹H NMR (400 MHz, Chloroform-d) δ 8.92 (s, 1H), 8.04 (s, 1H), 7.91 (br s, 1H), 7.57 (s, 1H), 6.18 (s, 1H), 6.06 (br s, 1H), 4.94 (hept, J=6.1 Hz, 1H), 4.62 (dd, J=11.1, 3.5 Hz, 1H), 4.39 (dd, J=11.1, 7.0 Hz, 1H), 4.23-4.18 (m, 1H), 2.50-2.36 (m, 4H), 2.08-1.96 (m, 1H), 1.51 (d, J=6.1 Hz, 6H); LRMS (M+H) m/z 422.4, 424.4.

To a DMSO (40 mL) solution of Compound 2 (1.5 g, 3.69 mmol) and K₂CO₃ (1.02 g, 7.4 mmol), H₂O₂ aq. solution (30%, 4.2 mL, 37 mmol) was added. The mixture was stirred at 35° C. for 5 hours until the starting material was not detected by LCMS. The reaction was quenched by addition of H₂O (200 mL) at room temperature, and the precipitate was collected after 30 minutes, which was further washed with H₂O, and then dried in vacuo. Compound I-1: (S)-4-bromo-7-isopropoxy-1-((5-oxopyrrolidin-2-yl)methoxy)isoquinoline-6-carboxamide was obtained as an off-white solid: 1.31 g (84% yield over 2 steps).

¹H NMR (400 MHz, Chloroform-d) δ 8.92 (s, 1H), 8.04 (s, 1H), 7.91 (br s, 1H), 7.57 (s, 1H), 6.18 (s, 1H), 6.06 (br s, 1H), 4.94 (hept, J=6.1 Hz, 1H), 4.62 (dd, J=11.1, 3.5 Hz, 1H), 4.39 (dd, J=11.1, 7.0 Hz, 1H), 4.23-4.18 (m, 1H), 2.50-2.36 (m, 4H), 2.08-1.96 (m, 1H), 1.51 (d, J=6.1 Hz, 6H); LRMS (M+H) m/z 422.4, 424.4.

### Example 2

### General Procedure B

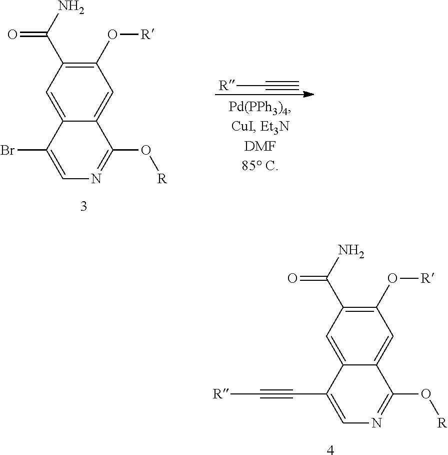

To a de-gassed DMF (1-2 mL) solution of compound 3 (0.05-0.5 mmol), was added Pd(PPh₃)₄(5 mol %) and CuI (10 mol %), followed by Et₃N (5 eq) and alkyne (1.5-2 eq). The reaction mixture was stirred under N₂ at 85° C. overnight, until compound 3 was not detected by LC-MS. Volatiles were removed in vacuo, and the products were purified by Silica Gel chromatography or revered-phase HPLC. When needed, products were dissolved in MeOH or other appropriate solvents, then passed through PL-HCO₃ column for free-basing.

### Example 3

### General Procedure C

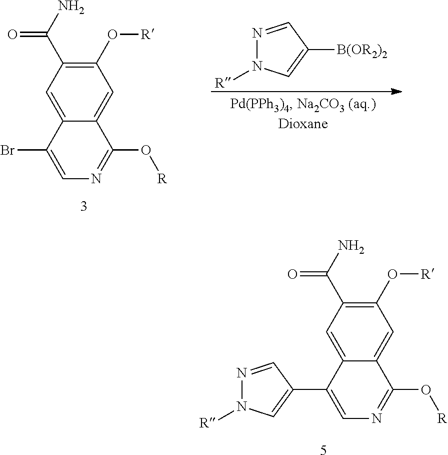

To a 1,4-Dioxane (1-2 mL) solution of compound 3 (0.05-0.5 mmol) was added the boronic acid or ester of choice (1.5 eq), and Pd(PPh₃)₄(5 mol %), followed by Na₂CO₃ aqueous solution (2 M, 3 eq). The reaction mixture was de-gassed and was stirred at 90° C. under N₂ overnight, until compound 3 was not detected by LCMS. Volatiles were removed in vacuo, and the products were purified by Silica Gel chromatography or revered-phase HPLC. When needed, products were dissolved in MeOH or other appropriate solvents, then passed through PL-HCO₃ column for free-basing.

### Example 4

### General Procedure D

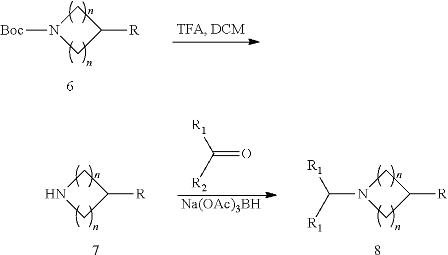

Step 1: Compound 6 (0.05-0.5 mmol) was stirred in TFA-DCM (1:5, 1-5 mL) solution at room temperature until the reaction went to completion as monitored by LC-MS. Volatiles were removed in vacuo, and the products were purified by Silica Gel chromatography or revered-phase HPLC. When needed, products were dissolved in MeOH or other appropriate solvents, then passed through PL-HCO₃ column for free-basing.

Step 2: a MeOH solution of compound 7 (0.05-0.5 mmol), and the aldehyde or ketone of choice (1.5-5 eq) was stirred at room temperature. After 15 minutes to 1 hour, Na(OAc)₃BH (1.5 eq) was added. After the reaction went to completion as monitored by LCMS, water was added to quench the reaction. Volatiles were removed in vacuo, and the products were purified by Silica Gel chromatography or revered-phase HPLC. When needed, products were dissolved in MeOH or other appropriate solvents, then passed through PL-HCO₃ column for free-basing.

### Example 5

### General Procedure E

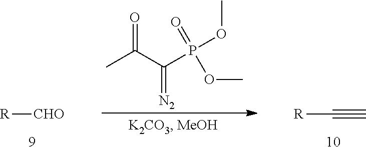

To a MeOH (0.5 mM) solution of compound 9 was added dimethyl (1-diazo-2-oxopropyl)phosphonate (1.3 eq), followed by K₂CO₃ (2.3 eq). Progress of the reaction was monitored by TLC or ¹H NMR after work-up of an aliquot. Upon completion of the reaction, NH₄Cl aqueous solution was added, and product was extracted with EtOAc. Organic layers were combined and further washed with NH₄Cl aqueous solution and brine, then dried (Na₂SO₄), filtered, and the solvent was removed in vacuo. Compound 10 was purified by Silica Gel chromatography.

### Example 6

### General Procedure F

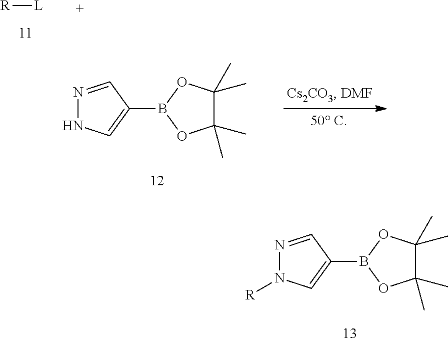

To a DMF (0.5 mM) solution of 4-(4,4,5,5-tetramethyl-1,3,2-dioxaborolan-2-yl)-1H-pyrazole was added the reagent/electrophile of choice and Cs₂CO₃ (1.5 eq). The reaction mixture was stirred at 50° C. under N₂ atmosphere until >50% conversion was achieved, as monitored by LCMS. Volatiles were removed in vacuo, and the products were purified by Silica Gel chromatography.

### Example 7

### General Procedure G

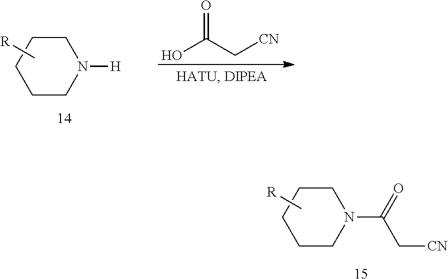

To a DMF (0.1-0.5 mM) solution of compound 14 was added 2-cyanoacetic acid (1 eq), HATU (1.1 eq) and N-ethyl-N-isopropylpropan-2-amine (DIPEA, 3 eq). The reaction mixture was stirred at room temperature until compound 14 was not detected by LCMS. Volatiles were removed in vacuo, and the products were purified by Silica Gel chromatography, or revered-phase HPLC. When needed, products were dissolved in MeOH or other appropriate solvents, then passed through PL-HCO₃ column for free-basing.

### Example 8

### (S)-4-(3-hydroxy-3-methylbut-1-yn-1-yl)-7-isopropoxy-1-((5-oxopyrrolidin-2-yl)methoxy)isoquinoline-6-carboxamide (I-3)

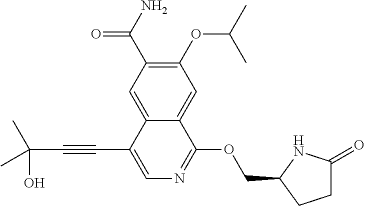 [SMILES: CC(C)Oc1cc2c(OC[C@@H]3CCC(=O)N3)ncc(C#CC(C)(C)O)c2cc1C(N)=O]

General procedures (B): 0.1 mmol scale, 69% yield.

¹H NMR (400 MHz, Chloroform-d) δ 8.82 (s, 1H), 7.96 (br s, 1H), 7.96 (s, 1H), 7.26 (s, overlapped with CHCl₃, 1H), 7.14 (br s, 1H), 6.06 (s, 1H), 4.82-4.75 (m, 1H), 4.50 (br d, J=11.1 Hz, 1H), 4.41 (dd, J=11.1, 5.4 Hz, 1H), 4.23-4.18 (m, 1H), 4.03 (br s, 1H), 2.54-2.35 (m, 3H), 2.05-1.97 (m, 1H), 1.75 (s, 3H), 1.70 (s, partially overlapped with H₂O, 3H), 1.52 (d, J=6.0 Hz, 3H), 1.47 (d, J=6.0 Hz, 3H; LRMS (M+H) m/z 426.5.

### Example 9

### (S)-4-((3-hydroxyoxetan-3-yl)ethynyl)-7-isopropoxy-1-((5-oxopyrrolidin-2-yl)methoxy)isoquinoline-6-carboxamide (I-4)

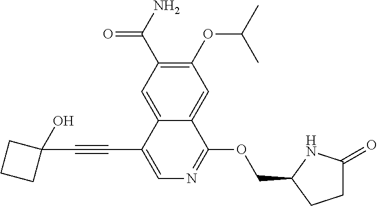 [SMILES: CC(C)Oc1cc2c(OC[C@@H]3CCC(=O)N3)ncc(C#CC3(O)CCC3)c2cc1C(N)=O]

General procedures (B): 0.1 mmol scale, 90% yield.

¹H NMR (400 MHz, DMSO-d₆) δ 8.31 (s, 1H), 8.12 (s, 1H), 8.11 (s, 1H), 7.77 (s, 1H), 7.76 (s, 1H), 7.68 (s, 1H), 4.92 (hept, J=6.0 Hz, 1H), 4.82 (d, J=6.7 Hz, 2H), 4.66 (d, J=6.7 Hz, 2H), 4.50 (dd, J=10.9, 3.8 Hz, 1H), 4.32 (dd, J=10.9, 6.5 Hz, 1H), 4.05-4.00 (m, 1H), 2.33-2.14 (m, 3H), 1.92-1.87 (m, 1H), 1.40 (d, J=6.0 Hz, 3H), 1.37 (d, J=6.0 Hz, 3H; LRMS (M+H) m/z 440.5.

### Example 10

### (S)-4-(4-hydroxy-3,3-dimethylbut-1-yn-1-yl)-7-isopropoxy-1-((5-oxopyrrolidin-2-yl)methoxy)isoquinoline-6-carboxamide (I-5)

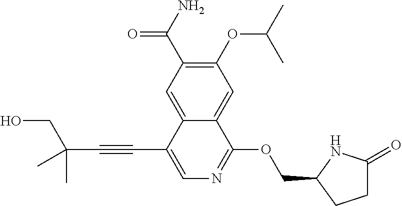 [SMILES: CC(C)Oc1cc2c(OC[C@@H]3CCC(=O)N3)ncc(C#CC(C)(C)CO)c2cc1C(N)=O]

General procedures (B): 0.1 mmol scale, 99% yield.

¹H NMR (400 MHz, Chloroform-d) δ 8.98 (s, 1H), 7.98 (s, 1H), 7.95 (s, 1H), 7.46 (s, 1H), 6.32 (s, 1H), 6.00 (s, 1H), 4.87 (hept, J=6.0 Hz, 1H), 4.62 (dd, J=11.1, 3.4 Hz, 1H), 4.42 (dd, J=11.1, 6.5 Hz, 1H), 4.23-4.18 (m, 1H), 3.67-3.63 (m, 2H), 2.74 (t, J=6.7 Hz, 1H), 2.50-2.36 (m, 3H), 2.09-1.97 (m, 1H), 1.50 (d, J=6.0 Hz, 3H), 1.49 (d, J=6.0 Hz, 3H), 1.41 (s, 6H; LRMS (M+H) m/z 440.5.

### Example 11

### (S)-7-isopropoxy-1-((5-oxopyrrolidin-2-yl)methoxy)-4-((tetrahydro-2H-pyran-4-yl)ethynyl)isoquinoline-6-carboxamide (I-6)

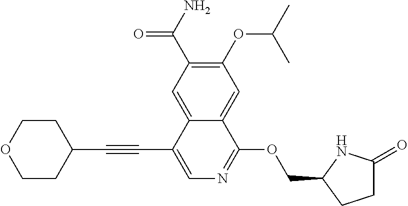 [SMILES: CC(C)Oc1cc2c(OC[C@@H]3CCC(=O)N3)ncc(C#CC3CCOCC3)c2cc1C(N)=O]

General procedures (B): 0.1 mmol scale, 49% yield.

¹H NMR (400 MHz, Chloroform-d) δ 9.03 (s, 1H), 8.02 (s, 1H), 7.93 (s, 1H), 7.56 (s, 1H), 6.06 (s, 1H), 5.93 (s, 1H), 4.98-4.88 (m, 1H), 4.65 (dd, J=11.1, 3.5 Hz, 1H), 4.41 (dd, J=11.1, 7.1 Hz, 1H), 4.24-4.18 (m, 1H), 4.01 (ddd, J=11.6, 5.6, 3.7 Hz, 2H), 3.60 (ddd, J=11.6, 8.5, 3.0 Hz, 2H), 3.00 (tt, J=8.4, 4.1 Hz, 1H), 2.52-2.36 (m, 3H), 2.05-1.98 (m, 3H), 1.92-1.83 (m, 2H), 1.51 (d, J=6.0 Hz, 6H); LRMS (M+H) m/z 452.6.

### Example 12

### (S)-7-isopropoxy-4-(3-morpholinoprop-1-yn-1-yl)-1-((5-oxopyrrolidin-2-yl)methoxy)isoquinoline-6-carboxamide (I-7)

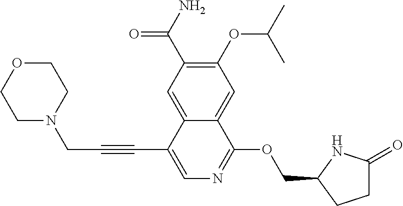 [SMILES: CC(C)Oc1cc2c(OC[C@@H]3CCC(=O)N3)ncc(C#CCN3CCOCC3)c2cc1C(N)=O]

General procedures (B): 0.1 mmol scale, 63% yield.

¹H NMR (400 MHz, Chloroform-d) δ 9.00 (s, 1H), 8.05 (s, 1H), 7.91 (br s, 1H), 7.55 (br s, 1H), 6.23 (br s, 1H), 6.02 (br s, 1H), 4.96-4.87 (m, 1H), 4.63 (dd, J=11.1, 3.5 Hz, 1H), 4.40 (dd, J=11.1, 7.1 Hz, 1H), 4.22-4.16 (m, 1H), 3.80-3.78 (m, 4H), 3.64 (s, 2H), 2.72-2.70 (m, 4H), 2.46-2.35 (m, 3H), 2.05-1.94 (m, 1H), 1.49 (d, J=6.0 Hz, 3H), 1.48 (d, J=6.0 Hz, 3H); LRMS (M+H) m/z 467.1.

### Example 13

### (S)-7-isopropoxy-4-(4-morpholinobut-1-yn-1-yl)-1-((5-oxopyrrolidin-2-yl)methoxy)isoquinoline-6-carboxamide (I-8)

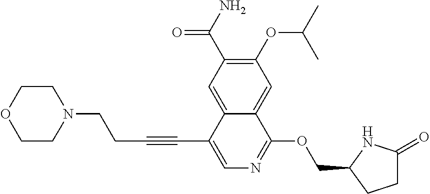 [SMILES: CC(C)Oc1cc2c(OC[C@@H]3CCC(=O)N3)ncc(C#CCCN3CCOCC3)c2cc1C(N)=O]

General procedures (B): 0.1 mmol scale, 11% yield.

¹H NMR (400 MHz, Chloroform-d) δ 8.99 (s, 1H), 8.00 (s, 1H), 7.91 (br s, 1H), 7.54 (br s, 1H), 6.04 (br s, 1H), 5.94 (br s, 1H), 4.95-4.86 (m, 1H), 4.62 (dd, J=11.1, 3.5 Hz, 1H), 4.39 (dd, J=11.1, 7.1 Hz, 1H), 4.22-4.16 (m, 1H), 3.75-3.73 (m, 4H), 2.78-2.70 (m, 4H), 2.58-2.56 (m, 4H), 2.46-2.34 (m, 3H), 2.05-1.95 (m, 1H), 1.50 (d, J=6.1 Hz, 3H), 1.49 (d, J=6.1 Hz, 3H); LRMS (M+H) m/z 481.2.

### Example 14

### (S)-4-((1-hydroxycyclopentyl)ethynyl)-7-isopropoxy-1-((5-oxopyrrolidin-2-yl)methoxy)isoquinoline-6-carboxamide (I-9)

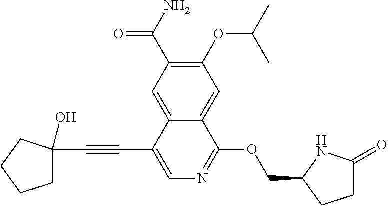 [SMILES: CC(C)Oc1cc2c(OC[C@@H]3CCC(=O)N3)ncc(C#CC3(O)CCCC3)c2cc1C(N)=O]

General procedures (B): 0.1 mmol scale, 43% yield.

¹H NMR (400 MHz, DMSO-d₆) δ 8.29 (s, 1H), 8.09 (br s, 1H), 8.00 (s, 1H), 7.74 (br s, 1H), 7.71 (br s, 1H), 7.65 (s, 1H), 5.46 (s, 1H), 4.94-4.85 (m, 1H), 4.47 (dd, J=10.9, 3.8 Hz, 1H), 4.29 (dd, J=10.9, 6.5 Hz, 1H), 4.03-3.97 (m, 1H), 2.31-2.13 (m, 3H), 1.99-1.85 (m, 5H), 1.80-1.67 (m, 3H), 1.38 (d, J=6.0 Hz, 3H), 1.35 (d, J=6.0 Hz, 3H); LRMS (M+Na) m/z 474.1.

### Example 15

### (S)-4-((4-hydroxytetrahydro-2H-pyran-4-yl)ethynyl)-7-isopropoxy-1-((5-oxopyrrolidin-2-yl)methoxy)isoquinoline-6-carboxamide (I-10)

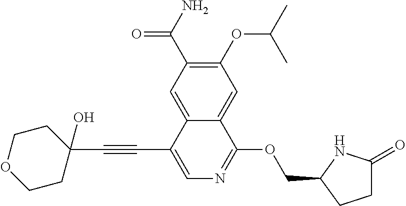 [SMILES: CC(C)Oc1cc2c(OC[C@@H]3CCC(=O)N3)ncc(C#CC3(O)CCOCC3)c2cc1C(N)=O]

General procedures (B): 0.1 mmol scale, 83% yield.

¹H NMR (400 MHz, DMSO-d₆) δ 8.33 (s, 1H), 8.09 (br s, 1H), 8.05 (s, 1H), 7.75 (br s, 1H), 7.71 (br s, 1H), 7.66 (s, 1H), 5.87 (s, 1H), 4.95-4.86 (m, 1H), 4.47 (dd, J=10.9, 3.8 Hz, 1H), 4.30 (dd, J=10.9, 6.5 Hz, 1H), 4.03-3.97 (m, 1H), 3.80 (ddd, J=11.5, 5.4, 3.8 Hz, 2H), 3.60 (ddd, J=11.6, 8.7, 3.0 Hz, 2H), 2.31-2.13 (m, 3H), 1.95-1.85 (m, 3H), 1.75 (ddd, J=12.7, 8.7, 3.8 Hz, 2H), 1.15 (d, J=6.0 Hz, 3H), 1.13 (d, J=6.0 Hz, 3H); LRMS (M+Na) m/z 490.1.

### Example 16

(S)-7-isopropoxy-1-((5-oxopyrrolidin-2-yl)methoxy)-4-(3-(tetrahydro-2H-pyran-4-yl)prop-1-yn-1-yl)isoquinoline-6-carboxamide (I-11)

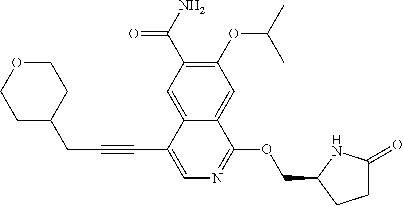 [SMILES: CC(C)Oc1cc2c(OC[C@@H]3CCC(=O)N3)ncc(C#CCC3CCOCC3)c2cc1C(N)=O]

General procedures (B): 0.1 mmol scale, 47% yield.

¹H NMR (400 MHz, Chloroform-d) δ 9.01 (s, 1H), 8.00 (s, 1H), 7.92 (br s, 1H), 7.54 (s, 1H), 6.07 (s, 1H), 5.94 (br s, 1H), 4.91 (hept, J=6.1 Hz, 1H), 4.63 (dd, J=11.1, 3.5 Hz, 1H), 4.39 (dd, J=11.1, 7.1 Hz, 1H), 4.22-4.16 (m, 1H), 4.01 (br dd, J=11.0, 4.1 Hz, 2H), 3.44 (td, J=11.8, 2.0 Hz, 2H), 2.50 (d, J=6.6 Hz, 2H), 2.46-2.36 (m, 3H), 2.03-1.82 (m, 4H), 1.55-1.44 (m, 8H); LRMS (M+H) m/z 466.6.

### Example 17

### tert-butyl (S)-4-((6-carbamoyl-7-isopropoxy-1-((5-oxopyrrolidin-2-yl)methoxy)isoquinolin-4-yl)ethynyl)-4-hydroxypiperidine-1-carboxylate (I-12)

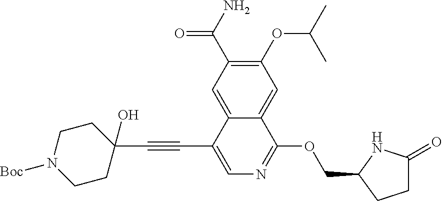 [SMILES: CC(C)Oc1cc2c(OC[C@@H]3CCC(=O)N3)ncc(C#CC3(O)CCN(C(=O)OC(C)(C)C)CC3)c2cc1C(N)=O]

General procedures (B): 0.2 mmol scale, 79% yield.

¹H NMR (400 MHz, Chloroform-d) δ 11.05 (br s, 0.5H), 8.75 (s, 1H), 7.98 (s, 0.5H), 7.97 (s, 0.5H), 7.91 (s, 1H), 7.53 (br s, 0.5H), 7.17 (s, 1H), 6.03 (s, 0.5H), 6.02 (s, 0.5H), 4.76-4.70 (m, 1H), 4.58 (br s, 1H), 4.47-4.38 (m, 2H), 4.21-4.16 (m, 1H), 3.84-3.76 (m, 2H), 3.48-3.39 (m, 2H), 2.54-2.34 (m, 3H), 2.15-1.86 (m, 5H), 1.51 (d, J=6.0 Hz, 3H), 1.46-1.45 (m, 12H); LRMS (M+Na) m/z 589.7.

### Example 18

### (S)-4-((4-hydroxypiperidin-4-yl)ethynyl)-7-isopropoxy-1-((5-oxopyrrolidin-2-yl)methoxy)isoquinoline-6-carboxamide (I-13)

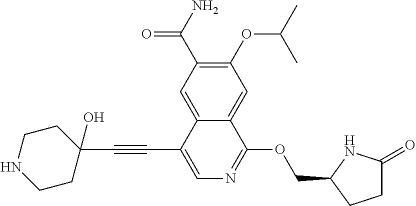 [SMILES: CC(C)Oc1cc2c(OC[C@@H]3CCC(=O)N3)ncc(C#CC3(O)CCNCC3)c2cc1C(N)=O]

General procedures (D), step 1: 0.1 mmol scale, 88% yield.

¹H NMR (400 MHz, Methanol-d₄) δ 8.70 (s, 1H), 8.05 (s, 1H), 7.73 (s, 1H), 4.98 (hept, J=6.0 Hz, 1H), 4.59 (dd, J=11.2, 3.6 Hz, 1H), 4.49 (dd, J=11.2, 5.4 Hz, 1H), 4.22-4.17 (m, 1H), 3.24-3.19 (m, 2H), 3.13-3.11 (m, 2H), 2.52-2.35 (m, 3H), 2.20-2.13 (m, 2H), 2.11-2.03 (m, 1H), 2.00-1.93 (m, 2H), 1.49 (d, J=6.0 Hz, 3H), 1.47 (d, J=6.0 Hz, 3H); LRMS (M+H) m/z 467.6.

### Example 19

### (S)-4-((4-hydroxy-1-methylpiperidin-4-yl)ethynyl)-7-isopropoxy-1-((5-oxopyrrolidin-2-yl)methoxy)isoquinoline-6-carboxamide (I-14)

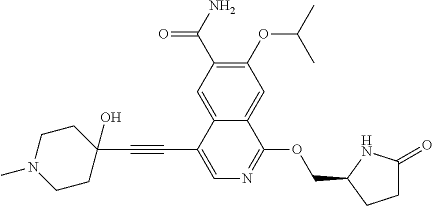 [SMILES: CC(C)Oc1cc2c(OC[C@@H]3CCC(=O)N3)ncc(C#CC3(O)CCN(C)CC3)c2cc1C(N)=O]

General procedures (D), step 2, formaldehyde was used: 0.06 mmol scale, 58% yield.

¹H NMR (400 MHz, Methanol-d₄) δ 8.69 (s, 1H), 8.05 (s, 1H), 7.73 (s, 1H), 4.97 (hept, J=6.0 Hz, 1H), 4.59 (dd, J=11.2, 3.6 Hz, 1H), 4.49 (dd, J=11.2, 5.4 Hz, 1H), 4.22-4.17 (m, 1H), 3.03-2.95 (m, 2H), 2.88-2.78 (m, 2H), 2.55-2.35 (m, 7H), 2.21-2.15 (m, 2H), 2.11-2.03 (m, 3H), 1.49 (d, J=6.0 Hz, 3H), 1.47 (d, J=6.0 Hz, 3H); LRMS (M+H) m/z 481.6.

### Example 20

### tert-butyl (S)-4-((6-carbamoyl-7-isopropoxy-1-((5-oxopyrrolidin-2-yl)methoxy)isoquinolin-4-yl)ethynyl)piperidine-1-carboxylate (I-15)

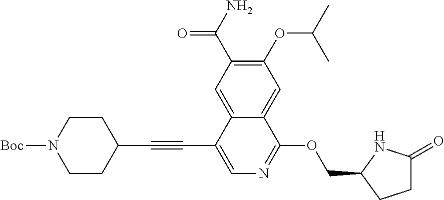 [SMILES: CC(C)Oc1cc2c(OC[C@@H]3CCC(=O)N3)ncc(C#CC3CCN(C(=O)OC(C)(C)C)CC3)c2cc1C(N)=O]

General procedures (B): 0.15 mmol scale, 57% yield.

¹H NMR (400 MHz, Chloroform-d) δ 8.97 (br s, 1H), 8.01 (s, 1H), 7.92 (d, J=3.8 Hz, 1H), 7.57 (s, 1H), 6.58-6.55 (m, 1H), 6.14 (br s, 1H), 1.49 (d, J=6.0 Hz, 3H), 4.64 (dd, J=11.1, 3.5 Hz, 1H), 4.40 (dd, J=11.1, 7.1 Hz, 1H), 4.24-4.18 (m, 1H), 3.86-3.80 (m, 2H), 3.24 (ddd, J=13.5, 8.7, 3.3 Hz, 2H), 2.92 (tt, J=8.2, 4.0 Hz, 1H), 2.49-2.36 (m, 3H), 2.04-1.92 (m, 2H), 1.82-1.73 (m, 3H), 1.49 (d, J=6.0 Hz, 3H), 1.48 (d, J=6.0 Hz, 3H), 1.47 (s, 9H); LRMS (M+Na) m/z 573.6.

### Example 21

### (S)-7-isopropoxy-1-((5-oxopyrrolidin-2-yl)methoxy)-4-(piperidin-4-ylethynyl)isoquinoline-6-carboxamide (I-17)

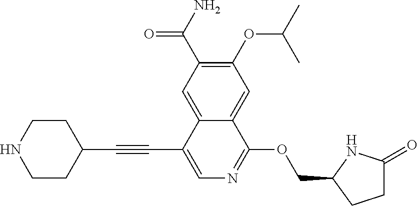 [SMILES: CC(C)Oc1cc2c(OC[C@@H]3CCC(=O)N3)ncc(C#CC3CCNCC3)c2cc1C(N)=O]

General procedures (D), step 1: 0.043 mmol scale, 83% yield.

¹H NMR (400 MHz, Methanol-d₄) δ 8.69 (s, 1H), 8.56 (br s, 1H), 8.01 (s, 1H), 7.69 (s, 1H), 4.98 (hept, J=6.1 Hz, 1H), 4.58 (dd, J=11.1, 3.6 Hz, 1H), 4.48 (dd, J=11.1, 5.4 Hz, 1H), 4.23-4.18 (m, 1H), 3.43 (ddd, J=12.8, 7.3, 3.7 Hz, 2H), 3.21-3.14 (m, 3H), 2.56-2.37 (m, 3H), 2.26-2.19 (m, 2H), 2.12-1.97 (m, 3H), 1.50 (d, J=6.1 Hz, 3H), 1.49 (d, J=6.1 Hz, 3H); LRMS (M+H) m/z 451.5.

### Example 22

### methyl (S)-4-((6-carbamoyl-7-isopropoxy-1-((5-oxopyrrolidin-2-yl)methoxy)isoquinolin-4-yl)ethynyl)piperidine-1-carboxylate (I-16)

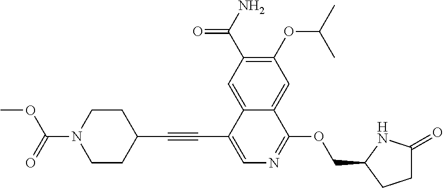 [SMILES: COC(=O)N1CCC(C#Cc2cnc(OC[C@@H]3CCC(=O)N3)c3cc(OC(C)C)c(C(N)=O)cc23)CC1]

To a THF-DCM (1:1, 1 mL) solution of (S)-7-isopropoxy-1-((5-oxopyrrolidin-2-yl)methoxy)-4-(piperidin-4-ylethynyl)isoquinoline-6-carboxamide trifluoroacetate (0.027 mmol), NaHCO₃ (4.5 mg, 0.054 mmol) and DIPEA (14 μL, 0.054 mmol) were added at room temperature. After 2 hours, methyl chloroformate (4 μL, 0.054 mmol) was added, and stirring was continued overnight. The reaction went to completion as monitored by LCMS, and was then quenched by MeOH. Volatiles were removed in vacuo, and title compound was purified by revered-phase HPLC: 11 mg (22% yield).

¹H NMR (400 MHz, Chloroform-d) δ 8.98 (s, 1H), 8.01 (s, 1H), 7.93 (br s, 1H), 7.57 (s, 1H), 6.36 (s, 1H), 6.06 (br s, 1H), 4.93 (hept, J=6.1 Hz, 1H), 4.64 (dd, J=11.1, 3.5 Hz, 1H), 4.40 (dd, J=11.1, 7.1 Hz, 1H), 4.24-4.18 (m, 1H), 3.89-3.82 (m, 2H), 3.71 (s, 3H), 3.35 (ddd, J=13.5, 8.4, 3.4 Hz, 2H), 3.00-2.94 (m, 1H), 2.50-2.35 (m, 3H), 2.04-1.93 (m, 3H), 1.84-1.76 (m, 2H), 1.50 (d, J=6.1 Hz, 6H); LRMS (M+H) m/z 509.5.

### Example 23

### (S)-7-isopropoxy-4-((1-methylpiperidin-4-yl)ethynyl)-1-((5-oxopyrrolidin-2-yl)methoxy)isoquinoline-6-carboxamide (I-18)

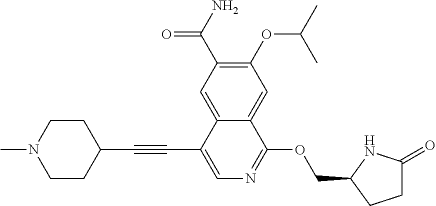 [SMILES: CC(C)Oc1cc2c(OC[C@@H]3CCC(=O)N3)ncc(C#CC3CCN(C)CC3)c2cc1C(N)=O]

General procedures (D), step 2, formaldehyde was used: 0.027 mmol scale, 83% yield.

¹H NMR (400 MHz, DMSO-d₆) δ 8.37 (s, 1H), 8.12 (s, 1H), 8.02 (s, 1H), 7.78 (s, 1H), 7.76 (s, 1H), 7.68 (s, 1H), 4.93 (hept, J=6.0 Hz, 1H), 4.49 (dd, J=10.9, 3.8 Hz, 1H), 4.31 (dd, J=10.9, 6.5 Hz, 1H), 4.05-4.00 (m, 1H), 2.79 (br s, 1H), 2.69-2.64 (m, 2H), 2.34-2.14 (m, 8H), 1.96-1.88 (m, 3H), 1.75-1.66 (m, 2H), 1.41 (d, J=6.0 Hz, 3H), 1.38 (d, J=6.0 Hz, 3H); LRMS (M+H) m/z 465.5.

### Example 24

### (S)-7-isopropoxy-4-((1-isopropylpiperidin-4-yl)ethynyl)-1-((5-oxopyrrolidin-2-yl)methoxy)isoquinoline-6-carboxamide (I-19)

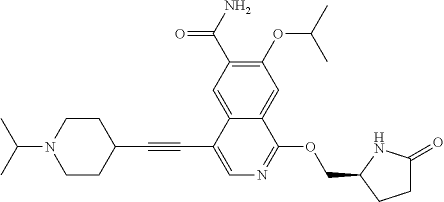 [SMILES: CC(C)Oc1cc2c(OC[C@@H]3CCC(=O)N3)ncc(C#CC3CCN(C(C)C)CC3)c2cc1C(N)=O]

General procedures (D), step 2, acetone was used: 0.027 mmol scale, 80% yield.

¹H NMR (400 MHz, Chloroform-d) δ 9.00 (s, 1H), 8.00 (s, 1H), 7.90 (d, J=4.0 Hz, 1H), 7.56 (s, 1H), 6.35 (s, 1H), 6.07 (d, J=4.0 Hz, 1H), 4.92 (hept, J=6.0 Hz, 1H), 4.64 (dd, J=11.1, 3.5 Hz, 1H), 4.40 (dd, J=11.1, 7.1 Hz, 1H), 4.23-4.18 (m, 1H), 2.85 (ddd, J=10.4, 6.0, 3.6 Hz, 2H), 2.77-2.70 (m, 2H), 2.50-2.31 (m, 5H), 2.06-1.98 (m, 2H), 1.91-1.83 (m, 2H), 1.49 (d, J=6.0 Hz, 6H), 1.06 (d, J=6.5 Hz, 6H); LRMS (M+H) m/z 493.5.

### Example 25

### trans-1-ethoxy-4-ethynylcyclohexane

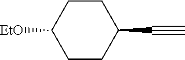

General procedures (E): 5 mmol scale, 51% yield.

¹H NMR (400 MHz, Chloroform-d) δ 3.50 (q, J=7.0 Hz, 2H), 3.25 (tt, J=9.6, 3.5 Hz, 1H), 2.31-2.24 (m, 1H), 2.03 (d, J=2.4 Hz, partially overlapped, 1H), 2.03-1.98 (m, partially overlapped, 4H), 1.46-1.37 (m, 2H), 1.32-1.25 (m, 2H), 1.19 (t, J=7.0 Hz, 3H).

### Example 26

### 4-(((1r,4S)-4-ethoxycyclohexyl)ethynyl)-7-isopropoxy-1-(((S)-5-oxopyrrolidin-2-yl)methoxy)isoquinoline-6-carboxamide (I-20)

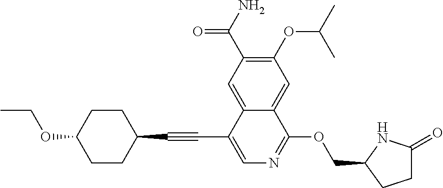 [SMILES: CCO[C@H]1CC[C@H](C#Cc2cnc(OC[C@@H]3CCC(=O)N3)c3cc(OC(C)C)c(C(N)=O)cc23)CC1]

General procedures (B): 0.05 mmol scale, 45% yield.

¹H NMR (400 MHz, DMSO-d₆) δ 8.33 (s, 1H), 8.11 (s, 1H), 8.00 (s, 1H), 7.77 (br s, 1H), 7.75 (br s, 1H), 7.67 (s, 1H), 4.93 (hept, J=6.0 Hz, 1H), 4.48 (dd, J=10.9, 3.8 Hz, 1H), 4.31 (dd, J=10.9, 6.5 Hz, 1H), 4.05-3.99 (m, 1H), 3.45 (q, J=7.0 Hz, 2H), 3.35-3.28 (partially overlapped with H₂O, 1H), 2.70 (tt, J=10.1, 3.6 Hz, 1H), 2.34-2.14 (m, 3H), 2.07-1.88 (m, 5H), 1.56-1.48 (m, 2H), 1.40 (d, J=6.0 Hz, 3H), 1.38 (d, J=6.0 Hz, 3H), 1.36-1.26 (partially overlapped, 2H), 1.10 (t, J=7.0 Hz, 3H); LRMS (M+H) m/z 494.3.

### Example 27

### cis-1-ethoxy-4-ethynylcyclohexane

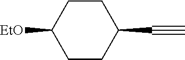

General procedures (E): 6.76 mmol scale, >90% yield.

¹H NMR (400 MHz, Chloroform-d) δ 3.50 (q, J=7.0 Hz, 2H), 3.31 (tt, J=7.3, 3.6 Hz, 1H), 2.58-2.52 (m, 1H), 2.06 (d, J=2.5 Hz, 1H), 1.87-1.65 (m, 5H), 1.60-1.53 (m, 3H), 1.21 (t, J=7.0 Hz, 3H).

### Example 28

### 4-((cis-4-ethoxycyclohexyl)ethynyl)-7-isopropoxy-1-(((S)-5-oxopyrrolidin-2-yl)methoxy)isoquinoline-6-carboxamide (I-21)

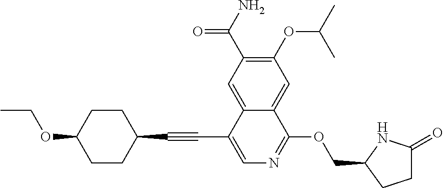 [SMILES: CCO[C@H]1CC[C@@H](C#Cc2cnc(OC[C@@H]3CCC(=O)N3)c3cc(OC(C)C)c(C(N)=O)cc23)CC1]

General procedures (B): 0.06 mmol scale, 29% yield.

¹H NMR (400 MHz, DMSO-d₆) δ 8.33 (s, 1H), 8.12 (s, 1H), 8.02 (s, 1H), 7.78 (br s, 1H), 7.74 (br s, 1H), 7.68 (s, 1H), 4.93 (hept, J=6.0 Hz, 1H), 4.49 (dd, J=10.9, 3.8 Hz, 1H), 4.31 (dd, J=10.9, 6.5 Hz, 1H), 4.05-4.00 (m, 1H), 3.44 (q, J=7.0 Hz, 2H), 3.39-3.35 (m, 2H), 2.93-2.88 (m, 1H), 2.34-2.16 (m, 3H), 1.93-1.77 (m, 2H), 1.75-1.66 (m, 6H), 1.40 (d, J=6.0 Hz, 3H), 1.38 (d, J=6.0 Hz, 3H), 1.11 (t, J=7.0 Hz, 3H); LRMS (M+H) m/z 494.5.

### Example 29

### (1r,4r)-4-ethynyl-1-methylcyclohexan-1-ol

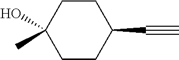

General procedures (E): 3.4 mmol scale, >44% yield.

¹H NMR (400 MHz, Chloroform-d) δ 2.61-2.55 (m, 1H), 2.02 (d, J=2.5 Hz, 1H), 1.94-1.86 (m, 2H), 1.77 (ddd, J=13.3, 9.8, 3.6 Hz, 2H), 1.61-1.54 (m, 2H), 1.49-1.43 (m, 2H), 1.25 (s, 3H).

### Example 30

### 4-(((1r,4S)-4-hydroxy-4-methylcyclohexyl)ethynyl)-7-isopropoxy-1-(((S)-5-oxopyrrolidin-2-yl)methoxy)isoquinoline-6-carboxamide (I-22)

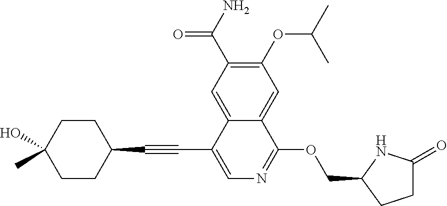 [SMILES: CC(C)Oc1cc2c(OC[C@@H]3CCC(=O)N3)ncc(C#C[C@H]3CC[C@](C)(O)CC3)c2cc1C(N)=O]

General procedures (B): 0.05 mmol scale, 48% yield.

¹H NMR (400 MHz, DMSO-d₆) δ 8.37 (s, 1H), 8.12 (s, 1H), 8.01 (s, 1H), 7.76 (br s, 2H), 7.68 (s, 1H), 4.94 (hept, J=6.1 Hz, 1H), 4.49 (dd, J=10.9, 3.8 Hz, 1H), 4.31 (dd, J=10.9, 6.5 Hz, 1H), 4.16 (s, 1H), 4.05-3.99 (m, 1H), 2.95-2.90 (m, 1H), 2.34-2.16 (m, 3H), 2.01-1.88 (m, 3H), 1.75-1.68 (m, 2H), 1.62-1.54 (m, 2H), 1.50-1.44 (m, 2H), 1.40 (d, J=6.1 Hz, 3H), 1.38 (d, J=6.1 Hz, 3H), 1.17 (s, 3H); LRMS (M+H) m/z 480.5.

### Example 31

### (1s,4s)-4-ethynyl-1-methylcyclohexan-1-ol

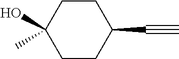

General procedures (E): 3.27 mmol scale, >35% yield.

¹H NMR (400 MHz, Chloroform-d) δ 2.31-2.23 (m, 1H), 2.05 (d, J=2.3 Hz, 1H), 1.79-1.73 (m, 4H), 1.72-1.66 (m, 2H), 1.43-1.36 (m, 2H), 1.22 (s, 3H).

### Example 32

### 4-(((1s,4R)-4-hydroxy-4-methylcyclohexyl)ethynyl)-7-isopropoxy-1-(((S)-5-oxopyrrolidin-2-yl)methoxy)isoquinoline-6-carboxamide (I-23)

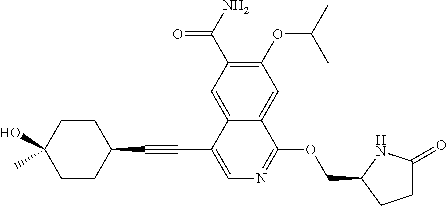 [SMILES: CC(C)Oc1cc2c(OC[C@@H]3CCC(=O)N3)ncc(C#C[C@H]3CC[C@@](C)(O)CC3)c2cc1C(N)=O]

General procedures (B): 0.05 mmol scale, 68% yield.

¹H NMR (400 MHz, DMSO-d₆) δ 8.34 (s, 1H), 8.12 (s, 1H), 8.01 (s, 1H), 7.78 (s, 1H), 7.75 (s, 1H), 7.67 (s, 1H), 4.93 (hept, J=6.0 Hz, 1H), 4.48 (dd, J=10.9, 3.8 Hz, 1H), 4.31 (dd, J=10.9, 6.4 Hz, 1H), 4.13 (s, 1H), 4.05-3.99 (m, 1H), 2.64-2.57 (m, 1H), 2.34-2.16 (m, 3H), 1.95-1.75 (m, 5H), 1.63-1.59 (m, 2H), 1.41-1.32 (m, 8H), 1.11 (s, 3H); LRMS (M−H₂O+H) m/z 462.4.

### Example 33

### tert-butyl (S)-3-((6-carbamoyl-7-isopropoxy-1-((5-oxopyrrolidin-2-yl)methoxy)isoquinolin-4-yl)ethynyl)azetidine-1-carboxylate (I-24)

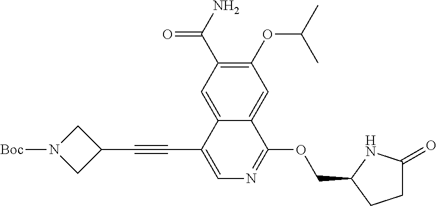 [SMILES: CC(C)Oc1cc2c(OC[C@@H]3CCC(=O)N3)ncc(C#CC3CN(C(=O)OC(C)(C)C)C3)c2cc1C(N)=O]

General procedures (B): 0.1 mmol scale, 50% yield.

¹H NMR (400 MHz, Chloroform-d) δ 8.95 (s, 1H), 8.05 (s, 1H), 7.92 (d, J=3.8 Hz, 1H), 7.57 (s, 1H), 6.14 (s, 1H), 5.99 (d, J=3.8 Hz, 1H), 4.93 (hept, J=6.0 Hz, 1H), 4.65 (dd, J=11.1, 3.5 Hz, 1H), 4.42 (dd, J=11.1, 7.1 Hz, 1H), 4.28 (dd, J=8.5, 8.5 Hz, 2H), 4.24-4.18 (m, 1H), 4.12 (dd, J=8.3, 6.4 Hz, 2H), 3.70 (tt, J=8.8, 6.4 Hz, 1H), 2.50-2.36 (m, 3H), 2.08-1.97 (m, 1H), 1.51 (d, J=6.0 Hz, 6H), 1.46 (s, 9H); LRMS (M+Na) m/z 545.1.

### Example 34

### (S)-4-(azetidin-3-ylethynyl)-7-isopropoxy-1-((5-oxopyrrolidin-2-yl)methoxy)isoquinoline-6-carboxamide (I-25)

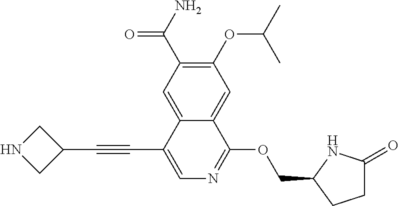 [SMILES: CC(C)Oc1cc2c(OC[C@@H]3CCC(=O)N3)ncc(C#CC3CNC3)c2cc1C(N)=O]

General procedures (D), step 1: 0.04 mmol scale, 99% yield.

¹H NMR (400 MHz, DMSO-d₆) δ 8.31 (s, 1H), 8.12 (s, 1H), 8.07 (s, 1H), 7.78 (br s, 1H), 7.77 (br s, 1H), 7.68 (s, 1H), 4.93 (hept, J=6.0 Hz, 1H), 4.50 (dd, J=10.9, 3.8 Hz, 1H), 4.32 (dd, J=10.9, 6.5 Hz, 1H), 4.03 (tt, J=7.8, 4.1 Hz, 1H), 3.94-3.78 (m, overlapped with H₂O, 6H), 2.34-2.14 (m, 3H), 1.95-1.85 (m, 1H), 1.41 (d, J=6.0 Hz, 3H), 1.39 (d, J=6.0 Hz, 3H); LRMS (M+H) m/z 423.3.

### Example 35

### (S)-7-isopropoxy-4-((1-methylazetidin-3-yl)ethynyl)-1-((5-oxopyrrolidin-2-yl)methoxy)isoquinoline-6-carboxamide (I-26)

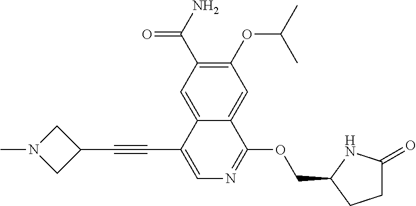 [SMILES: CC(C)Oc1cc2c(OC[C@@H]3CCC(=O)N3)ncc(C#CC3CN(C)C3)c2cc1C(N)=O]

General procedures (D), step 2, formaldehyde was used: 0.019 mmol scale, 69% yield.

¹H NMR (400 MHz, DMSO-d₆) δ 8.31 (s, 1H), 8.11 (s, 1H), 8.05 (s, 1H), 7.78 (s, 1H), 7.77 (s, 2H), 7.68 (s, 1H), 4.93 (hept, J=6.0 Hz, 1H), 4.49 (dd, J=10.9, 3.8 Hz, 1H), 4.32 (dd, J=10.9, 6.5 Hz, 1H), 4.05-4.00 (m, 1H), 3.64-3.60 (m, 2H), 3.55-3.48 (m, 1H), 3.12-3.08 (m, 2H), 2.34-2.16 (m, 6H), 1.93-1.88 (m, 1H), 1.41 (d, J=6.0 Hz, 3H), 1.39 (d, J=6.0 Hz, 3H); LRMS (M+H) m/z 437.1.

### Example 36

### (S)-7-isopropoxy-4-((1-isopropylazetidin-3-yl)ethynyl)-1-((5-oxopyrrolidin-2-yl)methoxy)isoquinoline-6-carboxamide (I-27)

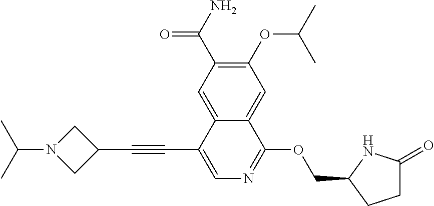 [SMILES: CC(C)Oc1cc2c(OC[C@@H]3CCC(=O)N3)ncc(C#CC3CN(C(C)C)C3)c2cc1C(N)=O]

General procedures (D), step 2, acetone was used: 0.05 mmol scale, 89% yield.

¹H NMR (400 MHz, Chloroform-d) δ 8.95 (s, 1H), 8.02 (s, 1H), 7.93 (d, J=4.0 Hz, 1H), 7.58 (s, 1H), 6.81 (s, 1H), 6.34 (d, J=4.0 Hz, 1H), 4.95 (hept, J=6.1 Hz, 1H), 4.64 (dd, J=11.1, 3.5 Hz, 1H), 4.40 (dd, J=11.1, 7.1 Hz, 1H), 4.25-4.19 (m, 1H), 3.76-3.73 (m, 2H), 3.57-3.49 (m, 1H), 3.23-3.19 (m, 2H), 2.50-2.35 (m, 4H), 2.06-1.97 (m, 1H), 1.50 (d, J=6.1 Hz, 3H), 1.49 (d, J=6.1 Hz, 3H), 0.96 (d, J=6.2 Hz, 6H); LRMS (M+H) m/z 465.5.

### Example 37

### methyl (S)-3-((6-carbamoyl-7-isopropoxy-1-((5-oxopyrrolidin-2-yl)methoxy)isoquinolin-4-yl)ethynyl)azetidine-1-carboxylate (I-28)

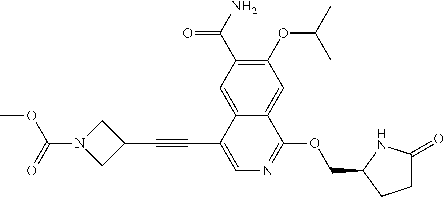 [SMILES: COC(=O)N1CC(C#Cc2cnc(OC[C@@H]3CCC(=O)N3)c3cc(OC(C)C)c(C(N)=O)cc23)C1]

Similar procedures as the synthesis of I-16: 0.05 mmol scale, 49% yield.

¹H NMR (400 MHz, Chloroform-d) δ 8.90 (s, 1H), 8.03 (s, 1H), 7.92 (d, J=4.0 Hz, 1H), 7.57 (s, 1H), 6.64 (s, 1H), 6.25 (d, J=4.0 Hz, 1H), 4.94 (hept, J=6.1 Hz, 1H), 4.65 (dd, J=11.1, 3.5 Hz, 1H), 4.41 (dd, J=11.1, 7.0 Hz, 1H), 4.36-3.32 (m, 2H), 4.25-4.16 (m, 3H), 3.78-3.70 (m, 1H), 2.50-2.36 (m, 3H), 2.07-1.96 (m, 1H), 1.49 (d, J=6.1 Hz, 6H); LRMS (M+H) m/z 481.5.

### Example 38

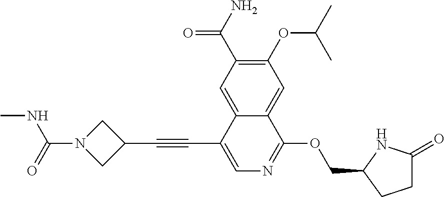 [SMILES: CNC(=O)N1CC(C#Cc2cnc(OC[C@@H]3CCC(=O)N3)c3cc(OC(C)C)c(C(N)=O)cc23)C1]

### (S)-7-isopropoxy-4-((1-(methylcarbamoyl)azetidin-3-yl)ethynyl)-1-((5-oxopyrrolidin-2-yl)methoxy)isoquinoline-6-carboxamide (I-29)

Similar procedures as the synthesis of I-16, methylcarbamic chloride was used (pre-heated in 2 mL of THF at 70° C. for 1 hour, before addition to the reaction mixture): 0.05 mmol scale, 70% yield.

¹H NMR (400 MHz, DMSO-d₆) δ 8.31 (s, 1H), 8.12 (s, 1H), 8.09 (s, 1H), 7.78 (br s, 1H), 7.77 (br s, 1H), 7.67 (s, 1H), 6.38 (q, J=4.5 Hz, 1H), 4.93 (hept, J=6.0 Hz, 1H), 4.49 (dd, J=10.9, 3.8 Hz, 1H), 4.32 (dd, J=10.9, 6.5 Hz, 1H), 4.19-4.15 (m, 2H), 4.06-4.00 (m, 1H), 3.87-3.75 (m, 3H), 2.55 (d, J=4.5 Hz, 3H), 2.34-2.16 (m, 3H), 1.93-1.88 (m, 1H), 1.41 (d, J=6.0 Hz, 3H), 1.39 (d, J=6.0 Hz, 3H); LRMS (M+H) m/z 480.5.

### Example 39

### (S)-4-((3,3-dimethoxycyclobutyl)ethynyl)-7-isopropoxy-1-((5-oxopyrrolidin-2-yl)methoxy)isoquinoline-6-carboxamide (I-30)

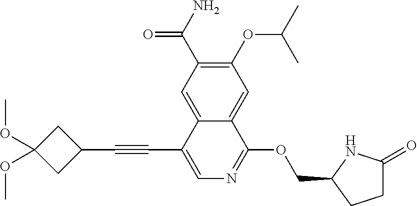 [SMILES: COC1(OC)CC(C#Cc2cnc(OC[C@@H]3CCC(=O)N3)c3cc(OC(C)C)c(C(N)=O)cc23)C1]

General procedures (B): 0.1 mmol scale, 25% yield.

¹H NMR (400 MHz, Chloroform-d) δ 8.96 (s, 1H), 8.01 (s, 1H), 7.90 (d, J=2.7 Hz, 1H), 7.56 (s, 1H), 7.27 (s, 1H), 6.44 (s, 1H), 6.11 (d, J=2.7 Hz, 1H), 4.93 (hept, J=6.1 Hz, 1H), 4.63 (dd, J=11.1, 3.5 Hz, 1H), 4.40 (dd, J=11.1, 7.1 Hz, 1H), 4.24-4.18 (m, 1H), 3.21 (s, 3H), 3.20 (s, 3H), 3.17-3.11 (m, 1H), 2.71-2.65 (m, 2H), 2.47-2.37 (m, 5H), 2.06-1.96 (m, 1H), 1.49 (d, J=6.1 Hz, 6H); LRMS (M+Na) m/z 504.1

### Example 40

### (S)-7-isopropoxy-4-((3-oxocyclobutyl)ethynyl)-1-((5-oxopyrrolidin-2-yl)methoxy)isoquinoline-6-carboxamide

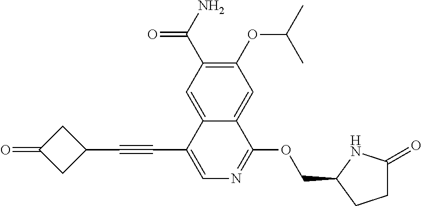 [SMILES: CC(C)Oc1cc2c(OC[C@@H]3CCC(=O)N3)ncc(C#CC3CC(=O)C3)c2cc1C(N)=O]

A TFA-CH₂Cl₂ (1:5, 1 mL) solution of (S)-4-((3,3-dimethoxycyclobutyl)ethynyl)-7-isopropoxy-1-((5-oxopyrrolidin-2-yl)methoxy)isoquinoline-6-carboxamide (16.8 mg, 0.035 mmol) was stirred at room temperature. The reaction went to completion after 3 hours, as monitored by LCMS. Volatiles were removed in vacuo, and the crude product was used in next step without further purification.

¹H NMR (400 MHz, Chloroform-d) δ 9.06 (s, 1H), 8.75 (s, 1H), 8.57 (s, 1H), 8.28 (s, 1H), 8.03 (s, 1H), 7.76 (s, 1H), 5.09 (hept, J=6.0 Hz, 1H), 4.91 (dd, J=10.4, 2.9 Hz, 1H), 4.56 (dd, J=10.4, 8.6 Hz, 1H), 4.50-4.43 (m, 1H), 3.68-3.58 (m, 3H), 3.54-3.45 (m, 2H), 2.74-2.69 (m, 2H), 2.58-2.48 (m, 1H), 2.11-2.02 (m, 1H), 1.55 (d, J=6.0 Hz, 3H), 1.53 (d, J=6.0 Hz, 3H); LRMS (M+H) m/z 436.0.

### Example 41

### 4-((cis-3-(dimethylamino)cyclobutyl)ethynyl)-7-isopropoxy-1-(((S)-5-oxopyrrolidin-2-yl)methoxy)isoquinoline-6-carboxamide (I-31)

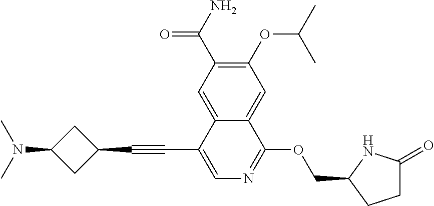 [SMILES: CC(C)Oc1cc2c(OC[C@@H]3CCC(=O)N3)ncc(C#C[C@H]3C[C@@H](N(C)C)C3)c2cc1C(N)=O]

A CH₂Cl₂ (2 mL) solution of (S)-7-isopropoxy-4-((3-oxocyclobutyl)ethynyl)-1-((5-oxopyrrolidin-2-yl)methoxy)isoquinoline-6-carboxamide (about 0.035 mmol) and dimethylamine hydrogen chloride (9.9 mg, 2.5 eq) was stirred at room temperature for 30 minutes. Na(OAc)₃BH (1.5 eq) was added with cooling in an ice bath. More reagents were added until the starting material was not detected by LCMS. Volatiles were removed in vacuo, product was purified by revered-phase HPLC, and was subsequently free-based by passing a MeOH solution through PL-HCO₃ column. The filtrate was collected and the solvent was removed in vacuo. 4-((cis-3-(dimethylamino)cyclobutyl)ethynyl)-7-isopropoxy-1-(((S)-5-oxopyrrolidin-2-yl)methoxy)isoquinoline-6-carboxamide (I-31) was obtained as a light beige color solid: 6.2 mg (38% yield).

¹H NMR (400 MHz, Chloroform-d) δ 8.97 (s, 1H), 7.99 (s, 1H), 7.91 (s, 1H), 7.54 (s, 1H), 6.15 (s, 1H), 5.98 (s, 1H), 4.92 (hept, J=6.0 Hz, 1H), 4.64 (dd, J=11.1, 3.5 Hz, 1H), 4.40 (dd, J=11.1, 7.1 Hz, 1H), 4.23-4.18 (m, 1H), 2.98 (tt, J=9.7, 7.6 Hz, 1H), 2.70-2.54 (m, 3H), 2.48-2.39 (m, 3H), 2.26-2.17 (m, partially overlapped, 1H), 2.22 (s, partially overlapped, 6H), 2.06-1.96 (m, partially overlapped with H₂O, 2H), 1.51 (d, J=6.0 Hz, 3H), 1.50 (d, J=6.0 Hz, 3H); LRMS (M+H) m/z 465.1.

### Example 42

### (S)-4-(1-cyclopropyl-1H-pyrazol-4-yl)-7-isopropoxy-1-((5-oxopyrrolidin-2-yl)methoxy)isoquinoline-6-carboxamide (I-32)

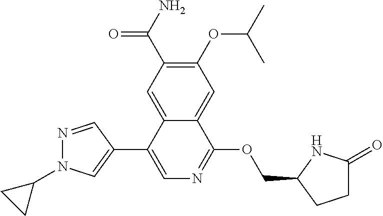 [SMILES: CC(C)Oc1cc2c(OC[C@@H]3CCC(=O)N3)ncc(-c3cnn(C4CC4)c3)c2cc1C(N)=O]

General procedures (C): 0.08 mmol scale, 74% yield.

¹H NMR (400 MHz, DMSO-d₆) δ 8.30 (s, 1H), 8.12-8.11 (m, 2H), 7.86 (d, J=0.8 Hz, 1H), 7.75 (br s, 2H), 7.72 (s, 1H), 7.68 (d, J=0.8 Hz, 1H), 4.95 (hept, J=6.0 Hz, 1H), 4.49 (dd, J=10.9, 3.8 Hz, 1H), 4.34 (dd, J=10.9, 6.3 Hz, 1H), 4.07-4.00 (m, 1H), 3.84 (tt, J=7.4, 3.8 Hz, 1H), 2.35-2.16 (m, 3H), 1.97-1.86 (m, 1H), 1.41 (d, J=6.0 Hz, 3H), 1.39 (d, J=6.0 Hz, 3H), 1.19-1.12 (m, 2H), 1.04-0.99 (m, 2H); LRMS (M+H) m/z 450.1.

### Example 43

### (S)-4-(1-cyclobutyl-1H-pyrazol-4-yl)-7-isopropoxy-1-((5-oxopyrrolidin-2-yl)methoxy)isoquinoline-6-carboxamide (I-33)

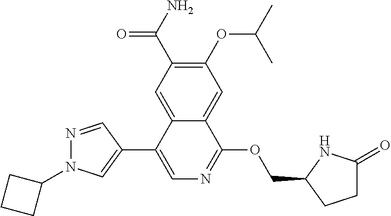 [SMILES: CC(C)Oc1cc2c(OC[C@@H]3CCC(=O)N3)ncc(-c3cnn(C4CCC4)c3)c2cc1C(N)=O]

General procedures (C): 0.06 mmol scale, 73% yield.

¹H NMR (400 MHz, Chloroform-d) δ 8.92 (s, 1H), 7.96 (br s, 1H), 7.87 (s, 1H), 7.72 (d, J=0.8 Hz, 1H), 7.68 (d, J=0.8 Hz, 1H), 7.63 (s, 1H), 6.05 (s, 1H), 5.91 (br s, 1H), 5.00-4.90 (m, 1H), 4.88-4.81 (m, 1H), 4.67 (dd, J=11.1, 3.4 Hz, 1H), 4.43 (dd, J=11.1, 7.1 Hz, 1H), 4.26-4.20 (m, 1H), 2.72-2.62 (m, 2H), 2.59-2.51 (m, 2H), 2.49-2.37 (m, 3H), 2.09-1.98 (m, 1H), 1.97-1.84 (m, 2H), 1.53 (d, J=6.0 Hz, 3H), 1.52 (d, J=6.0 Hz, 3H); LRMS (M+H) m/z 464.1.

### Example 44

### (S)-4-(1-cyclopentyl-1H-pyrazol-4-yl)-7-isopropoxy-1-((5-oxopyrrolidin-2-yl)methoxy)isoquinoline-6-carboxamide (I-34)

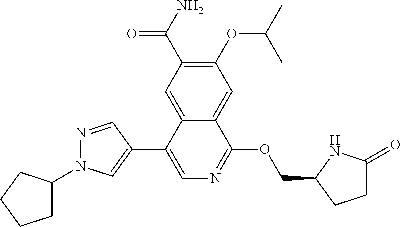 [SMILES: CC(C)Oc1cc2c(OC[C@@H]3CCC(=O)N3)ncc(-c3cnn(C4CCCC4)c3)c2cc1C(N)=O]

General procedures (C): 0.08 mmol scale, 70% yield.

¹H NMR (400 MHz, DMSO-d₆) δ 8.33 (s, 1H), 8.12 (s, 1H), 8.08 (d, J=0.8 Hz, 1H), 7. (87 (s, 1H), 7.74 (br s, 2H), 7.72 (s, 1H), 7.70 (d, J=0.8 Hz, 1H), 4.95 (hept, J=6.0 Hz, 1H), 4.80 (p, J=7.0 Hz, 1H), 4.49 (dd, J=10.9, 3.9 Hz, 1H), 4.34 (dd, J=10.9, 6.3 Hz, 1H), 4.07-4.00 (m, 1H), 2.35-2.08 (m, 5H), 2.06-1.97 (m, 2H), 1.95-1.81 (m, 3H), 1.70-1.64 (m, 2H), 1.42 (d, J=6.0 Hz, 3H), 1.40 (d, J=6.0 Hz, 3H); LRMS (M+H) m/z 478.2.

### Example 45

### (S)-7-isopropoxy-1-((5-oxopyrrolidin-2-yl)methoxy)-4-(1-(tetrahydro-2H-pyran-4-yl)-1H-pyrazol-4-yl)isoquinoline-6-carboxamide (I-35)

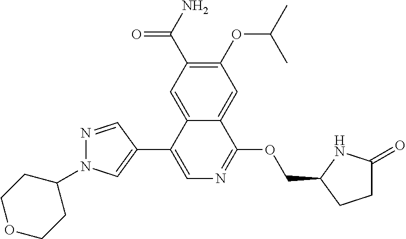 [SMILES: CC(C)Oc1cc2c(OC[C@@H]3CCC(=O)N3)ncc(-c3cnn(C4CCOCC4)c3)c2cc1C(N)=O]

General procedures (C): 0.08 mmol scale, 60% yield.

¹H NMR (400 MHz, Chloroform-d) δ 8.92 (s, 1H), 7.97 (d, J=3.9 Hz, 1H), 7.87 (s, 1H), 7.73 (d, J=0.7 Hz, 1H), 7.70 (d, J=0.8 Hz, 1H), 7.64 (s, 1H), 6.14 (s, 1H), 5.95 (d, J=4.1 Hz, 1H), 4.96 (hept, J=6.1 Hz, 1H), 4.67 (dd, J=11.1, 3.5 Hz, 1H), 4.49-4.41 (m, 2H), 4.26-4.20 (m, 1H), 4.17-4.14 (m, 2H), 3.62-3.55 (m, 2H), 2.52-2.39 (m, 3H), 2.23-2.14 (m, 4H), 2.09-2.00 (m, 1H), 1.52 (d, J=6.0 Hz, 6H); LRMS (M+H) m/z 494.2.

### Example 46

### (S)-7-isopropoxy-1-((5-oxopyrrolidin-2-yl)methoxy)-4-(1-((tetrahydro-2H-pyran-4-yl)methyl)-1H-pyrazol-4-yl)isoquinoline-6-carboxamide (I-36)

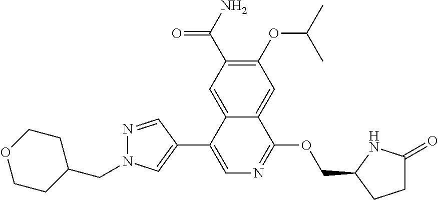 [SMILES: CC(C)Oc1cc2c(OC[C@@H]3CCC(=O)N3)ncc(-c3cnn(CC4CCOCC4)c3)c2cc1C(N)=O]

General procedures (C): 0.08 mmol scale, 24% yield.

¹H NMR (400 MHz, Chloroform-d) δ 8.91 (s, 1H), 7.98 (br s, 1H), 7.88 (s, 1H), 7.72 (d, J=0.7 Hz, 1H), 7.64-7.63 (m, 2H), 6.16 (s, 1H), 5.98 (br s, 1H), 4.96 (hept, J=6.0 Hz, 1H), 4.67 (dd, J=11.1, 3.4 Hz, 1H), 4.44 (dd, J=11.1, 7.0 Hz, 1H), 4.26-4.20 (m, 1H), 4.10 (d, J=7.2 Hz, 2H), 4.03-3.99 (m, 2H), 3.42 (td, J=11.8, 2.1 Hz, 2H), 2.52-2.39 (m, 3H), 2.26 (ttt, J=11.2, 7.3, 3.8 Hz, 1H), 2.09-2.00 (m, 1H), 1.64-1.59 (m, 2H), 1.523 (d, J=6.0 Hz, 3H), 1.521 (d, J=6.0 Hz, 3H), 1.43 (dtd, J=13.3, 11.8, 4.5 Hz, 2H); LRMS (M+H) m/z 508.2.

### Example 47

### 1-(2-(tetrahydro-2H-pyran-4-yl)ethyl)-4-(4,4,5,5-tetramethyl-1,3,2-dioxaborolan-2-yl)-1H-pyrazole

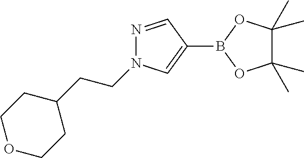 [SMILES: CC1(C)OB(c2cnn(CCC3CCOCC3)c2)OC1(C)C]

General procedures (F): 2 mmol scale, 4-(2-bromoethyl)tetrahydro-2H-pyran was used, 98% yield.

¹H NMR (400 MHz, Chloroform-d) δ 7.78 (d, J=0.7 Hz, 1H), 7.67 (d, J=0.7 Hz, 1H), 4.19-4.15 (m, 2H), 3.96-3.91 (m, 2H), 3.34 (ddd, J=11.7, 11.7, 2.1 Hz, 2H), 1.86-1.80 (m, 2H), 1.63-1.59 (m, 2H), 1.52-1.42 (m, 1H), 1.37-1.30 (m, 14H); LRMS (M+H) m/z 307.0.

### Example 48

### (S)-7-isopropoxy-1-((5-oxopyrrolidin-2-yl)methoxy)-4-(1-(2-(tetrahydro-2H-pyran-4-yl)ethyl)-1H-pyrazol-4-yl)isoquinoline-6-carboxamide (I-37)

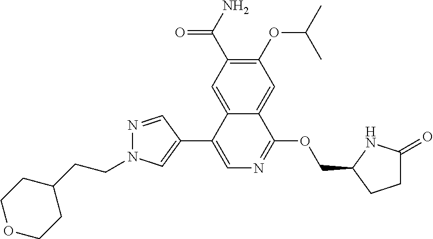 [SMILES: CC(C)Oc1cc2c(OC[C@@H]3CCC(=O)N3)ncc(-c3cnn(CCC4CCOCC4)c3)c2cc1C(N)=O]

General procedures (C): 0.08 mmol scale, 10% yield.

¹H NMR (400 MHz, Chloroform-d) δ 8.91 (s, 1H), 7.96 (br s, 1H), 7.87 (s, 1H), 7.69 (d, J=0.8 Hz, 1H), 7.64 (d, J=0.8 Hz, 1H), 7.62 (br s, 1H), 5.96 (s, 1H), 5.86 (br s, 1H), 4.94 (hept, J=6.1 Hz, 1H), 4.66 (dd, J=11.2, 3.4 Hz, 1H), 4.43 (dd, J=11.1, 7.1 Hz, 1H), 4.28-4.19 (m, 3H), 3.96 (br dd, J=11.2, 4.1 Hz, 2H), 3.39 (ddd, J=11.8, 11.8, 2.1 Hz, 2H), 2.48-2.37 (m, 3H), 2.06-1.98 (m, 1H), 1.92 (dd, J=6.9 Hz, 2H), 1.69-1.65 (m, 2H), 1.62-1.58 (m, partially overlapped with H₂O, 1H), 1.52 (d, J=6.1 Hz, 3H), 1.51 (d, J=6.1 Hz, 3H), 1.37 (ddd, J=12.1, 12.1, 4.5 Hz, 2H); LRMS (M+H) m/z 522.1.

### Example 49

### (S)-7-isopropoxy-4-(1-(2-morpholinoethyl)-1H-pyrazol-4-yl)-1-((5-oxopyrrolidin-2-yl)methoxy)isoquinoline-6-carboxamide (I-38)

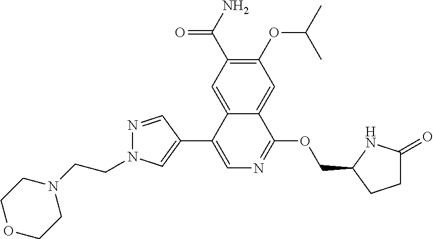 [SMILES: CC(C)Oc1cc2c(OC[C@@H]3CCC(=O)N3)ncc(-c3cnn(CCN4CCOCC4)c3)c2cc1C(N)=O]

General procedures (C): 0.06 mmol scale, 61% yield.

¹H NMR (400 MHz, Chloroform-d) δ 8.89 (s, 1H), 7.96 (d, J=3.8 Hz, 1H), 7.86 (s, 1H), 7.77 (d, J=0.8 Hz, 1H), 7.69 (d, J=0.8 Hz, 1H), 7.64 (s, 1H), 6.53-6.49 (m, 1H), 6.21 (br s, 1H), 4.96 (hept, J=6.1 Hz, 1H), 4.66 (dd, J=11.1, 3.5 Hz, 1H), 4.43 (dd, J=11.1, 7.0 Hz, 1H), 4.35 (t, J=6.5 Hz, 2H), 4.27-4.20 (m, 1H), 3.72-3.70 (m, 4H), 2.90 (t, J=6.5 Hz, 2H), 2.55-2.36 (m, 7H), 2.08-1.99 (m, 1H), 1.51 (d, J=6.0 Hz, 3H), 1.50 (d, J=6.0 Hz, 3H); LRMS (M+H) m/z 523.1.

### Example 50

### 1-(trans-4-ethoxycyclohexyl)-4-(4,4,5,5-tetramethyl-1,3,2-dioxaborolan-2-yl)-1H-pyrazole

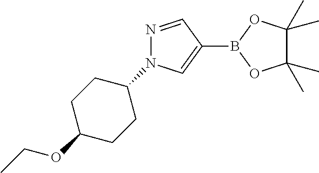 [SMILES: CCO[C@H]1CC[C@H](n2cc(B3OC(C)(C)C(C)(C)O3)cn2)CC1]

General procedures (F): 1 mmol scale, 4-ethoxycyclohexyl 4-nitrobenzenesulfonate (cis trans >4:1) was used, trans-product was obtained at ˜30% yield.

¹H NMR (400 MHz, Chloroform-d) δ 7.76 (s, 1H), 7.69 (s, 1H), 4.12 (tt, J=11.7, 3.5 Hz, 1H), 3.52 (q, J=7.0 Hz, 2H), 3.29 (tt, J=10.7, 3.9 Hz, 1H), 2.20-2.13 (m, 4H), 1.83-1.73 (m, 2H), 1.45-1.35 (m, 2H), 1.29 (s, 12H), 1.18 (t, J=7.0 Hz, 3H).

### Example 51

### 4-(1-(trans-4-ethoxycyclohexyl)-1H-pyrazol-4-yl)-7-isopropoxy-1-(((S)-5-oxopyrrolidin-2-yl)methoxy)isoquinoline-6-carboxamide (I-39)

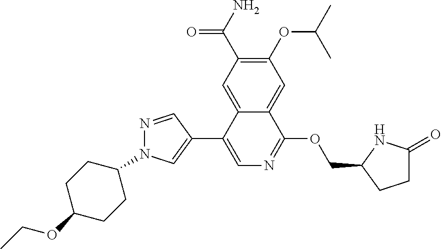 [SMILES: CCO[C@H]1CC[C@H](n2cc(-c3cnc(OC[C@@H]4CCC(=O)N4)c4cc(OC(C)C)c(C(N)=O)cc34)cn2)CC1]

General procedures (C): 0.1 mmol scale, 26% yield.

¹H NMR (400 MHz, Chloroform-d) δ 8.91 (s, 1H), 7.97 (d, J=3.9 Hz, 1H), 7.86 (s, 1H), 7.70 (d, J=0.8 Hz, 1H), 7.67 (d, J=0.8 Hz, 1H), 7.64 (s, 1H), 6.33 (s, 1H), 6.05 (d, J=3.8 Hz, 1H), 4.96 (hept, J=6.0 Hz, 1H), 4.66 (dd, J=11.1, 3.5 Hz, 1H), 4.43 (dd, J=11.1, 7.0 Hz, 1H), 4.26-4.18 (m, 2H), 3.56 (q, J=7.0 Hz, 2H), 3.38 (tt, J=10.7, 4.1 Hz, 1H), 2.49-2.38 (m, 3H), 2.34-2.28 (m, 2H), 2.26-2.20 (m, 2H), 2.08-1.89 (m, 3H), 1.51 (d, J=6.0 Hz, 3H), 1.50 (d, J=6.0 Hz, 3H), 1.50-1.41 (m, partially overlapped, 2H), 1.23 (t, J=7.0 Hz, 3H); LRMS (M+H) m/z 536.2.

### Example 52

### (1r,4r)-1-methyl-4-(4-(4,4,5,5-tetramethyl-1,3,2-dioxaborolan-2-yl)-1H-pyrazol-1-yl)cyclohexan-1-ol

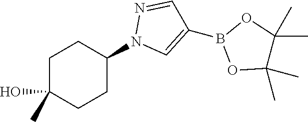 [SMILES: CC1(C)OB(c2cnn([C@H]3CC[C@](C)(O)CC3)c2)OC1(C)C]

General procedures (F): 0.75 mmol scale, (1s,4s)-4-hydroxy-4-methylcyclohexyl 4-methylbenzenesulfonate was used, 24% yield.

¹H NMR (400 MHz, Chloroform-d) δ 7.79 (s, 1H), 7.76 (s, 1H), 4.20 (tt, J=10.4, 4.1 Hz, 1H), 2.18-2.11 (m, 2H), 1.97 (dddd, J=13.7, 11.7, 10.3, 3.7 Hz, 2H), 1.83-1.77 (m, 2H), 1.68-1.61 (m, 2H), 1.33 (s, 3H), 1.32 (s, 12H).

### Example 53

### (1s,4s)-1-methyl-4-(4-(4,4,5,5-tetramethyl-1,3,2-dioxaborolan-2-yl)-1H-pyrazol-1-yl)cyclohexan-1-ol

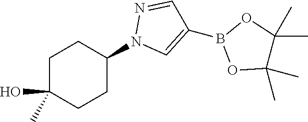 [SMILES: CC1(C)OB(c2cnn([C@H]3CC[C@@](C)(O)CC3)c2)OC1(C)C]

General procedures (F): 0.88 mmol scale, (1r,4r)-4-hydroxy-4-methylcyclohexyl 4-methylbenzenesulfonate was used, 40% yield.

¹H NMR (400 MHz, Chloroform-d) δ 7.80 (s, 1H), 7.79 (s, 1H), 4.13 (tt, J=11.8, 3.9 Hz, 1H), 2.23-2.12 (m, 2H), 2.00-1.97 (m, 2H), 1.83-1.78 (m, 2H), 1.60-1.52 (m, 2H), 1.33 (s, 12H), 1.32 (s, 3H).

### Example 54

### 4-(1-((1r,4S)-4-hydroxy-4-methylcyclohexyl)-1H-pyrazol-4-yl)-7-isopropoxy-1-(((S)-5-oxopyrrolidin-2-yl)methoxy)isoquinoline-6-carboxamide (I-40)

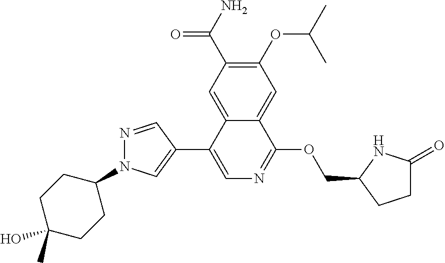 [SMILES: CC(C)Oc1cc2c(OC[C@@H]3CCC(=O)N3)ncc(-c3cnn([C@H]4CC[C@](C)(O)CC4)c3)c2cc1C(N)=O]

General procedures (C): 0.05 mmol scale, 72% yield.

¹H NMR (400 MHz, DMSO-d₆) δ 8.33 (s, 1H), 8.12 (s, 1H), 8.117 (s, 1H), 7.87 (s, 1H), 7.75 (br s, 1H), 7.74 (br s, 1H), 7.72 (s, 1H), 7.70 (s, 1H), 4.95 (hept, J=6.0 Hz, 1H), 4.49 (dd, J=10.9, 3.9 Hz, 1H), 4.43 (s, 1H), 4.33 (dd, J=10.9, 6.3 Hz, 1H), 4.31-4.24 (m, partially overlapped, 1H), 4.04 (tt, J=8.2, 4.4 Hz, 1H), 2.35-2.17 (m, 3H), 2.08-1.89 (m, 5H), 1.69-1.63 (m, 2H), 1.61-1.53 (m, 2H), 1.42 (d, J=6.0 Hz, 3H), 1.40 (d, J=6.0 Hz, 3H), 1.22 (s, 3H); LRMS (M+H) m/z 522.3.

### Example 55

### 4-(1-((1s,4R)-4-hydroxy-4-methylcyclohexyl)-1H-pyrazol-4-yl)-7-isopropoxy-1-(((S)-5-oxopyrrolidin-2-yl)methoxy)isoquinoline-6-carboxamide (I-41)

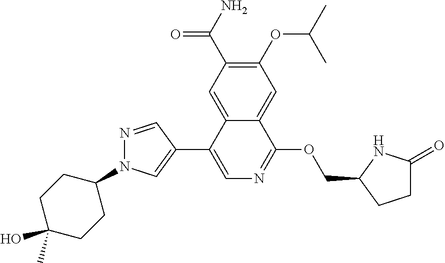 [SMILES: CC(C)Oc1cc2c(OC[C@@H]3CCC(=O)N3)ncc(-c3cnn([C@H]4CC[C@@](C)(O)CC4)c3)c2cc1C(N)=O]

General procedures (C): 0.05 mmol scale, 7% yield.

¹H NMR (400 MHz, DMSO-d₆) δ 8.33 (s, 1H), 8.12 (s, 1H), 8.06 (d, J=0.8 Hz, 1H), 7.87 (s, 1H), 7.75 (br s, 1H), 7.73 (br s, 1H), 7.70 (d, J=0.8 Hz, 1H), 4.95 (hept, J=6.0 Hz, 1H), 4.49 (dd, J=10.9, 3.9 Hz, 1H), 4.34 (dd, J=10.9, 6.3 Hz, 1H), 4.23-4.15 (m, 2H), 4.07-4.01 (m, 1H), 2.36-2.12 (m, 5H), 1.95-1.85 (m, 3H), 1.70-1.66 (m, 2H), 1.54-1.39 (m, 2H), 1.42 (d, J=6.0 Hz, 3H), 1.40 (d, J=6.0 Hz, 3H), 1.16 (s, 3H); LRMS (M+H) m/z 522.5.

### Example 56

### 4-(1-(cis-3-ethoxycyclobutyl)-1H-pyrazol-4-yl)-7-isopropoxy-1-(((S)-5-oxopyrrolidin-2-yl)methoxy)isoquinoline-6-carboxamide (I-42)

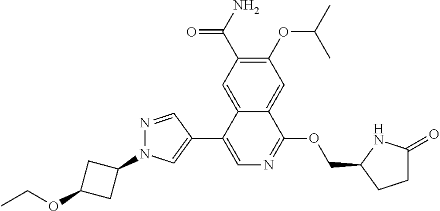 [SMILES: CCO[C@H]1C[C@@H](n2cc(-c3cnc(OC[C@@H]4CCC(=O)N4)c4cc(OC(C)C)c(C(N)=O)cc34)cn2)C1]

General procedures (C): 0.06 mmol scale, 40% yield.

¹H NMR (400 MHz, Chloroform-d) δ 8.89 (s, 1H), 7.95 (br s, 1H), 7.86 (s, 1H), 7.72 (s, 1H), 7.71 (s, 1H), 7.63 (s, 1H), 6.02 (s, 1H), 5.89 (br s, 1H), 4.95 (hept, J=6.1 Hz, 1H), 4.67 (dd, J=11.1, 3.4 Hz, 1H), 4.49 (tt, J=9.2, 7.5 Hz, 1H), 4.44 (dd, J=11.1, 7.1 Hz, 1H), 4.26-4.20 (m, 1H), 3.90 (tt, J=7.7, 6.5 Hz, 1H), 3.49 (q, J=7.0 Hz, 2H), 3.00-2.92 (m, 2H), 2.65-2.57 (m, 2H), 2.52-2.37 (m, 3H), 2.09-2.00 (m, 1H), 1.53 (d, J=6.1 Hz, 3H), 1.52 (d, J=6.1 Hz, 3H), 1.24 (t, J=7.0 Hz, 3H); LRMS (M+H) m/z 508.2.

### Example 57

### 4-(1-(cis-3-(dimethylamino)cyclobutyl)-1H-pyrazol-4-yl)-7-isopropoxy-1-(((S)-5-oxopyrrolidin-2-yl)methoxy)isoquinoline-6-carboxamide (I-43)

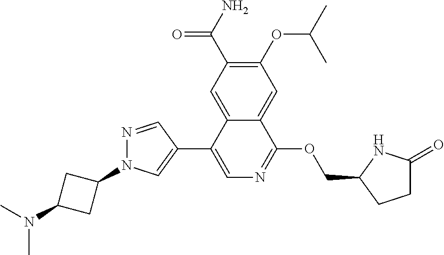 [SMILES: CC(C)Oc1cc2c(OC[C@@H]3CCC(=O)N3)ncc(-c3cnn([C@H]4C[C@@H](N(C)C)C4)c3)c2cc1C(N)=O]

General procedures (C): 0.08 mmol scale, 54% yield.

¹H NMR (400 MHz, Chloroform-d) δ 8.89 (s, 1H), 7.96 (d, J=3.6 Hz, 1H), 7.86 (s, 1H), 7.79 (d, J=0.8 Hz, 1H), 7.70 (d, J=0.8 Hz, 1H), 7.62 (s, 1H), 6.14 (s, 1H), 5.96 (d, J=3.6 Hz, 1H), 4.95 (hept, J=6.0 Hz, 1H), 4.66 (dd, J=11.1, 3.5 Hz, 1H), 4.65-4.56 (m, 1H), 4.43 (dd, J=11.1, 7.1 Hz, 1H), 4.26-4.20 (m, 1H), 2.81-2.73 (m, 2H), 2.62-2.37 (m, 6H), 2.21 (s, 6H), 2.07-1.99 (m, 1H), 1.52 (d, J=6.0 Hz, 3H), 1.51 (d, J=6.0 Hz, 3H); LRMS (M+H) m/z 507.1.

### Example 58

### tert-butyl (S)-4-(4-(6-carbamoyl-7-isopropoxy-1-((5-oxopyrrolidin-2-yl)methoxy)isoquinolin-4-yl)-1H-pyrazol-1-yl)piperidine-1-carboxylate (I-44)

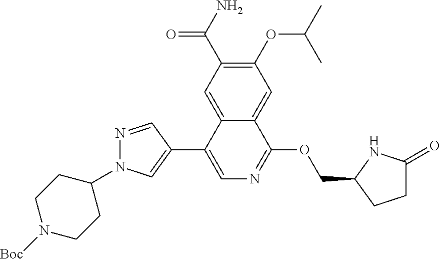 [SMILES: CC(C)Oc1cc2c(OC[C@@H]3CCC(=O)N3)ncc(-c3cnn(C4CCN(C(=O)OC(C)(C)C)CC4)c3)c2cc1C(N)=O]

General procedures (C): 0.1 mmol scale, 80% yield.

¹H NMR (400 MHz, Chloroform-d) δ 8.91 (s, 1H), 7.97 (d, J=4.0 Hz, 1H), 7.87 (s, 1H), 7.72 (d, J=0.8 Hz, 1H), 7.68 (d, J=0.8 Hz, 1H), 7.63 (s, 1H), 6.07 (s, 1H), 5.91 (d, J=4.0 Hz, 1H), 4.96 (hept, J=6.1 Hz, 1H), 4.67 (dd, J=11.1, 3.4 Hz, 1H), 4.44 (dd, J=11.1, 7.1 Hz, 1H), 4.36 (tt, J=11.6, 4.0 Hz, 1H), 4.31-4.20 (m, 3H), 2.96-2.89 (m, 2H), 2.52-2.37 (m, 3H), 2.26-2.22 (m, 2H), 2.09-1.97 (m, 3H), 1.53 (br d, J=6.1 Hz, 6H), 1.49 (s, 9H); LRMS (M-Boc+H) m/z 493.1, (M+Na) m/z 615.2.

### Example 59

### (S)-7-isopropoxy-1-((5-oxopyrrolidin-2-yl)methoxy)-4-(1-(piperidin-4-yl)-1H-pyrazol-4-yl)isoquinoline-6-carboxamide (I-45)

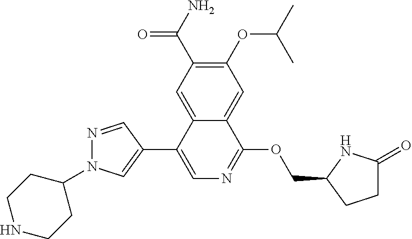 [SMILES: CC(C)Oc1cc2c(OC[C@@H]3CCC(=O)N3)ncc(-c3cnn(C4CCNCC4)c3)c2cc1C(N)=O]

General procedures (D), step 1: 0.067 mmol scale, >90% yield.

¹H NMR (400 MHz, Chloroform-d) δ 8.93 (s, 1H), 7.97 (br s, 1H), 7.88 (s, 1H), 7.72 (d, J=0.8 Hz, 1H), 7.69 (d, J=0.8 Hz, 1H), 7.63 (s, 1H), 6.07 (s, 1H), 5.91 (s, 1H), 4.96 (hept, J=6.1 Hz, 1H), 4.67 (dd, J=11.1, 3.5 Hz, 1H), 4.44 (dd, J=11.1, 7.1 Hz, 1H), 4.33 (tt, J=11.7, 4.1 Hz, 1H), 4.26-4.20 (m, 1H), 3.28 (br d, J=12.6 Hz, 2H), 2.84-2.77 (m, 2H), 2.50-2.37 (m, 3H), 2.26 (br d, J=12.4 Hz, 2H), 2.08-1.94 (m, 3H), 1.53 (d, J=6.1 Hz, 6H); LRMS (M+H) m/z 493.1.

### Example 60

### (S)-7-isopropoxy-4-(1-(1-methylpiperidin-4-yl)-1H-pyrazol-4-yl)-1-((5-oxopyrrolidin-2-yl)methoxy)isoquinoline-6-carboxamide (I-46)

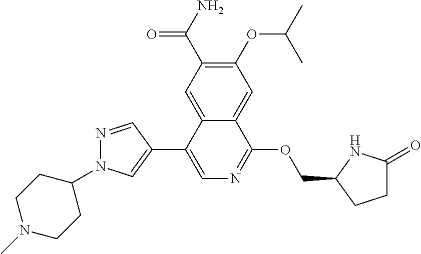 [SMILES: CC(C)Oc1cc2c(OC[C@@H]3CCC(=O)N3)ncc(-c3cnn(C4CCN(C)CC4)c3)c2cc1C(N)=O]

General procedures (D), step 2: 0.067 mmol scale, formaldehyde was used, 51% yield.

¹H NMR (400 MHz, DMSO-d₆) δ 8.32 (s, 1H), 8.12 (s, 1H), 8.11 (s, 1H), 7.87 (s, 1H), 7.75 (s, 1H), 7.73 (s, 1H), 7.72 (s, 2H), 4.95 (hept, J=6.1 Hz, 1H), 4.49 (dd, J=10.9, 3.9 Hz, 1H), 4.34 (dd, J=10.9, 6.3 Hz, 1H), 4.25-4.18 (m, 1H), 4.07-4.01 (m, 1H), 2.89-2.84 (m, 2H), 2.35-2.17 (m, 6H), 2.10-2.01 (m, 6H), 1.94-1.90 (m, 1H), 1.42 (d, J=6.1 Hz, 3H), 1.40 (d, J=6.1 Hz, 3H); LRMS (M+H) m/z 507.2.

### Example 61

### (S)-4-bromo-1-((4,4-difluoro-5-oxopyrrolidin-2-yl)methoxy)-7-isopropoxyisoquinoline-6-carboxamide (I-2)

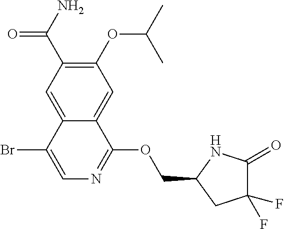 [SMILES: CC(C)Oc1cc2c(OC[C@@H]3CC(F)(F)C(=O)N3)ncc(Br)c2cc1C(N)=O]

Synthesized by following general procedures (A).

¹H NMR (400 MHz, Chloroform-d) δ 8.77 (s, 1H), 8.00 (s, 1H), 7.95 (s, 1H), 7.52 (s, 1H), 7.33 (s, 1H), 6.17 (s, 1H), 4.91 (hept, J=6.0 Hz, 1H), 4.74 (dd, J=11.6, 2.9 Hz, 1H), 4.41 (dd, J=11.6, 5.2 Hz, 1H), 4.33-4.28 (m, 1H), 2.93-2.80 (m, 1H), 2.65-2.53 (m, 1H), 1.49 (d, J=6.0 Hz, 3H), 1.46 (d, J=6.0 Hz, 3H); ¹⁹F NMR (376 MHz, Chloroform-d) δ −102.55-−104.84 (m); LRMS (M+Na) m/z 479.9, 481.9.

### Example 62

### (S)-1-((4,4-difluoro-5-oxopyrrolidin-2-yl)methoxy)-4-((1-hydroxycyclopentyl)ethynyl)-7-isopropoxyisoquinoline-6-carboxamide (I-48)

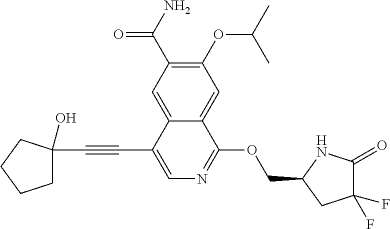 [SMILES: CC(C)Oc1cc2c(OC[C@@H]3CC(F)(F)C(=O)N3)ncc(C#CC3(O)CCCC3)c2cc1C(N)=O]

General procedures (B): 0.065 mmol scale, 66% yield.

¹H NMR (400 MHz, DMSO-d₆) δ 9.31 (s, 1H), 8.32 (s, 1H), 8.04 (s, 1H), 7.77 (br s, 1H), 7.74 (br s, 1H), 7.65 (s, 1H), 5.49 (s, 1H), 4.83 (hept, J=6.1 Hz, 1H), 4.59 (dd, J=11.3, 3.1 Hz, 1H), 4.36 (dd, J=11.3, 5.0 Hz, 1H), 4.24-4.18 (m, 1H), 2.94-2.79 (m, 1H), 2.70-2.57 (m, 1H), 2.02-1.93 (m, 4H), 1.83-1.72 (m, 4H), 1.39 (d, J=6.1 Hz, 3H), 1.38 (d, J=6.1 Hz, 3H); ¹⁹F NMR (376 MHz, DMSO-d₆) δ −100.95-−103.25 (m); LRMS (M+Na) m/z 510.1.

### Example 63

### (S)-1-((4,4-difluoro-5-oxopyrrolidin-2-yl)methoxy)-7-isopropoxy-4-((tetrahydro-2H-pyran-4-yl)ethynyl)isoquinoline-6-carboxamide (I-49)

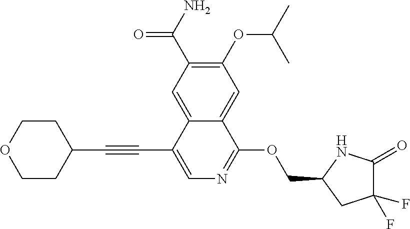 [SMILES: CC(C)Oc1cc2c(OC[C@@H]3CC(F)(F)C(=O)N3)ncc(C#CC3CCOCC3)c2cc1C(N)=O]

General procedures (B): 0.065 mmol scale, 63% yield.

¹H NMR (400 MHz, Chloroform-d) δ 8.92 (s, 1H), 7.99 (s, 1H), 7.96 (d, J=4.1 Hz, 1H), 7.55 (s, 1H), 7.39 (s, 1H), 6.07 (d, J=4.1 Hz, 1H), 4.91 (hept, J=6.0 Hz, 1H), 4.75 (dd, J=11.6, 3.0 Hz, 1H), 4.42 (dd, J=11.6, 5.6 Hz, 1H), 4.32-4.26 (m, 1H), 4.01 (ddd, J=11.7, 5.6, 3.7 Hz, 2H), 3.60 (ddd, J=11.6, 8.5, 2.9 Hz, 2H), 3.03-2.95 (m, 1H), 2.92-2.79 (m, 1H), 2.57 (dddd, J=15.0, 15.0, 13.0, 4.3 Hz, 1H), 2.04-1.98 (m, 2H), 1.87 (dddd, J=12.6, 8.6, 8.6, 3.6 Hz, 2H), 1.48 (d, J=6.0 Hz, 3H), 1.47 (d, J=6.0 Hz, 3H); ¹⁹F NMR (376 MHz, Chloroform-d) δ −102.82-−104.94 (m); LRMS (M+H) m/z 488.1.

### Example 64

### (S)-1-((4,4-difluoro-5-oxopyrrolidin-2-yl)methoxy)-7-isopropoxy-4-(3-(tetrahydro-2H-pyran-4-yl)prop-1-yn-1-yl)isoquinoline-6-carboxamide (I-50)

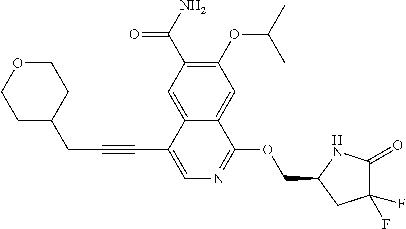 [SMILES: CC(C)Oc1cc2c(OC[C@@H]3CC(F)(F)C(=O)N3)ncc(C#CCC3CCOCC3)c2cc1C(N)=O]

General procedures (B): 0.065 mmol scale, 48% yield.

¹H NMR (400 MHz, DMSO-d₆) δ 9.30 (s, 1H), 8.38 (s, 1H), 8.04 (s, 1H), 7.78, (s, 1H), 7.77 (s, 1H), 7.65 (s, 1H), 4.85 (hept, J=6.0 Hz, 1H), 4.58 (dd, J=11.3, 3.2 Hz, 1H), 4.36 (dd, J=11.3, 5.1 Hz, 1H), 4.24-4.18 (m, 1H), 3.88 (ddd, J=11.3, 4.9, 1.7 Hz, 2H), 3.33 (ddd, J=11.7, 11.7, 2.0 Hz, 2H), 2.86 (dddd, J=19.6, 15.3, 15.3, 8.3 Hz, 1H)), 2.63 (dddd, J=19.6, 15.9, 12.1, 3.9 Hz, 1H), 2.54 (br d, J=6.5 Hz, 2H), 1.89-1.75 (m, 3H), 1.43-1.32 (m, 8H); ¹⁹F NMR (376 MHz, DMSO-d₆) δ −100.96-−103.26 (m); LRMS (M+H) m/z 502.1.

### Example 65

### (S)-1-((4,4-difluoro-5-oxopyrrolidin-2-yl)methoxy)-4-((4-hydroxytetrahydro-2H-pyran-4-yl)ethynyl)-7-isopropoxyisoquinoline-6-carboxamide (I-51)

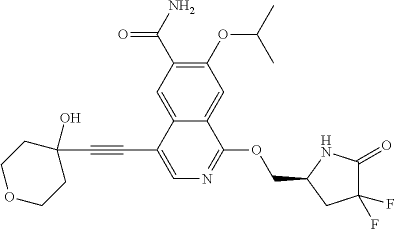 [SMILES: CC(C)Oc1cc2c(OC[C@@H]3CC(F)(F)C(=O)N3)ncc(C#CC3(O)CCOCC3)c2cc1C(N)=O]

General procedures (B): 0.08 mmol scale, 71% yield

¹H NMR (400 MHz, DMSO-d₆) δ 9.31 (s, 1H), 8.35 (s, 1H), 8.08 (s, 1H), 7.77 (br s, 1H), 7.75 (br s, 1H), 7.66 (s, 1H), 5.91 (s, 1H), 4.85 (hept, J=6.0 Hz, 1H), 4.60 (dd, J=11.3, 3.1 Hz, 1H), 4.37 (dd, J=11.3, 5.1 Hz, 1H), 4.25-4.19 (m, 1H), 3.83 (ddd, J=11.7, 4.3, 4.3 Hz, 2H), 3.63 (ddd, J=11.6, 8.6, 3.0 Hz, 2H), 2.94-2.79 (m, 1H), 2.70-2.57 (m, 1H), 1.99-1.93 (m, 2H), 1.81-1.74 (m, 2H), 1.39 (d, J=6.0 Hz, 3H), 1.38 (d, J=6.0 Hz, 3H); ¹⁹F NMR (376 MHz, DMSO-d₆) δ −100.98-−103.26 (m); LRMS (M+H) m/z 486.1 (M−H₂O+H), 526.1 (M+Na).

### Example 66

### tert-butyl (S)-4-((6-carbamoyl-1-((4,4-difluoro-5-oxopyrrolidin-2-yl)methoxy)-7-isopropoxyisoquinolin-4-yl)ethynyl)-4-hydroxypiperidine-1-carboxylate (I-52)

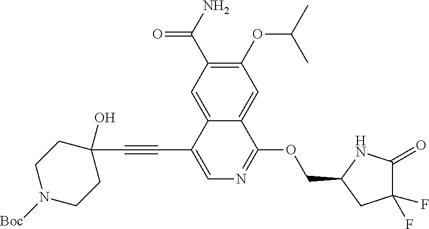 [SMILES: CC(C)Oc1cc2c(OC[C@@H]3CC(F)(F)C(=O)N3)ncc(C#CC3(O)CCN(C(=O)OC(C)(C)C)CC3)c2cc1C(N)=O]

General procedures (B): 0.1 mmol scale, 81% yield.

¹H NMR (400 MHz, Chloroform-d) δ 8.81 (br s, 1H), 8.60 (s, 1H), 8.04 (s, 1H), 7.88 (s, 1H), 7.13 (s, 1H), 6.27 (s, 1H), 4.89 (br s, 1H), 4.66 (hept, J=6.0 Hz, 1H), 4.55 (br d, J=11.7 Hz, 1H), 4.42 (dd, J=11.7, 2.9 Hz, 1H), 4.31 (br d, J=8.4 Hz, 1H), 3.88-3.79 (m, 2H), 3.48-3.40 (m, 2H), 2.93-2.79 (m, 1H), 2.66-2.54 (m, 1H), 2.19-2.03 (m, 2H), 1.99-1.88 (m, 2H), 1.48 (s, 9H), 1.46 (d, J=6.0 Hz, 3H), 1.37 (d, J=6.0 Hz, 3H); LRMS (M-Boc+H) m/z 503.1.

### Example 67

### (S)-1-((4,4-difluoro-5-oxopyrrolidin-2-yl)methoxy)-4-((4-hydroxypiperidin-4-yl)ethynyl)-7-isopropoxyisoquinoline-6-carboxamide (I-53)

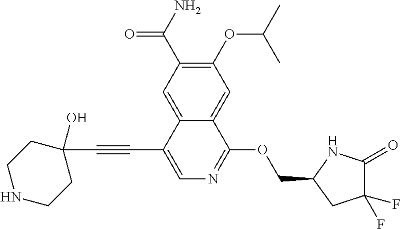 [SMILES: CC(C)Oc1cc2c(OC[C@@H]3CC(F)(F)C(=O)N3)ncc(C#CC3(O)CCNCC3)c2cc1C(N)=O]

General procedures (D), step 1: 0.069 mmol scale, >95% yield.

¹H NMR (400 MHz, DMSO-d₆) δ 9.31 (s, 1H), 8.35 (s, 1H), 8.06 (s, 1H), 7.77 (br s, 1H), 7.74 (br s, 1H), 7.65 (s, 1H), 5.71 (s, 1H), 4.84 (hept, J=6.0 Hz, 1H), 4.60 (dd, J=11.3, 3.2 Hz, 1H), 4.37 (dd, J=11.3, 5.1 Hz, 1H), 4.25-4.18 (m, 1H), 2.95-2.73 (m, 5H), 2.70-2.57 (m, 1H), 1.93-1.88 (m, 2H), 1.65 (ddd, J=12.8, 9.2, 3.8 Hz, 2H), 1.39 (d, J=6.0 Hz, 3H), 1.38 (d, J=6.0 Hz, 3H); ¹⁹F NMR (376 MHz, DMSO-d₆) δ −100.94-−103.25 (m); LRMS (M+H) m/z 503.1.

### Example 68

### (S)-1-((4,4-difluoro-5-oxopyrrolidin-2-yl)methoxy)-4-((4-hydroxy-1-methylpiperidin-4-yl)ethynyl)-7-isopropoxyisoquinoline-6-carboxamide (I-57)

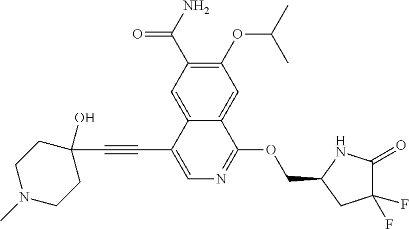 [SMILES: CC(C)Oc1cc2c(OC[C@@H]3CC(F)(F)C(=O)N3)ncc(C#CC3(O)CCN(C)CC3)c2cc1C(N)=O]

General procedures (D), step 2, formaldehyde was used: 0.06 mmol scale, 8% yield.

¹H NMR (400 MHz, DMSO-d₆) δ 9.33 (br s, 1H), 8.39 (s, 1H), 8.05 (s, 1H), 7.77 (br s, 1H), 7.75 (br s, 1H), 7.66 (s, 1H), 5.74 (br s, 1H), 4.85 (hept, J=6.1 Hz, 1H), 4.60 (dd, J=11.3, 3.2 Hz, 1H), 4.37 (dd, J=11.3, 5.1 Hz, 1H), 4.25-4.19 (m, 1H), 2.94-2.79 (m, 1H), 2.70-2.57 (m, 3H), 2.37-2.32 (m, 2H), 2.19 (s, 3H), 1.95-1.90 (m, 2H), 1.79 (ddd, J=12.8, 9.4, 3.6 Hz, 2H), 1.39 (d, J=6.1 Hz, 3H), 1.38 (d, J=6.1 Hz, 3H); ¹⁹F NMR (376 MHz, DMSO-d₆) δ −100.92-−103.25 (m); LRMS (M+H) m/z 517.1.

### Example 69

### tert-butyl (S)-4-((6-carbamoyl-1-((4,4-difluoro-5-oxopyrrolidin-2-yl)methoxy)-7-isopropoxyisoquinolin-4-yl)ethynyl)piperidine-1-carboxylate (I-54)

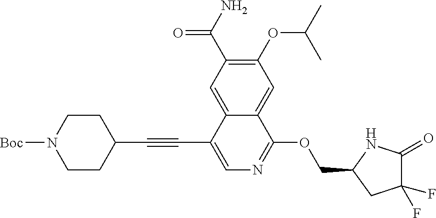 [SMILES: CC(C)Oc1cc2c(OC[C@@H]3CC(F)(F)C(=O)N3)ncc(C#CC3CCN(C(=O)OC(C)(C)C)CC3)c2cc1C(N)=O]

General procedures (B): 0.1 mmol scale, 54% yield.

¹H NMR (400 MHz, Chloroform-d) δ 8.77 (br s, 1H), 8.42-8.35 (m, 1H), 7.97 (d, J=4.0 Hz, 1H), 7.95 (s, 1H), 7.51 (s, 1H), 7.27 (s, OH), 6.40 (br s, 1H), 4.89 (hept, J=6.1 Hz, 1H), 4.76 (dd, J=11.4, 2.9 Hz, 1H), 4.38 (dd, J=11.4, 5.6 Hz, 1H), 4.33-4.27 (m, 1H), 3.85-3.79 (m, 2H), 3.26 (ddd, J=12.7, 8.5, 3.3 Hz, 2H), 2.92 (tt, J=8.0, 3.9 Hz, 1H), 2.88-2.77 (m, 1H), 2.56 (dddd, J=17.1, 14.9, 12.7, 4.1 Hz, 1H), 1.98-1.92 (m, 2H), 1.82-1.73 (m, 2H), 1.47 (s, 9H), 1.45 (d, J=6.1 Hz, 3H), 1.42 (d, J=6.1 Hz, 3H); ¹⁹F NMR (376 MHz, Chloroform-d) δ −102.54-−104.83 (m); LRMS (M+Na) m/z 609.6.

### Example 70

### (S)-1-((4,4-difluoro-5-oxopyrrolidin-2-yl)methoxy)-7-isopropoxy-4-(piperidin-4-ylethynyl)isoquinoline-6-carboxamide (I-55)

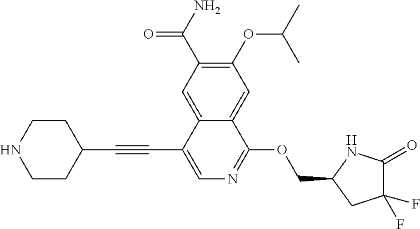 [SMILES: CC(C)Oc1cc2c(OC[C@@H]3CC(F)(F)C(=O)N3)ncc(C#CC3CCNCC3)c2cc1C(N)=O]

General procedures (D), step 1: 0.018 mmol scale, 75% yield.

¹H NMR (400 MHz, Methanol-d₄) δ 8.72 (s, 1H), 8.55 (br s, 1H), 8.03 (s, 1H), 7.73 (s, 1H), 4.96 (hept, J=6.1 Hz, 1H), 4.67 (dd, J=11.5, 3.0 Hz, 1H), 4.50 (dd, J=11.5, 4.0 Hz, 1H), 4.33-4.27 (m, 1H), 3.47-3.41 (m, 2H), 3.23-3.15 (m, 3H), 2.99-2.87 (m, 1H), 2.84-2.62 (m, 1H), 2.26-2.19 (m, 2H), 2.07-1.98 (m, 2H), 1.49 (d, J=6.1 Hz, 3H), 1.48 (d, J=6.1 Hz, 3H); ¹⁹F NMR (376 MHz, Methanol-d₄) δ −102.71-−105.44 (m); LRMS (M+H) m/z 487.5.

### Example 71

### (S)-1-((4,4-difluoro-5-oxopyrrolidin-2-yl)methoxy)-7-isopropoxy-4-((1-methylpiperidin-4-yl)ethynyl)isoquinoline-6-carboxamide (I-56)

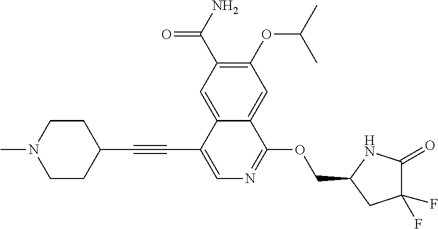 [SMILES: CC(C)Oc1cc2c(OC[C@@H]3CC(F)(F)C(=O)N3)ncc(C#CC3CCN(C)CC3)c2cc1C(N)=O]

General procedures (D), step 2, formaldehyde was used: 0.0156 mmol scale, 54% yield.

¹H NMR (400 MHz, DMSO-d₆) δ 9.31 (s, 1H), 8.36 (s, 1H), 8.03 (s, 1H), 7.77 (s, 1H), 7.75 (s, 1H), 7.65 (s, 1H), 4.84 (hept, J=6.0 Hz, 1H), 4.59 (dd, J=11.3, 3.2 Hz, 1H), 4.36 (dd, J=11.3, 5.1 Hz, 1H), 4.24-4.18 (m, 1H), 2.93-2.79 (m, 2H), 2.69-2.61 (m, 3H), 2.22-2.15 (m, 5H), 1.96-1.89 (m, 2H), 1.71 (dddd, J=13.6, 8.9, 4.3, 4.3 Hz, 2H), 1.39 (d, J=6.0 Hz, 3H), 1.38 (d, J=6.0 Hz, 3H); ¹⁹F NMR (376 MHz, DMSO-d₆) δ −100.98-−103.24 (m); LRMS (M+H) m/z 501.4.

### Example 72

### 1-(((S)-4,4-difluoro-5-oxopyrrolidin-2-yl)methoxy)-4-(((1r,4S)-4-ethoxycyclohexyl)ethynyl)-7-isopropoxyisoquinoline-6-carboxamide (I-58)

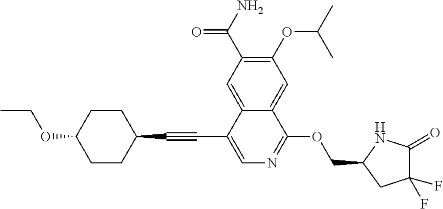 [SMILES: CCO[C@H]1CC[C@H](C#Cc2cnc(OC[C@@H]3CC(F)(F)C(=O)N3)c3cc(OC(C)C)c(C(N)=O)cc23)CC1]

General procedures (B): 0.06 mmol scale, 50% yield.

¹H NMR (400 MHz, DMSO-d₆) δ 9.30 (s, 1H), 8.33 (s, 1H), 8.01 (s, 1H), 7.76 (s, 1H), 7.75 (s, 1H), 7.64 (s, 1H), 4.84 (hept, J=6.1 Hz, 1H), 4.58 (dd, J=11.3, 3.2 Hz, 1H), 4.35 (dd, J=11.3, 5.1 Hz, 1H), 4.24-4.18 (m, 1H), 3.45 (q, J=7.0 Hz, 2H), 3.35-3.28 (m, partially overlapped with H₂O, 1H), 2.93-2.79 (m, 1H), 2.74-2.56 (m, 2H), 2.08-1.96 (m, 4H), 1.57-1.48 (m, 2H), 1.39 (d, J=6.1 Hz, 3H), 1.38 (d, J=6.1 Hz, 3H), 1.37-1.26 (m, partially overlapped, 2H), 1.10 (t, J=7.0 Hz, 3H); ¹⁹F NMR (376 MHz, DMSO-d₆) δ −100.95-−103.26 (m); LRMS (M+H) m/z 530.3.

### Example 73

### 1-(((S)-4,4-difluoro-5-oxopyrrolidin-2-yl)methoxy)-4-((cis-4-ethoxycyclohexyl)ethynyl)-7-isopropoxyisoquinoline-6-carboxamide (I-59)

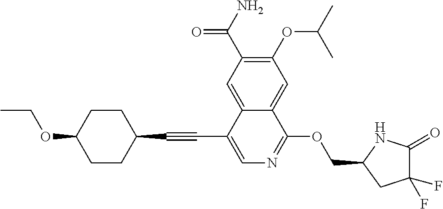 [SMILES: CCO[C@H]1CC[C@@H](C#Cc2cnc(OC[C@@H]3CC(F)(F)C(=O)N3)c3cc(OC(C)C)c(C(N)=O)cc23)CC1]

General procedures (B): 0.06 mmol scale, 13% yield

¹H NMR (400 MHz, DMSO-d₆) δ 9.31 (s, 1H), 8.33 (s, 1H), 8.03 (s, 1H), 7.77 (br s, 1H), 7.74 (br s, 1H), 7.65 (s, 1H), 4.84 (hept, J=6.0 Hz, 1H), 4.59 (dd, J=11.3, 3.2 Hz, 1H), 4.36 (dd, J=11.3, 5.1 Hz, 1H), 4.24-4.18 (m, 1H), 3.44 (q, J=7.0 Hz, 2H), 3.39-3.35 (m, 1H), 2.94-2.87 (m, 2H), 2.69-2.60 (m, 1H), 1.85-1.78 (m, 2H), 1.74-1.67 (m, 6H), 1.39 (d, J=6.0 Hz, 3H), 1.38 (d, J=6.0 Hz, 3H), 1.11 (t, J=7.0 Hz, 3H); ¹⁹F NMR (376 MHz, DMSO-d₆) δ −100.98-−103.26 (m); LRMS (M+H) m/z 530.5.

### Example 74

### 1-(((S)-4,4-difluoro-5-oxopyrrolidin-2-yl)methoxy)-4-(((1r,4S)-4-hydroxy-4-methylcyclohexyl)ethynyl)-7-isopropoxyisoquinoline-6-carboxamide (I-60)

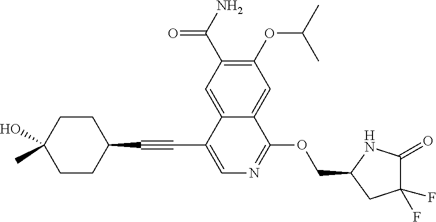 [SMILES: CC(C)Oc1cc2c(OC[C@@H]3CC(F)(F)C(=O)N3)ncc(C#C[C@H]3CC[C@](C)(O)CC3)c2cc1C(N)=O]

General procedures (B): 0.05 mmol scale, 24% yield.

¹H NMR (400 MHz, DMSO-d₆) δ 9.31 (s, 1H), 8.36 (s, 1H), 8.02 (s, 1H), 7.76 (br s, 2H), 7.65 (s, 1H), 4.85 (hept, J=6.0 Hz, 1H), 4.58 (dd, J=11.3, 3.2 Hz, 1H), 4.35 (dd, J=11.3, 5.1 Hz, 1H), 4.24-4.18 (m, 1H), 4.16 (s, 1H), 2.95-2.79 (m, 2H), 2.69-2.57 (m, 1H), 2.01-1.93 (m, 2H), 1.75-1.68 (m, 2H), 1.62-1.54 (m, 2H), 1.50-1.44 (m, 2H), 1.39 (d, J=6.0 Hz, 3H), 1.38 (d, J=6.0 Hz, 3H), 1.17 (s, 3H); ¹⁹F NMR (376 MHz, DMSO-d₆) δ −100.96-−103.27 (m); LRMS (M+H) m/z 516.3.

### Example 75

### 1-(((S)-4,4-difluoro-5-oxopyrrolidin-2-yl)methoxy)-4-(((1s,4R)-4-hydroxy-4-methylcyclohexyl)ethynyl)-7-isopropoxyisoquinoline-6-carboxamide (I-61)

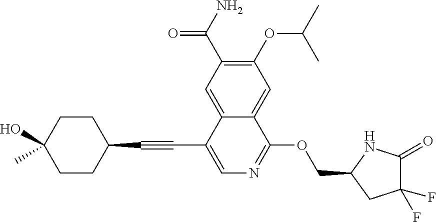 [SMILES: CC(C)Oc1cc2c(OC[C@@H]3CC(F)(F)C(=O)N3)ncc(C#C[C@H]3CC[C@@](C)(O)CC3)c2cc1C(N)=O]

General procedures (B): 0.05 mmol scale, 33% yield.

¹H NMR (400 MHz, DMSO-d₆) δ 9.31 (s, 1H), 8.33 (s, 1H), 8.02 (s, 1H), 7.78 (br s, 1H), 7.75 (br s, 1H), 7.64 (s, 1H), 4.84 (hept, J=6.0 Hz, 1H), 4.58 (dd, J=11.3, 3.2 Hz, 1H), 4.35 (dd, J=11.3, 5.1 Hz, 1H), 4.24-4.18 (m, 1H), 4.13 (s, 1H), 2.93-2.79 (m, 1H), 2.69-2.57 (m, 2H), 1.88-1.73 (m, 4H), 1.62-1.59 (m, 2H), 1.40-1.32 (m, 8H), 1.11 (s, 3H); ¹⁹F NMR (376 MHz, DMSO-d₆) δ −100.95-−103.25 (m); LRMS (M+H) m/z 516.4.

### Example 76

### tert-butyl (S)-3-((6-carbamoyl-1-((4,4-difluoro-5-oxopyrrolidin-2-yl)methoxy)-7-isopropoxyisoquinolin-4-yl)ethynyl)azetidine-1-carboxylate (I-62)

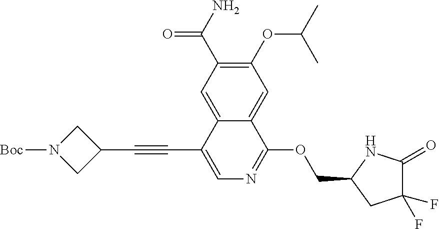 [SMILES: CC(C)Oc1cc2c(OC[C@@H]3CC(F)(F)C(=O)N3)ncc(C#CC3CN(C(=O)OC(C)(C)C)C3)c2cc1C(N)=O]

General procedures (B): 0.1 mmol scale, 43% yield.

¹H NMR (400 MHz, Chloroform-d) δ 8.82 (s, 1H), 8.00 (s, 1H), 7.95 (d, J=4.0 Hz, 1H), 7.53 (s, 1H), 7.28 (s, 1H), 6.10 (d, J=4.0 Hz, 1H), 4.90 (hept, J=6.0 Hz, 1H), 4.76 (dd, J=11.6, 3.0 Hz, 1H), 4.44 (dd, J=11.6, 5.2 Hz, 1H), 4.31-4.26 (m, 3H), 4.12 (ddd, J=8.1, 6.4, 3.5 Hz, 2H), 3.69 (tt, J=8.8, 6.4 Hz, 1H), 2.93-2.80 (m, 1H), 2.65-2.53 (m, 1H), 1.49 (d, J=6.0 Hz, 3H), 1.48 (d, J=6.0 Hz, 3H), 1.46 (s, 9H); ¹⁹F NMR (376 MHz, Chloroform-d) δ −102.69-−104.80 (m); LRMS (M+Na) m/z 581.1.

### Example 77

### (S)-4-(azetidin-3-ylethynyl)-1-((4,4-difluoro-5-oxopyrrolidin-2-yl)methoxy)-7-isopropoxyisoquinoline-6-carboxamide (I-63)

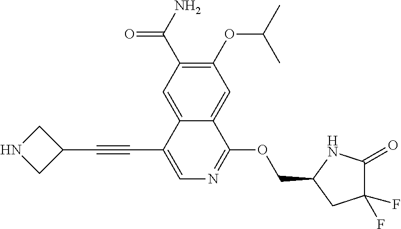 [SMILES: CC(C)Oc1cc2c(OC[C@@H]3CC(F)(F)C(=O)N3)ncc(C#CC3CNC3)c2cc1C(N)=O]

General procedures (D), step 1: 0.034 mmol scale, 93% yield.

¹H NMR (400 MHz, DMSO-d₆) δ 9.31 (s, 1H), 8.30 (s, 1H), 8.08 (s, 1H), 7.77 (br s, 2H), 7.65 (s, 1H), 4.84 (hept, J=6.0 Hz, 1H), 4.59 (dd, J=11.3, 3.2 Hz, 1H), 4.36 (dd, J=11.3, 5.0 Hz, 1H), 4.21 (tt, J=8.5, 4.2 Hz, 1H), 3.97-3.49 (m, overlapped with H₂O, 6H), 2.94-2.79 (m, 1H), 2.69-2.57 (m, 1H), 1.39 (d, J=6.0 Hz, 3H), 1.38 (d, J=6.0 Hz, 3H); ¹⁹F NMR (376 MHz, DMSO-d₆) δ −100.91-−103.24 (m); LRMS (M+H) m/z 459.2.

### Example 78

### (S)-1-((4,4-difluoro-5-oxopyrrolidin-2-yl)methoxy)-7-isopropoxy-4-((1-methylazetidin-3-yl)ethynyl)isoquinoline-6-carboxamide (I-64)

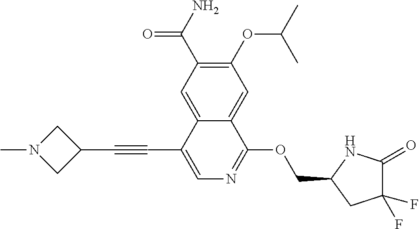 [SMILES: CC(C)Oc1cc2c(OC[C@@H]3CC(F)(F)C(=O)N3)ncc(C#CC3CN(C)C3)c2cc1C(N)=O]

General procedures (D), step 2: formaldehyde was used: 0.019 mmol scale, 69% yield.

¹H NMR (400 MHz, DMSO-d₆) δ 9.30 (s, 1H), 8.30 (s, 1H), 8.06 (s, 1H), 7.77 (br s, 2H), 7.65 (s, 1H), 4.84 (hept, J=6.1 Hz, 1H), 4.59 (dd, J=11.3, 3.2 Hz, 1H), 4.36 (dd, J=11.3, 5.1 Hz, 1H), 4.23-4.19 (m, 1H), 3.64-3.60 (m, 2H), 3.55-3.48 (m, 1H), 3.12-3.09 (m, 2H), 2.94-2.79 (m, 1H), 2.69-2.57 (m, 1H), 2.24 (s, 3H), 1.39 (d, J=6.1 Hz, 3H), 1.38 (d, J=6.1 Hz, 3H); ¹⁹F NMR (376 MHz, DMSO-d₆) δ −100.94-−103.24 (m); LRMS (M+H) m/z 473.1.

### Example 79

### (S)-1-((4,4-difluoro-5-oxopyrrolidin-2-yl)methoxy)-4-((3,3-dimethoxycyclobutyl)ethynyl)-7-isopropoxyisoquinoline-6-carboxamide (I-65)

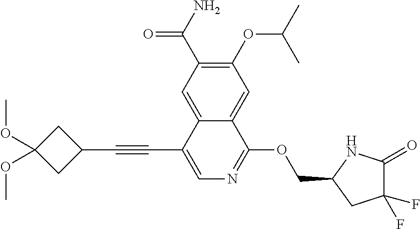 [SMILES: COC1(OC)CC(C#Cc2cnc(OC[C@@H]3CC(F)(F)C(=O)N3)c3cc(OC(C)C)c(C(N)=O)cc23)C1]

General procedures (B): 0.15 mmol scale, 26% yield.

¹H NMR (400 MHz, Chloroform-d) δ 8.76 (s, 1H), 8.02 (s, 1H), 7.96 (s, 1H), 7.95 (s, 1H), 7.48 (s, 1H), 6.28 (d, J=4.1 Hz, 1H), 4.88 (hept, J=6.0 Hz, 1H), 4.74 (dd, J=11.5, 3.0 Hz, 1H), 4.40 (dd, J=11.5, 5.5 Hz, 1H), 4.32-4.26 (m, 1H), 3.21 (s, 3H), 3.20 (s, 3H), 3.18-3.10 (m, 1H), 2.84 (dddd, J=17.9, 15.0, 12.5, 8.1 Hz, 1H), 2.72-2.66 (m, 2H), 2.56 (dddd, J=17.3, 15.0, 13.0, 4.3 Hz, 1H), 2.47-2.40 (m, 2H), 1.46 (d, J=6.0 Hz, 3H), 1.43 (d, J=6.0 Hz, 3H); ¹⁹F NMR (376 MHz, Chloroform-d) δ −102.67-−104.87 (m); LRMS (M+Na) m/z 540.0.

### Example 80

### (S)-1-((4,4-difluoro-5-oxopyrrolidin-2-yl)methoxy)-7-isopropoxy-4-((3-oxocyclobutyl)ethynyl)isoquinoline-6-carboxamide (with TFA)

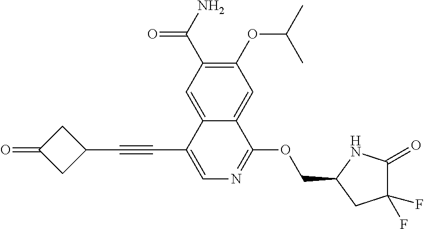 [SMILES: CC(C)Oc1cc2c(OC[C@@H]3CC(F)(F)C(=O)N3)ncc(C#CC3CC(=O)C3)c2cc1C(N)=O]

General procedures (D), step 1.

¹H NMR (400 MHz, Chloroform-d) δ 9.03 (s, 1H), 8.58 (s, 1H), 8.35 (s, 1H), 8.26 (s, 1H), 8.03 (s, 1H), 7.70 (s, 1H), 5.03-4.93 (m, 2H), 4.58 (dd, J=11.2, 5.4 Hz, 1H), 4.49-4.43 (m, 1H), 3.68-3.58 (m, 3H), 3.53-3.42 (m, 2H), 3.04-2.90 (m, 1H), 2.73-2.61 (m, 1H), 1.53 (d, J=6.0 Hz, 3H); 1.52 (d, J=6.0 Hz, 3H); ¹⁹F NMR (376 MHz, Chloroform-d) 6-101.02-−104.38 (m); LRMS (M+H) m/z 472.0.

### Example 81

### 1-(((S)-4,4-difluoro-5-oxopyrrolidin-2-yl)methoxy)-4-((cis-3-(dimethylamino)cyclobutyl)ethynyl)-7-isopropoxyisoquinoline-6-carboxamide (I-66)

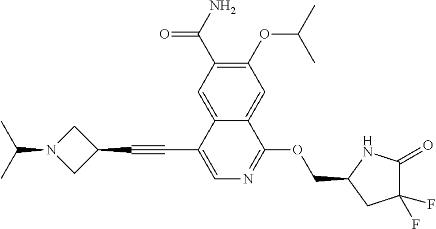

Similar procedures as the synthesis of I-31: 0.025 mmol scale, 62% yield.

¹H NMR (400 MHz, DMSO-d₆) δ 9.30 (s, 1H), 8.30 (s, 1H), 8.03 (s, 1H), 7.77 (s, 2H), 7.64 (s, 1H), 4.84 (hept, J=6.0 Hz, 1H), 4.58 (dd, J=11.3, 3.2 Hz, 1H), 4.36 (dd, J=11.3, 5.1 Hz, 1H), 4.24-4.18 (m, 1H), 3.04 (tt, J=9.7, 7.7 Hz, 1H), 2.93-2.79 (m, 1H), 2.69-2.47 (m, overlapped with DMSO, 4H), 2.05 (s, 6H), 2.01-1.95 (m, 2H), 1.39 (d, J=6.0 Hz, 3H), 1.38 (d, J=6.0 Hz, 3H); ¹⁹F NMR (376 MHz, DMSO-d₆) δ −100.95-−103.24 (m); LRMS (M+H) m/z 501.1.

### Example 82

### (S)-4-(1-cyclopropyl-1H-pyrazol-4-yl)-1-((4,4-difluoro-5-oxopyrrolidin-2-yl)methoxy)-7isopropoxyisoquinoline-6-carboxamide (I-67)

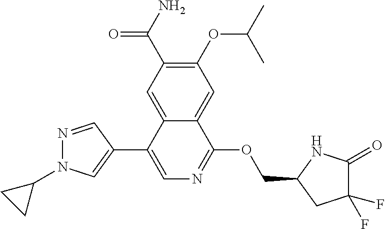 [SMILES: CC(C)Oc1cc2c(OC[C@@H]3CC(F)(F)C(=O)N3)ncc(-c3cnn(C4CC4)c3)c2cc1C(N)=O]

General procedures (C): 0.08 mmol scale, 66% yield.

¹H NMR (400 MHz, DMSO-d₆) δ 9.32 (s, 1H), 8.30 (s, 1H), 8.12 (s, 1H), 7.87 (s, 1H), 7.74 (br s, 2H), 7.69 (s, 1H), 7.69 (s, 1H), 4.87 (hept, J=6.0 Hz, 1H), 4.59 (dd, J=11.3, 3.3 Hz, 1H), 4.37 (dd, J=11.3, 5.0 Hz, 1H), 4.25-4.20 (m, 1H), 3.84 (tt, J=7.4, 3.8 Hz, 1H), 2.87 (dddd, J=19.7, 15.2, 15.2, 8.3 Hz, 1H), 2.69-2.57 (m, 1H), 1.40 (d, J=6.0 Hz, 3H), 1.39 (d, J=6.0 Hz, 3H), 1.16-1.12 (m, 2H), 1.04-0.99 (m, 2H); ¹⁹F NMR (376 MHz, DMSO-d₆) δ −100.93-−103.23 (m); LRMS (M+H) m/z 486.1.

### Example 83

### (S)-4-(1-cyclobutyl-1H-pyrazol-4-yl)-1-((4,4-difluoro-5-oxopyrrolidin-2-yl)methoxy)-7-isopropoxyisoquinoline-6-carboxamide (I-68)

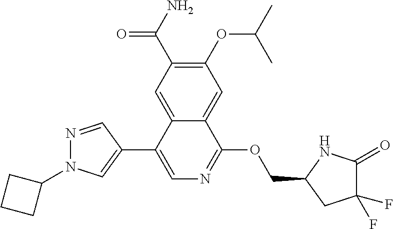 [SMILES: CC(C)Oc1cc2c(OC[C@@H]3CC(F)(F)C(=O)N3)ncc(-c3cnn(C4CCC4)c3)c2cc1C(N)=O]

General procedures (C): 0.08 mmol scale, 37% yield.

¹H NMR (400 MHz, Chloroform-d) δ 8.87 (s, 1H), 7.98 (d, J=4.0 Hz, 1H), 7.82 (s, 1H), 7.70 (d, J=0.8 Hz, 1H), 7.67 (d, J=0.8 Hz, 1H), 7.62 (s, 1H), 7.15 (s, 1H), 5.99 (d, J=4.1 Hz, 1H), 4.95 (hept, J=6.0 Hz, 1H), 4.86 (dddd, J=9.6, 8.3, 8.3, 7.2 Hz, 1H), 4.77 (dd, J=11.6, 3.0 Hz, 1H), 4.44 (dd, J=11.6, 5.6 Hz, 1H), 4.33-4.27 (m, 1H), 2.93-2.80 (m, 1H), 2.71-2.52 (m, 5H), 1.99-1.84 (m, 2H), 1.49 (d, J=6.0 Hz, 3H), 1.48 (d, J=6.0 Hz, 3H); ¹⁹F NMR (376 MHz, Chloroform-d) δ −102.95-−104.94 (m); LRMS (M+H) m/z 500.1.

### Example 84

### (S)-4-(1-cyclopentyl-1H-pyrazol-4-yl)-1-((4,4-difluoro-5-oxopyrrolidin-2-yl)methoxy)-7-isopropoxyisoquinoline-6-carboxamide (I-69)

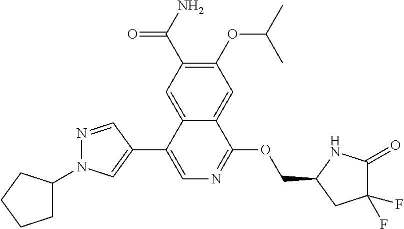 [SMILES: CC(C)Oc1cc2c(OC[C@@H]3CC(F)(F)C(=O)N3)ncc(-c3cnn(C4CCCC4)c3)c2cc1C(N)=O]

General procedures (C): 0.08 mmol scale, 73% yield.

¹H NMR (400 MHz, Chloroform-d) δ 8.88 (s, 1H), 7.98 (d, J=4.0 Hz, 1H), 7.81 (s, 1H), 7.68 (d, J=0.8 Hz, 1H), 7.66 (d, J=0.8 Hz, 1H), 7.61 (s, 1H), 7.36 (s, 1H), 6.04 (d, J=4.0 Hz, 1H), 4.94 (hept, J=6.0 Hz, 1H), 4.79-4.71 (m, 2H), 4.43 (dd, J=11.6, 5.6 Hz, 1H), 4.33-4.27 (m, 1H), 2.93-2.79 (m, 1H), 2.60-2.55 (m, 1H), 2.29-2.20 (m, 2H), 2.17-2.08 (m, 2H), 1.99-1.89 (m, 2H), 1.80-1.70 (m, 2H), 1.48 (d, J=6.0 Hz, 3H), 1.48 (d, J=6.0 Hz, 3H); ¹⁹F NMR (376 MHz, Chloroform-d) δ −102.90-−104.90 (m); LRMS (M+H) m/z 514.1.

### Example 85

### (S)-1-((4,4-difluoro-5-oxopyrrolidin-2-yl)methoxy)-7-isopropoxy-4-(1-(tetrahydro-2H-pyran-4-yl)-1H-pyrazol-4-yl)isoquinoline-6-carboxamide (I-70)

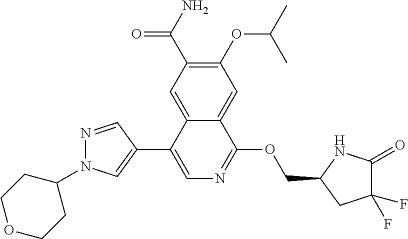 [SMILES: CC(C)Oc1cc2c(OC[C@@H]3CC(F)(F)C(=O)N3)ncc(-c3cnn(C4CCOCC4)c3)c2cc1C(N)=O]

General procedures (C): 0.08 mmol scale, 72% yield.

¹H NMR (400 MHz, DMSO-d₆) δ 9.32 (s, 1H), 8.32 (s, 1H), 8.14 (s, 1H), 7.88 (s, 1H), 7.74 (s, 1H), 7.73 (br s, 2H), 7.69 (s, 1H), 4.86 (hept, J=6.0 Hz, 1H), 4.59 (dd, J=11.3, 3.2 Hz, 1H), 4.56-4.48 (m, 1H), 4.38 (dd, J=11.3, 5.0 Hz, 1H), 4.26-4.19 (m, 1H), 4.02-3.97 (m, 2H), 3.53-3.47 (m, 2H), 2.95-2.80 (m, 1H), 2.70-2.57 (m, 1H), 2.08-2.01 (m, 4H), 1.40 (d, J=6.0 Hz, 3H), 1.39 (d, J=6.0 Hz, 3H); ¹⁹F NMR (376 MHz, DMSO-d₆) δ −100.95-−103.23 (m); LRMS (M+H) m/z 530.1.

### Example 86

### (S)-1-((4,4-difluoro-5-oxopyrrolidin-2-yl)methoxy)-7-isopropoxy-4-(1-((tetrahydro-2H-pyran-4-yl)methyl)-1H-pyrazol-4-yl)isoquinoline-6-carboxamide (I-71)

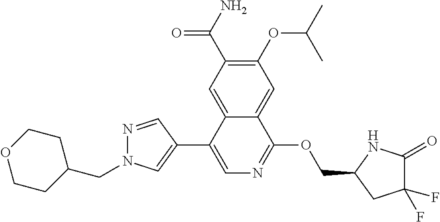 [SMILES: CC(C)Oc1cc2c(OC[C@@H]3CC(F)(F)C(=O)N3)ncc(-c3cnn(CC4CCOCC4)c3)c2cc1C(N)=O]

General procedures (C): 0.08 mmol scale, 80% yield.

¹H NMR (400 MHz, DMSO-d₆) δ 9.32 (s, 1H), 8.32 (s, 1H), 8.06 (d, J=0.8 Hz, 1H), 7.87 (s, 1H), 7.74 (br s, 2H), 7.71 (d, J=0.8 Hz, 1H), 7.69 (s, 1H), 4.87 (hept, J=6.0 Hz, 1H), 4.59 (dd, J=11.3, 3.2 Hz, 1H), 4.38 (dd, J=11.3, 5.0 Hz, 1H), 4.27-4.18 (m, 1H), 4.11 (d, J=7.1 Hz, 2H), 3.85 (ddd, J=11.5, 4.5, 1.9 Hz, 2H), 3.32-3.25 (m, partially overlapped with H₂O, 2H), 2.87 (dddd, J=19.7, 15.2, 15.2, 8.3 Hz, 1H), 2.63 (dddd, J=19.0, 15.4, 12.1, 3.6 Hz, 1H), 2.18-2.07 (m, 1H), 1.49-1.45 (m, 2H), 1.40 (d, J=6.0 Hz, 3H), 1.39 (d, J=6.0 Hz, 3H), 1.34-1.24 (m, 2H); ¹⁹F NMR (376 MHz, DMSO-d₆) δ −100.93-−103.22 (m); LRMS (M+H) m/z 544.1.

### Example 87

### (S)-1-((4,4-difluoro-5-oxopyrrolidin-2-yl)methoxy)-7-isopropoxy-4-(1-(2-(tetrahydro-2H-pyran-4-yl)ethyl)-1H-pyrazol-4-yl)isoquinoline-6-carboxamide (I-72)

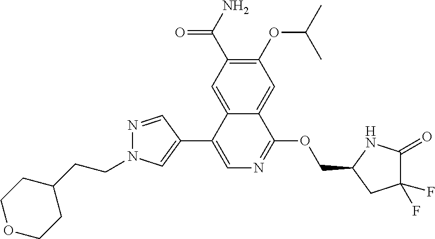 [SMILES: CC(C)Oc1cc2c(OC[C@@H]3CC(F)(F)C(=O)N3)ncc(-c3cnn(CCC4CCOCC4)c3)c2cc1C(N)=O]

General procedures (C): 0.08 mmol scale, 63% yield.

¹H NMR (400 MHz, Chloroform-d) δ 8.86 (s, 1H), 7.99 (d, J=4.1 Hz, 1H), 7.82 (s, 1H), 7.67 (d, J=0.8 Hz, 1H), 7.64 (d, J=0.8 Hz, 1H), 7.63 (s, 1H), 7.53 (s, 1H), 6.07 (d, J=4.1 Hz, 1H), 4.95 (hept, J=6.0 Hz, 1H), 4.78 (dd, J=11.5, 3.0 Hz, 1H), 4.42 (dd, J=11.6, 5.7 Hz, 1H), 4.33-4.25 (m, 3H), 3.97 (ddd, J=11.4, 4.6, 1.8 Hz, 2H), 3.40 (ddd, J=11.8, 11.8, 2.0 Hz, 2H), 2.86 (dddd, J=17.7, 15.0, 12.4, 8.1 Hz, 1H), 2.57 (dddd, J=17.4, 15.0, 13.2, 4.4 Hz, 1H), 1.93 (ddd, J=8.0, 6.7, 6.7 Hz, 2H), 1.70-1.56 (m, 3H), 1.48 (d, J=6.0 Hz, 6H), 1.43-1.33 (m, 2H); ¹⁹F NMR (376 MHz, Chloroform-d) δ −102.85-−104.90 (m); LRMS (M+H) m/z 558.1.

### Example 88

### 1-(((S)-4,4-difluoro-5-oxopyrrolidin-2-yl)methoxy)-4-(1-(cis-3-(dimethylamino)cyclobutyl)-1H-pyrazol-4-yl)-7-isopropoxyisoquinoline-6-carboxamide (I-73)

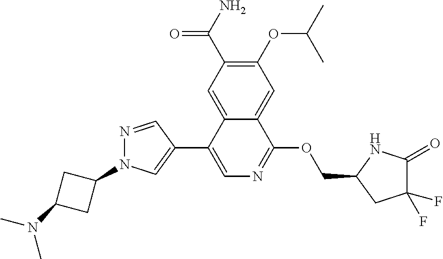 [SMILES: CC(C)Oc1cc2c(OC[C@@H]3CC(F)(F)C(=O)N3)ncc(-c3cnn([C@H]4C[C@@H](N(C)C)C4)c3)c2cc1C(N)=O]

General procedures (C): 0.08 mmol scale, 32% yield.

¹H NMR (400 MHz, DMSO-d₆) δ 9.33 (s, 1H), 8.30 (s, 1H), 8.11 (d, J=0.8 Hz, 1H), 7.88 (s, 1H), 7.75-7.74 (m, 3H), 7.69 (s, 1H), 4.87 (hept, J=6.1 Hz, 1H), 4.69-4.57 (m, 2H), 4.38 (dd, J=11.3, 5.0 Hz, 1H), 4.26-4.19 (m, 1H), 2.95-2.80 (m, 1H), 2.70-2.52 (m, 3H), 2.51-2.48 (m, overlapped with DMSO, 1H), 2.39-2.31 (m, 2H), 2.09 (s, 6H), 1.41 (d, J=6.1 Hz, 3H), 1.40 (d, J=6.1 Hz, 3H); LRMS (M+H) m/z 543.1.

### Example 89

### tert-butyl (S)-4-(4-(6-carbamoyl-1-((4,4-difluoro-5-oxopyrrolidin-2-yl)methoxy)-7-isopropoxyisoquinolin-4-yl)-1H-pyrazol-1-yl)piperidine-1-carboxylate (I-74)

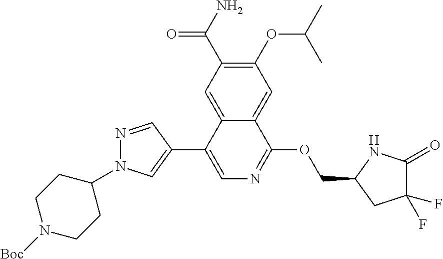 [SMILES: CC(C)Oc1cc2c(OC[C@@H]3CC(F)(F)C(=O)N3)ncc(-c3cnn(C4CCN(C(=O)OC(C)(C)C)CC4)c3)c2cc1C(N)=O]

General procedures (C): 0.1 mmol scale, 71% yield.

¹H NMR (400 MHz, Chloroform-d) δ 8.87 (s, 1H), 7.99 (d, J=4.1 Hz, 1H), 7.82 (s, 1H), 7.70 (d, J=0.8 Hz, 1H), 7.66 (d, J=0.8 Hz, 1H), 7.63 (s, 1H), 7.17 (s, 1H), 5.98 (d, J=4.1 Hz, 1H), 4.94 (hept, J=6.0 Hz, 1H), 4.78 (dd, J=11.6, 3.0 Hz, 1H), 4.44 (dd, J=11.6, 5.5 Hz, 1H), 4.37 (ddd, J=11.6, 4.0, 4.0 Hz, 1H), 4.40-4.27 (m, 4H), 2.95-2.80 (m, 3H), 2.65-2.52 (m, 1H), 2.25-2.22 (m, 2H), 2.07-1.97 (m, 2H), 1.49 (d, J=6.0 Hz, 6H), 1.48 (s, 9H); ¹⁹F NMR (376 MHz, Chloroform-d) δ −102.90-−104.88 (m); LRMS (M+Na) m/z 651.2.

### Example 90

### (S)-1-((4,4-difluoro-5-oxopyrrolidin-2-yl)methoxy)-7-isopropoxy-4-(1-(piperidin-4-yl)-1H-pyrazol-4-yl)isoquinoline-6-carboxamide (I-75)

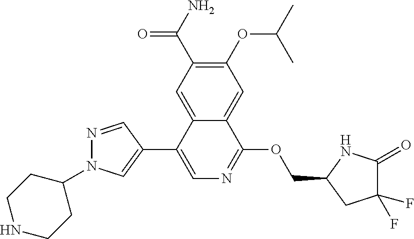 [SMILES: CC(C)Oc1cc2c(OC[C@@H]3CC(F)(F)C(=O)N3)ncc(-c3cnn(C4CCNCC4)c3)c2cc1C(N)=O]

General procedures (D), step 1: 0.068 mmol scale, 38% yield.

¹H NMR (400 MHz, DMSO-d₆) δ 9.33 (s, 1H), 8.32 (s, 1H), 8.10 (d, J=0.8 Hz, 1H), 7.88 (s, 1H), 7.74-7.73 (m, 2H), 7.69 (s, 1H), 4.87 (hept, J=6.1 Hz, 1H), 4.59 (dd, J=11.3, 3.2 Hz, 1H), 4.42-4.34 (m, 2H), 4.26-4.19 (m, 1H), 3.19-3.14 (m, 2H), 2.95-2.80 (m, 1H), 2.74 (td, J=12.4, 2.6 Hz, 2H), 2.70-2.57 (m, 1H), 2.11-2.07 (m, 2H), 2.01-1.91 (m, 2H), 1.40 (d, J=6.1 Hz, 3H), 1.39 (d, J=6.1 Hz, 3H); ¹⁹F NMR (376 MHz, DMSO-d₆) δ −100.93-−103.22 (m); LRMS (M+H) m/z 529.1.

### Example 91

### (S)-1-((4,4-difluoro-5-oxopyrrolidin-2-yl)methoxy)-7-isopropoxy-4-(1-(1-methylpiperidin-4-yl)-1H-pyrazol-4-yl)isoquinoline-6-carboxamide (I-76)

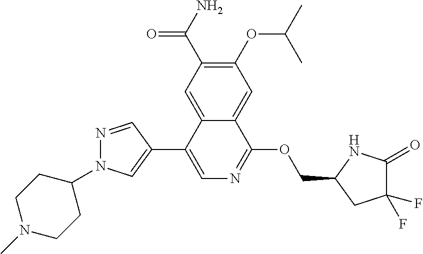 [SMILES: CC(C)Oc1cc2c(OC[C@@H]3CC(F)(F)C(=O)N3)ncc(-c3cnn(C4CCN(C)CC4)c3)c2cc1C(N)=O]

General procedures (D), step 2: formaldehyde was used: 0.034 mmol scale, 47% yield.

¹H NMR (400 MHz, DMSO-d₆) δ 9.32 (s, 1H), 8.32 (s, 1H), 8.12 (d, J=0.8 Hz, 1H), 7.88 (s, 1H), 7.74 (s, 1H), 7.73 (s, 1H), 7.72 (d, J=0.8 Hz, 1H), 7.69 (s, 1H), 4.87 (hept, J=6.1 Hz, 1H), 4.59 (dd, J=11.3, 3.2 Hz, 1H), 4.38 (dd, J=11.3, 5.0 Hz, 1H), 4.26-4.19 (m 2H), 2.95-2.80 (m, 3H), 2.70-2.57 (m, 1H), 2.23 (s, 3H), 2.12-2.01 (m, 6H), 1.40 (d, J=6.1 Hz, 3H), 1.39 (d, J=6.1 Hz, 3H); ¹⁹F NMR (376 MHz, DMSO-d₆) δ −100.91-−103.21 (m); LRMS (M+H) m/z 543.1.

### Example 92

### 1-(((S)-4,4-difluoro-5-oxopyrrolidin-2-yl)methoxy)-4-(1-(trans-4-ethoxycyclohexyl)-1H-pyrazol-4-yl)-7-isopropoxyisoquinoline-6-carboxamide (I-77)

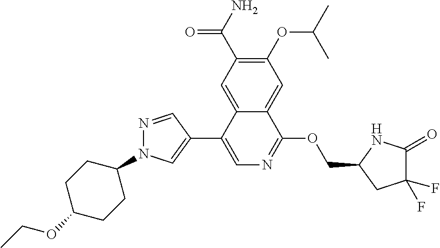 [SMILES: CCO[C@H]1CC[C@H](n2cc(-c3cnc(OC[C@@H]4CC(F)(F)C(=O)N4)c4cc(OC(C)C)c(C(N)=O)cc34)cn2)CC1]

General procedures (C): 0.1 mmol scale, 24% yield.

¹H NMR (400 MHz, DMSO-d₆) δ 9.32 (s, 1H), 8.32 (s, 1H), 8.08 (d, J=0.8 Hz, 1H), 7.87 (s, 1H), 7.74 (s, 1H), 7.74 (s, 1H), 7.71 (d, J=0.8 Hz, 1H), 7.69 (s, 1H), 4.87 (hept, J=6.0 Hz, 1H), 4.59 (dd, J=11.3, 3.2 Hz, 1H), 4.37 (dd, J=11.3, 5.0 Hz, 1H), 4.31-4.20 (m, 2H), 3.49 (q, J=7.0 Hz, 2H), 3.37-3.30 (m, overlapped with H₂O, 1H), 2.95-2.80 (m, 1H), 2.70-2.57 (m, 1H), 2.14-2.07 (m, 4H), 1.92-1.82 (m, 2H), 1.41-1.31 (m, 8H), 1.11 (t, J=7.0 Hz, 3H); ¹⁹F NMR (376 MHz, DMSO-d₆) δ −100.91-−103.20 (m); LRMS (M+H) m/z 572.3.

### Example 93

### 1-(((S)-4,4-difluoro-5-oxopyrrolidin-2-yl)methoxy)-4-(1-((1r,4S)-4-hydroxy-4-methylcyclohexyl)-1H-pyrazol-4-yl)-7-isopropoxyisoquinoline-6-carboxamide (I-78)

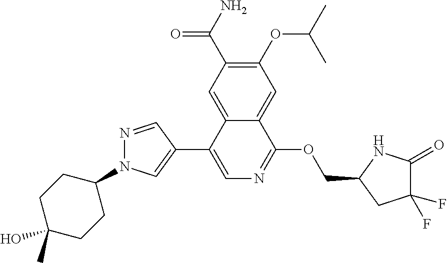 [SMILES: CC(C)Oc1cc2c(OC[C@@H]3CC(F)(F)C(=O)N3)ncc(-c3cnn([C@H]4CC[C@](C)(O)CC4)c3)c2cc1C(N)=O]

General procedures (C): 0.048 mmol scale, 38% yield.

¹H NMR (400 MHz, DMSO-d₆) δ 9.33 (s, 1H), 8.33 (s, 1H), 8.13 (s, 1H), 7.88 (s, 1H), 7.74 (br s, 2H), 7.71 (s, 1H), 7.69 (s, 1H), 4.87 (hept, J=6.0 Hz, 1H), 4.59 (dd, J=11.3, 3.2 Hz, 1H), 4.43 (s, 1H), 4.37 (dd, J=11.3, 5.0 Hz, 1H), 4.31-4.19 (m, 2H), 2.95-2.80 (m, 1H), 2.70-2.57 (m, 1H), 2.07-1.90 (m, 4H), 1.69-1.63 (m, 2H), 1.61-1.53 (m, 2H), 1.40 (d, J=6.0 Hz, 3H), 1.39 d, J=6.0 Hz, 3H), 1.22 (s, 3H); ¹⁹F NMR (376 MHz, DMSO-d₆) δ −100.92-−103.21 (m); LRMS (M+H) m/z 558.5.

### Example 94

### 4-bromo-1-(((2S,3S,4S)-3-ethyl-4-fluoro-5-oxopyrrolidin-2-yl)methoxy)-7-isopropoxyisoquinoline-6-carboxamide

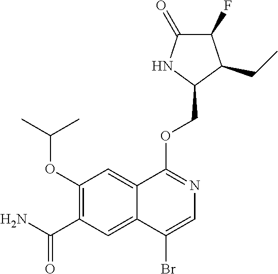 [SMILES: CC[C@@H]1[C@H](F)C(=O)N[C@@H]1COc1ncc(Br)c2cc(C(N)=O)c(OC(C)C)cc12]

Synthesized by following General procedures (A).

¹H NMR (400 MHz, Chloroform-d) δ 8.82 (s, 1H), 8.01 (s, 1H), 7.95 (s, 1H), 7.66 (s, 1H), 6.71 (s, 1H), 6.12 (s, 1H), 4.98-4.83 (m, 2H), 4.74 (dd, J=11.7, 3.3 Hz, 1H), 4.41 (dd, J=11.7, 5.6 Hz, 1H), 4.20-4.16 (m, 1H), 2.68-2.52 (m, 1H), 1.90-1.79 (m, 1H), 1.73-1.65 (m, 1H), 1.50 (d, J=6.0 Hz, 3H), 1.47 (d, J=6.0 Hz, 4H), 1.13 (t, J=7.4 Hz, 3H); ¹⁹F NMR (376 MHz, Chloroform-d) δ −199.52 (ddd, J=53.2, 35.3, 3.2 Hz); LRMS (M+H) m/z 426.3, 428.3.

### Example 95

### 1-(((2S,3S,4S)-3-ethyl-4-fluoro-5-oxopyrrolidin-2-yl)methoxy)-7-isopropoxy-4-((tetrahydro-2H-pyran-4-yl)ethynyl)isoquinoline-6-carboxamide (I-79)

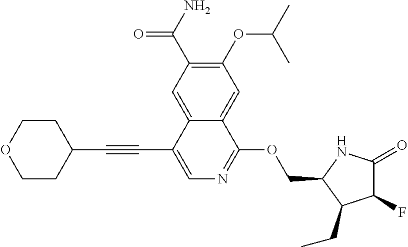 [SMILES: CC[C@@H]1[C@H](F)C(=O)N[C@@H]1COc1ncc(C#CC2CCOCC2)c2cc(C(N)=O)c(OC(C)C)cc12]

General procedures (B): 0.05 mmol scale, 36% yield.

¹H NMR (400 MHz, Chloroform-d) δ 8.94 (s, 1H), 8.00 (s, 1H), 7.97 (d, J=4.1 Hz, 1H), 7.66 (s, 1H), 6.81 (s, 1H), 6.07 (d, J=4.1 Hz, 1H), 4.98-4.83 (m, 2H), 4.77 (dd, J=11.7, 3.3 Hz, 1H), 4.41 (dd, J=11.6, 6.0 Hz, 1H), 4.20-4.15 (m, 1H), 4.01 (ddd, J=11.7, 5.6, 3.7 Hz, 2H), 3.60 (ddd, J=11.6, 8.5, 3.0 Hz, 2H), 3.00 (tt, J=8.4, 4.1 Hz, 1H), 2.67-2.52 (m, 1H), 2.04-1.97 (m, 2H), 1.91-1.81 (m, 3H), 1.74-1.66 (m, overlapped with H₂O, 1H), 1.49 (d, J=6.0 Hz, 3H), 1.48 (d, J=6.0 Hz, 3H), 1.13 (t, J=7.3 Hz, 3H); ¹⁹F NMR (376 MHz, Chloroform-d) δ −199.50 (ddd, J=53.5, 35.4, 3.3 Hz); LRMS (M+H) m/z 498.5.

### Example 96

### 4-(((1r,4S)-4-ethoxycyclohexyl)ethynyl)-1-(((2S,3S,4S)-3-ethyl-4-fluoro-5-oxopyrrolidin-2-yl)methoxy)-7-isopropoxyisoquinoline-6-carboxamide (I-80)

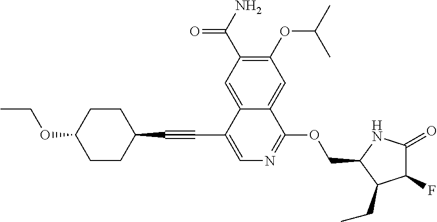 [SMILES: CCO[C@H]1CC[C@H](C#Cc2cnc(OC[C@H]3NC(=O)[C@@H](F)[C@H]3CC)c3cc(OC(C)C)c(C(N)=O)cc23)CC1]

General procedures (B): 0.05 mmol scale, 36% yield.

¹H NMR (400 MHz, Chloroform-d) δ 8.96 (s, 1H), 7.97 (s, 1H), 7.96 (d, J=4.1 Hz, 1H), 7.65 (s, 1H), 6.74 (s, 1H), 6.01 (d, J=4.1 Hz, 1H), 4.99-4.82 (m, 2H), 4.76 (dd, J=11.6, 3.3 Hz, 1H), 4.40 (dd, J=11.6, 6.1 Hz, 1H), 4.19-4.14 (m, 1H), 3.52 (q, J=7.0 Hz, 2H), 3.33 (tt, J=9.5, 3.8 Hz, 1H), 2.68-2.50 (m, 2H), 2.20-2.15 (m, 2H), 2.13-2.07 (m, 2H), 1.90-1.79 (m, 1H), 1.74-1.56 (m, overlapped with H₂O, 3H), 1.48 (d, J=6.0 Hz, 3H), 1.47 (d, J=6.0 Hz, 3H), 1.43-1.33 (m, 2H), 1.21 (t, J=7.0 Hz, 3H), 1.12 (t, J=7.3 Hz, 3H); ¹⁹F NMR (376 MHz, Chloroform-d) δ −199.50 (ddd, J=53.1, 35.1, 3.3 Hz); LRMS (M+H) m/z 540.5.

### Example 97

### 4-(((1s,4R)-4-ethoxycyclohexyl)ethynyl)-1-(((2S,3S,4S)-3-ethyl-4-fluoro-5-oxopyrrolidin-2-yl)methoxy)-7-isopropoxyisoquinoline-6-carboxamide (I-81)

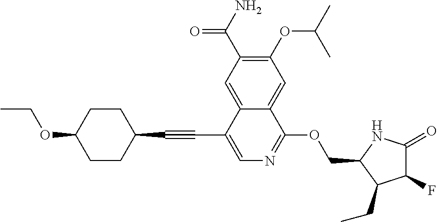 [SMILES: CCO[C@H]1CC[C@@H](C#Cc2cnc(OC[C@H]3NC(=O)[C@@H](F)[C@H]3CC)c3cc(OC(C)C)c(C(N)=O)cc23)CC1]

General procedures (B): 0.05 mmol scale, 45% yield.

¹H NMR (400 MHz, Chloroform-d) δ 8.93 (s, 1H), 7.99 (s, 1H), 7.92 (d, J=4.1 Hz, 1H), 7.64 (s, 1H), 6.95 (d, J=4.1 Hz, 1H), 6.08 (s, 1H), 4.98-4.82 (m, 2H), 4.77 (dd, J=11.6, 3.4 Hz, 1H), 4.39 (dd, J=11.6, 6.2 Hz, 1H), 4.20-4.14 (m, 1H), 3.51 (q, J=7.0 Hz, 2H), 3.36 (tt, J=7.5, 3.4 Hz, 1H), 2.93-2.87 (m, 1H), 2.65-2.49 (m, 1H), 2.04-1.97 (m, 2H), 1.92-1.65 (m, 8H), 1.47 (d, J=6.0 Hz, 3H), 1.46 (d, J=6.0 Hz, 3H), 1.20 (t, J=7.0 Hz, 3H), 1.12 (t, J=7.3 Hz, 3H); ¹⁹F NMR (376 MHz, Chloroform-d) δ −199.45 (ddd, J=53.1, 35.3, 3.2 Hz); LRMS (M+H) m/z 540.5.

### Example 98

### 4-bromo-1-(((2S,3S,4S)-3-ethyl-4-fluoro-5-oxopyrrolidin-2-yl)methoxy)-7-methoxyisoquinoline-6-carboxamide

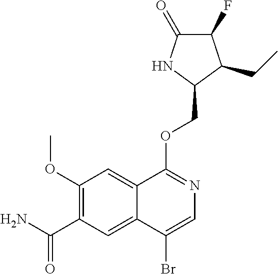 [SMILES: CC[C@@H]1[C@H](F)C(=O)N[C@@H]1COc1ncc(Br)c2cc(C(N)=O)c(OC)cc12]

Synthesized by following general procedures (A).

¹H NMR (400 MHz, Chloroform-d) δ 8.75 (s, 1H), 8.03 (s, 1H), 7.70 (s, 1H), 7.63 (s, 1H), 7.04-6.93 (m, 1H), 6.13 (s, 1H), 4.91 (dd, J=53.3, 6.2 Hz, 1H), 4.71 (dd, J=11.6, 3.2 Hz, 1H), 4.43 (dd, J=11.6, 5.8 Hz, 1H), 4.22-4.17 (m, 1H), 4.06 (s, 3H), 2.68-2.52 (m, 1H), 1.88-1.77 (m, 1H), 1.72-1.61 (m, 1H), 1.13 (t, J=7.3 Hz, 3H); ¹⁹F NMR (376 MHz, Chloroform-d) δ −199.52 (dd, J=53.3, 35.0 Hz); LRMS (M+H) m/z 440.3, 442.2.

### Example 99

### 1-(((2S,3S,4S)-3-ethyl-4-fluoro-5-oxopyrrolidin-2-yl)methoxy)-7-methoxy-4-((tetrahydro-2H-pyran-4-yl)ethynyl)isoquinoline-6-carboxamide (I-82)

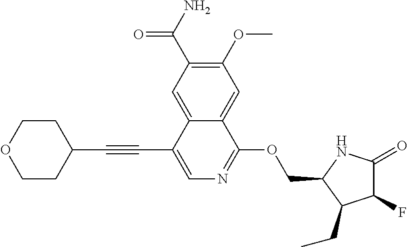 [SMILES: CC[C@@H]1[C@H](F)C(=O)N[C@@H]1COc1ncc(C#CC2CCOCC2)c2cc(C(N)=O)c(OC)cc12]

General procedures (B): 0.06 mmol scale, 33% yield.

¹H NMR (400 MHz, DMSO-d₆) δ 8.87 (s, 1H), 8.32 (s, 1H), 8.06 (s, 1H), 7.90 (s, 1H), 7.77 (s, 1H), 7.74 (s, 1H), 4.91 (dd, J=53.9, 6.1 Hz, 1H), 4.55 (dd, J=11.2, 3.5 Hz, 1H), 4.27 (dd, J=11.2, 6.3 Hz, 1H), 4.12-4.06 (m, 1H), 3.98 (s, 3H), 3.86 (dt, J=11.5, 4.3 Hz, 2H), 3.50 (ddd, J=11.6, 9.0, 2.8 Hz, 2H), 3.05 (tt, J=8.7, 4.1 Hz, 1H), 2.70-2.53 (m, 1H), 1.96-1.89 (m, 1H), 1.73-1.53 (m, 4H), 1.01 (t, J=7.3 Hz, 3H); ¹⁹F NMR (376 MHz, DMSO-d₆) δ −199.29 (ddd, J=53.9, 37.4, 3.6 Hz); LRMS (M+H) m/z 470.5.

### Example 100

### 4-((trans-4-ethoxycyclohexyl)ethynyl)-1-(((2S,3S,4S)-3-ethyl-4-fluoro-5-oxopyrrolidin-2-yl)methoxy)-7-methoxyisoquinoline-6-carboxamide (I-83)

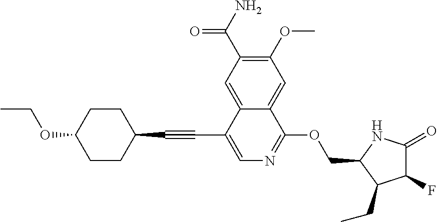 [SMILES: CCO[C@H]1CC[C@H](C#Cc2cnc(OC[C@H]3NC(=O)[C@@H](F)[C@H]3CC)c3cc(OC)c(C(N)=O)cc23)CC1]

General procedures (B): 0.06 mmol scale, 49% yield.

¹H NMR (400 MHz, DMSO-d₆) δ 8.86 (s, 1H), 8.29 (s, 1H), 8.02 (s, 1H), 7.89 (s, 1H), 7.76 (s, 1H), 7.73 (s, 1H), 4.91 (dd, J=53.9, 6.1 Hz, 1H), 4.54 (dd, J=11.3, 3.5 Hz, 1H), 4.26 (dd, J=11.2, 6.3 Hz, 1H), 4.12-4.06 (m, 1H), 3.98 (s, 3H), 3.45 (q, J=7.0 Hz, 2H), 3.35-3.28 (m, overlapped with H₂O, 1H), 2.70 (tt, J=10.5, 3.7 Hz, 1H), 2.67-2.53 (m, 1H), 2.07-1.96 (m, 4H), 1.63-1.47 (m, 4H), 1.35-1.25 (m, 2H), 1.10 (t, J=7.0 Hz, 3H), 1.01 (t, J=7.3 Hz, 3H); ¹⁹F NMR (376 MHz, DMSO-d₆) δ −199.29 (ddd, J=53.9, 37.3, 3.6 Hz); LRMS (M+H) m/z 512.5.

### Example 101

### 4-((cis-4-ethoxycyclohexyl)ethynyl)-1-(((2S,3S,4S)-3-ethyl-4-fluoro-5-oxopyrrolidin-2-yl)methoxy)-7-methoxyisoquinoline-6-carboxamide (I-84)

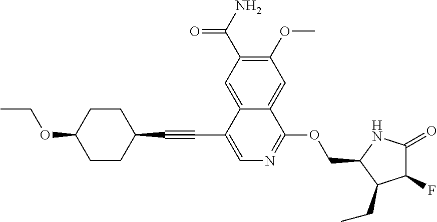 [SMILES: CCO[C@H]1CC[C@@H](C#Cc2cnc(OC[C@H]3NC(=O)[C@@H](F)[C@H]3CC)c3cc(OC)c(C(N)=O)cc23)CC1]

General procedures (B): 0.06 mmol scale, 51% yield.

¹H NMR (400 MHz, DMSO-d₆) δ 8.87 (s, 1H), 8.30 (s, 1H), 8.04 (s, 1H), 7.89 (s, 1H), 7.76 (s, 1H), 7.73 (s, 1H), 4.91 (dd, J=53.8, 6.1 Hz, 1H), 4.54 (dd, J=11.3, 3.5 Hz, 1H), 4.27 (dd, J=11.2, 6.3 Hz, 1H), 4.12-4.06 (m, 1H), 3.98 (s, 3H), 3.44 (q, J=7.0 Hz, 2H), 3.40-3.35 (m, 1H), 2.93-2.88 (m, 1H), 2.70-2.53 (m, 1H), 1.86-1.54 (m, 10H), 1.11 (t, J=7.0 Hz, 3H), 1.01 (t, J=7.3 Hz, 3H); ¹⁹F NMR (376 MHz, DMSO-d₆) δ −199.29 (ddd, J=53.8, 37.3, 3.6 Hz); LRMS (M+H) m/z 512.5.

### Example 102

### (R)-4-bromo-7-isopropoxy-1-(piperidin-3-yloxy)isoquinoline-6-carboxamide

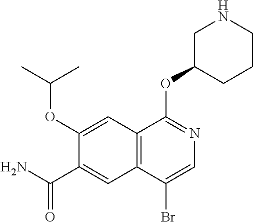 [SMILES: CC(C)Oc1cc2c(O[C@@H]3CCCNC3)ncc(Br)c2cc1C(N)=O]

General procedures (A), followed by general procedures (D), step 1.

¹H NMR (400 MHz, DMSO-d₆) δ 8.29 (s, 1H), 8.16 (s, 1H), 7.87 (br s, 1H), 7.81 (s, 1H), 7.80 (br s, 1H), 5.57-5.54 (m, 1H), 4.97 (hept, J=6.0 Hz, 1H), 3.97 (v br s, 1H), 3.52-3.42 (m, 2H), 3.26-3.20 (m, 1H), 3.14-3.06 (m, 1H), 2.03-1.96 (m, 3H), 1.81-1.74 (m, 1H), 1.43 (d, J=6.0 Hz, 3H), 1.40 (d, J=6.0 Hz, 3H); LRMS (M+H) m/z 408.2, 410.2.

### Example 103

### (R)-4-bromo-1-((1-(2-cyanoacetyl)piperidin-3-yl)oxy)-7-isopropoxyisoquinoline-6-carboxamide

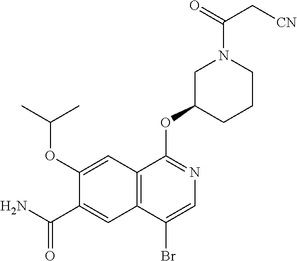 [SMILES: CC(C)Oc1cc2c(O[C@@H]3CCCN(C(=O)CC#N)C3)ncc(Br)c2cc1C(N)=O]

General procedures (G): 0.3 mmol scale, 87% yield.

¹H NMR (400 MHz, Chloroform-d) δ 8.96 (s, 0.6H), 8.92 (s, 0.4H), 8.06 (s, 0.6H), 8.04 (s, 0.4H), 7.98 (br s, 0.4H), 7.93 (br s, 0.6H), 7.63 (s, 0.4H), 7.52 (s, 0.6H), 5.97 (br s, 1H), 5.45-5.40 (m, 1H), 5.05-4.90 (m, 1H), 4.47 (dd, J=13.6, 4.6 Hz, 0.4H), 4.09 (dd, J=13.6, 5.4 Hz, 0.6H), 3.78-3.67 (m, 2H), 3.62-3.37 (m, 2.6H), 3.34 (ddd, J=13.0, 9.4, 3.5 Hz, 0.4H), 2.35-2.29 (m, 0.4H), 2.18-1.90 (m, 2.6H), 1.79-1.64 (m, 1H), 1.56-1.51 (m, 6H); LRMS (M+H) m/z 475.2, 477.2.

### Example 104

### (R)-4-((1-hydroxycyclopentyl)ethynyl)-7-isopropoxy-1-(piperidin-3-yloxy)isoquinoline-6-carboxamide (I-85)

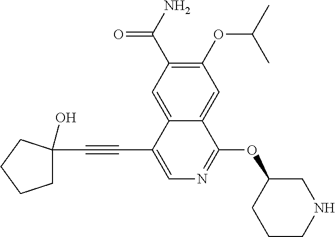 [SMILES: CC(C)Oc1cc2c(O[C@@H]3CCCNC3)ncc(C#CC3(O)CCCC3)c2cc1C(N)=O]

General procedures (B), followed by general procedures (D), step 1.

¹H NMR (400 MHz, DMSO-d₆) δ 8.32 (s, 1H), 8.11 (s, 1H), 7.79 (br s, 2H), 7.78 (s, 1H), 6.30-6.27 (m, 1H), 5.52-5.47 (m, 1H), 4.97 (hept, J=6.0 Hz, 1H), 3.39-3.27 (m, partially overlapped with H₂O, 5H), 3.10-2.97 (m, 2H), 2.60-2.55 (m, 2H), 2.50-2.47 (m, partially overlapped with DMSO, 2H), 2.05-1.89 (m, 5H), 1.73-1.67 (m, 1H), 1.41 (d, J=6.0 Hz, 3H), 1.40 (d, J=6.0 Hz, 3H); LRMS (M−H₂O+Na) m/z 442.1.

### Example 105

### (R)-1-((1-(2-cyanoacetyl)piperidin-3-yl)oxy)-4-((1-hydroxycyclopentyl)ethynyl)-7-isopropoxyisoquinoline-6-carboxamide (I-88)

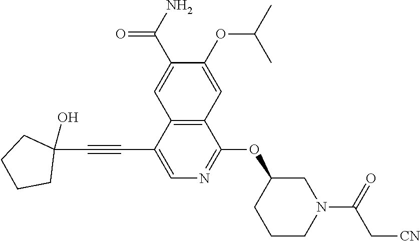 [SMILES: CC(C)Oc1cc2c(O[C@@H]3CCCN(C(=O)CC#N)C3)ncc(C#CC3(O)CCCC3)c2cc1C(N)=O]

General procedures (G): 0.042 mmol scale, 30% yield.

¹H NMR (400 MHz, Chloroform-d) δ 9.04 (s, 0.6H), 9.00 (s, 0.4H), 8.07 (s, 0.6H), 8.05 (s, 0.4H), 7.96 (br s, 0.4H), 7.91 (br s, 0.6H), 7.60 (s, 0.4H), 7.50 (s, 0.6H), 6.29-6.24 (m, 1H), 5.96-5.93 (m, 1H), 5.48-5.45 (m, 1H), 5.02-4.88 (m, 1H), 4.42 (dd, J=13.6, 4.8 Hz, 0.4H), 4.10-4.04 (m, 1H), 3.81-3.32 (m, 4.6H), 2.68-2.63 (m, 2H), 2.55-2.49 (m, 2H), 2.32-1.92 (m, 6H), 1.80-1.66 (m, overlapped with H₂O, 2H), 1.55-1.51 (m, 6H); LRMS (M−H₂O+H) m/z 487.4.

### Example 106

### (R)-7-isopropoxy-1-(piperidin-3-yloxy)-4-((tetrahydro-2H-pyran-4-yl)ethynyl)isoquinoline-6-carboxamide (I-86)

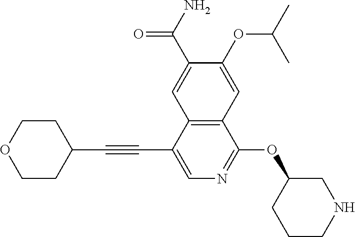 [SMILES: CC(C)Oc1cc2c(O[C@@H]3CCCNC3)ncc(C#CC3CCOCC3)c2cc1C(N)=O]

General procedures (B): followed by general procedures (D), step 1.

¹H NMR (400 MHz, DMSO-d₆) δ 8.36 (s, 1H), 8.05 (s, 1H), 7.80 (s, 1H), 7.78 (br s, 1H), 7.77 (br s, 1H), 5.51 (br s, 1H), 4.98 (hept, J=6.0 Hz, 1H), 3.86 (ddd, J=11.5, 4.2, 4.2 Hz, 2H), 3.50 (ddd, J=11.6, 8.9, 2.8 Hz, 3H), 3.33-3.00 (m, partially overlapped with H₂O, 6H), 2.01-1.89 (m, 5H), 1.74-1.64 (m, 3H), 1.40 (d, J=6.0 Hz, 3H), 1.39 (d, J=6.0 Hz, 3H); LRMS (M+H) m/z 438.2.

### Example 107

### (R)-1-((1-(2-cyanoacetyl)piperidin-3-yl)oxy)-7-isopropoxy-4-((tetrahydro-2H-pyran-4-yl)ethynyl)isoquinoline-6-carboxamide (I-89)

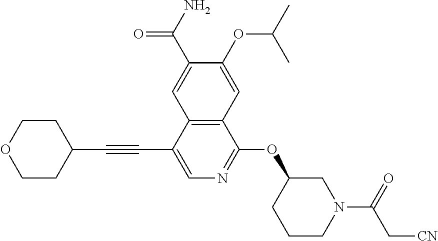 [SMILES: CC(C)Oc1cc2c(O[C@@H]3CCCN(C(=O)CC#N)C3)ncc(C#CC3CCOCC3)c2cc1C(N)=O]

General procedures (G): 0.085 mmol scale, 36% yield.

¹H NMR (400 MHz, Chloroform-d) δ 9.03 (s, 0.6H), 9.00 (s, 0.4H), 8.04 (s, 0.6H), 8.02 (s, 0.4H), 7.99 (br s, 0.4H), 7.95 (br s, 0.6H), 7.60 (s, 0.4H), 7.50 (s, 0.6H), 6.19 (br s, 0.6H), 6.16 (br s, 0.4H), 5.47-5.42 (m, 1H), 5.03-4.89 (m, 1H), 4.40 (dd, J=13.7, 4.9 Hz, 0.4H), 4.07-3.99 (m, 2.6H), 3.81-3.34 (m, 8H), 3.02 (tt, J=8.3, 4.1 Hz, 1H), 2.37-1.64 (m, 7H), 1.55-1.51 (m, 6H); LRMS (M+H) m/z 505.4.

### Example 108

### (R)-7-isopropoxy-1-(piperidin-3-yloxy)-4-(3-(tetrahydro-2H-pyran-4-yl)prop-1-yn-1-yl)isoquinoline-6-carboxamide (I-87)

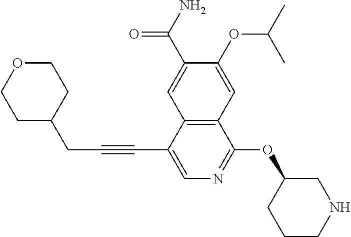 [SMILES: CC(C)Oc1cc2c(O[C@@H]3CCCNC3)ncc(C#CCC3CCOCC3)c2cc1C(N)=O]

General procedures (B), followed by general procedures (D), step 1.

¹H NMR (400 MHz, DMSO-d₆) δ 8.39 (s, 1H), 8.05 (s, 1H), 7.81 (s, 1H), 7.80 (br s, 1H), 7.78 (br s, 1H), 5.61-5.57 (m, 1H), 4.98 (hept, J=6.0 Hz, 1H), 3.88 (ddd, J=11.4, 4.6, 1.8 Hz, 2H), 3.51-3.41 (m, 2H), 3.37-3.30 (m, partially overlapped with H₂O, 2H), 3.27-3.21 (m, 1H), 3.11-3.06 (m, 1H), 2.55 (d, J=6.5 Hz, 2H), 2.02-1.96 (m, 3H), 1.88-1.75 (m, 4H), 1.41 (d, J=6.0 Hz, 3H), 1.40 (d, J=6.0 Hz, 3H), 1.42-1.33 (m, partially overlapped, 2H); LRMS (M+H) m/z 452.2.

### Example 109

### (R)-1-((1-(2-cyanoacetyl)piperidin-3-yl)oxy)-7-isopropoxy-4-(3-(tetrahydro-2H-pyran-4-yl)prop-1-yn-1-yl)isoquinoline-6-carboxamide (I-90)

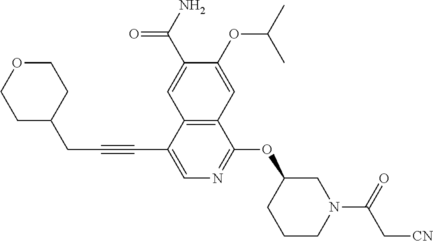 [SMILES: CC(C)Oc1cc2c(O[C@@H]3CCCN(C(=O)CC#N)C3)ncc(C#CCC3CCOCC3)c2cc1C(N)=O]

General procedures (G): 0.096 mmol scale, 47% yield.

¹H NMR (400 MHz, Chloroform-d) δ 9.02 (s, 0.6H), 8.99 (s, 0.4H), 8.02 (s, 0.6H), 8.00 (s, 0.4H), 7.96 (br d, J=3.8 Hz, 1H), 7.60 (s, 0.4H), 7.50 (s, 0.6H), 6.29 (d, J=3.8 Hz, 0.6H), 6.26 (d, J=3.8 Hz, 0.4H), 5.46-5.40 (m, 1H), 5.03-4.88 (m, 1H), 4.37 (dd, J=13.6, 4.9 Hz, 0.4H), 4.05-4.00 (m, 2.6H), 3.81-3.35 (m, 8.4H), 3.18-3.11 (m, 0.6H), 2.53-2.50 (m, 2H), 2.42-1.65 (m, 7H), 1.55-1.49 (m, 6H); LRMS (M+H) m/z 519.4.

### Example 110

### (R)-1-((1-(2-cyanoacetyl)piperidin-3-yl)oxy)-4-((4-hydroxytetrahydro-2H-pyran-4-yl)ethynyl)-7-isopropoxyisoquinoline-6-carboxamide (I-91)

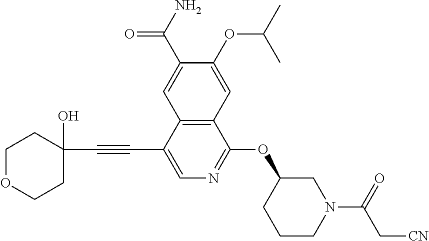 [SMILES: CC(C)Oc1cc2c(O[C@@H]3CCCN(C(=O)CC#N)C3)ncc(C#CC3(O)CCOCC3)c2cc1C(N)=O]

General procedures (B): 0.08 mmol scale, 41% yield.

¹H NMR (400 MHz, DMSO-d₆) δ 8.37 (s, 0.4H), 8.35 (s, 0.6H), 8.10 (s, 0.4H), 8.07 (s, 0.6H), 7.76 (br s, 1H), 7.74 (br s, 1H), 7.54 (s, 0.6H), 7.47 (s, 0.4H), 5.90 (s, 1H), 5.45-5.40 (m, 0.4H), 5.39-5.35 (m, 0.6H), 4.91-4.78 (m, 1H), 4.31 (dd, J=13.4, 4.3 Hz, 0.6H), 4.22-4.06 (m, 2H), 4.00-3.95 (m, 0.4H), 3.91-3.77 (m, 2H), 3.71-3.60 (m, 3H), 3.38 (dd, J=13.5, 2.3 Hz, 0.6H), 3.33-3.25 (m, partially overlapped with H₂O, 1H), 3.18 (ddd, J=13.1, 9.9, 3.3 Hz, 0.4H), 2.16-2.09 (m, 0.6H), 1.97-1.49 (m, 7.4H), 1.42-1.38 (m, 6H); LRMS (M+H) m/z 521.1.

### Example 111

### tert-butyl (R)-4-((6-carbamoyl-1-((1-(2-cyanoacetyl)piperidin-3-yl)oxy)-7-isopropoxyisoquinolin-4-yl)ethynyl)-4-hydroxypiperidine-1-carboxylate (I-92)

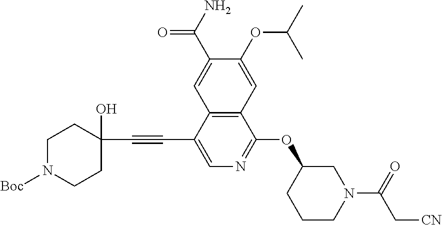 [SMILES: CC(C)Oc1cc2c(O[C@@H]3CCCN(C(=O)CC#N)C3)ncc(C#CC3(O)CCN(C(=O)OC(C)(C)C)CC3)c2cc1C(N)=O]

General procedures (B): 0.15 mmol scale, 85% yield.

¹H NMR (400 MHz, Chloroform-d) δ 8.96 (s, 0.6H), 8.94 (s, 0.4H), 8.05 (s, 0.6H), 8.04-8.03 (m, 0.8H), 7.98 (d, J=4.1 Hz, 0.6H), 7.59 (s, 0.4H), 7.49 (s, 0.6H), 6.05 (d, J=4.1 Hz, 0.6H), 6.01 (d, J=4.1 Hz, 0.4H), 5.49-5.44 (m, 1H), 5.03-4.89 (m, 1H), 4.47 (dd, J=13.7, 4.6 Hz, 0.4H), 4.12-4.06 (m, 0.6H), 3.86-3.83 (m, 2H), 3.77 (d, J=3.7 Hz, 1.2H), 3.69 (br d, J=13.7 Hz, 0.4H), 3.62-3.30 (m, 6.4H), 2.33-2.27 (m, 0.4H), 2.18-1.86 (m, 6.6H), 1.76-1.66 (m, partially overlapped with H₂O, 1H), 1.55-1.50 (m, 6H), 1.47 (s, 9H); LRMS (M-Boc+H) m/z 520.1, (M+Na) m/z 642.2.

### Example 112

### (R)-1-((1-(2-cyanoacetyl)piperidin-3-yl)oxy)-4-((4-hydroxypiperidin-4-yl)ethynyl)-7-isopropoxyisoquinoline-6-carboxamide (I-93)

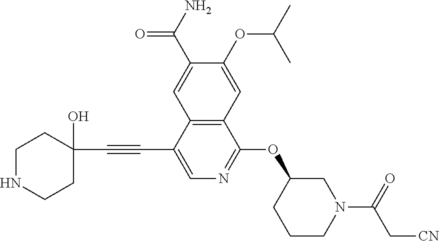 [SMILES: CC(C)Oc1cc2c(O[C@@H]3CCCN(C(=O)CC#N)C3)ncc(C#CC3(O)CCNCC3)c2cc1C(N)=O]

General procedures (D), step 1: 0.11 mmol scale, 27% yield.

¹H NMR (400 MHz, DMSO-d₆) δ 8.35 (s, 0.4H), 8.33 (s, 0.6H), 8.07 (s, 0.4H), 8.03 (s, 0.6H), 7.84-7.73 (m, 2H), 7.53 (s, 0.6H), 7.46 (s, 0.4H), 7.24-7.23 (m, 1H), 5.64 (br s, 1H), 5.43 (br s, 0.4H), 5.36 (br s, 0.6H), 4.89-4.79 (m, 1H), 4.30 (br d, J=12.2 Hz, 0.6H), 4.22-4.06 (m, 1.6H), 3.97 (br d, J=13.2 Hz, 0.4H), 3.91-3.61 (m, 3.4H), 3.40-3.29 (m, overlapped with H₂O, 2H), 3.20-2.73 (m, 3H), 2.14-2.10 (m, 0.6H), 1.99-1.50 (m, 6.4H), 1.41-1.38 (m, 6H); LRMS (M+H) m/z 520.2.

### Example 113

### (R)-1-((1-(2-cyanoacetyl)piperidin-3-yl)oxy)-4-((4-hydroxy-1-methylpiperidin-4-yl)ethynyl)-7-isopropoxyisoquinoline-6-carboxamide (I-94)

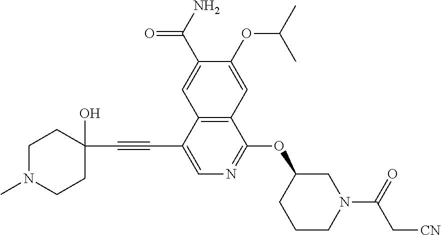 [SMILES: CC(C)Oc1cc2c(O[C@@H]3CCCN(C(=O)CC#N)C3)ncc(C#CC3(O)CCN(C)CC3)c2cc1C(N)=O]

General procedures (D), step 2, formaldehyde was used: 0.019 mmol scale, 68% yield.

¹H NMR (400 MHz, DMSO-d₆) δ 8.40 (s, 0.4H), 8.38 (s, 0.6H), 8.18 (s, 0.6H), 8.04 (s, 0.4H), 7.75-7.74 (m, 2H), 7.54 (s, 0.6H), 7.46 (s, 0.4H), 5.74 (br s, 1H), 5.45-5.41 (m, 0.4H), 5.38-5.35 (m, 0.6H), 4.91-4.79 (m, 1H), 4.30 (dd, J=13.5, 4.4 Hz, 0.6H), 4.22-3.61 (m, 3.8H), 3.38 (dd, J=13.4, 2.3 Hz, 0.6H), 3.29 (ddd, J=13.6, 10.4, 3.0 Hz, 0.6H), 3.18 (ddd, J=13.1, 9.9, 3.4 Hz, 0.4H), 2.67-2.61 (m, 2H), 2.42-2.37 (m, 2H), 2.23 (s, 1.8H), 2.23 (s, 1.2H), 2.15-2.10 (m, 0.6H), 1.99-1.49 (m, 7.4H), 1.49-1.38 (m, 6H); LRMS (M+H) m/z 534.2.

### Example 114

### tert-butyl (R)-4-((6-carbamoyl-1-((1-(2-cyanoacetyl)piperidin-3-yl)oxy)-7-isopropoxyisoquinolin-4-yl)ethynyl)piperidine-1-carboxylate (I-95)

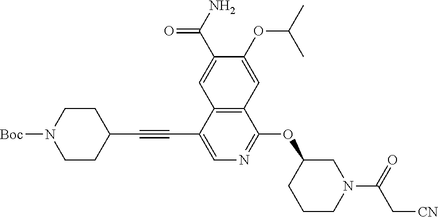 [SMILES: CC(C)Oc1cc2c(O[C@@H]3CCCN(C(=O)CC#N)C3)ncc(C#CC3CCN(C(=O)OC(C)(C)C)CC3)c2cc1C(N)=O]

General procedures (B): 0.1 mmol scale, 75% yield.

¹H NMR (400 MHz, Chloroform-d) δ 9.02 (s, 0.6H), 8.99 (s, 0.4H), 8.03 (s, 0.6H), 8.01 (s, 0.4H), 7.99 (d, J=3.9 Hz, 0.4H), 7.94 (d, J=3.9 Hz, 0.6H), 7.60 (s, 0.4H), 7.50 (s, 0.6H), 6.05-6.01 (m, 1H), 5.48-5.42 (m, 1H), 5.03-4.88 (m, 1H), 4.42 (dd, J=13.5, 4.7 Hz, 0.4H), 4.07 (ddd, J=13.7, 5.4, 5.4 Hz, 0.6H), 3.85-3.76 (m, 2.4H), 3.67 (br d, J=13.8 Hz, 0.4H), 3.62-3.40 (m, 2.4H), 3.35 (ddd, J=13.0, 9.3, 3.5 Hz, 0.6H), 3.29-3.22 (m, 2H), 2.94 (tt, J=8.2, 4.0 Hz, 1H), 2.32-2.26 (m, 0.4H), 2.18-1.90 (m, 4.6H), 1.83-1.67 (m, 4H), 1.55-1.51 (m, 6H), 1.47 (s, 9H); LRMS (M+Na) m/z 626.7.

### Example 115

### (R)-1-((1-(2-cyanoacetyl)piperidin-3-yl)oxy)-7-isopropoxy-4-(piperidin-4-ylethynyl)isoquinoline-6-carboxamide (I-96)

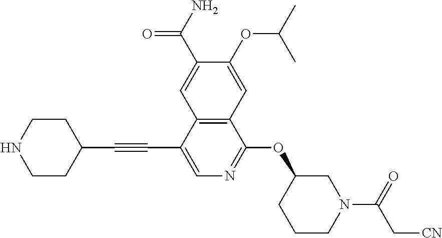 [SMILES: CC(C)Oc1cc2c(O[C@@H]3CCCN(C(=O)CC#N)C3)ncc(C#CC3CCNCC3)c2cc1C(N)=O]

General procedures (D), step 1: 0.029 mmol scale, 86% yield.

¹H NMR (400 MHz, Methanol-d₄) δ 8.72 (s, 0.4H), 8.70 (s, 0.6H), 8.55 (br s, 1H), 8.06 (s, 0.4H), 8.02 (s, 0.6H), 7.67 (s, 0.6H), 7.59 (s, 0.4H), 5.54-5.50 (m, 0.4H), 5.47-5.43 (m, 0.6H), 5.06-4.94 (m, 1H), 4.55-4.50 (m, 0.6H), 4.17 (ddd, J=13.2, 4.5, 4.5 Hz, 0.4H), 3.89 (dd, J=14.4, 4.7 Hz, 0.4H), 3.81-3.74 (m, 1H), 3.46-3.37 (m, 3.6H), 3.27-3.14 (m, 4H), 2.31-1.84 (m, 8H), 1.77-1.61 (m, 1H), 1.53-1.49 (m, 6H); LRMS (M+H) m/z 504.5.

### Example 116

### (R)-1-((1-(2-cyanoacetyl)piperidin-3-yl)oxy)-7-isopropoxy-4-((1-methylpiperidin-4-yl)ethynyl)isoquinoline-6-carboxamide (I-97)

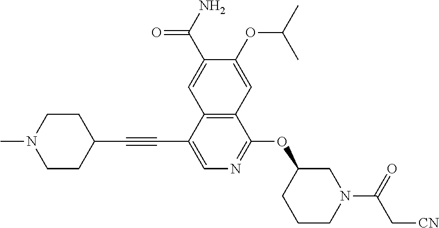 [SMILES: CC(C)Oc1cc2c(O[C@@H]3CCCN(C(=O)CC#N)C3)ncc(C#CC3CCN(C)CC3)c2cc1C(N)=O]

General procedures (D), step 2, formaldehyde was used: 0.02 mmol scale, 53% yield.

¹H NMR (400 MHz, DMSO-d₆) δ 8.38 (s, 0.4H), 8.36 (s, 0.6H), 8.05 (s, 0.4H), 8.01 (s, 0.6H), 7.76 (br s, 2H), 7.53 (s, 0.6H), 7.46 (s, 0.4H), 5.43-5.39 (m, 0.4H), 5.37-5.33 (m, 0.6H), 4.91-4.77 (m, 1H), 4.29 (dd, J=13.6, 4.4 Hz, 0.6H), 4.21-4.06 (m, 1.6H), 4.00-3.94 (m, 0.4H), 3.89-3.84 (m, 0.4H), 3.78 (dd, J=14.3, 4.8 Hz, 0.4H), 3.70-3.61 (m, 1.6H), 3.40-3.14 (m, 2H), 2.79 (br s, 1H), 2.68-2.64 (m, 2H), 2.22-2.09 (m, 5H), 1.98-1.89 (m, 4H), 1.71 (dddd, J=13.4, 9.0, 4.3, 4.3 Hz, 2H), 1.62-1.48 (m, 1H), 1.42-1.38 (m, 6H); LRMS (M+H) m/z 518.5.

### Example 117

### 1-(((R)-1-(2-cyanoacetyl)piperidin-3-yl)oxy)-4-(((1r,4R)-4-ethoxycyclohexyl)ethynyl)-7-isopropoxyisoquinoline-6-carboxamide (I-98)

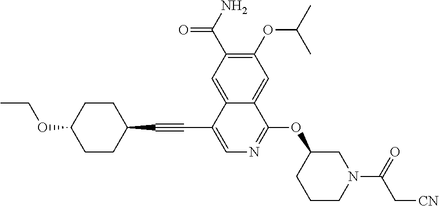 [SMILES: CCO[C@H]1CC[C@H](C#Cc2cnc(O[C@@H]3CCCN(C(=O)CC#N)C3)c3cc(OC(C)C)c(C(N)=O)cc23)CC1]

General procedures (B): 0.06 mmol scale, 37% yield.

¹H NMR (400 MHz, DMSO-d₆) δ 8.34 (s, 0.4H), 8.32 (s, 0.6H), 8.03 (s, 0.4H), 7.99 (s, 0.6H), 7.76-7.75 (m, 2H), 7.53 (s, 0.6H), 7.45 (s, 0.4H), 5.43-5.39 (m, 0.4H), 5.36-5.33 (m, 0.6H), 4.91-4.78 (m, 1H), 4.29 (dd, J=13.6, 4.4 Hz, 0.6H), 4.21-4.06 (m, 1.6H), 3.96 (dd, J=11.2, 6.6 Hz, 0.4H), 3.89-3.84 (m, 0.4H), 3.78 (dd, J=14.3, 4.9 Hz, 0.4H), 3.69-3.59 (m, 1.6H), 3.45 (q, J=7.0 Hz, 2H), 3.39-3.26 (m, partially overlapped with H₂O, 1.6H), 3.17 (ddd, J=13.0, 9.9, 3.3 Hz, 0.4H), 2.74-2.67 (m, 1H), 2.13-1.80 (m, 7.4H), 1.73-1.47 (m, 2.6H), 1.42-1.38 (m, 6H), 1.35-1.26 (m, 2H), 1.10 (t, J=7.0 Hz, 3H); LRMS (M+H) m/z 547.3.

### Example 118

### 1-(((R)-1-(2-cyanoacetyl)piperidin-3-yl)oxy)-4-((cis-4-ethoxycyclohexyl)ethynyl)-7-isopropoxyisoquinoline-6-carboxamide (I-99)

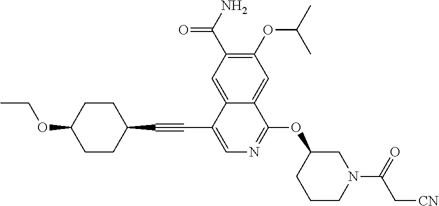 [SMILES: CCO[C@H]1CC[C@@H](C#Cc2cnc(O[C@@H]3CCCN(C(=O)CC#N)C3)c3cc(OC(C)C)c(C(N)=O)cc23)CC1]

General procedures (B): 0.06 mmol scale, 14% yield.

¹H NMR (400 MHz, DMSO-d₆) δ 8.34 (s, 0.4H), 8.32 (s, 0.6H), 8.05 (s, 0.4H), 8.01 (s, 0.6H), 7.76 (br s, 1.2H), 7.74 (br s, 0.8H), 7.53 (s, 0.6H), 7.46 (s, 0.4H), 5.44-5.39 (m, 0.4H), 5.37-5.33 (m, 0.6H), 4.91-4.78 (m, 1H), 4.29 (dd, J=13.6, 4.3 Hz, 0.6H), 4.22-4.06 (m, 1.6H), 3.97 (br d, J=13.2 Hz, 0.4H), 3.90-3.85 (m, 0.4H), 3.78 (dd, J=14.2, 4.8 Hz, 0.4H), 3.70-3.61 (m, 1.6H), 3.44 (q, J=7.0 Hz, 2H), 3.41-3.36 (m, 0.6H), 3.32-3.27 (m, partially overlapped with H₂O, 1H), 3.21-3.15 (m, 0.4H), 2.93-2.88 (m, 1H), 2.14-2.09 (m, 0.6H), 1.98-1.48 (m, 11.4H), 1.42-1.38 (m, 6H), 1.11 (t, J=7.0 Hz, 3H); LRMS (M+H) m/z 547.3.

### Example 119

### 1-(((R)-1-(2-cyanoacetyl)piperidin-3-yl)oxy)-4-(((1r,4R)-4-hydroxy-4-methylcyclohexyl)ethynyl)-7-isopropoxyisoquinoline-6-carboxamide (I-100)

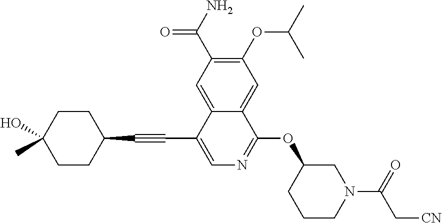 [SMILES: CC(C)Oc1cc2c(O[C@@H]3CCCN(C(=O)CC#N)C3)ncc(C#C[C@H]3CC[C@](C)(O)CC3)c2cc1C(N)=O]

General procedures (B): 0.05 mmol scale, 33% yield.

¹H NMR (400 MHz, DMSO-d₆) δ 8.38 (s, 0.4H), 8.36 (s, 0.6H), 8.04 (s, 0.4H), 8.00 (s, 0.6H), 7.75 (br s, 2H), 7.53 (s, 0.6H), 7.46 (s, 0.4H), 5.43-5.39 (m, 0.4H), 5.36-5.33 (m, 0.6H), 4.91-4.78 (m, 1H), 4.29 (dd, J=13.5, 4.3 Hz, 0.6H), 4.21-4.06 (m, 2.4H), 4.00-3.94 (m, 0.4H), 3.89-3.60 (m, 1.6H), 3.39-3.26 (m, overlapped with H₂O, 3.6H), 3.20-3.14 (m, 0.4H), 2.95-2.90 (m, 1H), 2.15-2.07 (m, 0.6H), 2.01-1.80 (m, 3.4H), 1.75-1.68 (m, 2H), 1.61-1.54 (m, 2H), 1.50-1.44 (m, 2H), 1.42-1.38 (m, 6H), 1.17 (s, 3H); LRMS (M+H) m/z 533.5.

### Example 120

### 1-(((R)-1-(2-cyanoacetyl)piperidin-3-yl)oxy)-4-(((1s,4S)-4-hydroxy-4-methylcyclohexyl)ethynyl)-7-isopropoxyisoquinoline-6-carboxamide (I-101)

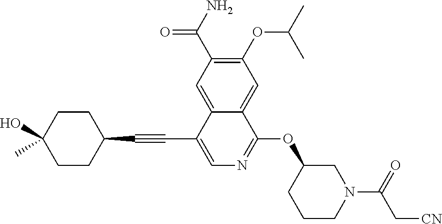 [SMILES: CC(C)Oc1cc2c(O[C@@H]3CCCN(C(=O)CC#N)C3)ncc(C#C[C@H]3CC[C@@](C)(O)CC3)c2cc1C(N)=O]

General procedures (B): 0.05 mmol scale, 34% yield.

¹H NMR (400 MHz, Chloroform-d) δ 9.13 (s, 0.6H), 9.11 (s, 0.4H), 8.13 (br s, 0.4H), 8.08-8.07 (m, 0.6H), 8.01 (s, 0.6H), 7.99 (s, 0.4H), 7.60 (s, 0.4H), 7.49 (s, 0.6H), 6.31 (br s, 1H), 5.47-5.42 (m, 1H), 5.04-4.89 (m, 1H), 4.42 (dd, J=13.7, 4.7 Hz, 0.4H), 4.10-4.04 (m, 0.6H), 3.78-3.76 (m, 1.2H), 3.70-3.64 (m, 0.4H), 3.58-3.39 (m, 3H), 3.37-3.31 (m, 0.4H), 2.83-2.80 (m, 1H), 2.33-2.26 (m, 0.4H), 2.18-1.60 (m, 12.6H), 1.55-1.51 (m, 6H), 1.27 (br s, 3H); LRMS (M+H) m/z 533.5.

### Example 121

### tert-butyl (R)-3-((6-carbamoyl-1-((1-(2-cyanoacetyl)piperidin-3-yl)oxy)-7-isopropoxyisoquinolin-4-yl)ethynyl)azetidine-1-carboxylate (I-102)

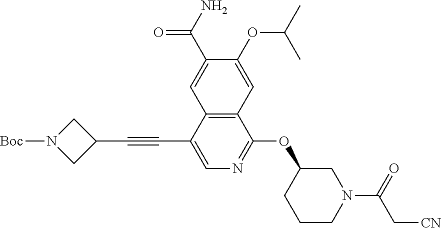 [SMILES: CC(C)Oc1cc2c(O[C@@H]3CCCN(C(=O)CC#N)C3)ncc(C#CC3CN(C(=O)OC(C)(C)C)C3)c2cc1C(N)=O]

General procedures (B): 0.1 mmol scale, 58% yield.

¹H NMR (400 MHz, Chloroform-d) δ 8.99 (s, 0.6H), 8.95 (s, 0.4H), 8.07 (s, 0.6H), 8.05 (s, 0.4H), 7.99 (br s, 0.4H), 7.94 (br s, 0.6H), 7.62 (s, 0.4H), 7.52 (s, 0.6H), 5.89-5.86 (m, 1H), 5.50-5.46 (m, 1H), 5.03-4.91 (m, 1H), 4.46 (dd, J=13.6, 4.6 Hz, 0.4H), 4.31-4.26 (m, 2H), 4.14-4.08 (m, 2.6H), 3.79-3.78 (m, 1H), 3.75-3.67 (m, 1.4H), 3.62-3.31 (m, 3.6H), 2.33-1.95 (m, 3H), 1.81-1.66 (m, 1H), 1.56-1.52 (m, 6H), 1.47 (s, 9H); LRMS (M+H) m/z 576.2.

### Example 122

### (R)-4-(azetidin-3-ylethynyl)-1-((1-(2-cyanoacetyl)piperidin-3-yl)oxy)-7-isopropoxyisoquinoline-6-carboxamide (I-103)

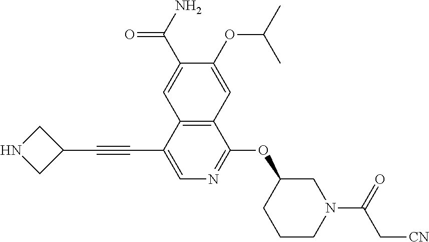 [SMILES: CC(C)Oc1cc2c(O[C@@H]3CCCN(C(=O)CC#N)C3)ncc(C#CC3CNC3)c2cc1C(N)=O]

General procedures (D), step 1: 0.034 mmol scale, 93% yield.

¹H NMR (400 MHz, DMSO-d₆) δ 8.32 (s, 0.4H), 8.31 (br s, 1H), 8.30 (s, 0.6H), 8.10 (s, 0.4H), 8.07 (s, 0.6H), 7.77 (br s, 2H), 7.53 (s, 0.6H), 7.46 (s, 0.4H), 5.44-5.41 (m, 0.4H), 5.38-5.35 (m, 0.6H), 4.91-4.79 (m, 1H), 4.52 (v br s, 1H), 4.30 (dd, J=13.5, 4.4 Hz, 0.6H), 4.21-4.06 (m, 1.6H), 4.01-3.77 (m, 5.8H), 3.70-3.61 (m, 1.4H), 3.38 (dd, J=13.4, 2.3 Hz, 0.6H), 3.29 (ddd, J=13.6, 10.3, 3.0 Hz, 0.6H), 3.17 (ddd, J=13.1, 10.0, 3.3 Hz, 0.4H), 2.14-2.07 (m, 0.6H), 2.00-1.48 (m, 3.4H), 1.44-1.38 (m, 6H); LRMS (M+H) m/z 476.3.

### Example 123

### (R)-1-((1-(2-cyanoacetyl)piperidin-3-yl)oxy)-7-isopropoxy-4-((1-methylazetidin-3-yl)ethynyl)isoquinoline-6-carboxamide (I-104)

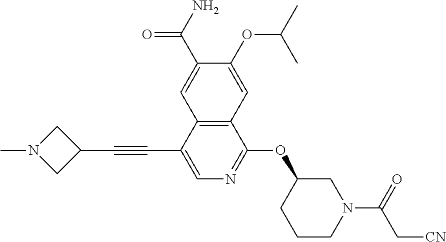 [SMILES: CC(C)Oc1cc2c(O[C@@H]3CCCN(C(=O)CC#N)C3)ncc(C#CC3CN(C)C3)c2cc1C(N)=O]

General procedures (D), step 2, formaldehyde was used: 0.019 mmol scale, 72% yield.

¹H NMR (400 MHz, DMSO-d₆) δ 8.32 (s, 0.4H), 8.30 (s, 0.6H), 8.07 (s, 0.4H), 8.04 (s, 0.6H), 7.77 (br s, 2H), 7.53 (s, 0.6H), 7.46 (s, 0.4H), 5.44-5.40 (m, 0.4H), 5.37-5.34 (m, 0.6H), 4.91-4.79 (m, 1H), 4.29 (dd, J=13.4, 4.3 Hz, 0.6H), 4.21-4.06 (m, 1.6H), 4.00-3.95 (m, 0.4H), 3.90-3.86 (m, 0.4H), 3.79 (dd, J=14.2, 4.8 Hz, 0.4H), 3.70-3.00 (m, 3.2H), 3.55-3.48 (m, 1H), 3.40-3.27 (m, partially overlapped with H₂O, 1H), 3.20-3.14 (m, 0.4H), 3.11-3.08 (m, 2H), 2.24 (s, 3H), 2.16-2.08 (m, 0.4H), 2.01-1.47 (m, 3.6H), 1.44-1.35 (m, 6H); LRMS (M+H) m/z 490.1.

### Example 124

### (R)-1-((1-(2-cyanoacetyl)piperidin-3-yl)oxy)-4-((3,3-dimethoxycyclobutyl)ethynyl)-7-isopropoxyisoquinoline-6-carboxamide (I-105)

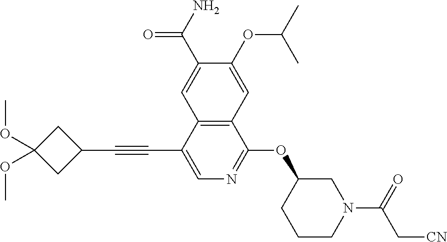 [SMILES: COC1(OC)CC(C#Cc2cnc(O[C@@H]3CCCN(C(=O)CC#N)C3)c3cc(OC(C)C)c(C(N)=O)cc23)C1]

General procedures (B): 0.15 mmol scale, 13% yield.

¹H NMR (400 MHz, Chloroform-d) δ 9.01 (s, 0.6H), 8.98 (s, 0.4H), 8.03 (s, 0.6H), 8.01 (s, 0.4H), 7.97 (br s, 0.4H), 7.92 (br s, 0.6H), 7.60 (s, 0.4H), 7.49 (s, 0.6H), 5.89-5.86 (m, 1H), 5.47-5.43 (m, 1H), 5.02-4.88 (m, 1H), 4.42 (dd, J=13.6, 4.8 Hz, 0.4H), 4.10-4.05 (m, 0.6H), 3.77-3.76 (m, 1H), 3.67 (br d, J=13.3 Hz, 0.4H), 3.61-3.31 (m, 3.6H), 3.209 (s, 1.8H), 3.206 (s, 1.2H), 3.198 (s, 1.8H), 3.196 (s, 1.2H), 3.18-3.13 (m, 1H), 2.71-2.65 (m, 2H), 2.44-2.38 (m, 2H), 2.32-2.28 (m, 0.4H), 2.18-1.92 (m, 2.6H), 1.78-1.65 (m, 1H), 1.55-1.51 (m, 6H); LRMS (M+H) m/z 535.1.

### Example 125

### (R)-1-((1-(2-cyanoacetyl)piperidin-3-yl)oxy)-7-isopropoxy-4-((3-oxocyclobutyl)ethynyl)isoquinoline-6-carboxamide (with TFA)

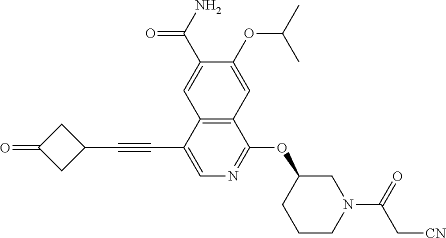 [SMILES: CC(C)Oc1cc2c(O[C@@H]3CCCN(C(=O)CC#N)C3)ncc(C#CC3CC(=O)C3)c2cc1C(N)=O]

General procedures (D), step 1.

¹H NMR (400 MHz, Chloroform-d) δ 9.05 (s, 0.6H), 9.03 (s, 0.4H), 8.53 (br s, 0.4H), 8.50 (br s, 0.6H), 8.19 (br s, 0.4H), 8.17 (br s, 0.6H), 8.04 (s, 0.4H), 7.99 (s, 0.6H), 7.73 (s, 0.6H), 7.63 (s, 0.4H), 5.60 (br s, 1H), 5.08 (hept, J=6.0 Hz, 0.6H), 4.99 (hept, J=6.0 Hz, 0.4H), 4.64 (br d, J=15.3 Hz, 0.6H), 4.22 (br d, J=15.3 Hz, 0.4H), 3.99 (dd, J=14.5, 4.8 Hz, 0.4H), 3.85-3.42 (m, 9H), 3.36-3.29 (m, 0.4H), 241-1.75 (m, 5H), 1.57-1.53 (m, 6H); LRMS (M+H) m/z 489.1.

### Example 126

### 1-(((R)-1-(2-cyanoacetyl)piperidin-3-yl)oxy)-4-((cis-3-(dimethylamino)cyclobutyl)ethynyl)-7-isopropoxyisoquinoline-6-carboxamide (I-106)

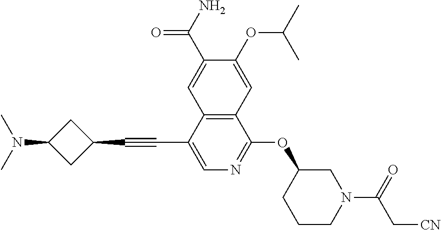 [SMILES: CC(C)Oc1cc2c(O[C@@H]3CCCN(C(=O)CC#N)C3)ncc(C#C[C@H]3C[C@@H](N(C)C)C3)c2cc1C(N)=O]

Similar procedures as the synthesis of I-31: 0.016 mmol scale, 34% yield.

¹H NMR (400 MHz, Chloroform-d) δ 8.99 (s, 0.6H), 8.96 (s, 0.4H), 8.01 (s, 0.6H), 7.99 (s, 0.4H), 7.98 (br s, 0.4H), 7.92 (br s, 0.6H), 7.59 (s, 0.4H), 7.48 (s, 0.6H), 5.92-5.89 (m, 1H), 5.47-5.43 (m, 1H), 5.01-4.87 (m, 1H), 4.43 (dd, J=13.6, 4.9 Hz, 0.4H), 4.10 (dd, J=13.4, 5.3 Hz, 0.6H), 3.77-3.76 (m, 1H), 3.70-3.64 (m, 0.4H), 3.61-3.39 (m, 3H), 3.36-3.30 (m, 0.6H), 3.05-2.96 (m, 1H), 2.80-2.72 (m, 1H), 2.65-2.37 (m, 3H), 2.33-2.23 (m, partially overlapped, 1H), 2.24 (s, 6H), 2.16-1.90 (m, 3H), 1.79-1.64 (m, 1H), 1.58-1.48 (m, 6H); LRMS (M+H) m/z 518.1.

### Example 127

### tert-butyl (R)-3-((6-carbamoyl-4-(1-cyclopropyl-1H-pyrazol-4-yl)-7-isopropoxyisoquinolin-1-yl)oxy)piperidine-1-carboxylate (I-107)

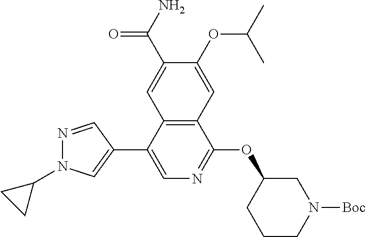 [SMILES: CC(C)Oc1cc2c(O[C@@H]3CCCN(C(=O)OC(C)(C)C)C3)ncc(-c3cnn(C4CC4)c3)c2cc1C(N)=O]

General procedures (C): 0.1 mmol scale, 88% yield.

¹H NMR (400 MHz, Chloroform-d) δ 8.89 (s, 1H), 8.00 (br s, 1H), 7.88 (s, 1H), 7.70 (s, 2H), 7.66 (s, 1H), 5.97 (br s, 1H), 5.35-5.31 (m, 1H), 4.92 (hept, J=6.1 Hz, 1H), 4.15 (br s, 1H), 3.87 (br s, 1H), 3.70 (tt, J=7.4, 3.8 Hz, 1H), 3.44 (br s, 1H), 3.18 (br s, 1H), 2.09-1.99 (m, 3H), 1.66 (br s, 3H), 1.51 (d, J=6.1 Hz, 3H), 1.50 (d, J=6.1 Hz, 3H), 1.46 (br s, 1H), 1.28-1.22 (m, 2H), 1.10-1.04 (m, 8H); LRMS (M+H) m/z 536.1.

### Example 128

### (R)-4-(1-cyclopropyl-1H-pyrazol-4-yl)-7-isopropoxy-1-(piperidin-3-yloxy)isoquinoline-6-carboxamide (I-108)

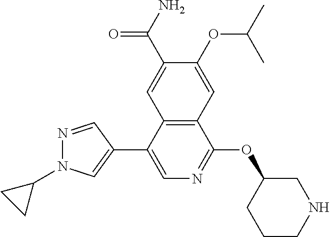 [SMILES: CC(C)Oc1cc2c(O[C@@H]3CCCNC3)ncc(-c3cnn(C4CC4)c3)c2cc1C(N)=O]

General procedures (C), followed by general procedures (D), step 1: 0.1 mmol scale, 61% yield over 2 steps.

¹H NMR (400 MHz, DMSO-d₆) δ 9.06 (br s, 1H), 8.82 (br s, 1H), 8.31 (s, 1H), 8.12 (s, 1H), 7.88 (s, 1H), 7.85 (s, 1H), 7.77 (br s, 1H), 7.69 (s, 1H), 5.62-5.56 (m, 1H), 5.00 (hept, J=6.1 Hz, 1H), 3.85 (tt, J=7.4, 3.9 Hz, 1H), 3.52-3.42 (m, overlapped with H₂O, 2H), 3.28-3.22 (m, 1H), 3.14-3.07 (m, 1H), 2.03-1.98 (m, 3H), 1.82-1.77 (m, 1H), 1.42 (d, J=6.1 Hz, 3H), 1.40 (d, J=6.1 Hz, 3H), 1.16-1.12 (m, 2H), 1.05-1.00 (m, 2H); LRMS (M+H) m/z 436.2.

### Example 129

### (R)-4-(1-cyclobutyl-1H-pyrazol-4-yl)-7-isopropoxy-1-(piperidin-3-yloxy)isoquinoline-6-carboxamide (I-109)

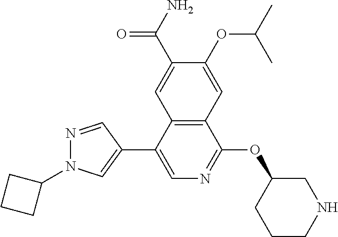 [SMILES: CC(C)Oc1cc2c(O[C@@H]3CCCNC3)ncc(-c3cnn(C4CCC4)c3)c2cc1C(N)=O]

General procedures (C), followed by general procedures (D), step 1: 0.1 mmol scale, 69% yield over 2 steps.

¹H NMR (400 MHz, DMSO-d₆) δ 9.03 (br s, 1H), 8.79 (br s, 1H), 8.31 (s, 1H), 8.13 (s, 1H), 7.89 (s, 1H), 7.84 (s, 1H), 7.76 (br s, 1H), 7.74 (s, 1H), 5.63-5.59 (m, 1H), 5.02-4.90 (m, 2H), 3.48-3.40 (m, overlapped with H₂O, 2H), 3.27-3.24 (m, 1H), 3.11 (br s, 1H), 2.61-2.52 (m, 2H), 2.48-2.38 (m, 2H), 2.03-1.98 (m, 3H), 1.86-1.75 (m, 3H), 1.42 (d, J=6.1 Hz, 3H), 1.40 (d, J=6.1 Hz, 3H); LRMS (M+H) m/z 450.2.

### Example 130

### (R)-4-(1-cyclopentyl-1H-pyrazol-4-yl)-7-isopropoxy-1-(piperidin-3-yloxy)isoquinoline-6-carboxamide (I-110)

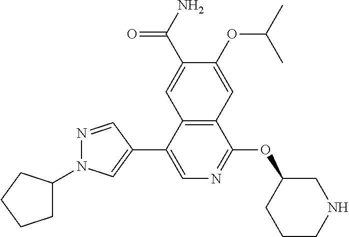 [SMILES: CC(C)Oc1cc2c(O[C@@H]3CCCNC3)ncc(-c3cnn(C4CCCC4)c3)c2cc1C(N)=O]

General procedures (C), followed by general procedures (D), step 1: 0.1 mmol scale, 86% yield over 2 steps.

¹H NMR (400 MHz, DMSO-d₆) δ 8.98 (br s, 1H), 8.75 (br s, 1H), 8.33 (s, 1H), 8.09 (s, 1H), 7.89 (s, 1H), 7.83 (s, 1H), 7.76 (br s, 1H), 7.71 (s, 1H), 5.62-5.59 (m, 1H), 4.97 (hept, J=6.1 Hz, 1H), 4.80 (tt, J=7.0, 7.0 Hz, 1H), 3.48-4.39 (m, overlapped with H₂O, 2H), 3.27-3.23 (m, 1H), 3.14-3.08 (m, 1H), 2.18-2.08 (m, 2H), 2.06-1.97 (m, 5H), 1.89-1.76 (m, 3H), 1.72-1.62 (m, 2H), 1.42 (d, J=6.1 Hz, 3H), 1.40 (d, J=6.1 Hz, 3H); LRMS (M+H) m/z 464.2.

### Example 131

### (R)-7-isopropoxy-1-(piperidin-3-yloxy)-4-(1-(tetrahydro-2H-pyran-4-yl)-1H-pyrazol-4-yl)isoquinoline-6-carboxamide (I-111)

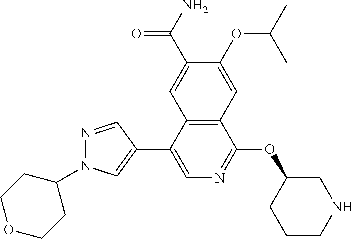 [SMILES: CC(C)Oc1cc2c(O[C@@H]3CCCNC3)ncc(-c3cnn(C4CCOCC4)c3)c2cc1C(N)=O]

General procedures (C), followed by general procedures (D), step 1: 0.1 mmol scale, 78% yield over 2 steps.

¹H NMR (400 MHz, DMSO-d₆) δ 8.34 (s, 1H), 8.14 (s, 1H), 7.89 (s, 1H), 7.85 (s, 1H), 7.77 (br s, 1H), 7.76 (br s, 1H), 7.74 (s, 1H), 5.59-5.55 (m, 1H), 5.01 (hept, J=6.1 Hz, 1H), 4.55-4.46 (m, 1H), 4.01-3.97 (m, 2H), 3.54-3.45 (m, 2H), 3.43-3.38 (m, 2H), 3.21-3.18 (m, 1H), 3.09-3.04 (m, 1H), 2.07-1.94 (m, 7H), 1.79-1.71 (m, 1H), 1.41 (d, J=6.1 Hz, 3H), 1.40 (d, J=6.1 Hz, 3H); LRMS (M+H) m/z 480.2.

### Example 132

### (R)-7-isopropoxy-1-(piperidin-3-yloxy)-4-(1-((tetrahydro-2H-pyran-4-yl)methyl)-1H-pyrazol-4-yl)isoquinoline-6-carboxamide (I-112)

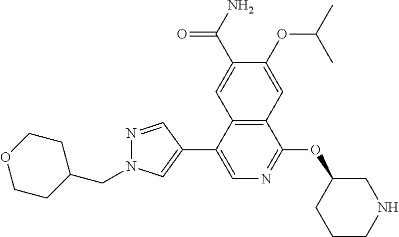 [SMILES: CC(C)Oc1cc2c(O[C@@H]3CCCNC3)ncc(-c3cnn(CC4CCOCC4)c3)c2cc1C(N)=O]

General procedures (C), followed by general procedures (D), step 1: 0.1 mmol scale, 87% yield over 2 steps.

¹H NMR (400 MHz, DMSO-d₆) δ 8.36-8.34 (m, 1H), 8.06 (br s, 1H), 7.87 (s, 1H), 7.87 (s, 1H), 7.79 (br s, 1H), 7.77 (br s, 1H), 7.71 (s, 1H), 5.64-5.60 (m, 1H), 5.06-4.99 (m, 1H), 4.11-4.09 (m, 2H), 3.87-3.83 (m, 2H), 3.70-3.48 (m, partially overlapped with H₂O, 3H), 3.31-3.24 (m, 3H), 3.10 (br s, 1H), 2.17-1.98 (m, 4H), 1.80-1.74 (m, 1H), 1.46 (br d, J=13.0 Hz, 2H), 1.40 (d, J=6.1 Hz, 3H), 1.39 (d, J=6.1 Hz, 3H), 1.34-1.23 (m, 2H); LRMS (M+H) m/z 494.2.

### Example 133

### (R)-7-isopropoxy-1-(piperidin-3-yloxy)-4-(1-(2-(tetrahydro-2H-pyran-4-yl)ethyl)-1H-pyrazol-4-yl)isoquinoline-6-carboxamide (I-113)

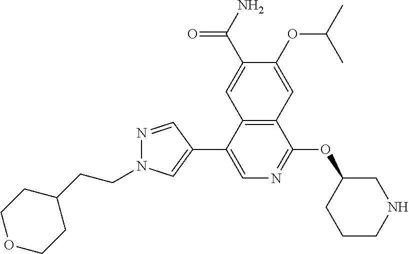 [SMILES: CC(C)Oc1cc2c(O[C@@H]3CCCNC3)ncc(-c3cnn(CCC4CCOCC4)c3)c2cc1C(N)=O]

General procedures (C), followed by general procedures (D), step 1: 0.1 mmol scale, 74% yield over 2 steps.

¹H NMR (400 MHz, DMSO-d₆) δ 8.29 (s, 1H), 7.85 (s, 1H), 7.82 (s, 1H), 7.73 (br s, 1H), 7.72 (br s, 1H), 7.67 (s, 1H), 5.60-5.56 (m, 1H), 4.97 (hept, J=6.1 Hz, 1H), 4.22 (t, J=7.1 Hz, 2H), 3.79 (dd, J=11.5, 4.5 Hz, 2H), 3.46-3.40 (m, partially overlapped with H₂O, 3H), 3.27-3.20 (m, 3H), 3.08 (br s, 1H), 2.00-1.95 (m, 3H), 1.80-1.74 (m, 3H), 1.60 (br d, J=12.6 Hz, 2H), 1.51-1.36 (m, 1H), 1.39 (d, J=6.1 Hz, 3H), 1.37 (d, J=6.1 Hz, 4H), 1.23-1.13 (m, 2H); LRMS (M+H) m/z 508.2.

### Example 134

### (R)-1-((1-(2-cyanoacetyl)piperidin-3-yl)oxy)-4-(1-cyclopropyl-1H-pyrazol-4-yl)-7-isopropoxyisoquinoline-6-carboxamide (I-114)

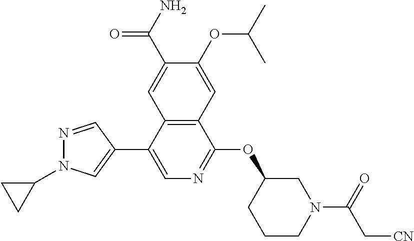 [SMILES: CC(C)Oc1cc2c(O[C@@H]3CCCN(C(=O)CC#N)C3)ncc(-c3cnn(C4CC4)c3)c2cc1C(N)=O]

General procedures (C): 0.07 mmol scale, 57% yield.

¹H NMR (400 MHz, DMSO-d₆) δ 8.32 (s, 0.4H), 8.29 (s, 0.6H), 8.12 (d, J=0.8 Hz, 0.4H), 8.11 (d, J=0.8 Hz, 0.6H), 7.89 (s, 0.4H), 7.85 (s, 0.6H), 7.76-7.74 (m, 2H), 7.69-7.68 (m, 1H), 7.58 (s, 0.6H), 7.51 (s, 0.4H), 5.46-5.42 (m, 0.4H), 5.38-5.34 (m, 0.6H), 4.93-4.80 (m, 1H), 4.28 (dd, J=13.4, 4.5 Hz, 0.6H), 4.22-4.07 (m, 1.6H), 4.02-3.96 (m, 0.4H), 3.90 (d, J=19.1 Hz, 0.4H), 3.87-3.60 (m, 2H), 3.41 (dd, J=13.4, 2.4 Hz, 0.6H), 3.32-3.27 (m, m, partially overlapped with H₂O, 1H), 3.18 (ddd, J=13.1, 9.7, 3.3 Hz, 0.4H), 2.16-2.09 (m, 0.6H), 2.00-1.49 (m, 3.4H), 1.43-1.39 (m, 6H), 1.16-1.12 (m, 2H), 1.04-0.99 (m, 2H); LRMS (M+H) m/z 503.1.

### Example 135

### (R)-1-((1-(2-cyanoacetyl)piperidin-3-yl)oxy)-4-(1-cyclobutyl-1H-pyrazol-4-yl)-7-isopropoxyisoquinoline-6-carboxamide (I-115)

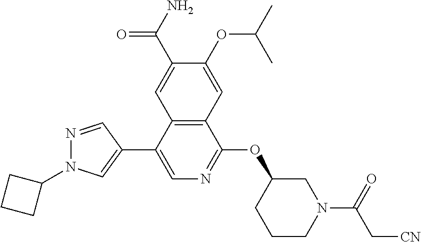 [SMILES: CC(C)Oc1cc2c(O[C@@H]3CCCN(C(=O)CC#N)C3)ncc(-c3cnn(C4CCC4)c3)c2cc1C(N)=O]

General procedures (C): 0.07 mmol scale, 35% yield.

¹H NMR (400 MHz, DMSO-d₆) δ 8.32 (s, 0.4H), 8.29 (s, 0.6H), 8.13 (s, 0.4H), 8.12 (s, 0.6H), 7.90 (s, 0.4H), 7.86 (s, 0.6H), 7.75-7.73 (m, 3H), 7.58 (s, 0.6H), 7.51 (s, 0.4H), 5.46-5.42 (m, 0.4H), 5.40-5.34 (m, 0.6H), 4.98-4.80 (m, 2H), 4.28 (dd, J=13.4, 4.4 Hz, 0.6H), 4.22-4.07 (m, 1.6H), 4.02-3.96 (m, 0.4H), 3.90 (d, J=19.0 Hz, 0.4H), 3.79 (dd, J=14.2, 4.8 Hz, 0.4H), 3.72-3.61 (m, 1.6H), 3.41 (dd, J=13.4, 2.4 Hz, 0.6H), 3.34-3.27 (m, partially overlapped with H₂O, 1H), 3.22-3.15 (m, 0.4H), 2.61-2.51 (m, 2H), 2.48-2.40 (m, 2H), 2.15-2.11 (m, 0.6H), 2.00-1.49 (m, 4.4H), 1.43-1.39 (m, 6H); LRMS (M+H) m/z 517.2.

### Example 136

### (R)-1-((1-(2-cyanoacetyl)piperidin-3-yl)oxy)-4-(1-cyclopentyl-1H-pyrazol-4-yl)-7-isopropoxyisoquinoline-6-carboxamide (I-116)

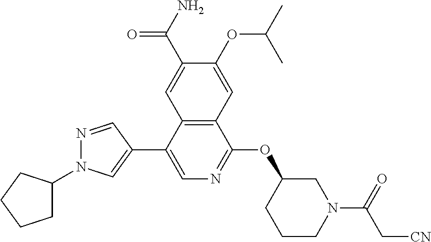 [SMILES: CC(C)Oc1cc2c(O[C@@H]3CCCN(C(=O)CC#N)C3)ncc(-c3cnn(C4CCCC4)c3)c2cc1C(N)=O]

General procedures (G): 0.06 mmol scale, 24% yield.

¹H NMR (400 MHz, DMSO-d₆) δ 8.34 (s, 0.4H), 8.31 (s, 0.6H), 8.09 (d, J=0.8 Hz, 0.4H), 8.08 (d, J=0.8 Hz, 0.6H), 7.90 (s, 0.4H), 7.86 (s, 0.6H), 7.74 (br s, 2H), 7.71 (d, J=0.8 Hz, 0.4H), 7.70 (d, J=0.8 Hz, 0.6H), 7.58 (s, 0.6H), 7.51 (s, 0.4H), 5.43 (br s, 0.4H), 5.37 (br s, 0.6H), 4.93-4.76 (m, 2H), 4.28 (dd, J=13.4, 4.5 Hz, 0.6H), 4.22-3.61 (m, 3.4H), 3.41 (dd, J=13.4, 2.4 Hz, 0.6H), 3.31-3.27 (m, partially overlapped with H₂O, 1H), 3.22-3.15 (m, 0.4H), 2.18-2.10 (m, 2.4H), 2.06-1.94 (m, 3.6H), 1.89-1.79 (m, 2.4H), 1.74-1.49 (m, 3.6H), 1.43-1.39 (m, 6H); LRMS (M+H) m/z 531.2.

### Example 137

### (R)-1-((1-(2-cyanoacetyl)piperidin-3-yl)oxy)-7-isopropoxy-4-(1-(tetrahydro-2H-pyran-4-yl)-1H-pyrazol-4-yl)isoquinoline-6-carboxamide (I-117)

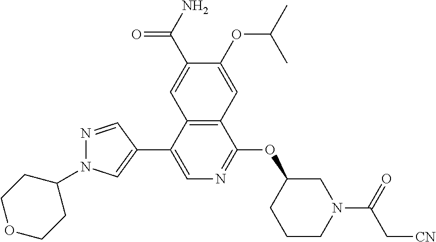 [SMILES: CC(C)Oc1cc2c(O[C@@H]3CCCN(C(=O)CC#N)C3)ncc(-c3cnn(C4CCOCC4)c3)c2cc1C(N)=O]

General procedures (G): 0.085 mmol scale, 36% yield.

¹H NMR (400 MHz, DMSO-d₆) δ 8.34 (s, 0.4H), 8.31 (s, 0.6H), 8.14 (d, J=0.8 Hz, 0.4H), 8.14 (d, J=0.8 Hz, 0.6H), 7.90 (s, 0.4H), 7.86 (s, 0.6H), 7.75-7.73 (m, 3H), 7.59 (s, 0.6H), 7.51 (s, 0.4H), 5.46-5.42 (m, 0.4H), 5.39-5.35 (m, 0.6H), 4.92-4.82 (m, 1H), 4.56-4.48 (m, 1H), 4.30-4.25 (m, 0.6H), 4.22-4.07 (m, 1.4H), 4.07-3.96 (m, 2H), 3.92-3.60 (m, 2H), 3.53-3.47 (m, 2H), 3.44-3.10 (m, 4H), 2.15-1.50 (m, 8H), 1.43-1.39 (m, 6H), 1.26-1.24 (m, 1H); LRMS (M+H) m/z 547.4.

### Example 138

### (R)-1-((1-(2-cyanoacetyl)piperidin-3-yl)oxy)-7-isopropoxy-4-(1-((tetrahydro-2H-pyran-4-yl)methyl)-1H-pyrazol-4-yl)isoquinoline-6-carboxamide (I-118)

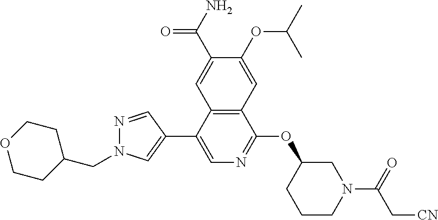 [SMILES: CC(C)Oc1cc2c(O[C@@H]3CCCN(C(=O)CC#N)C3)ncc(-c3cnn(CC4CCOCC4)c3)c2cc1C(N)=O]

General procedures (G): 0.05 mmol scale, 9% yield.

¹H NMR (400 MHz, DMSO-d₆) δ 8.33 (s, 0.4H), 8.31 (s, 0.6H), 8.06 (s, 0.4H), 8.05 (s, 0.6H), 7.90 (s, 0.4H), 7.86 (s, 0.6H), 7.75-7.71 (m, 3H), 7.58 (s, 0.6H), 7.51 (s, 0.4H), 5.45-5.42 (m, 0.4H), 5.38-5.35 (m, 0.6H), 4.93-4.80 (m, 1H), 4.28 (dd, J=13.3, 4.5 Hz, 0.6H), 4.22-4.07 (m, 2.4H), 4.02-3.61 (m, 5H), 3.41 (dd, J=13.4, 2.4 Hz, 0.6H), 3.30-3.13 (m, partially overlapped with H₂O, 2.4H), 2.18-1.83 (m, 3H), 1.73-1.44 (m, 4H), 1.43-1.39 (m, 7H), 1.34-1.24 (m, 2H); LRMS (M+H) m/z 561.2.

### Example 139

### (R)-1-((1-(2-cyanoacetyl)piperidin-3-yl)oxy)-7-isopropoxy-4-(1-(2-(tetrahydro-2H-pyran-4-yl)ethyl)-1H-pyrazol-4-yl)isoquinoline-6-carboxamide (I-119)

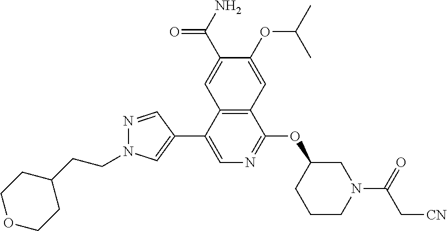 [SMILES: CC(C)Oc1cc2c(O[C@@H]3CCCN(C(=O)CC#N)C3)ncc(-c3cnn(CCC4CCOCC4)c3)c2cc1C(N)=O]

General procedures (G): 0.09 mmol scale, 23% yield.

¹H NMR (400 MHz, DMSO-d₆) δ 8.33 (s, 0.4H), 8.30 (s, 0.6H), 8.09 (d, J=0.8 Hz, 0.4H), 8.08 (d, J=0.8 Hz, 0.6H), 7.89 (s, 0.4H), 7.85 (s, 0.6H), 7.75 (br s, 0.4H), 7.73 (br s, 0.6H), 7.70 (d, J=0.8 Hz, 0.4H), 7.69 (d, J=0.8 Hz, 0.6H), 7.59 (s, 0.6H), 7.51 (s, 0.4H), 5.46-5.42 (m, 0.4H), 5.38-5.34 (m, 0.6H), 4.93-4.81 (m, 1H), 4.31-4.07 (m, 4H), 3.99 (dt, J=12.9, 4.5 Hz, 0.4H), 3.92-3.60 (m, 3.6H), 3.42 (dd, J=13.3, 2.4 Hz, 0.6H), 3.34-3.15 (m, 4H), 3.19 (ddd, J=13.0, 10.0, 3.3 Hz, 0.4H), 2.16-2.11 (m, 0.6H), 2.01-1.78 (m, 3.4H), 1.75-1.44 (m, 5H), 1.43-1.40 (m, 6H), 1.26-1.16 (m, 2H); LRMS (M+H) m/z 575.4.

### Example 140

### 1-(((R)-1-(2-cyanoacetyl)piperidin-3-yl)oxy)-4-(1-(cis-3-(dimethylamino)cyclobutyl)-1H-pyrazol-4-yl)-7-isopropoxyisoquinoline-6-carboxamide (I-120)

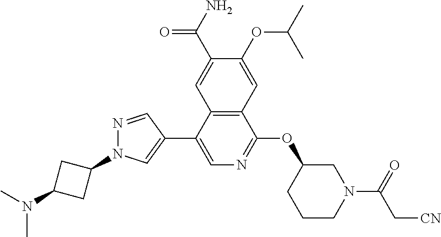 [SMILES: CC(C)Oc1cc2c(O[C@@H]3CCCN(C(=O)CC#N)C3)ncc(-c3cnn([C@H]4C[C@@H](N(C)C)C4)c3)c2cc1C(N)=O]

General procedures (C): 0.08 mmol scale, 40% yield.

¹H NMR (400 MHz, DMSO-d₆) δ 8.32 (s, 0.4H), 8.29 (s, 0.6H), 8.11 (d, J=0.8 Hz, 0.4H), 8.10 (d, J=0.8 Hz, 0.6H), 7.90 (s, 0.4H), 7.86 (s, 0.6H), 7.77-7.74 (m, 3H), 7.59 (s, 0.6H), 7.51 (s, 0.4H), 5.46-5.42 (m, 0.4H), 5.39-5.35 (m, 0.6H), 4.92-4.81 (m, 1H), 4.69-4.60 (m, 1H), 4.28 (dd, J=13.4, 4.5 Hz, 0.6H), 4.22-4.07 (m, 1.6H), 4.02-3.96 (m, 0.4H), 3.92-3.88 (m, 0.4H), 3.79 (dd, J=14.1, 4.8 Hz, 0.4H), 3.72-3.61 (m, 1.6H), 3.42 (dd, J=13.4, 2.4 Hz, 0.6H), 3.31-3.27 (m, partially overlapped with H₂O, 1H), 3.18 (ddd, J=13.1, 9.9, 3.4 Hz, 0.4H), 2.62-2.47 (m, partially overlapped with DMSO, 2H), 2.39-2.32 (m, 2H), 2.09 (s, 6H), 2.00-1.49 (m, 4H), 1.43-1.40 (m, 6H); LRMS (M+H) m/z 560.2.

### Example 141

### tert-butyl (R)-4-(4-(6-carbamoyl-1-((1-(2-cyanoacetyl)piperidin-3-yl)oxy)-7-isopropoxyisoquinolin-4-yl)-1H-pyrazol-1-yl)piperidine-1-carboxylate (I-121)

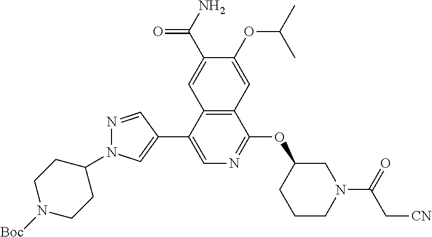 [SMILES: CC(C)Oc1cc2c(O[C@@H]3CCCN(C(=O)CC#N)C3)ncc(-c3cnn(C4CCN(C(=O)OC(C)(C)C)CC4)c3)c2cc1C(N)=O]

General procedures (C): 0.1 mmol scale, 75% yield.

¹H NMR (400 MHz, Chloroform-d) δ 8.93 (s, 0.6H), 8.89 (s, 0.4H), 8.03 (d, J=4.1 Hz, 0.4H), 7.98 (d, J=4.1 Hz, 0.6H), 7.88 (s, 0.6H), 7.86 (s, 0.4H), 7.73 (d, J=0.7 Hz, 0.6H), 7.72 (d, J=0.7 Hz, 0.4H), 7.69 (d, J=0.7 Hz, 0.6H), 7.68 (s, 0.4H), 7.67 (d, J=0.7 Hz, 0.4H), 7.58 (s, 0.6H), 5.89-5.85 (m, 1H), 5.50-5.45 (m, 1H), 5.06-4.91 (m, 1H), 4.45 (dd, J=13.5, 4.8 Hz, 0.4H), 4.41-4.46 (m, 3H), 4.10 (ddd, J=13.1, 4.7, 4.7 Hz, 0.6H), 3.80-3.77 (m, 1.4H), 3.69 (br d, J=13.7 Hz, 0.4H), 3.62-3.41 (m, 2.6H), 3.35 (ddd, J=9.3, 9.3, 4.6 Hz, 0.6H), 2.96-2.90 (m, 2H), 2.36-1.94 (m, 7H), 1.80-1.67 (m, 1H), 1.55 (d, J=6.1 Hz, 2H), 1.53 (d, J=6.1 Hz, 4H), 1.49 (s, 9H); LRMS (M+Na) m/z 646.3.

### Example 142

### (R)-1-((1-(2-cyanoacetyl)piperidin-3-yl)oxy)-7-isopropoxy-4-(1-(piperidin-4-yl)-1H-pyrazol-4-yl)isoquinoline-6-carboxamide (I-122)

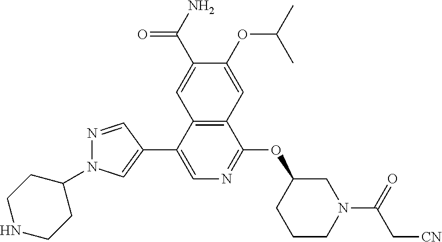 [SMILES: CC(C)Oc1cc2c(O[C@@H]3CCCN(C(=O)CC#N)C3)ncc(-c3cnn(C4CCNCC4)c3)c2cc1C(N)=O]

General procedures (D), step 1: 0.06 mmol scale, 34% yield.

¹H NMR (400 MHz, DMSO-d₆) δ 8.35 (s, 1H), 8.33 (s, 0.4H), 8.31 (s, 0.6H), 8.10 (br s, 0.4H), 8.097 (br s, 0.6H), 7.91 (s, 0.4H), 7.87 (s, 0.6H), 7.74-7.73 (m, 3H), 7.59 (s, 0.6H), 7.51 (s, 0.4H), 5.45-5.42 (m, 0.4H), 5.39-5.35 (m, 0.6H), 4.93-4.80 (m, 1H), 4.43 (tt, J=11.2, 4.1 Hz, 1H), 4.28 (dd, J=13.3, 4.5 Hz, 0.6H), 4.22-4.07 (m, 1.6H), 4.01-3.96 (m, 0.4H), 3.92-3.87 (m, 0.4H), 3.80 (dd, J=14.2, 4.8 Hz, 0.4H), 3.70 (dd, J=14.2, 2.4 Hz, 0.4H), 3.63 (br d, J=14.2 Hz, 0.6H), 3.42 (dd, J=13.3, 2.4 Hz, 0.6H), 3.31 (ddd, J=13.5, 10.1, 3.0 Hz, 0.6H), 3.23-3.15 (m, 2.4H), 2.85-2.78 (m, 2H), 2.15-2.11 (m, 3H), 2.07-1.83 (m, 3.4H), 1.73-1.49 (m, 1.6H), 1.43-1.39 (m, 6H); LRMS (M+H) m/z 546.1.

### Example 143

### (R)-1-((1-(2-cyanoacetyl)piperidin-3-yl)oxy)-7-isopropoxy-4-(1-(1-methylpiperidin-4-yl)-1H-pyrazol-4-yl)isoquinoline-6-carboxamide (I-123)

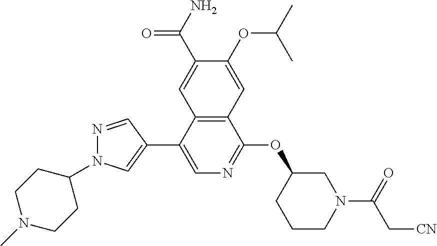 [SMILES: CC(C)Oc1cc2c(O[C@@H]3CCCN(C(=O)CC#N)C3)ncc(-c3cnn(C4CCN(C)CC4)c3)c2cc1C(N)=O]

General procedures (D), step 2, formaldehyde was used: 0.041 mmol scale, 20% yield.

¹H NMR (400 MHz, DMSO-d₆) δ 8.33 (s, 0.4H), 8.30 (s, 0.6H), 8.12 (d, J=0.8 Hz, 0.4H), 8.12 (d, J=0.8 Hz, 0.6H), 7.90 (s, 0.4H), 7.86 (s, 0.6H), 7.74-7.73 (m, 3H), 7.58 (s, 0.6H), 7.51 (s, 0.4H), 5.45-5.42 (m, 0.4H), 5.38-5.35 (m, 0.6H), 4.93-4.82 (m, 1H), 4.30-4.23 (m, 1.6H), 4.22-4.07 (m, 1.6H), 4.01-3.96 (m, 0.4H), 3.92-3.87 (m, 0.4H), 3.79 (dd, J=14.3, 4.8 Hz, 0.4H), 3.70 (dd, J=14.2, 2.4 Hz, 0.4H), 3.63 (br d, J=14.0 Hz, 0.6H), 3.42 (dd, J=13.3, 2.4 Hz, 0.6H), 3.34-3.27 (m, overlapped with H₂O, 0.6H), 3.22-3.15 (m, 0.4H), 2.99-2.97 (m, 2H), 2.31 (s, 3H), 2.24-1.83 (m, 8H), 1.73-1.49 (m, 2H), 1.43-1.39 (m, 6H); LRMS (M+H) m/z 560.2.

### Example 144

### 1-(((R)-1-(2-cyanoacetyl)piperidin-3-yl)oxy)-4-(1-(trans-4-ethoxycyclohexyl)-1H-pyrazol-4-yl)-7-isopropoxyisoquinoline-6-carboxamide (I-124)

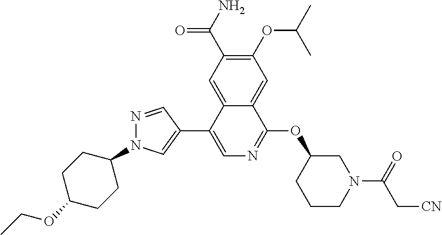 [SMILES: CCO[C@H]1CC[C@H](n2cc(-c3cnc(O[C@@H]4CCCN(C(=O)CC#N)C4)c4cc(OC(C)C)c(C(N)=O)cc34)cn2)CC1]

General procedures (C): 0.1 mmol scale, 20% yield.

¹H NMR (400 MHz, Chloroform-d) δ 8.95 (s, 0.6H), 8.91 (s, 0.4H), 8.03 (d, J=4.0 Hz, 0.4H), 7.98 (d, J=4.0 Hz, 0.6H), 7.88 (s, 0.6H), 7.87 (s, 0.4H), 7.72 (d, J=0.8 Hz, 0.6H), 7.70-7.65 (m, 1.8H), 7.58-7.57 (m, 0.6H), 5.97-5.93 (m, 1H), 5.50-5.45 (m, 1H), 5.05-4.91 (m, 1H), 4.44 (dd, J=13.6, 4.8 Hz, 0.4H), 4.27-4.18 (m, 1H), 4.12-4.07 (m, 0.6H), 3.83-3.75 (m, 1.4H), 3.69 (br d, J=14.1 Hz, 0.4H), 3.63-3.33 (m, 7.2H), 2.34-2.30 (m, 2H), 2.25-1.89 (m, 7H), 1.80-1.68 (m, overlapped with H₂O, 2H), 1.56-1.42 (m, 8H), 1.23 (t, J=7.0 Hz, 3H); LRMS (M+H) m/z 589.3.

### Example 145

### LPS Induced IL23p19 in THP-1 Cells (with IFNγ Primed) Assay

### Materials and Equipment

THP-1 Cells (ATCC, Cat #TIB-202), Dimethyl Sulfoxide (DMSO) (Sigma-Aldrich, Cat #D2650), RPMI 1640 (Cellgro, Cat #10-040-CM), Fetal Bovine Serum (Sigma, Cat #F4135), Albumin From Bovine Serum (BSA) (Sigma-Aldrich, Cat #A7906), LPS (Serotype K-235, Sigma, Product Number L 2143), IFNγ (Peprotech, Cat #300-02), Capture antibody: Human IL-23p19 ELISA (e-Bioscience, Cat. #14-7238-85), Detection antibody: Primary Mouse Biotinylated anti-human IL-12(p40/p70) (e-Bioscience, Cat. #13-7129-85), Secondary HRP-conjugated Streptavidin (R&D Systems, Cat #DY998), 1×PBST Washing Buffer (PBS-Tween tablet) (VWR International, Cat #80058-558), ELISA Blocking Buffer (PBS with 1% BSA), ELISA Dilution Buffer (PBS with 1% BSA), 384 Well Flat-Bottom, MaxiSorp Black Immuno Plates (Thermo Scientific, Cat #12-565-346), 384 Well Flat-Bottom, White Tissue Culture Plates (Thermo Scientific, Cat #12-565-343), Super Signal ELISA Pico Chemiluminescent Substrate (Thermo Scientific, Cat #37070), Cell Titer Glo reagent (Promega, Cat #G7573), Positive control, IKK2VI inhibitor (Calbiochem, Cat #401483), AquaMax 4000 plate washer (Molecular Devices), Luminometer, Wallac Victor2 1420 Multilabel Counter.

### Method

### THP-1 Cells Stimulation:

On day 1, 50K/well THP-1 cells were seeded and primed with IFNγ (50 ng/mL) in 384-well plates for about 18 hours in RPMI media with 10% FBS. On day 2, the compound was serially diluted in DMSO from 5 mM in 3-fold dilutions, and then diluted 1:125 in RPMI media with 10% FBS. 50 μL/well 2× compound was added to 50 μL/well THP-1 cells (with IFNγ primed) in duplicate in 384 well tissue culture plates. The cells were pre-incubated with compound for 1 hour at 37° C., 5% CO₂ before addition of 10 μL/well 1×LPS to give a final concentration of 1 μg/mL LPS. Day 3, after stimulation for 18 hours at 37° C., 5% CO₂, the assay plate was centrifuged and 70 μL/well supernatant was harvested. IL-23p19 protein in 70 L/well of supernatant was measured by sandwich ELISA, and 25 μl/well Cell Titer Glo reagent was added to the remaining cells to measure compound toxicity.

### Human IL-23p19 Sandwich ELISA:

Maxisorp immuno ELISA plates were pre-coated with 25 μL/well of anti-IL-23p19 capture antibody (2.5 ug/mL) in PBS overnight at room temperature. After washing with 1×PBST, the plates were blocked using 100 μL/well of 1% BSA in PBS for 2 hours at room temperature. The plates were washed three times with 1×PBST and 70 μL/well supernatant were added. The plates were incubated at room temperature for 2 hours with shaking and washed three times with 1×PBST. 25 μL/well of biotin labeled anti-IL-12(p40/p70) detection antibody (100 ng/mL) in PBS with 1% BSA was added and the plates were incubated at room temperature for 2 hours with shaking. After washing three times with 1×PBST, 25 μL/well of streptavidin-HRP (1:200) in PBS with 1% BSA was added and the plates were incubated at room temperature for 20 minutes with shaking. The plates were washed three times with 1×PBST and 25 μL/well of Super Signal ELISA Pico Chemiluminescent Substrate were added. The plates were read with a luminometer, and the chemiluminescence values were entered into Athena (Rigel) for curve fitting, EC₅₀ calculation, and database storage. The results are shown in Table 1.

### Example 146

### IRAK4 ADP-Glo Assay

### Materials

IRAK4 Kinase Enzyme (Signalchem, 112-10G-20); 0.1M DTT (Signalchem, D86-09B); MBP Substrate (Signalchem, M42-51N); ADP Glo (Promega, V9101); 1M MgCl₂ (Teknova, M03304); 1M Tris-HCl pH7.4 (Teknova, T1074); BSA (Sigma (A3059); Distilled H₂O

### Equipment

Wallac Victor2 1420 Multilabel Counter

### Method

The ADP-Glo™ reagents were thawed at ambient temperature. The Kinase Detection Reagent was prepared by mixing kinase detection buffer with the lyophilized kinase detection substrate, and set aside.

A stock volume of 5× Reaction Kinase Buffer was made with a final concentration of 100 mM MgCl₂, 200 mM Tris-HCl, and 0.5 mg/ml of BSA, in distilled H₂O with a final pH7.4. A 2× working stock volume of Reaction Kinase Buffer was made containing a final concentration of 100 μM DTT.

The components of IRAK4 Enzyme were thawed on ice. Diluted IRAK4 in 1× Kinase Reaction Buffer (diluted from 2× buffer) was prepared at 5.0 ng/μl. A 250 μM working stock ATP Assay Solution was prepared in 1× Kinase Reaction Buffer (diluted from 2× buffer).

The compound was diluted in DMSO from 250 μM in 4-fold series dilutions for 8 points. Then diluted 1:5 in 2× Reaction Buffer in a 96 well plate. 1.0 μl was transferred to a 384 well plate in duplicate. 2 μl of diluted Active IRAK4 was added (do not add to column 1) and 2× reaction buffer was added to column 1. 1 μl of 1 mg/ml stock solution of MBP substrate was added NOTE: MBP can be combined with ATP mix with equal volume and then added at 2 μl/well. Final reaction volume was 5 μl.

The plate was centrifuged and the reaction mixture was incubated at room temperature for 60 minutes or at 30° C. for 30 minutes.

The reaction was terminated and the remaining ATP was depleted by adding 5 μl of ADP-Glo™ Reagent. The 384-well plate was centrifuged and then the reaction mixture was incubated for another 40 minutes at ambient temperature.

10 μl of the Kinase Detection Reagent was added. The plate was centrifuged and then the reaction mixture was incubated for another 30 minutes at ambient temperature.

The 384-well reaction plate was read using the WALLAC plate reader (Luminescence 0.1s). The results are provided in Table 1.

### Example 147

### R848-induced TNF-α in human PBMCs Assay

### Materials

Dimethyl Sulfoxide (DMSO) (Sigma-Aldrich, Cat #D2650)

Fetal Bovine Serum (Sigma, Cat #F4135)

Human PBMC cells (BioIVT, USA)

RPMI growth media containing 10% FBS

R848 (Resiquimod) (Sigma-Aldrich, USA)

White clear bottom 96 well plates (Fisher, Cat No. 07-200-587, Corning #3903)

Albumin From Bovine Serum (BSA) (Sigma-Aldrich, Cat #A7906)

1×PBST Washing Buffer (PBS-Tween tablet) (VWR International, Cat #80058-558)

ELISA Blocking Buffer (PBS with 2% BSA)

ELISA Dilution Buffer (0.2% BSA in PBST)

Human TNF-α ELISA Kit (R&D Systems Cat No. DY210)

Cell Titer Glo reagent (Promega, Cat No. G7573)

### Equipment

Scan Washer 400 plate washer (Molecular Devices)

Luminometer, Wallac Victor2 1420 Multilabel Counter

### Method

### PBMCs Stimulation:

100 μL/well PBMCs (200K/well) were seeded in 96-well flat-bottom tissue culture plates. Compound was serially diluted in DMSO from 2.5 mM in 4-fold dilutions, and then diluted 1:125 in RPMI 1640 complete media with 10% FBS. 100 μL/well 2× compound was added to the PBMC in the tissue culture plates. The cells were pre-incubated with compound for 1 hour at 37° C., 5% CO₂ and before addition of 10 μL/well 21× R848 to give a final concentration of 65 ng/mL for R848. The cells were stimulated with R848 for an additional 4 hours at 37° C., 5% CO₂. The supernatant (100 μl/well) was harvested to measure TNF-α by ELISA. Cell Titer Glo reagent (100 μl/well) was added to the remaining 100 μl/well of the cell culture plate, incubated for 1-2 minutes on a shaker, and the plate was read for luminescence intensity to determine the compound cytotoxicity. The chemiluminescence values were entered into Athena (Rigel) for curve fitting, EC₅₀ calculation, and database storage.

### Human TNF-α Sandwich ELISA:

TNF-α ELISA was performed according to the vendor protocol, using 96 Well Flat-Bottom, MaxiSorp Black Immuno plates and 100 μL/well of supernatant. The plates were developed using 100 μL/well of Super Signal ELISA Pico Chemiluminescent Substrate. The plates were read with a luminometer, and the chemiluminescence values were entered into Athena (Rigel) for curve fitting, EC₅₀ calculation, and database storage. The results are provided in Table 1.

### Example 148

### Gardiquimod-Induced TNF-α in Human Primary Monocyte-Derived Dendritic Cells

### Materials

Dimethyl Sulfoxide (DMSO) (Sigma-Aldrich, Cat #D2650)

Fetal Bovine Serum (Sigma, Cat #F4135)

Human PBMC cells (BioIVT, USA)

RPMI growth media containing 10% FBS

IFNγ (Peprotech, Cat No. 300-02)

GMCSF (Peprotech, Cat No. 300-03) and IL-4 (Peprotech Cat No. 200-04)

Gardiquimod NEW Imidazoquinoline compound (InvivoGen, Cat #tlrl-gdqs)

White clear bottom 96 well plates (Fisher, Cat No. 07-200-587, Corning #3903)

Albumin From Bovine Serum (BSA) (Sigma-Aldrich, Cat #A7906)

1×PBST Washing Buffer (PBS-Tween tablet) (VWR International, Cat #80058-558)

ELISA Blocking Buffer (PBS with 2% BSA)

ELISA Dilution Buffer (0.2% BSA in PBST)

Human TNF-α ELISA Kit (R&D Systems Cat No. DY210)

Cell Titer Glo reagent (Promega, Cat No. G7573)

### Equipment

Scan Washer 400 plate washer (Molecular Devices)

Luminometer, Wallac Victor2 1420 Multilabel Counter

### Method

### Differentiation and IFNγ-Priming of Dendritic Cells:

Human peripheral blood mononuclear cells (PBMC) (500 million) were allowed to adhere in a T-175 flask containing 15 ml RPMI media (10% FBS) for 2 hours at 37° C., 5% CO₂ to capture the monocytes. After 2 hours, the media including floating cells was carefully removed and 12 ml of fresh RPMI media (10% FBS) containing GM-CSF (100 ng/ml) and IL-4 (20 ng/ml) were added to the adherent monocytes. The monocytes were allowed to differentiate for 7 days at 37° C., 5% CO₂ for 7 days, supplementing with fresh GM-CSF and IL-4 after 3 days. The differentiated dendritic cells (DC) were harvested by centrifugation (1200 rpm/5 min). The DCs were primed at 37° C., 5% CO₂ for 24 hours with 50 ng/ml IFN-γ (1000 U/ml) at 50K/well in 100 μl/well of fresh RPMI media (10% FBS) in a white clear bottom 96 well plate.

### Compound Pre-Incubation:

Compound was serially diluted in DMSO from 2.5 mM in 4-fold dilutions, and then diluted 1:125 in RPMI 1640 complete media with 10% FBS. The primed DCs were pre-incubated with test compound for 1 hour at 37° C., 5% CO₂ by adding 100 μl of RPMI media containing 2× concentrated test compound per well (0.5% final DMSO concentration).

### Gardiguimod Stimulation:

DCs were stimulated with Gardiquimod (10 μl per well of 20× concentrated Gardiquimod solution in RPMI media to give a final concentration of 4 μg/ml) at 37° C., 5% CO₂ for an additional 6 hours. The supernatant (100 μl/well) was harvested to measure TNF-α by ELISA. Cell Titer Glo reagent (100 μl/well) was added to the remaining 100 μl/well of the cell culture plate, incubated for 1-2 minutes on a shaker, and the plate was read for luminescence intensity to determine the compound cytotoxicity. The chemiluminescence values were entered into Athena (Rigel) for curve fitting, EC₅₀ calculation, and database storage.

### Human TNF-α Sandwich ELISA:

TNF-α ELISA was performed according to the vendor protocol, using 96 Well Flat-Bottom, MaxiSorp Black Immuno plates and 100 μL/well of supernatant. The plates were developed using 100 μL/well of Super Signal ELISA Pico Chemiluminescent Substrate. The plates were read with a luminometer, and the chemiluminescence values were entered into Athena (Rigel) for curve fitting, EC₅₀ calculation, and database storage. The results are provided in Table 1.

TABLE 1
In vitro results.

| | IRAK4 | IL23 | TNFα | TNFα |
|---|---|---|---|---|
| | (IC₅₀, | (THP, LPS) | (PBMC, R848) | (DC, GARD) |
| Compound | μM) | (IC₅₀, μM) | (IC₅₀, μM) | (IC₅₀, μM) |
| I-1 | 0.034 | 9.481 | 2.61 | 2.644 |
| I-2 | 0.0044 | 5.532 | 1.151 | 5.336 |
| I-3 | 0.0125 | 0.8789 | 0.3596 | 0.4107 |
| I-4 | 0.0053 | 1.817 | 0.2424 | 0.5326 |
| I-5 | 0.1201 | 6.51 | 0.4326 | 0.4877 |
| I-6 | 0.0051 | 0.1951 | 0.1986 | 0.3342 |
| I-7 | 0.0082 | 0.527 | 0.402 | 0.3829 |
| I-8 | 0.0123 | 0.3321 | 0.411 | 0.3129 |
| I-9 | 0.0099 | 0.3941 | 0.3187 | 0.21 |
| I-10 | 0.0116 | 3.276 | 0.8734 | 2.926 |
| I-11 | 0.0105 | 0.4737 | 0.3199 | 0.1987 |
| I-12 | 0.0412 | 1.252 | 0.4477 | 6.093 |
| I-13 | 0.0149 | 4.64 | 4.669 | 3.503 |
| I-14 | 0.0054 | ND* | 3.932 | 7.868 |
| I-15 | 0.0159 | 0.5249 | 0.0874 | 0.0719 |
| I-16 | 0.0119 | 0.0886 | 0.0474 | 0.2446 |
| I-17 | 0.0017 | 11.2 | 1.331 | 17.17 |
| I-18 | 0.0023 | 0.6061 | 0.3062 | 0.2033 |
| I-19 | 0.0067 | 0.124 | | 0.6181 |
| I-20 | 0.007 | 0.1096 | 0.0688 | 0.0634 |
| I-21 | 0.0056 | 0.1937 | 0.104 | 0.1014 |
| I-22 | 0.0123 | 0.2083 | 0.049 | 0.0729 |
| I-23 | 0.0114 | 0.7123 | 2.12 | 0.5664 |
| I-24 | 0.0081 | 0.1248 | 0.0763 | 0.0224 |
| I-25 | 0.0033 | 12.72 | 2.356 | 1.353 |
| I-26 | 0.0026 | 0.7264 | 0.3635 | 0.1609 |
| I-27 | 0.0056 | 0.6336 | 0.1757 | 0.5137 |
| I-28 | 0.0063 | 0.14 | 0.0331 | 0.1109 |
| I-29 | 0.0055 | 4.248 | 0.6862 | 1.748 |
| I-30 | 0.0064 | 0.1418 | 0.0947 | 0.071 |
| I-31 | 0.0035 | 0.1355 | 0.0637 | 0.0898 |
| I-32 | 0.0085 | 2.205 | 0.4502 | 0.5693 |
| I-33 | 0.0075 | 0.2002 | 0.2715 | 0.218 |
| I-34 | 0.0077 | 1.301 | 0.2947 | 0.4701 |
| I-35 | 0.0257 | 7.516 | 1.303 | 1.623 |
| I-36 | 0.0135 | 0.976 | 0.3768 | 0.3981 |
| I-37 | 0.0137 | 1.344 | 0.3243 | 0.2963 |
| I-38 | 0.021 | 3.407 | 1.486 | 4.031 |
| I-39 | 0.0333 | 0.7749 | 0.6927 | 3.52 |
| I-40 | 0.0171 | 0.8165 | 0.1877 | 2.421 |
| I-41 | 0.0376 | 4.612 | 0.5093 | 9.995 |
| I-42 | 0.0064 | 0.7203 | 0.2414 | 0.135 |
| I-43 | 0.005 | 3.102 | 0.7007 | 0.3549 |
| I-44 | 0.0209 | 0.9374 | 0.727 | 1.054 |
| I-45 | 0.0186 | 10.87 | 10.03 | 1.362 |
| I-46 | 0.0156 | 5.425 | 1.253 | 0.466 |
| I-47 | 0.0076 | 2.726 | 0.8161 | 1.87 |
| I-48 | 0.0044 | 0.1654 | 0.0569 | 0.1202 |
| I-49 | 0.0076 | 0.2728 | 0.0853 | 0.0737 |
| I-50 | 0.0048 | 1.617 | 0.1149 | 0.2296 |
| I-51 | 0.0053 | 1.271 | 0.3912 | 1.031 |
| I-52 | 0.0183 | 4.572 | 0.3382 | 0.9269 |
| I-53 | 0.0063 | 10.97 | 3.056 | 14.16 |
| I-54 | 0.1134 | 2.973 | 2.752 | 1.713 |
| I-55 | 0.0035 | 4.001 | 1.203 | 1.215 |
| I-56 | 0.0033 | 0.422 | 0.1364 | 0.0249 |
| I-57 | 0.0045 | 7.39 | 0.4678 | 0.6343 |
| I-58 | 0.7291 | 2964 | 1.561 | 0.0225 |
| I-59 | 0.0039 | 0.0466 | 0.0472 | 0.032 |
| I-60 | 0.0093 | 0.0246 | 0.0778 | 0.0213 |
| I-61 | 0.0088 | 0.1017 | 0.0807 | 0.013 |
| I-62 | 0.0193 | 0.0219 | 0.001 | 0.0021 |
| I-63 | 0.0028 | 5.9 | 1.208 | 0.5231 |
| I-64 | 0.0036 | ND* | 0.0215 | 0.0286 |
| I-65 | 0.0087 | 0.0465 | 0.0065 | 0.029 |
| I-66 | 0.002 | 0.0846 | 0.0037 | 0.0041 |
| I-67 | 0.0063 | 0.6079 | 0.0472 | 0.5928 |
| I-68 | 0.0095 | 0.2863 | 0.4056 | 0.5449 |
| I-69 | 0.0051 | 0.3909 | 0.2407 | 0.0523 |
| I-70 | 0.0068 | 2.263 | 0.2919 | 0.0827 |
| I-71 | 0.0064 | 0.8484 | 0.1741 | 0.1484 |
| I-72 | 0.007 | 0.8718 | 0.1827 | 0.2729 |
| I-73 | 0.003 | 1.043 | 0.2029 | 0.2586 |
| I-74 | 0.0112 | 0.3951 | 0.1408 | 0.4934 |
| I-75 | 0.004 | 85.41 | 6.641 | 4.575 |
| I-76 | 0.0024 | 1.11 | 0.3747 | 0.452 |
| I-77 | 0.007 | 0.1519 | 0.12 | 0.3492 |
| I-78 | 0.0036 | 0.1617 | 0.234 | 0.6516 |
| I-79 | 0.0181 | 0.0072 | | |
| I-80 | 0.3897 | 0.0105 | | |
| I-81 | 0.4632 | 0.0163 | | |
| I-85 | 0.035 | 1.963 | 2.082 | 4.857 |
| I-86 | 0.0913 | 5.291 | 1.741 | 1.032 |
| I-87 | 0.0522 | 5.016 | 1.974 | 0.776 |
| I-88 | 0.0297 | 2.067 | 0.9034 | 0.6956 |
| I-89 | 0.0152 | 1.712 | 0.556 | 0.5301 |
| I-90 | 0.0205 | 1.423 | 0.4333 | 0.6162 |
| I-91 | 0.0207 | 10.18 | 3.756 | 15.4 |
| I-92 | 0.1415 | 4.925 | 3.697 | 13.88 |
| I-93 | 0.023 | ND* | ND* | ND* |
| I-94 | 0.0568 | 18.81 | 8.077 | ND* |
| I-95 | 0.0685 | 1.559 | 2.102 | 0.8421 |
| I-96 | 0.008 | 7.845 | 1.714 | 1.171 |
| I-97 | 0.0154 | 1.982 | 0.7519 | 0.2503 |
| I-98 | 0.0255 | 0.4845 | 0.2196 | 0.1362 |
| I-99 | 0.0231 | 0.4828 | 0.555 | 0.3655 |
| I-100 | 0.0348 | 0.9599 | 3.303 | 1.412 |
| I-101 | 0.0137 | 0.2701 | 3.328 | 0.75 |
| I-102 | 0.0353 | 0.1604 | 0.19 | 0.063 |
| I-103 | 0.0052 | 9.346 | 0.4405 | 0.828 |
| I-104 | 0.0071 | 1.76 | 0.237 | 0.1589 |
| I-105 | 0.01 | 0.6033 | 0.2753 | 0.2855 |
| I-106 | 0.0032 | 0.4359 | 0.3628 | 0.0535 |
| I-107 | ND* | 102.6 | 38.17 | |
| I-108 | 0.1417 | 10.04 | 5.503 | 1.366 |
| I-109 | 0.1709 | 34.86 | 2.55 | 1.436 |
| I-110 | 0.1229 | 24.7 | 2.44 | 1.787 |
| I-111 | 1.439 | 15.51 | 3.463 | 2.268 |
| I-112 | 0.2795 | 13.55 | 3.95 | 9.222 |
| I-113 | 0.3636 | 8.82 | 0.6775 | 0.8529 |
| I-114 | 0.0123 | 1.422 | 0.5487 | 0.6592 |
| I-115 | 0.0132 | 0.9113 | 0.4432 | 0.4505 |
| I-116 | 0.0077 | 1.372 | 0.2712 | 0.2193 |
| I-117 | 0.0253 | 3.91 | 7.237 | 1.03 |
| I-118 | 0.0204 | 4.196 | 6.505 | 1.496 |
| I-119 | 0.0184 | 2.219 | 2.506 | 1.022 |
| I-120 | 0.0084 | 2.835 | 0.2266 | 0.4234 |
| I-121 | 0.0129 | 1.902 | 1.758 | 1.02 |
| I-122 | 0.024 | ND* | 2.162 | 1.005 |
| I-123 | 0.0192 | 4.904 | 0.1453 | 0.2169 |
| I-124 | 0.0068 | 0.6092 | 12.19 | 0.3519 |

*ND indicates that an accurate inhibition curve may not have been produced due to compound insolubility, artifacts in the assay, and/or other factors.

In view of the many possible embodiments to which the principles of the disclosure may be applied, it should be recognized that the illustrated embodiments are only preferred examples of the disclosure and should not be taken as limiting the scope of the disclosure. Rather, the scope of the disclosure is defined by the following claims. We therefore claim as our technology all that comes within the scope and spirit of these claims.

We claim:

1. A compound having a structure according to Formula I 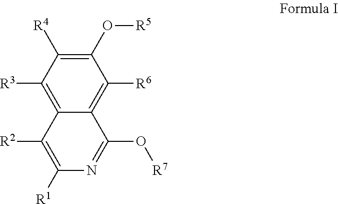
or a pharmaceutically acceptable salt or solvate thereof, wherein:
each of R¹, R³ and R⁶ independently are H or C₁₋₆alkyl;
R² is alkynyl, heteroaryl, or halogen;
R⁴ is —C(O)NH₂;
R⁵ is C₁₋₆alkyl;
R⁷ is —(CH₂)ₙ-heterocycloaliphatic; and
n is 0, 1 or 2.

2. The compound of claim 1, wherein each of R¹, R³ and R⁶ is H.

3. The compound of claim 1, wherein R⁵ is isopropyl.

4. The compound of claim 1, wherein R⁵ is methyl.

5. The compound of claim 1, wherein R² is halogen.

6. The compound of claim 5, wherein R² is Br.

7. The compound of claim 1, wherein R² is 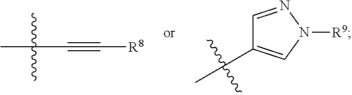
R⁸ is C₁₋₆alkyl optionally substituted with 1, 2 or 3 substituents selected from OH or 4- to 6-membered heterocycloaliphatic, 4- to 6-membered heterocycloaliphatic optionally substituted with 1, 2, or 3 substituents selected from R^{a}, or C₃₋₆cycloalkyl optionally substituted with 1, 2 or 3 R^{b};
R⁹ is C₃₋₆cycloalkyl optionally substituted with 1, 2 or 3 R^{b}, C₁₋₆alkyl optionally substituted with 4- to 6-membered heterocycloaliphatic, or 4- to 6-membered heterocycloaliphatic optionally substituted with 1, 2, or 3 R^{a};
R^{a} is OH, C(O)OR^{c}, C₁₋₆alkyl, or C(O)NH(R^{c});
R^{b} is OR^{c}, C₁₋₆alkyl, or N(R^{c})₂; and
R^{c} is H or C₁₋₆alkyl.

8. The compound of claim 7, wherein R² is selected from OH

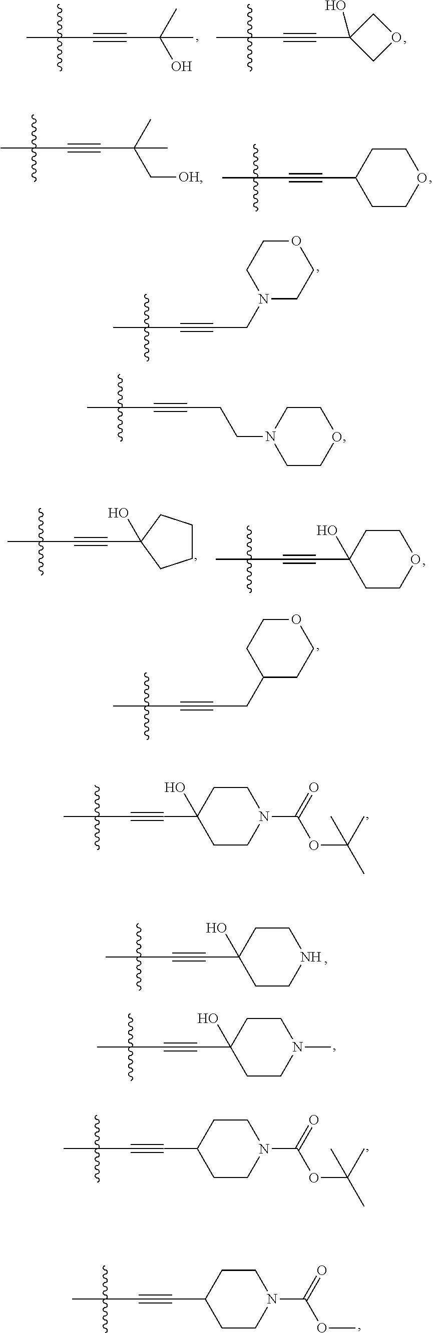

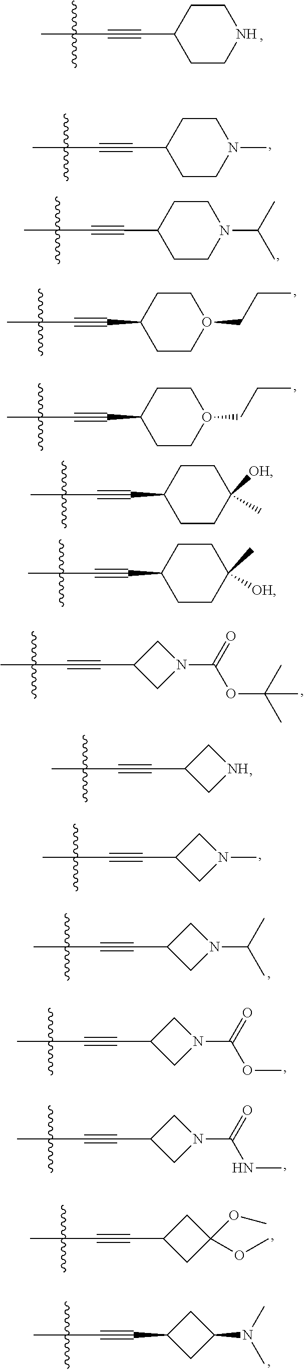

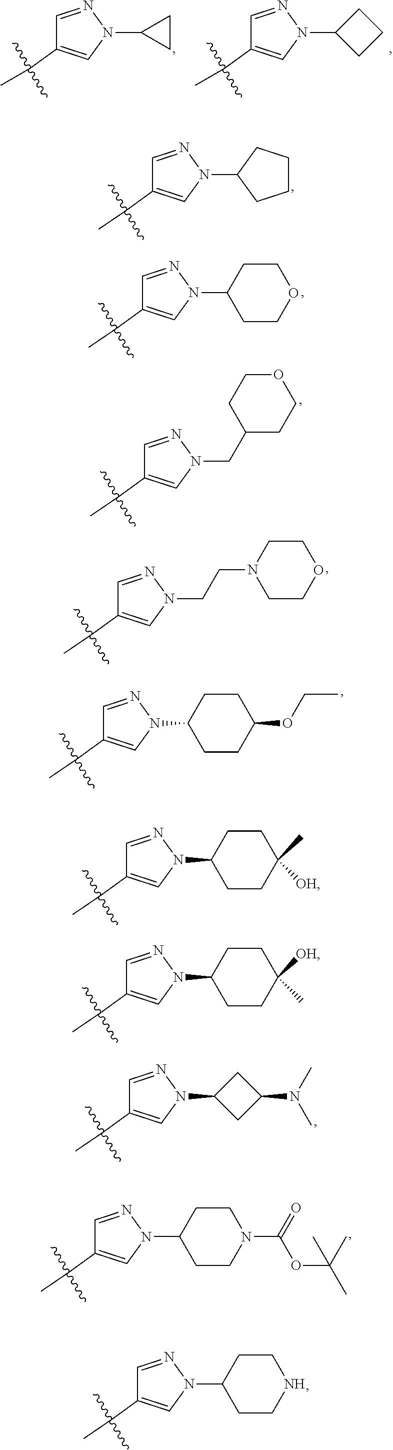

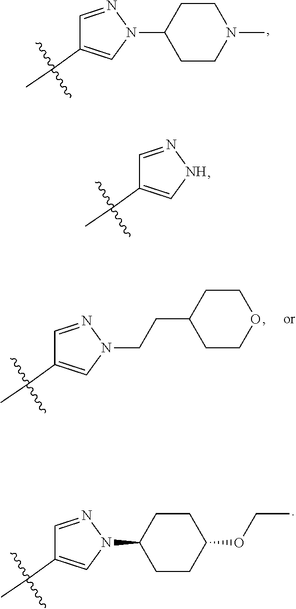 [SMILES: *c1cnn(C2CCN(C)CC2)c1 ; *c1cn[nH]c1 ; *c1cnn(CCC2CCOCC2)c1 ; *c1cnn([C@H]2CC[C@H](OCC)CC2)c1]

9. The compound of claim 1, wherein R⁷ is —(CH₂)ₙ-(5-membered or 6-membered heterocycloaliphatic) optionally substituted with oxo (═O), —C(O)CH₂CN, halogen (such as F), C₁₋₆alkyl, —C(O)O—C₁₋₆alkyl), or a combination thereof.

10. The compound of claim 9, wherein the 5-membered or 6-membered heterocycloaliphatic is a 5-membered or 6-membered nitrogen-containing heterocycloaliphatic.

11. The compound of claim 10, wherein R⁷ is 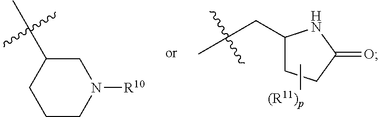
R¹⁰ is H, C(O)OC₁₋₆alkyl, or C(O)CH₂CN;
each R¹¹ independently is halogen or C₁₋₆alkyl; and
p is from 0 to 4.

12. The compound of claim 11, wherein p is 0, 1 or 2.

13. The compound of claim 11, wherein p is 1, 2, 3, or 4 and each R¹¹ independently is F or ethyl.

14. The compound of claim 11, wherein the compound has a structure according to Formula III or a pharmaceutically acceptable salt or solvate thereof:

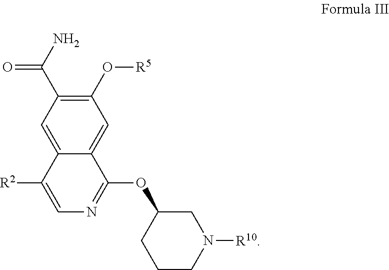

15. The compound of claim 11, wherein the compound has a structure according to Formula IV or a pharmaceutically acceptable salt or solvate thereof:

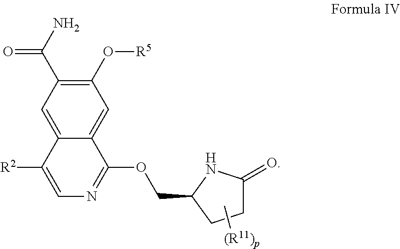

16. The compound of claim 1, wherein the compound is selected from
I:1 (S)-4-bromo-7-isopropoxy-1-((5-oxopyrrolidin-2-yl)methoxy)isoquinoline-6-carboxamide;
I-2: (S)-4-bromo-1-((4,4-difluoro-5-oxopyrrolidin-2-yl)methoxy)-7-isopropoxyisoquinoline-6-carboxamide;
I-3: (S)-4-(3-hydroxy-3-methylbut-1-yn-1-yl)-7-isopropoxy-1-((5-oxopyrrolidin-2-yl)methoxy)isoquinoline-6-carboxamide;
I-4: (S)-4-((3-hydroxyoxetan-3-yl)ethynyl)-7-isopropoxy-1-((5-oxopyrrolidin-2-yl)methoxy)isoquinoline-6-carboxamide;
I-5: (S)-4-(4-hydroxy-3,3-dimethylbut-1-yn-1-yl)-7-isopropoxy-1-((5-oxopyrrolidin-2-yl)methoxy)isoquinoline-6-carboxamide;
I-6: (S)-7-isopropoxy-1-((5-oxopyrrolidin-2-yl)methoxy)-4-((tetrahydro-2H-pyran-4-yl)ethynyl)isoquinoline-6-carboxamide;
I-7: (S)-7-isopropoxy-4-(3-morpholinoprop-1-yn-1-yl)-1-((5-oxopyrrolidin-2-yl)methoxy)isoquinoline-6-carboxamide;
I-8: (S)-7-isopropoxy-4-(4-morpholinobut-1-yn-1-yl)-1-((5-oxopyrrolidin-2-yl)methoxy)isoquinoline-6-carboxamide;
I-9: (S)-4-((1-hydroxycyclopentyl)ethynyl)-7-isopropoxy-1-((5-oxopyrrolidin-2-yl)methoxy)isoquinoline-6-carboxamide;
I-10: (S)-4-((4-hydroxytetrahydro-2H-pyran-4-yl)ethynyl)-7-isopropoxy-1-((5-oxopyrrolidin-2-yl)methoxy)isoquinoline-6-carboxamide;
I-11: (S)-7-isopropoxy-1-((5-oxopyrrolidin-2-yl)methoxy)-4-(3-(tetrahydro-2H-pyran-4-yl)prop-1-yn-1-yl)isoquinoline-6-carboxamide;
I-12: tert-butyl (S)-4-((6-carbamoyl-7-isopropoxy-1-((5-oxopyrrolidin-2-yl)methoxy)isoquinolin-4-yl)ethynyl)-4-hydroxypiperidine-1-carboxylate;
I-13: (S)-4-((4-hydroxypiperidin-4-yl)ethynyl)-7-isopropoxy-1-((5-oxopyrrolidin-2-yl)methoxy)isoquinoline-6-carboxamide;
I-14: (S)-4-((4-hydroxy-1-methylpiperidin-4-yl)ethynyl)-7-isopropoxy-1-((5-oxopyrrolidin-2-yl)methoxy)isoquinoline-6-carboxamide;
I-15: tert-butyl (S)-4-((6-carbamoyl-7-isopropoxy-1-((5-oxopyrrolidin-2-yl)methoxy)isoquinolin-4-yl)ethynyl)piperidine-1-carboxylate;
I-16: methyl (S)-4-((6-carbamoyl-7-isopropoxy-1-((5-oxopyrrolidin-2-yl)methoxy)isoquinolin-4-yl)ethynyl)piperidine-1-carboxylate;
I-17: (S)-7-isopropoxy-1-((5-oxopyrrolidin-2-yl)methoxy)-4-(piperidin-4-ylethynyl)isoquinoline-6-carboxamide;
I-18: (S)-7-isopropoxy-4-((1-methylpiperidin-4-yl)ethynyl)-1-((5-oxopyrrolidin-2-yl)methoxy)isoquinoline-6-carboxamide;
I-19: (S)-7-isopropoxy-4-((1-isopropylpiperidin-4-yl)ethynyl)-1-((5-oxopyrrolidin-2-yl)methoxy)isoquinoline-6-carboxamide;
I-20: 4-(((1r,4S)-4-ethoxycyclohexyl)ethynyl)-7-isopropoxy-1-(((S)-5-oxopyrrolidin-2-yl)methoxy)isoquinoline-6-carboxamide;
I-21: 4-(((1s,4R)-4-ethoxycyclohexyl)ethynyl)-7-isopropoxy-1-(((S)-5-oxopyrrolidin-2-yl)methoxy)isoquinoline-6-carboxamide;
I-22: 4-(((1r,4S)-4-hydroxy-4-methylcyclohexyl)ethynyl)-7-isopropoxy-1-(((S)-5-oxopyrrolidin-2-yl)methoxy)isoquinoline-6-carboxamide;
I-23: 4-(((1s,4R)-4-hydroxy-4-methylcyclohexyl)ethynyl)-7-isopropoxy-1-(((S)-5-oxopyrrolidin-2-yl)methoxy)isoquinoline-6-carboxamide;
I-24: tert-butyl (S)-3-((6-carbamoyl-7-isopropoxy-1-((5-oxopyrrolidin-2-yl)methoxy)isoquinolin-4-yl)ethynyl)azetidine-1-carboxylate;
I-25: (S)-4-(azetidin-3-ylethynyl)-7-isopropoxy-1-((5-oxopyrrolidin-2-yl)methoxy)isoquinoline-6-carboxamide;
I-26: (S)-7-isopropoxy-4-((1-methylazetidin-3-yl)ethynyl)-1-((5-oxopyrrolidin-2-yl)methoxy)isoquinoline-6-carboxamide;
I-27: (S)-7-isopropoxy-4-((1-isopropylazetidin-3-yl)ethynyl)-1-((5-oxopyrrolidin-2-yl)methoxy)isoquinoline-6-carboxamide;
I-28: methyl (S)-3-((6-carbamoyl-7-isopropoxy-1-((5-oxopyrrolidin-2-yl)methoxy)isoquinolin-4-yl)ethynyl)azetidine-1-carboxylate;
I-29: (S)-7-isopropoxy-4-((1-(methylcarbamoyl)azetidin-3-yl)ethynyl)-1-((5-oxopyrrolidin-2-yl)methoxy)isoquinoline-6-carboxamide;
I-30: (S)-4-((3,3-dimethoxycyclobutyl)ethynyl)-7-isopropoxy-1-((5-oxopyrrolidin-2-yl)methoxy)isoquinoline-6-carboxamide;
I-31: 4-(((1s,3R)-3-(dimethylamino)cyclobutyl)ethynyl)-7-isopropoxy-1-(((S)-5-oxopyrrolidin-2-yl)methoxy)isoquinoline-6-carboxamide;
I-32: (S)-4-(1-cyclopropyl-1H-pyrazol-4-yl)-7-isopropoxy-1-((5-oxopyrrolidin-2-yl)methoxy)isoquinoline-6-carboxamide;
I-33: (S)-4-(1-cyclobutyl-1H-pyrazol-4-yl)-7-isopropoxy-1-((5-oxopyrrolidin-2-yl)methoxy)isoquinoline-6-carboxamide;
I-34: (S)-4-(1-cyclopentyl-1H-pyrazol-4-yl)-7-isopropoxy-1-((5-oxopyrrolidin-2-yl)methoxy)isoquinoline-6-carboxamide;
I-35: (S)-7-isopropoxy-1-((5-oxopyrrolidin-2-yl)methoxy)-4-(1-(tetrahydro-2H-pyran-4-yl)-1H-pyrazol-4-yl)isoquinoline-6-carboxamide;
I-36: (S)-7-isopropoxy-1-((5-oxopyrrolidin-2-yl)methoxy)-4-(1-((tetrahydro-2H-pyran-4-yl)methyl)-1H-pyrazol-4-yl)isoquinoline-6-carboxamide;
I-37: (S)-7-isopropoxy-1-((5-oxopyrrolidin-2-yl)methoxy)-4-(1-(2-(tetrahydro-2H-pyran-4-yl)ethyl)-1H-pyrazol-4-yl)isoquinoline-6-carboxamide;
I-38: (S)-7-isopropoxy-4-(1-(2-morpholinoethyl)-1H-pyrazol-4-yl)-1-((5-oxopyrrolidin-2-yl)methoxy)isoquinoline-6-carboxamide;
I-39: 4-(1-((1r,4S)-4-ethoxycyclohexyl)-1H-pyrazol-4-yl)-7-isopropoxy-1-(((S)-5-oxopyrrolidin-2-yl)methoxy)isoquinoline-6-carboxamide;
I-40: 4-(1-((1 r,4S)-4-hydroxy-4-methylcyclohexyl)-1H-pyrazol-4-yl)-7-isopropoxy-1-(((S)-5-oxopyrrolidin-2-yl)methoxy)isoquinoline-6-carboxamide;
I-41: 4-(1-((1s,4R)-4-hydroxy-4-methylcyclohexyl)-1H-pyrazol-4-yl)-7-isopropoxy-1-(((S)-5-oxopyrrolidin-2-yl)methoxy)isoquinoline-6-carboxamide;
I-42: 4-(1-((1s,3R)-3-ethoxycyclobutyl)-1H-pyrazol-4-yl)-7-isopropoxy-1-(((S)-5-oxopyrrolidin-2-yl)methoxy)isoquinoline-6-carboxamide;
I-43: 4-(1-((1s,3R)-3-(dimethylamino)cyclobutyl)-1H-pyrazol-4-yl)-7-isopropoxy-1-(((S)-5-oxopyrrolidin-2-yl)methoxy)isoquinoline-6-carboxamide;
I-44: tert-butyl (S)-4-(4-(6-carbamoyl-7-isopropoxy-1-((5-oxopyrrolidin-2-yl)methoxy)isoquinolin-4-yl)-1H-pyrazol-1-yl)piperidine-1-carboxylate;
I-45: (S)-7-isopropoxy-1-((5-oxopyrrolidin-2-yl)methoxy)-4-(1-(piperidin-4-yl)-1H-pyrazol-4-yl)isoquinoline-6-carboxamide;
I-46: (S)-7-isopropoxy-4-(1-(1-methylpiperidin-4-yl)-1H-pyrazol-4-yl)-1-((5-oxopyrrolidin-2-yl)methoxy)isoquinoline-6-carboxamide;
I-47: (S)-7-isopropoxy-1-((5-oxopyrrolidin-2-yl)methoxy)-4-(1H-pyrazol-4-yl)isoquinoline-6-carboxamide;
I-48: (S)-1-((4,4-difluoro-5-oxopyrrolidin-2-yl)methoxy)-4-((1-hydroxycyclopentyl)ethynyl)-7-isopropoxyisoquinoline-6-carboxamide;
I-49: (S)-1-((4,4-difluoro-5-oxopyrrolidin-2-yl)methoxy)-7-isopropoxy-4-((tetrahydro-2H-pyran-4-yl)ethynyl)isoquinoline-6-carboxamide;
I-50: (S)-1-((4,4-difluoro-5-oxopyrrolidin-2-yl)methoxy)-7-isopropoxy-4-(3-(tetrahydro-2H-pyran-4-yl)prop-1-yn-1-yl)isoquinoline-6-carboxamide;
I-51: (S)-1-((4,4-difluoro-5-oxopyrrolidin-2-yl)methoxy)-4-((4-hydroxytetrahydro-2H-pyran-4-yl)ethynyl)-7-isopropoxyisoquinoline-6-carboxamide;
I-52: tert-butyl (S)-4-((6-carbamoyl-1-((4,4-difluoro-5-oxopyrrolidin-2-yl)methoxy)-7-isopropoxyisoquinolin-4-yl)ethynyl)-4-hydroxypiperidine-1-carboxylate;
I-53: (S)-1-((4,4-difluoro-5-oxopyrrolidin-2-yl)methoxy)-4-((4-hydroxypiperidin-4-yl)ethynyl)-7-isopropoxyisoquinoline-6-carboxamide;
I-54: tert-butyl (S)-4-((6-carbamoyl-1-((4,4-difluoro-5-oxopyrrolidin-2-yl)methoxy)-7-isopropoxyisoquinolin-4-yl)ethynyl)piperidine-1-carboxylate;
I-55: (S)-1-((4,4-difluoro-5-oxopyrrolidin-2-yl)methoxy)-7-isopropoxy-4-(piperidin-4-ylethynyl)isoquinoline-6-carboxamide;
I-56: (S)-1-((4,4-difluoro-5-oxopyrrolidin-2-yl)methoxy)-7-isopropoxy-4-((1-methylpiperidin-4-yl)ethynyl)isoquinoline-6-carboxamide;
I-57: (S)-1-((4,4-difluoro-5-oxopyrrolidin-2-yl)methoxy)-4-((4-hydroxy-1-methylpiperidin-4-yl)ethynyl)-7-isopropoxyisoquinoline-6-carboxamide;
I-58: 1-(((S)-4,4-difluoro-5-oxopyrrolidin-2-yl)methoxy)-4-(((1r,4S)-4-ethoxycyclohexyl)ethynyl)-7-isopropoxyisoquinoline-6-carboxamide;
I-59: 1-(((S)-4,4-difluoro-5-oxopyrrolidin-2-yl)methoxy)-4-(((1s,4R)-4-ethoxycyclohexyl)ethynyl)-7-isopropoxyisoquinoline-6-carboxamide;
I-60: 1-(((S)-4,4-difluoro-5-oxopyrrolidin-2-yl)methoxy)-4-(((1r,4S)-4-hydroxy-4-methylcyclohexyl)ethynyl)-7-isopropoxyisoquinoline-6-carboxamide;
I-61: 1-(((S)-4,4-difluoro-5-oxopyrrolidin-2-yl)methoxy)-4-(((1s,4R)-4-hydroxy-4-methylcyclohexyl)ethynyl)-7-isopropoxyisoquinoline-6-carboxamide;
I-62: tert-butyl (S)-3-((6-carbamoyl-1-((4,4-difluoro-5-oxopyrrolidin-2-yl)methoxy)-7-isopropoxyisoquinolin-4-yl)ethynyl)azetidine-1-carboxylate;
I-63: (S)-4-(azetidin-3-ylethynyl)-1-((4,4-difluoro-5-oxopyrrolidin-2-yl)methoxy)-7-isopropoxyisoquinoline-6-carboxamide;
I-64: (S)-1-((4,4-difluoro-5-oxopyrrolidin-2-yl)methoxy)-7-isopropoxy-4-((1-methylazetidin-3-yl)ethynyl)isoquinoline-6-carboxamide;
I-65: (S)-1-((4,4-difluoro-5-oxopyrrolidin-2-yl)methoxy)-4-((3,3-dimethoxycyclobutyl)ethynyl)-7-isopropoxyisoquinoline-6-carboxamide;
I-66: 1-(((S)-4,4-difluoro-5-oxopyrrolidin-2-yl)methoxy)-4-(((1s,3R)-3-(dimethylamino)cyclobutyl)ethynyl)-7-isopropoxyisoquinoline-6-carboxamide;
I-67: (S)-4-(1-cyclopropyl-1H-pyrazol-4-yl)-1-((4,4-difluoro-5-oxopyrrolidin-2-yl)methoxy)-7-isopropoxyisoquinoline-6-carboxamide;
I-68: (S)-4-(1-cyclobutyl-1H-pyrazol-4-yl)-1-((4,4-difluoro-5-oxopyrrolidin-2-yl)methoxy)-7-isopropoxyisoquinoline-6-carboxamide;
I-69: (S)-4-(1-cyclopentyl-1H-pyrazol-4-yl)-1-((4,4-difluoro-5-oxopyrrolidin-2-yl)methoxy)-7-isopropoxyisoquinoline-6-carboxamide;
I-70: (S)-1-((4,4-difluoro-5-oxopyrrolidin-2-yl)methoxy)-7-isopropoxy-4-(1-(tetrahydro-2H-pyran-4-yl)-1H-pyrazol-4-yl)isoquinoline-6-carboxamide;
I-71: (S)-1-((4,4-difluoro-5-oxopyrrolidin-2-yl)methoxy)-7-isopropoxy-4-(1-((tetrahydro-2H-pyran-4-yl)methyl)-1H-pyrazol-4-yl)isoquinoline-6-carboxamide;
I-72: (S)-1-((4,4-difluoro-5-oxopyrrolidin-2-yl)methoxy)-7-isopropoxy-4-(1-(2-(tetrahydro-2H-pyran-4-yl)ethyl)-1H-pyrazol-4-yl)isoquinoline-6-carboxamide;
I-73: 1-(((S)-4,4-difluoro-5-oxopyrrolidin-2-yl)methoxy)-4-(1-((1s,3R)-3-(dimethylamino)cyclobutyl)-1H-pyrazol-4-yl)-7-isopropoxyisoquinoline-6-carboxamide;
I-74: tert-butyl (S)-4-(4-(6-carbamoyl-1-((4,4-difluoro-5-oxopyrrolidin-2-yl)methoxy)-7-isopropoxyisoquinolin-4-yl)-1H-pyrazol-1-yl)piperidine-1-carboxylate;
I-75: (S)-1-((4,4-difluoro-5-oxopyrrolidin-2-yl)methoxy)-7-isopropoxy-4-(1-(piperidin-4-yl)-1H-pyrazol-4-yl)isoquinoline-6-carboxamide;
I-76: (S)-1-((4,4-difluoro-5-oxopyrrolidin-2-yl)methoxy)-7-isopropoxy-4-(1-(1-methylpiperidin-4-yl)-1H-pyrazol-4-yl)isoquinoline-6-carboxamide;
I-77: 1-(((S)-4,4-difluoro-5-oxopyrrolidin-2-yl)methoxy)-4-(1-((1r,4S)-4-ethoxycyclohexyl)-1H-pyrazol-4-yl)-7-isopropoxyisoquinoline-6-carboxamide;
I-78: 1-(((S)-4,4-difluoro-5-oxopyrrolidin-2-yl)methoxy)-4-(1-((1r,4S)-4-hydroxy-4-methylcyclohexyl)-1H-pyrazol-4-yl)-7-isopropoxyisoquinoline-6-carboxamide;
I-79: 1-(((2S,3S,4S)-3-ethyl-4-fluoro-5-oxopyrrolidin-2-yl)methoxy)-7-isopropoxy-4-((tetrahydro-2H-pyran-4-yl)ethynyl)isoquinoline-6-carboxamide;
I-80: 4-(((1r,4S)-4-ethoxycyclohexyl)ethynyl)-1-(((2S,3S,4S)-3-ethyl-4-fluoro-5-oxopyrrolidin-2-yl)methoxy)-7-isopropoxyisoquinoline-6-carboxamide;
I-81: 4-(((1s,4R)-4-ethoxycyclohexyl)ethynyl)-1-(((2S,3S,4S)-3-ethyl-4-fluoro-5-oxopyrrolidin-2-yl)methoxy)-7-isopropoxyisoquinoline-6-carboxamide;
I-82: 1-(((2S,3S,4S)-3-ethyl-4-fluoro-5-oxopyrrolidin-2-yl)methoxy)-7-methoxy-4-((tetrahydro-2H-pyran-4-yl)ethynyl)isoquinoline-6-carboxamide;
I-83: 4-(((1r,4S)-4-ethoxycyclohexyl)ethynyl)-1-(((2S,3S,4S)-3-ethyl-4-fluoro-5-oxopyrrolidin-2-yl)methoxy)-7-methoxyisoquinoline-6-carboxamide;
I-84: 4-(((1s,4R)-4-ethoxycyclohexyl)ethynyl)-1-(((2S,3S,4S)-3-ethyl-4-fluoro-5-oxopyrrolidin-2-yl)methoxy)-7-methoxyisoquinoline-6-carboxamide;
I-85: (R)-4-((1-hydroxycyclopentyl)ethynyl)-7-isopropoxy-1-(piperidin-3-yloxy)isoquinoline-6-carboxamide;
I-86: (R)-7-isopropoxy-1-(piperidin-3-yloxy)-4-((tetrahydro-2H-pyran-4-yl)ethynyl)isoquinoline-6-carboxamide;
I-87: (R)-7-isopropoxy-1-(piperidin-3-yloxy)-4-(3-(tetrahydro-2H-pyran-4-yl)prop-1-yn-1-yl)isoquinoline-6-carboxamide;
I-88: (R)-1-((1-(2-cyanoacetyl)piperidin-3-yl)oxy)-4-((1-hydroxycyclopentyl)ethynyl)-7-isopropoxyisoquinoline-6-carboxamide;
I-89: (R)-1-((1-(2-cyanoacetyl)piperidin-3-yl)oxy)-7-isopropoxy-4-((tetrahydro-2H-pyran-4-yl)ethynyl)isoquinoline-6-carboxamide;
I-90: (R)-1-((1-(2-cyanoacetyl)piperidin-3-yl)oxy)-7-isopropoxy-4-(3-(tetrahydro-2H-pyran-4-yl)prop-1-yn-1-yl)isoquinoline-6-carboxamide;
I-91: (R)-1-((1-(2-cyanoacetyl)piperidin-3-yl)oxy)-4-((4-hydroxytetrahydro-2H-pyran-4-yl)ethynyl)-7-isopropoxyisoquinoline-6-carboxamide;
I-92: tert-butyl (R)-4-((6-carbamoyl-1-((1-(2-cyanoacetyl)piperidin-3-yl)oxy)-7-isopropoxyisoquinolin-4-yl)ethynyl)-4-hydroxypiperidine-1-carboxylate;
I-93: (R)-1-((1-(2-cyanoacetyl)piperidin-3-yl)oxy)-4-((4-hydroxypiperidin-4-yl)ethynyl)-7-isopropoxyisoquinoline-6-carboxamide;
I-94: (R)-1-((1-(2-cyanoacetyl)piperidin-3-yl)oxy)-4-((4-hydroxy-1-methylpiperidin-4-yl)ethynyl)-7-isopropoxyisoquinoline-6-carboxamide;
I-95: tert-butyl (R)-4-((6-carbamoyl-1-((1-(2-cyanoacetyl)piperidin-3-yl)oxy)-7-isopropoxyisoquinolin-4-yl)ethynyl)piperidine-1-carboxylate;
I-96: (R)-1-((1-(2-cyanoacetyl)piperidin-3-yl)oxy)-7-isopropoxy-4-(piperidin-4-ylethynyl)isoquinoline-6-carboxamide;
I-97: (R)-1-((1-(2-cyanoacetyl)piperidin-3-yl)oxy)-7-isopropoxy-4-((1-methylpiperidin-4-yl)ethynyl)isoquinoline-6-carboxamide;
I-98: 1-(((R)-1-(2-cyanoacetyl)piperidin-3-yl)oxy)-4-(((1r,4R)-4-ethoxycyclohexyl)ethynyl)-7-isopropoxyisoquinoline-6-carboxamide;
I-99: 1-(((R)-1-(2-cyanoacetyl)piperidin-3-yl)oxy)-4-(((1s,4S)-4-ethoxycyclohexyl)ethynyl)-7-isopropoxyisoquinoline-6-carboxamide;
I-100: 1-(((R)-1-(2-cyanoacetyl)piperidin-3-yl)oxy)-4-(((1r,4R)-4-hydroxy-4-methylcyclohexyl)ethynyl)-7-isopropoxyisoquinoline-6-carboxamide;
I-101: 1-(((R)-1-(2-cyanoacetyl)piperidin-3-yl)oxy)-4-(((1s,4S)-4-hydroxy-4-methylcyclohexyl)ethynyl)-7-isopropoxyisoquinoline-6-carboxamide;
I-102: tert-butyl (R)-3-((6-carbamoyl-1-((1-(2-cyanoacetyl)piperidin-3-yl)oxy)-7-isopropoxyisoquinolin-4-yl)ethynyl)azetidine-1-carboxylate;
I-103: (R)-4-(azetidin-3-ylethynyl)-1-((1-(2-cyanoacetyl)piperidin-3-yl)oxy)-7-isopropoxyisoquinoline-6-carboxamide;
I-104: (R)-1-((1-(2-cyanoacetyl)piperidin-3-yl)oxy)-7-isopropoxy-4-((1-methylazetidin-3-yl)ethynyl)isoquinoline-6-carboxamide;
I-105: (R)-1-((1-(2-cyanoacetyl)piperidin-3-yl)oxy)-4-((3,3-dimethoxycyclobutyl)ethynyl)-7-isopropoxyisoquinoline-6-carboxamide;
I-106: 1-(((R)-1-(2-cyanoacetyl)piperidin-3-yl)oxy)-4-(((1s,3S)-3-(dimethylamino)cyclobutyl)ethynyl)-7-isopropoxyisoquinoline-6-carboxamide;
I-107: tert-butyl (R)-3-((6-carbamoyl-4-(1-cyclopropyl-1H-pyrazol-4-yl)-7-isopropoxyisoquinolin-1-yl)oxy)piperidine-1-carboxylate;
I-108: (R)-4-(1-cyclopropyl-1H-pyrazol-4-yl)-7-isopropoxy-1-(piperidin-3-yloxy)isoquinoline-6-carboxamide;
I-109: (R)-4-(1-cyclobutyl-1H-pyrazol-4-yl)-7-isopropoxy-1-(piperidin-3-yloxy)isoquinoline-6-carboxamide;
I-110: (R)-4-(1-cyclopentyl-1H-pyrazol-4-yl)-7-isopropoxy-1-(piperidin-3-yloxy)isoquinoline-6-carboxamide;
I-111: (R)-7-isopropoxy-1-(piperidin-3-yloxy)-4-(1-(tetrahydro-2H-pyran-4-yl)-1H-pyrazol-4-yl)isoquinoline-6-carboxamide;
I-112: (R)-7-isopropoxy-1-(piperidin-3-yloxy)-4-(1-((tetrahydro-2H-pyran-4-yl)methyl)-1H-pyrazol-4-yl)isoquinoline-6-carboxamide;
I-113: (R)-7-isopropoxy-1-(piperidin-3-yloxy)-4-(1-(2-(tetrahydro-2H-pyran-4-yl)ethyl)-1H-pyrazol-4-yl)isoquinoline-6-carboxamide;
I-114: (R)-1-((1-(2-cyanoacetyl)piperidin-3-yl)oxy)-4-(1-cyclopropyl-1H-pyrazol-4-yl)-7-isopropoxyisoquinoline-6-carboxamide;
I-115: (R)-1-((1-(2-cyanoacetyl)piperidin-3-yl)oxy)-4-(1-cyclobutyl-1H-pyrazol-4-yl)-7-isopropoxyisoquinoline-6-carboxamide;
I-116: (R)-1-((1-(2-cyanoacetyl)piperidin-3-yl)oxy)-4-(1-cyclopentyl-1H-pyrazol-4-yl)-7-isopropoxyisoquinoline-6-carboxamide;
I-117: (R)-1-((1-(2-cyanoacetyl)piperidin-3-yl)oxy)-7-isopropoxy-4-(1-(tetrahydro-2H-pyran-4-yl)-1H-pyrazol-4-yl)isoquinoline-6-carboxamide;
I-118: (R)-1-((1-(2-cyanoacetyl)piperidin-3-yl)oxy)-7-isopropoxy-4-(1-((tetrahydro-2H-pyran-4-yl)methyl)-1H-pyrazol-4-yl)isoquinoline-6-carboxamide;
I-119: (R)-1-((1-(2-cyanoacetyl)piperidin-3-yl)oxy)-7-isopropoxy-4-(1-(2-(tetrahydro-2H-pyran-4-yl)ethyl)-1H-pyrazol-4-yl)isoquinoline-6-carboxamide;
I-120: 1-(((R)-1-(2-cyanoacetyl)piperidin-3-yl)oxy)-4-(1-((1s,3S)-3-(dimethylamino)cyclobutyl)-1H-pyrazol-4-yl)-7-isopropoxyisoquinoline-6-carboxamide;
I-121: tert-butyl (R)-4-(4-(6-carbamoyl-1-((1-(2-cyanoacetyl)piperidin-3-yl)oxy)-7-isopropoxyisoquinolin-4-yl)-1H-pyrazol-1-yl)piperidine-1-carboxylate;
I-122: (R)-1-((1-(2-cyanoacetyl)piperidin-3-yl)oxy)-7-isopropoxy-4-(1-(piperidin-4-yl)-1H-pyrazol-4-yl)isoquinoline-6-carboxamide;
I-123: (R)-1-((1-(2-cyanoacetyl)piperidin-3-yl)oxy)-7-isopropoxy-4-(1-(1-methylpiperidin-4-yl)-1H-pyrazol-4-yl)isoquinoline-6-carboxamide; or
I-124: 1-(((R)-1-(2-cyanoacetyl)piperidin-3-yl)oxy)-4-(1-((1r,4R)-4-ethoxycyclohexyl)-1H-pyrazol-4-yl)-7-isopropoxyisoquinoline-6-carboxamide.

17. A pharmaceutical composition comprising a compound according to claim 1, and a pharmaceutically acceptable excipient.

18. A method for inhibiting an IRAK enzyme, comprising contacting the enzyme with an effective amount of a compound of claim 1.

19. The method of claim 18 wherein contacting comprises administering the compound to a subject.

20. A method for treating a subject for a disease or condition wherein an IRAK inhibitor is indicated, comprising administering to the subject an effective amount of a compound of claim 1.

21. The method of claim 20, where the disease or condition comprises an auto-immune disease, inflammatory disorder, cardiovascular disease, neurodegenerative disorder, allergic disorder, multi-organ failure, kidney disease, platelet aggregation, a hyperproliferative disorder, transplantation, sperm motility, erythrocyte deficiency, graft rejection, lung injury, respiratory disease, ischemic condition, bacterial infection, viral infection, immune regulatory disorder, sickle cell disease, chemical- or radiation-induced lung injury, hemorrhagic fever, or a combination thereof.

22. The method of claim 20, wherein the disease or condition is rheumatoid arthritis, systemic lupus erythematosus, Hashimoto's thyroiditis, multiple sclerosis, systemic sclerosis, myasthenia gravis, type I diabetes, uveitis, posterior uveitis, allergic encephalomyelitis, glomerulonephritis, postinfectious autoimmune diseases including rheumatic fever and post-infectious glomerulonephritis, inflammatory and hyperproliferative skin diseases, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, alopecia areata, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, coeliac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, chronic lymphocytic leukemia, arteriosclerosis, atherosclerosis, aortitis syndrome, polyarteritis nodosa, myocardosis, scleroderma, Wegener's granuloma, Sjogren's syndrome, adiposis, eosinophilic fascitis, lesions of gingiva, periodontium, alveolar bone, substantia ossea dentis, glomerulonephritis, male pattern alopecia or alopecia senilis by preventing epilation or providing hair germination and/or promoting hair generation and hair growth, muscular dystrophy, pyoderma and Sezary's syndrome, Addison's disease, ischemia-reperfusion injury of organs which occurs upon preservation, transplantation or ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA ballous dermatitis and cement dermatitis, gingivitis, periodontitis, sepsis, pancreatitis, diseases caused by environmental pollution, aging, carcinogenesis, metastasis of carcinoma and hypobaropathy, disease caused by histamine or leukotriene-C4 release, Behcet's disease, autoimmune hepatitis, primary biliary cirrhosis, sclerosing cholangitis, partial liver resection, acute liver necrosis, necrosis caused by toxin, viral hepatitis, shock, or anoxia, B-virus hepatitis, non-A/non-B hepatitis, cirrhosis, alcoholic cirrhosis, hepatic failure, fulminant hepatic failure, late-onset hepatic failure, “acute-on-chronic” liver failure, augmentation of chemotherapeutic effect, cytomegalovirus infection, HCMV infection, AIDS, cancer, senile dementia, Parkinson's disease, trauma, CRS, ARDS, AKI, or chronic bacterial infection.

23. The method of claim 20, wherein the disease or condition comprises a lymphoid neoplasm selected from myeloproliferative neoplasms (MPN) excluding polycythemia vera, myeloid/lymphoid neoplasms with PDGFRA rearrangement, myeloid/lymphoid neoplasms with PDGFRB rearrangement, myeloid/lymphoid neoplasms with FGFR1 rearrangement, myeloid/lymphoid neoplasms with PCM1-JAK2, myelodysplastic/myeloproliferative neoplasms (MDS/MPN), myeloid sarcoma, myeloid proliferations related to Down syndrome, blastic plasmacytoid dendritic cell neoplasm, B-lymphoblastic leukemia/lymphoma; and/or T-lymphoblastic leukemia/lymphoma.

24. The method of claim 23, wherein the lymphoid neoplasm is a myeloproliferative neoplasm selected from chronic myeloid leukemia (CML), chronic neutrophilic leukemia (CNL), primary myelofibrosis (PMF), essential thrombocythemia, chronic eosinophilic leukemia, or a combination thereof.

25. The method of claim 24, wherein the lymphoid neoplasm is chronic myeloid leukemia.

26. The method of claim 23, wherein the lymphoid neoplasm is a myelodysplastic/myeloproliferative neoplasm selected from chronic myelomonocytic leukemia, atypical chronic myeloid leukemia (aCML), juvenile myelomonocytic leukemia (JMML), MDS/MPN with ring sideroblasts and thrombocytosis (MDS/MPN-RS-T), or a combination thereof.

27. The method of claim 23, wherein the method further comprises identifying the subject having from the lymphoid neoplasm.

28. The method of claim 27, wherein the lymphoid neoplasm is chronic myelomonocytic leukemia and identifying the subject comprises identifying a subject having a persistent peripheral blood monocytosis of ≥1×10⁹/L and monocytes accounting for ≥10% of the white blood cell (WBC) differential count, and rearrangements in the PDGFRA, PDGFRB or FGFR1 genes and the PCM1-JAK2 fusion gene are not observed.
